(19) United States
(12) Patent Application Publication
(10) Pub. No.: US 2026/0232840 A1
(43) Pub. Date: Aug. 13, 2026

(54) ANTIBODIES AND UBIQUITIN LIGASE FUSION PROTEINS FOR MISFOLDED SUPEROXIDE DISMUTASE-1 (SOD1)

(71) Applicants: THE UNIVERSITY OF BRITISH COLUMBIA, Vancouver (CA); ProMIS Neurosciences Inc., Toronto (CA)

(72) Inventors: Neil R. CASHMAN, Vancouver (CA); Justin J. YERBURY, Toronto (CA); Christen G. CHISHOLM, Gerringong (AU)

(21) Appl. No.: 19/157,620

(22) PCT Filed: Feb. 19, 2024
(86) PCT No.: PCT/CA2024/050200

§ 371 Date: Aug. 18, 2025

Related U.S. Application Data
(60) Provisional application No. 63/446,485, filed on Feb. 17, 2023.

(51) Int. Cl. A61K 48/00; A61K 38/00; A61P 25/28; C07K 16/40; C12N 9/10; C12N 15/62; C12N 15/86

(52) U.S. Cl. CPC A61K 48/005; A61P 25/28; C07K 16/40; C12N 9/104; C12N 15/62; C12N 15/86; A61K 38/00; C07K 2317/40; C07K 2317/565; C07K 2317/622; C12N 2750/14143; C12Y 203/02

(57) ABSTRACT

Antibody or binding fragment thereof, fusion protein, or pharmaceutical composition directed against misfolded SOD1 epitope, or nucleic acid encoding the antibody or binding fragment thereof, fusion protein, or pharmaceutical composition, are described. Also provided are methods for using and manufacturing such antibody or binding fragment thereof, fusion protein, nucleic acid, or pharmaceutical composition, as well as methods of their use for treating a disorder in a human subject in need thereof.

### RELATED APPLICATION

This disclosure claims benefit and priority of U.S. Provisional Patent Application Ser. No. 63/446,485 filed Feb. 17, 2023, incorporated herein by reference in its entirety.

### INCORPORATION OF SEQUENCE LISTING

A computer readable form of the Sequence Listing “P58515PC01_ST26” (272,118 bytes), was created on Feb. 19, 2024, is herein incorporated by reference.

### FIELD

The disclosure relates to antibodies and ubiquitin ligase fusion proteins thereof for misfolded superoxide dismutase-1 (SOD1), for treating conditions, diseases and disorders mediated by misfolded SOD1, including amyotrophic lateral sclerosis, Alzheimer's disease and Parkinson's disease.

### BACKGROUND

Proteins can fold into complex and close-packed structures. Folding is not only crucial for biological activity but failure of proteins to fold properly or remain folded can give rise to disease (reviewed in 48). Misfolding can in some cases cause protein aggregation which can further give rise to discrete deposits extracellularly (e.g., plaques) or intracellularly (e.g., inclusions in the cytosol or nucleus).

Neurodegenerative diseases such as Alzheimer's disease (AD), Parkinson's disease/Lewy body dementia (PD/LBD), Huntington's disease (HD), amyotrophic lateral sclerosis (ALS) and prion diseases are characterized by neural deposits of misfolded aggregated protein (reviewed in 49). These diseases pose major challenges to our aging population and health care system.

Sporadic AD, ALS, and PD/LBD are all associated with neural accumulation of pathological multimers of misfolded polypeptides, which could potentially be fibrils, protofilaments, and amorphous aggregates, including the amyloid-beta (Abeta) fragment of the amyloid precursor protein (APP) in AD; superoxide dismutase-1 (SOD1) in ALS, AD, and PD, and alpha-synuclein in PD and LBD. Additionally, familial amyloidotic polyneuropathy (FAP) results from the aggregation of transthyretin to form amyloid deposits. As with prion diseases, mutations in genes encoding these polypeptides are associated with autosomal dominant familial forms of ALS, AD, and PD.

Oxidative stress has been implicated in ALS, PD and AD. Reactive oxygen and nitrogen species (ROS and RNS respectively) generated in these environments may participate in cell injury including the abnormal oxidation of proteins or lipids. Cytoskeletal debris accumulations and selective neuronal death that occur in these diseases can also be attributed to oxidative stress and the accumulated insoluble protein. SOD1 is known to have antioxidant roles and alterations in its activity may contribute to a neurodegenerative disease state. SOD1 is a major target of oxidative damage in AD and PD brains. The total level of SOD1 is increased in both AD and PD and that SOD1 forms proteinaceous aggregates that are associated with amyloid senile plaques and neurofibrillary tangles in AD brains. It has been suggested that AD, PD and ALS may share a common pathogenic mechanism (Choi et al, 2005).

ALS is a fatal neuromuscular disease characterized by selective loss of motor neurons causing muscle atrophy. It is the most common motor neuron disease in adults and the third most common neurodegenerative disease after Alzheimer's disease and Parkinson's disease. The number of people worldwide who develop ALS annually is estimated to be 1.9 people per 100,000 per year, while the number of people who have ALS at any given time is estimated to be about 4.5 people per 100,000. ALS often begins with muscle twitching and weakness in a limb, or slurred speech, and eventually the disease affects control of the muscles needed to move, speak, eat and breathe. The average survival from onset to death is two to four years, and about 10% survive longer than 10 years. Death is usually due to respiratory failure.

Familial (inherited) ALS is usually said to account for 10% of all cases of ALS, though estimates range from 5% to 20%. About 20% of familial ALS is associated with mutations in the gene encoding superoxide dismutase 1 (SOD1), an intracellular free radical defense enzyme (see Table 1 and Mathis, S., et al. (2019), herein incorporated by reference). Intracellular aggregations of misfolded SOD1 that inhibit protein degradation have been observed in familial ALS, and also in the more common non-familial (sporadic) ALS, suggesting that SOD1 aggregation may underlie all ALS.

Misfolded SOD1 is exported from the cell by both secretory and constitutive mechanisms. Extracellular misfolded SOD1 is highly toxic for motor neurons through activation of killing pathways by local immune cells, and may also participate in the cell-to-cell propagation of disease throughout the nervous system by a prion-like templated misfolding process. The implication that extracellular misfolded SOD1 plays a role in ALS pathogenesis provides an opportunity for the antibody treatment of neurodegenerative diseases, as this compartment is accessible to antibody neutralization. Nonetheless, treatment of human subjects with antibodies targeted to accessible extracellular epitopes on ubiquitous proteins may lead to deleterious autoimmune effects such as those seen with Abeta in Alzheimer disease. There remains a need in the art for compositions and methods for treatment of misfolded SOD1-related diseases, such as ALS, AD and PD.

### SUMMARY

Disclosed herein an isolated antibody or binding fragment thereof, a fusion protein, or a nucleic acid, as well as a pharmaceutical composition comprising the same that targets misfolded SOD1 as well as methods of making and using thereof.

Accordingly, herein provided in an aspect is an isolated antibody or binding fragment thereof that binds to misfolded superoxide dismutase 1 (SOD1) epitope having an amino acid sequence shown in SEQ ID NO: 144, the antibody or binding fragment thereof comprises:
(i) a heavy chain comprising a heavy chain variable region, wherein the heavy chain variable region comprises CDR1, CDR2, and CDR3 having the amino acid sequence of SEQ ID NO: 1, SEQ ID NO: 2, and SEQ ID NO: 3, and a light chain comprising a light chain variable region, wherein the light chain variable region comprises CDR1, CDR2, and CDR3 having the amino acid sequence of SEQ ID NO: 4, SEQ ID NO: 5, and SEQ ID NO: 6;
(ii) a heavy chain comprising a heavy chain variable region, wherein the heavy chain variable region comprises CDR1, CDR2, and CDR3 having the amino acid sequence of SEQ ID NO: 7, SEQ ID NO: 8, and SEQ ID NO: 9, and a light chain comprising a light chain variable region, wherein the light chain variable region comprises CDR1, CDR2 and CDR3 having the amino acid sequence of SEQ ID NO: 10, SEQ ID NO: 11 and SEQ ID NO: 12;
(iii) a heavy chain comprising a heavy chain variable region, wherein the heavy chain variable region comprises CDR1, CDR2 and CDR3 having the amino acid sequence of SEQ ID NO: 14, SEQ ID NO: 15, and SEQ ID NO: 16, and a light chain comprising a light chain variable region, wherein the light chain variable region comprises CDR1, CDR2 and CDR3 having the amino acid sequence of SEQ ID NO: 10, SEQ ID NO: 11, and SEQ ID NO: 12;
(iv) a heavy chain comprising a heavy chain variable region, wherein the heavy chain variable region comprises CDR1, CDR2, and CDR3 having the amino acid sequence of SEQ ID NO: 7, SEQ ID NO: 8, and SEQ ID NO: 9, and a light chain comprising a light chain variable region, wherein the light chain variable region comprises CDR1, CDR2 and CDR3 having the amino acid sequence of SEQ ID NO: 10, SEQ ID NO: 11 and SEQ ID NO: 13;
(v) a heavy chain comprising a heavy chain variable region, wherein the heavy chain variable region comprises CDR1, CDR2 and CDR3 having the amino acid sequence of SEQ ID NO: 14, SEQ ID NO: 15, and SEQ ID NO: 16, and a light chain comprising a light chain variable region, wherein the light chain variable region comprises CDR1, CDR2 and CDR3 having the amino acid sequence of SEQ ID NO: 10, SEQ ID NO: 11, and SEQ ID NO: 13;
(vi) a heavy chain comprising a heavy chain variable region, wherein the heavy chain variable region comprises CDR1, CDR2 and CDR3 having the amino acid sequence of SEQ ID NO: 53, SEQ ID NO: 54, and SEQ ID NO: 55, and a light chain comprising a light chain variable region, wherein the light chain variable region comprises CDR1, CDR2 and CDR3 having the amino acid sequence of SEQ ID NO: 56, SEQ ID NO: 57, and SEQ ID NO: 58;
(vii) a heavy chain comprising a heavy chain variable region, wherein the heavy chain variable region comprises CDR1, CDR2 and CDR3 having the amino acid sequence of SEQ ID NO: 23, SEQ ID NO: 24, and SEQ ID NO: 25, and a light chain comprising a light chain variable region, wherein the light chain variable region comprises CDR1, CDR2 and CDR3 having the amino acid sequence of SEQ ID NO: 26, SEQ ID NO: 27, and SEQ ID NO: 28; or
(viii) a heavy chain comprising a heavy chain variable region, wherein the heavy chain variable region comprises CDR1, CDR2 and CDR3 having the amino acid sequence of SEQ ID NO: 29, SEQ ID NO: 30, and SEQ ID NO: 31, and a light chain comprising a light chain variable region, wherein the light chain variable region comprises CDR1, CDR2 and CDR3 having the amino acid sequence of SEQ ID NO: 32, SEQ ID NO: 33, and SEQ ID NO: 34.

In some embodiments, the antibody or binding fragment thereof comprises:
(i) the heavy chain comprises the heavy chain variable region comprising an amino acid sequence having at least 85%, 90%, 95%, 97%, 98%, 99%, 99.5%, 99.5% or 100% identity to the amino acid sequence of SEQ ID NO: 65, and the light chain comprises the light chain variable region comprising an amino acid sequence having at least 85%, 90%, 95%, 97%, 98%, 99%, 99.5%, 99.5% or 100% identity to the amino acid sequence of SEQ ID NO: 70;
(ii) the heavy chain comprises the heavy chain variable region comprising an amino acid sequence having at least 85%, 90%, 95%, 97%, 98%, 99%, 99.5%, 99.5% or 100% identity to the amino acid sequence of SEQ ID NO: 68, and the light chain comprises the light chain variable region comprising an amino acid sequence having at least 85%, 90%, 95%, 97%, 98%, 99%, 99.5%, 99.5% or 100% identity to the amino acid sequence of SEQ ID NO: 70;
(iii) the heavy chain comprises the heavy chain variable region comprising an amino acid sequence having at least 85%, 90%, 95%, 97%, 98%, 99%, 99.5%, 99.5% or 100% identity to the amino acid sequence of SEQ ID NO: 72, and the light chain comprises the light chain variable region comprising an amino acid sequence having at least 85%, 90%, 95%, 97%, 98%, 99%, 99.5%, 99.5% or 100% identity to the amino acid sequence of SEQ ID NO: 76;
(iv) the heavy chain comprises the heavy chain variable region comprising an amino acid sequence having at least 85%, 90%, 95%, 97%, 98%, 99%, 99.5%, 99.5% or 100% identity to the amino acid sequence of SEQ ID NO: 74, and the light chain comprises the light chain variable region comprising an amino acid sequence having at least 85%, 90%, 95%, 97%, 98%, 99%, 99.5%, 99.5% or 100% identity to the amino acid sequence of SEQ ID NO: 76;
(v) the heavy chain comprises the heavy chain variable region comprising an amino acid sequence having at least 85%, 90%, 95%, 97%, 98%, 99%, 99.5%, 99.5% or 100% identity to the amino acid sequence of SEQ ID NO: 78, and the light chain comprises the light chain variable region comprising an amino acid sequence having at least 85%, 90%, 95%, 97%, 98%, 99%, 99.5%, 99.5% or 100% identity to the amino acid sequence of SEQ ID NO: 82;
(vi) the heavy chain comprises the heavy chain variable region comprising an amino acid sequence having at least 85%, 90%, 95%, 97%, 98%, 99%, 99.5%, 99.5% or 100% identity to the amino acid sequence of SEQ ID NO: 80, and the light chain comprises the light chain variable region comprising an amino acid sequence having at least 85%, 90%, 95%, 97%, 98%, 99%, 99.5%, 99.5% or 100% identity to the amino acid sequence of SEQ ID NO: 82;
(vii) the heavy chain comprises the heavy chain variable region comprising an amino acid sequence having at least 85%, 90%, 95%, 97%, 98%, 99%, 99.5%, 99.5% or 100% identity to the amino acid sequence of SEQ ID NO: 84, and the light chain comprises the light chain variable region comprising an amino acid sequence having at least 85%, 90%, 95%, 97%, 98%, 99%, 99.5%, 99.5% or 100% identity to the amino acid sequence of SEQ ID NO: 86; or
(viii) the heavy chain comprises the heavy chain variable region comprising an amino acid sequence having at least 85%, 90%, 95%, 97%, 98%, 99%, 99.5%, 99.5% or 100% identity to the amino acid sequence of SEQ ID NO: 88, and the light chain comprises the light chain variable region comprising an amino acid sequence having at least 85%, 90%, 95%, 97%, 98%, 99%, 99.5%, 99.5% or 100% identity to the amino acid sequence of SEQ ID NO: 91.

In some embodiments, the percentage identity is outside of the CDRs described herein and the antibody or antigen-binding fragment thereof maintains specificity in binding to mutant SOD1.

In some embodiments, the binding fragment is a Fab, Fab′, F(ab′)2, scFv, dsFv, ds-scFv, dimer, minibody, diabody, or bispecific antibody binding fragment. In some embodiments, the binding fragment is a scFv. In some embodiments, the antibody or binding fragment thereof comprises one or more amino acids selected from the group consisting of D-amino acids, modified amino acids, amino acid analogs or combinations thereof. In some embodiments, the modified amino acids comprise a modification selected from the group consisting of methylation, amidation, acetylation, and/or substitution with other chemical groups. In some embodiments, the antibody or binding fragment thereof is modified by pegylation, acetylation, glycosylation, biotinylation, or prenylation.

Also provided in another aspect is a fusion protein comprising an antibody or binding fragment thereof described herein and an E3 ligase or active fragment thereof. In some embodiments, the E3 ligase is CHIP, UBE4A, NEDD4L, UBR5, RNF4, UBOX5, BTrCP, or Parkin, or an active fragment thereof.

Also provided in a further aspect is a nucleic acid encoding an antibody or binding fragment thereof described herein, or the fusion protein described herein.

Also provided in another aspect is a vector comprising a nucleic acid described herein.

Also provided in another aspect is a composition, optionally a pharmaceutical composition comprising an antibody or binding fragment thereof described herein, a fusion protein described herein, a nucleic acid descried herein, or a vector described herein, and optionally at least one carrier such as pharmaceutical carrier.

In another aspect, provided herein is a method for treating a medical condition, disease, or disorder mediated by a misfolded form of SOD1 in a subject in need thereof, the method comprises administering to the subject an antibody or binding fragment thereof described herein, a fusion protein described herein, a nucleic acid described herein, a vector described herein, or a pharmaceutical composition described herein. In some embodiments, the medical condition, disease or disorder is a neurodegenerative condition, disease or disorder. In some embodiments, the neurodegenerative condition, disease or disorder is amyotrophic lateral sclerosis (ALS), Alzheimer's disease, Parkinson's disease, or frontotemporal dementia. In some embodiments, the ALS is sporadic ALS. In some embodiments, the ALS is familial ALS. In some embodiments, the neurodegenerative condition, disease or disorder is Alzheimer's disease. In some embodiments, the neurodegenerative condition, disease or disorder is Parkinson's disease. In some embodiments, the neurodegenerative condition, disease or disorder is frontotemporal dementia. In some embodiments, the misfolded form of SOD1 comprises SOD1 monomer, a dimer comprising misfolded SOD1 monomers, or an aggregate comprising SOD1 monomers and/or dimers, or toxic trimers. In some embodiments, the SOD1 monomer comprises a mutant SOD1 monomer. In some embodiments, the mutant SOD1 monomer comprise one or more mutations selected from the group consisting of A4V, G93A, G85R, D90A, G127X, V148G, H46R, G37R, C6G, and E100G, and wherein X is truncation mutation.

Also provided in another aspect is a fusion protein that specifically binds to mutant SOD1, comprising a heavy chain variable region comprising CDR1, CDR2, and CDR3 having the amino acid sequence of SEQ ID NO: 1, SEQ ID NO: 2, and SEQ ID NO: 3, a first linker, a light chain variable region comprising CDR1, CDR2, and CDR3 having the amino acid sequence of SEQ ID NO: 4, SEQ ID NO: 5, and SEQ ID NO: 6, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 186, or a truncated E3 ligase comprising an amino acid sequence having at least 85%, 90%, 95%, 97%, 98%, 99%, 99.5%, 99.5% or 100% identity to the amino acid sequence of SEQ ID NO: 223.

In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 68, a first linker, a light chain variable region comprising an amino acid sequence having at least 85%, 90%, 95%, 97%, 98%, 99%, 99.5%, 99.5% or 100% identity to the amino acid sequence of SEQ ID NO: 70, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 186, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein is formulated for viral delivery into a subject. In some embodiments, the fusion protein is expressed and delivered by an AAV vector.

Also provided in another aspect, is a method for treating neurodegenerative condition, disease, or disorder mediated by a misfolded form of SOD1 in a subject in need thereof, the method comprises administering to a subject the fusion protein described herein. In some embodiments, the neurodegenerative condition, disease or disorder is amyotrophic lateral sclerosis (ALS), Alzheimer's disease, Parkinson's disease, or frontotemporal dementia. In some embodiments, the ALS is sporadic ALS. In some embodiments, the ALS is familial ALS. In some embodiments, the SOD1 comprises a mutant SOD1. In some embodiments, the mutant SOD1 comprises one or more mutations selected from the group consisting of A4V, G93A, G85R, D90A, G127X, V148G, H46R, G37R, C6G, and E100G, and wherein X is truncation mutation. In some embodiments, the mutant SOD1 comprises G93A.

These and other objects and features of the disclosure will become more fully apparent when the following detailed description of the disclosure is read in conjunction with the accompanying drawings.

### BRIEF DESCRIPTION OF THE DRAWINGS

Embodiments of the disclosure will be described in relation to the drawings in which:

FIG. 1A shows the results of the epitope mapping experiment with hybridoma clone 3H1.

FIG. 1B shows the results of the epitope mapping experiment with hybridoma clone 8D1.

FIG. 2 is a representation of the location of the epitope of the anti-SOD1 antibodies.

FIG. 3 A shows the design of misfold specific Ubiquitin Ligase fusion proteins (mUbL).

FIG. 3B is a graph showing gene expression data from human postmortem anterior horn in control and ALS donors.

FIG. 4A are images showing HEK293 cells transfected with mUbL and SOD1-A4V-GFP, and stained for DAPI and mUbL.

FIG. 4B is an immunoblot showing mUbLs expression in HEK293 transfected with mUbLs and a graph showing signal intensity representing protein expression levels.

FIG. 4C is an immunoblot showing that mUbLs do not degrade endogenous SOD1.

FIG. 4D shows mUbL 2 interacts with misfolded SOD1.

FIG. 5A are graphs of results showing that all mUbLs were able to reduce the amount of SOD1-A4V-GFP in HEK293, Neuro2A, and SHSY5Y cells.

FIG. 5B are graphs of results showing that mUbL 2 was consistently effective in reducing fluorescence in HEK293, Neuro2A, and SHSY5Y cells.

FIG. 5C are graphs of results showing that mUbL 2 was the most effective at reducing the number of insoluble fluorescent aggregates present in transfected HEK293, Neuro2A, and SHSY5Y cells by maintaining SOD1 at levels where it could diffuse out through saponin induced pores in the cell membrane. FIG. 5C shows mUbLs reduce the number of insoluble SOD1^{G93A}-EGFP aggregates in HEK293 cells.

FIG. 6A are graphs of results showing that mUbLs can reduce misfolded SOD1 levels across a range of SOD1 mutations.

FIG. 6B are graphs of results showing that mUbL action is prevented when the proteasome is inhibited by treatment with MG132.

FIG. 6C is a graph of results showing mUbLs effectiveness at reducing the intensity of SOD1^{A4V}-EGFP fluorescence when expression of mUbLs is normalized.

FIG. 7A shows the design of E3 ligase panel. The binding domain was removed and the scFv from mUbL 2 was fused to the truncated ligase.

FIG. 7B shows ligases in the mUbL panel have varying effectiveness at reducing the intensity of SOD1^{G93A}-EGFP fluorescence when expression of mUbL is controlled.

FIG. 8A are graphs of results showing that E3 ligases were able to reduce SOD1-A4V and G93A total fluorescence in HEK293 and also SOD1-A4V in SHSY5Y cells.

FIG. 8B are graphs of results showing that E3 ligases were effective at reducing the number of insoluble fluorescent aggregates in transfected cells after saponin treatment.

FIG. 8C are graphs of results showing that E3 ligases were effective at reducing the level of soluble fluorescence in the media after saponin treatment.

FIG. 9A shows expression of mUbLs in neuronal cells in brain tissue.

FIG. 9B shows expression of mUbLs in brain and spinal cord but not liver in WT/mUbL mice.

FIG. 10 shows mUbL transgene was effective in attenuating and delaying weight loss in the early symptomatic phase of ALS in male but not female SOD1^{G93A} mice.

FIG. 11 shows mUbL transgene was effective in delaying disease progression in SOD1^{G93A} mice.

FIG. 12 shows the mUbL transgene was effective attenuating clinical phenotype at end stage of the disease.

FIG. 13 shows mUbL affecting phenotype of SOD1^{G93A} at endpoint. Survival was analysed using log rank tests.

### DETAILED DESCRIPTION

### I. Definitions

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. Finally, terms of degree such as “substantially”, “about” and “approximately” as used herein mean a reasonable amount of deviation of the modified term such that the end result is not significantly changed. These terms of degree should be construed as including a deviation of at least 5% of the modified term if this deviation would not negate the meaning of the word it modifies.

Where a term is provided in the singular, the inventors also contemplate aspects of the disclosure described by the plural of that term. As used in this specification and in the appended claims, the singular forms “a”, “an” and “the” include plural references unless the context clearly dictates otherwise, e.g., “an antibody” includes a plurality of antibodies. Thus, for example, a reference to “a method” includes one or more methods, and/or steps of the type described herein and/or which will become apparent to those persons skilled in the art upon reading this disclosure.

The term “administer”, “administering” or “administered” means the act of giving an agent or therapeutic treatment to a physiological system (e.g., a subject or in vivo, in vitro, or ex vivo cells, tissues, and organs).

The term “affinity”, as used herein, refers to the strength of the sum total of noncovalent interactions between a single binding site of a molecule and its binding partner. Unless indicated otherwise, as used herein, “binding affinity” refers to intrinsic binding affinity which reflects a 1:1 interaction between members of a binding pair. The affinity of a molecule X for its partner Y can generally be represented by the equilibrium dissociation constant (K_{D}). Affinity can be measured by common methods known in the art.

The term “amino acid” refers to naturally occurring amino acids, as well as non-naturally occurring or non-standard amino acids such as amino acid analogs, synthetic amino acids, and amino acid mimetics. These amino acids may be in the L- or D-(isomeric) configuration, or may include both dextrorotary forms. Amino acids that have been incorporated into antibodies are termed “residues”. Amino acids may be referred to herein by either the commonly known three letter symbols or by the one-letter symbols recommended by the IUPAC-IUB Biochemical Nomenclature Commission.

The term “antibody” as used herein is intended to include monoclonal antibodies, polyclonal antibodies, chimeric antibodies, and humanized antibodies. The antibody may be from recombinant sources and/or produced in transgenic animals. The term “antibody binding fragment” or “antibody fragment” as used herein is intended to include without limitations Fv (a molecule comprising the VL and VH), single-chain variable fragment (scFv; a molecule comprising the VL and VH connected by a peptide linker), Fab, Fab′, F(ab′)2, dsFv, ds-scFv, single domain antibodies (sdAB; molecules comprising a single variable domain having 3 or less CDRs, such as V_{H}H, V_{H}, V_{L}, and IgNAR antigen binding variable domain (VNAR)), and multivalent presentations of these. Also included are dimers, minibodies, diabodies, and multimers thereof, bi-specific and multi-specific antibody fragments, and domain antibodies. Antibodies can be fragmented using conventional techniques. For example, F(ab′)2 fragments can be generated by treating the antibody with pepsin. The resulting F(ab′)2 fragment can be treated to reduce disulfide bridges to produce Fab′ fragments. Papain digestion can lead to the formation of Fab fragments. Fab, Fab′ and F(ab′)2, scFv, dsFv, ds-scFv, sdAB, dimers, minibodies, diabodies, bispecific antibody fragments and other fragments can also be synthesized by recombinant techniques.

In the present disclosure, scFv antibodies that bind to misfolded SOD1 were recombinantly generated based on the heavy chain variable region (VH) and light chain variable region (VL) sequence of rabbit monoclonal antibodies raised against SOD1 peptide NEESTKTGN (SEQ ID NO: 142), located in the electrostatic loop that is normally inaccessible in the well-folded SOD1 structure, and this alpha-helical sequence linearizes when SOD1 is misfolded. Generation of rabbit monoclonal antibodies can be carried out using techniques known in the art and as described herein.

A scFv is a fusion of the variable regions of the heavy (VH) and light chains (VL) of immunoglobulins, connected with a short linker peptide of, for example, ten to about 25 amino acids. The linker is usually rich in glycine for flexibility, as well as serine or threonine for solubility, and can either connect the N-terminus of the VH with the C-terminus of the VL, or vice versa. scFv has been used to facilitate phage display, where it is highly convenient to express the antigen-binding domain as a single peptide. As an alternative, scFv can be created directly from subcloned heavy and light chains derived from a hybridoma. scFv is versatile and can be used in a myriad of applications including, e.g., flow cytometry, immunohistochemistry, as antigen-binding domains of chimeric antigen receptor, and therapeutic uses. In some embodiments, the antibody or binding fragment thereof described herein comprises a heavy chain comprising a heavy chain variable region and a light chain comprising a light chain variable region, wherein the heavy chain and the light chain are connected by a linker. In some embodiments, the linker is a peptide linker. In some embodiments, the linker comprises GSGSG (SEQ ID NO: 222) or GGGGSGGGGSGGGGS (SEQ ID NO: 221).

Antibodies to misfolded SOD1 may also be prepared using techniques known in the art such as those described by Kohler and Milstein, Nature 256, 495 (1975) and Kuroiwa et al, (2002) and in U.S. Pat. Nos. RE 32,011; 4,902,614; 4,543,439; 4,411,993; and 9,133,272; and PCT Application Nos. 2010/004438; and 2014/031694, which are incorporated herein by reference. (See also Monoclonal Antibodies, Hybridomas: A New Dimension in Biological Analyses, Plenum Press, Kennett, McKearn, and Bechtol (eds.), 1980; Monoclonal Antibodies: Methods and Protocols, Humana Press, Albitar (ed.), 2007; and Antibodies: A Laboratory Manual, 2^{nd} Edition, Greenfield (ed), Cold Spring Harbor Laboratory Press, 2014, which are also incorporated herein by reference). Within the context of the present disclosure, antibodies are understood to include monoclonal antibodies, polyclonal antibodies, chimeric antibodies, humanized antibodies, antibody fragments (e.g., Fab, and F(ab′)₂) and recombinantly produced binding partners.

For producing polyclonal antibodies in a host, such as a rabbit or goat, the host is immunized with the immunogen or immunogen fragment, generally with an adjuvant and, if necessary, coupled to a carrier; antibodies to the immunogen are collected from the sera. Further, the polyclonal antibody can be absorbed such that it is monospecific. That is, the sera can be absorbed against related immunogens so that no cross-reactive antibodies remain in the sera rendering it monospecific. To produce monoclonal antibodies, antibody producing cells (lymphocytes) can be harvested from an immunized animal and fused with myeloma cells by standard somatic cell fusion procedures thus immortalizing these cells and yielding hybridoma cells. Such techniques are well known in the art (e.g., the hybridoma technique originally developed by Kohler and Milstein (1975) as well as other techniques such as the human B-cell hybridoma technique (Kozbor, D, and Roder, J, 1983), the EBV-hybridoma technique to produce human monoclonal antibodies (Cole et al., 1985) and screening of combinatorial antibody libraries (Huse, W et al., 1989). Hybridoma cells can be screened immunochemically for production of antibodies specifically reactive with the protein or binding fragment thereof and the monoclonal antibodies can be isolated.

Chimeric antibodies, e.g. antibody molecules that combine a non-human animal variable region and a human constant region, are also contemplated within the scope of the disclosure. Chimeric antibody molecules can include, for example, the variable region or domain from an antibody of a mouse, rat, rabbit, or other species, with a human constant region. Conventional methods may be used to make chimeric antibodies containing the immunoglobulin variable region which recognizes the target (See, Antibodies: A Laboratory Manual, 2^{nd} Edition, Greenfield (ed), Cold Spring Harbor Laboratory Press, 2014).

Monoclonal or chimeric antibodies specifically reactive with a target as described herein can be further humanized by producing human constant region chimeras, in which parts of the variable regions, particularly the conserved framework regions of the antigen-binding domain, are of human origin and only the hypervariable regions are of non-human origin. Such immunoglobulin molecules may be made by techniques known in the art (e.g., Teng et al., 1983; Kozbor, D, and Roder, J, 1983; Olsson and Kaplan; 1982; PCT Publication WO92/06193; Antibodies: A Laboratory Manual, 2^{nd} Edition, Greenfield (ed), Cold Spring Harbor Laboratory Press, 2014; each of which herein incorporated by reference). Humanized antibodies can also be commercially produced.

Rabbit antibodies are suitable for humanization. Rabbit antibodies have limited framework variability as one VH germ line segment is preferentially used (80-90% of VDJ genes) and combined with multiple homologous VJ genes. The fact that rabbit variable domain frameworks are highly homologous to each other, makes them more amenable to a generic human acceptor framework (Borras et al., 2010). Humanized antibodies can be generated by grafting CDRs onto a human antibody acceptor framework. Rabbit variable regions or domains can be humanized by grafting antigen-binding loops to a human framework (i.e. human framework FW1.4). Affinity and stability can be optimized by substituting residues that are highly conserved in rabbit variable domains (i.e. human framework FW1.4gen), which are involved in CDR conformation (Borras et al., 2010 and U.S. Pat. No. 8,293,235 each of which herein incorporated by reference).

Rabbit monoclonal antibodies can also be humanized by grafting the combined CDRs (Kabat, IMGT and Paratome) to a stable human Ig germline framework (i.e. IGHV3-66*01, IGHJ4*01, IGKV1-27*01 and IGKJ4*01). The combined CDR grafting strategy includes antigen-contacting residues as well as supporting residues (Zhang and Ho, 2017, herein incorporated by reference).

Humanization can also be achieved by grafting only the specificity determining residues (SDRs) onto a human antibody framework. Doing so reduces the number of non-human residues while maintaining the residues critical for antigen-antibody interaction and thereby reducing immunogenicity (Kashmiri et al., 2005, herein incorporated by reference). Similarly, selectivity determining residues contained in the rabbit CDR regions can be transferred onto a homologous acceptor human antibody variable heavy and light chain sequence (US 20090104187, herein incorporated by reference).

An alternative CDR grafting technique involves human framework sequences from human germline genes based on CDR similarity. Antibody humanization is accomplished based on the similarity of CDR sequences rather than framework similarity. A human antibody with a similarly structured CDR would support the CDR of another species with retention of affinity. This is accomplished by comparing residue-to residue homology, and the residues in the human CDR not already identical to the rabbit are converted to the rabbit sequence (U.S. Pat. No. 6,881,557, herein incorporated by reference).

Humanized rabbit monoclonal antibodies may be generated using mutational lineage guided (MLG) humanization. The heavy and light chain variable region sequences are aligned with comparable human germline VH and VK sequences. The rabbit residues in the framework regions involved in CDR contacts are not changed. Structurally related and interchangeable residues as well as non-critical structural residues are humanized. Solvent facing residues are also substituted with human germline residues. Selective replacement of non-human residues in both frameworks, and within the CDR generates humanized antibodies guided by biological and sequence information (Yu et al., 2010; WO 2005016950; U.S. Pat. No. 7,462,697, each of which herein incorporated by reference).

The antibody can also be humanized by resurfacing, which substitutes surface accessible residues in the variable region. Replacement of these exposed residues reduces the immunogenicity in humans (US 20040086979, each of which herein incorporated by reference). Similarly, a rabbit monoclonal antibody may be humanized by altering amino acids within the framework regions of the variable region or domains in order to generate a similar sequence to the framework region of a similar human antibody (WO 2005016950, incorporated by reference).

Alternatively, using a phage display approach, chimeric rabbit/human antibodies may be generated, which contain rabbit variable regions or domains and human constant domains and thus already partially humanized. The rabbit variable regions or domains can then be humanized. Humanization may be accomplished using CDR grafting with framework fine tuning by phage display. The rabbit CDR sequences can be grafted into the appropriate human framework. Framework fine tuning can then alter residues in the human framework involved in antigen binding (Rader et al., 2000; U.S. Pat. No. 6,346,269, each of which herein incorporated by reference). Chimeric rabbit/human Fab can also be converted to chimeric rabbit/human IgG1 (Steinberger et al., 2000, US 20030049251 each of which herein incorporated by reference).

For producing recombinant antibodies (see generally Huston et al, 1991; Johnson and Bird, 1991; Mernaugh and Mernaugh, 1995), messenger RNAs from antibody producing B-lymphocytes of animals, or hybridoma are reverse-transcribed to obtain complementary DNAs (cDNAs). Antibody cDNA, which can be full or partial length, is amplified and cloned into a phage or a plasmid. The cDNA can be a partial length of heavy and light chain cDNA, separated or connected by a linker. The antibody, or binding fragment thereof, is expressed using a suitable expression system to obtain recombinant antibody. Antibody cDNA can also be obtained by screening pertinent expression libraries.

The term “signal peptide” or “signal sequence” as used herein refers to a short amino acid sequence at the beginning (N-terminus) of a newly synthesized polypeptide, for example, heavy chain or light chain of an antibody or a fusion protein comprising variable region of these chains. Spanning typically between 15-30 amino acids, this sequence targets the nascent antibody molecules towards the endoplasmic reticulum (ER) or other relevant cellular organelles, setting the stage for their secretion, membrane integration, or specific compartmentalization. Upon reaching the target site, the signal peptide is cleaved, processing the antibody into its mature form, ready to fold and attain its functional configuration. When a signal peptide is not at the N-terminus, it may lose its functionality as a signal peptide and would not provide a cleavage site for processing. An internal signal peptide may or may be serve as a linker. The antibodies and fusion proteins described herein can be with or without a signal peptide, whether at the N-terminus or internally.

As used herein, the term “E3 ligase” or “E3 ubiquitin ligase” refers to a protein in the ubiquitin proteasome pathway that recruits an E2 ubiquitin-conjugating enzyme that has been loaded with ubiquitin, recognizes a protein substrate, and assists or directly catalyzes the transfer of ubiquitin from the E2 to the protein substrate. An E3 ligase interacts with both the target protein and the E2 enzyme, and so imparts substrate specificity to the E2. An E3 ligase usually polyubiquitinates its substrate with Lys48-linked chains of ubiquitin, targeting the substrate for destruction by the proteasome, but many other types of linkages are possible and alter a protein's activity, interactions, or localization. Ubiquitination by E3 ligases regulates diverse areas such as cell cycle control, cell trafficking, DNA repair, and signaling. The human genome encodes over 600 putative E3 ligases, including CHIP (UniProtKB ID: Q9UNE7), UBE4A (UniProtKB ID: Q14139), NEDD4L (UniProtKB ID: Q96PU5), UBR5 (UniProtKB ID: O95071), RNF4 (UniProtKB ID: P78317), UBOX5 (UniProtKB ID: O94941), Parkin (UniProtKB ID: O60260) and BTrCP (UniProtKB ID: Q9Y297), selected here as representatives of the different families of E3 ligases. An E3 ligase or an active fragment thereof can be fused to misfolded SOD1-specific antibody or binding fragment thereof described herein to create a fusion protein termed misfold specific Ubiquitin Ligase fusion protein (mUbL or MisfoldUbL). The binding domain of E3 ligase gives the E3 its substrate specificity. This specificity may be changed when the binding domain is removed or replaced. A truncated form of E3 ligase without the binding domain is useful for targeting misfolded SOD1 when fused to an antibody or binding fragment thereof disclosed herein. An active fragment of E3 ligase retains the catalytic function of E3 ligase and is useful for targeting misfolded SOD1 when fused to an antibody or binding fragment thereof disclosed herein. The molecular biology techniques for making such a fusion protein are known to the skilled person in the art.

As used herein, the term “effective amount” refers to the amount of a therapy (e.g. a prophylactic or therapeutic agent) which is sufficient to effect beneficial or desired results, including clinical results. An effective amount can be administered in one or more administrations.

The term “isolated”, as used herein, refers to a material is removed from its original environment (e.g., the natural environment, if it is naturally occurring). For example, a naturally-occurring antibody present in a living animal is not isolated, but the same antibody, separated from some or all of the coexisting materials in the natural system, is isolated.

The term specific binding, as used herein, includes both low and high affinity specific binding. Specific binding can be exhibited, for example, by a low affinity misfolded SOD1 antibody or binding fragment thereof, or fusion protein having a K_{D} for misfolded SOD1 of about 10⁻⁴ M to about 10⁻⁷ M. Specific binding also can be exhibited by a high affinity misfolded SOD1 antibody or binding fragment thereof, or fusion protein for example, a misfolded SOD1 antibody or binding fragment thereof, or fusion protein having a K_{D} for misfolded SOD1 of at least about 10⁻⁷ M, at least about 10⁻⁸ M, at least about 10⁻⁹ M, at least about 10⁻¹⁰ M, or at least about 10⁻¹¹ M or 10⁻¹² M or greater. A misfolded SOD1 antibody or binding fragment thereof, or fusion protein can have, for example, a K_{D} for misfolded SOD1 of about 2×10⁻⁵ M to 10⁻⁷ M, for example, a K_{D} of about 10⁻⁶ to 10⁷ M, or from about 10⁻⁸ M to 10⁻¹⁰M measured by SPR, or from about 10⁻⁹ M to 5×10⁻⁷ M measured by flow cytometry. Both low and high affinity misfolded SOD1 antibodies or binding fragments thereof, or fusion proteins that selectively bind to misfolded SOD1 can be useful in the methods described herein.

The term “pharmaceutically acceptable carrier” refers to any such carriers known to those skilled in the art to be suitable for the particular mode of administration. For example, the term “pharmaceutically acceptable carrier” includes any and all solvents, dispersion media, coatings, antibacterial and antifungal agents, isotonic and absorption delaying agents and the like, that may be used as a media for a pharmaceutically acceptable substance. In addition, the active materials can also be mixed with other active materials that do not impair the desired action, or with materials that supplement the desired action, or have another action.

The term “pharmaceutically acceptable salt” as used herein refers to salts which are known to be non-toxic and are commonly used in the pharmaceutical literature. Typical inorganic acids used to form such salts include hydrochloric, hydrobromic, hydroiodic, nitric, sulfuric, phosphoric, hypophosphoric, and the like. Salts derived from organic acids, such as aliphatic mono and dicarboxylic acids, phenylsubstituted alkanoic acids, hydroxyalkanoic and hydroxyalkandioic acids, aromatic acids, aliphatic and aromatic sulfonic acids, may also be used. Such pharmaceutically acceptable salts include acetate, phenylacetate, trifluoroacetate, acrylate, ascorbate, benzoate, chlorobenzoate, dinitrobenzoate, hydroxybenzoate, methoxybenzoate, methylbenzoate, o-acetoxybenzoate, naphthalene-2-benzoate, bromide, isobutyrate, phenylbutyrate, beta-hydroxybutyrate, chloride, cinnamate, citrate, formate, fumarate, glycolate, heptanoate, lactate, maleate, hydroxymaleate, malonate, mesylate, nitrate, oxalate, phthalate, phosphate, monohydrogen phosphate, dihydrogenphosphate, metaphosphate, pyrophosphate, propionate, phenylpropionate, salicylate, succinate, sulfate, bisulfate, pyrosulfate, sulfite, bisulfite, sulfonate, benzenesulfonate, p-bromophenylsulfonate, chlorobenzenesulfonate, ethanesulfonate, 2-hydroxyethanesulfonate, methanesulfonate, naphthalene-1-sulfonate, naphthalene-2-sulfonate, p-toluenesulfonate, xylenesulfonate, tartarate, and the like.

As used herein, the terms “treat”, “treatment” and “treating” refer to the prevention, reduction or amelioration of the progression, severity, and/or duration of at least one pathology and/or symptom of any condition or disease. The term “treatment” or “treating” refers to any administration of a compound disclosed herein and includes (i) inhibiting the disease, or the disease state in an individual that is experiencing or displaying the pathology or symptomatology of the disease, or the disease state (e.g., arresting further development of the pathology and/or symptomatology) or (ii) ameliorating the disease in an individual that is experiencing or displaying the pathology or symptomatology of the disease, or the disease state (e.g., reversing the pathology and/or symptomatology). The term “controlling” includes preventing, treating, eradicating, ameliorating or otherwise reducing the severity of symptoms of the disease, or the disease state.

The terms “reducing,” “reduce,” or “reduction” in the context of a disease or condition herein refers to a decrease in the cause, symptoms, or effects of a disease or condition. Therefore, in the disclosed methods, “reducing” can refer to a 10%, 20%, 30%, 40%, 50%, 60%, 70%, 80%, 90%, or 100% decrease, or any value or range there between, in the amount of injury due to reperfusion.

The term “subject” or “patient” or synonym thereto, as used herein includes all members of the animal kingdom, especially mammals, including human. The subject or patient is suitably a human.

The recitation of numerical ranges by endpoints herein includes all numbers and fractions subsumed within that range (e.g. 1 to 5 includes for example 1, 1.5, 2, 2.75, 3, 3.90, 4, and 5). It is also to be understood that all numbers and fractions thereof are presumed to be modified by the term “about”.

### II. Misfolded SOD1 Antibody or Binding Fragment Thereof, Fusion Protein, or Nucleic Acid and Composition

Misfolded SOD1-specific antibody or binding fragment thereof, or fusion protein, has utility for treating a medical condition, disease, or disorder mediated by a misfolded form of SOD1 in a subject in need of treatment.

### A. Misfolded SOD1 Binding Antibodies

Disclosed herein are antibodies or binding fragments thereof that bind to misfolded SOD1. Variants and modified embodiments of these antibodies that are capable of being used in these methods are also provided.

The present disclosure provides the misfolded SOD1 epitope ESTKTGN (SEQ ID NO: 144) which is the core of the epitope that is recognized by misfolded SOD1 antibodies. In an embodiment, the antibody or binding fragment thereof bind to the epitope ESTKTGN (SEQ ID NO: 144).

In an embodiment, the antibody binding fragment is a Fv, scFv, Fab, Fab′, F(ab′)2, dsFv, ds-scFv, sdAB, dimer, minibody, diabody, or multimer thereof. In an embodiment, the antibody binding fragment is scFv.

In some embodiments, the antibody or binding fragment thereof that binds to misfolded SOD1 contains one or more modifications to increase protease resistance, serum stability and/or bioavailability. In some embodiments, the modification is selected from pegylation, acetylation, glycosylation, biotinylation, prenylation or substitution with D-amino acid and/or unnatural amino acid of the antibody or binding fragment thereof. The antibody or binding fragment thereof can contain one or more non-canonical disulphide linkages, e.g., at IMGT (ImMunoGeneTics) positions 54 and 78, to increase stability, protease resistance, serum stability and/or bioavailability. Accordingly, in some embodiments, the antibody or binding fragment thereof that binds to misfolded SOD1 contains one or more non-canonical disulphide linkages to increase stability, protease resistance, serum stability and/or bioavailability. In some embodiments one or more non-canonical disulphide linkages are at IMGT positions 54 and 78.

As is understood in the art, the amino acid position or boundary delineating the CDR regions of an antibody can vary, depending on the context and the different definitions known in the art. Some positions within the variable regions can be viewed as hybrid CDRs in that the positions can be within a CDR region under one set of criteria while being deemed to be outside a CDR region under another set of criteria. In some embodiments, the CDRs in variable light and variable heavy chains can be delineated using the IMGT, Kabat, Chothia, AbM, Contact, or Paratome schemes, or another scheme known in art. The “Kabat” approach for defining CDRs uses sequence variability (Kabat et al. (1991); herein incorporated by reference). “Chothia” uses the location of structural loops (Chothia and Lesk, (1987), Chothia et al. (1992); herein incorporated by reference). The IMGT numbering scheme is an adaptation of the numbering scheme of Chothia (Lefranc et al., (1999); see also http://imgt.cines.fr; herein incorporated by reference). CDRs defined by “AbM” is a compromise between the Kabat and Chothia and is delineated using Oxford Molecular AbM antibody modeling software (see, Martin et al. (1989); see also www.bioinf-org.uk/abs; herein incorporated by reference). The antibody numbering scheme developed by the Chemical Computing Group (CCG) combines several antibody numbering schemes and offers a broader definition of CDR boundaries based on Martin and collaborators' CDR definitions (see Maier et al. (2014); herein incorporated by reference). The “Contact” CDR delineations are based on analysis of known antibody-antigen crystal structures (see, e.g., MacCallum et al. (1996)). The “Paratome” approach involves computational programs based on a set of consensus regions derived from a structural alignment of a non-redundant set of known antibody-antigen complexes (Kunik et al. (2012); see also www.ofranlab.org/paratome/; herein incorporated by reference). The “Aho” or Honegger scheme numbers the variable domains of the immunoglobulin superfamily in a homogenized format. This system is based on structural alignments of the 3D structures of the immunoglobulin variable regions covering the observed length variation. It allows to define structurally conserved Ca positions and therefore deduces appropriate framework regions and CDR lengths (see Honegger, A. and Pluckthun A. (2001); herein incorporated by reference). The CDRs described herein include those based on CCG, Paratome, Kabat, Chothia, or Aho definition (see Table 7), and it is to be understood that CDRs based on other methods are to be encompassed herein. In some embodiments, the CDRs described herein are based on IMGT, CCG, Paratome, Kabat, Chothia, or Aho.

In an exemplary embodiment of the present disclosure, the isolated antibody or binding fragment thereof binds to misfolded superoxide dismutase 1 (SOD1) epitope having an amino acid sequence shown in SEQ ID NO: 142 or 144, the antibody or binding fragment thereof comprises:
(i) a heavy chain comprising a heavy chain variable region, wherein the heavy chain variable region comprises CDR1, CDR2, and CDR3 having the amino acid sequence of SEQ ID NO: 1, SEQ ID NO: 2, and SEQ ID NO: 3, and a light chain comprising a light chain variable region, wherein the light chain variable region comprises CDR1, CDR2, and CDR3 having the amino acid sequence of SEQ ID NO: 4, SEQ ID NO: 5, and SEQ ID NO: 6;
(ii) a heavy chain comprising a heavy chain variable region, wherein the heavy chain variable region comprises CDR1, CDR2, and CDR3 having the amino acid sequence of SEQ ID NO: 7, SEQ ID NO: 8, and SEQ ID NO: 9, and a light chain comprising a light chain variable region, wherein the light chain variable region comprises CDR1, CDR2 and CDR3 having the amino acid sequence of SEQ ID NO: 10, SEQ ID NO: 11 and SEQ ID NO: 12;
(iii) a heavy chain comprising a heavy chain variable region, wherein the heavy chain variable region comprises CDR1, CDR2 and CDR3 having the amino acid sequence of SEQ ID NO: 14, SEQ ID NO: 15, and SEQ ID NO: 16, and a light chain comprising a light chain variable region, wherein the light chain variable region comprises CDR1, CDR2 and CDR3 having the amino acid sequence of SEQ ID NO: 10, SEQ ID NO: 11, and SEQ ID NO: 12;
(iv) a heavy chain comprising a heavy chain variable region, wherein the heavy chain variable region comprises CDR1, CDR2, and CDR3 having the amino acid sequence of SEQ ID NO: 7, SEQ ID NO: 8, and SEQ ID NO: 9, and a light chain comprising a light chain variable region, wherein the light chain variable region comprises CDR1, CDR2 and CDR3 having the amino acid sequence of SEQ ID NO: 10, SEQ ID NO: 11 and SEQ ID NO: 13;
(v) a heavy chain comprising a heavy chain variable region, wherein the heavy chain variable region comprises CDR1, CDR2 and CDR3 having the amino acid sequence of SEQ ID NO: 14, SEQ ID NO: 15, and SEQ ID NO: 16, and a light chain comprising a light chain variable region, wherein the light chain variable region comprises CDR1, CDR2 and CDR3 having the amino acid sequence of SEQ ID NO: 10, SEQ ID NO: 11, and SEQ ID NO: 13;
(vi) a heavy chain comprising a heavy chain variable region, wherein the heavy chain variable region comprises CDR1, CDR2 and CDR3 having the amino acid sequence of SEQ ID NO: 53, SEQ ID NO: 54, and SEQ ID NO: 55, and a light chain comprising a light chain variable region, wherein the light chain variable region comprises CDR1, CDR2 and CDR3 having the amino acid sequence of SEQ ID NO: 56, SEQ ID NO: 57, and SEQ ID NO: 58;
(vii) a heavy chain comprising a heavy chain variable region, wherein the heavy chain variable region comprises CDR1, CDR2 and CDR3 having the amino acid sequence of SEQ ID NO: 23, SEQ ID NO: 24, and SEQ ID NO: 25, and a light chain comprising a light chain variable region, wherein the light chain variable region comprises CDR1, CDR2 and CDR3 having the amino acid sequence of SEQ ID NO: 26, SEQ ID NO: 27, and SEQ ID NO: 28; or
(viii) a heavy chain comprising a heavy chain variable region, wherein the heavy chain variable region comprises CDR1, CDR2 and CDR3 having the amino acid sequence of SEQ ID NO: 29, SEQ ID NO: 30, and SEQ ID NO: 31, and a light chain comprising a light chain variable region, wherein the light chain variable region comprises CDR1, CDR2 and CDR3 having the amino acid sequence of SEQ ID NO: 32, SEQ ID NO: 33, and SEQ ID NO: 34.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region comprises CDR1, CDR2, and CDR3 having the amino acid sequence of SEQ ID NO: 1, SEQ ID NO: 2, and SEQ ID NO: 3, and a light chain comprising a light chain variable region, wherein the light chain variable region comprises CDR1, CDR2, and CDR3 having the amino acid sequence of SEQ ID NO: 4, SEQ ID NO: 5, and SEQ ID NO: 6.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region comprises CDR1, CDR2, and CDR3 having the amino acid sequence of SEQ ID NO: 7, SEQ ID NO: 8, and SEQ ID NO: 9, and a light chain comprising a light chain variable region, wherein the light chain variable region comprises CDR1, CDR2 and CDR3 having the amino acid sequence of SEQ ID NO: 10, SEQ ID NO: 11 and SEQ ID NO: 12.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region comprises CDR1, CDR2 and CDR3 having the amino acid sequence of SEQ ID NO: 14, SEQ ID NO: 15, and SEQ ID NO: 16, and a light chain comprising a light chain variable region, wherein the light chain variable region comprises CDR1, CDR2 and CDR3 having the amino acid sequence of SEQ ID NO: 10, SEQ ID NO: 11, and SEQ ID NO: 12.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region comprises CDR1, CDR2, and CDR3 having the amino acid sequence of SEQ ID NO: 7, SEQ ID NO: 8, and SEQ ID NO: 9, and a light chain comprising a light chain variable region, wherein the light chain variable region comprises CDR1, CDR2 and CDR3 having the amino acid sequence of SEQ ID NO: 10, SEQ ID NO: 11 and SEQ ID NO: 13.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region comprises CDR1, CDR2 and CDR3 having the amino acid sequence of SEQ ID NO: 14, SEQ ID NO: 15, and SEQ ID NO: 16, and a light chain comprising a light chain variable region, wherein the light chain variable region comprises CDR1, CDR2 and CDR3 having the amino acid sequence of SEQ ID NO: 10, SEQ ID NO: 11, and SEQ ID NO: 13.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region comprises CDR1, CDR2 and CDR3 having the amino acid sequence of SEQ ID NO: 53, SEQ ID NO: 54, and SEQ ID NO: 55, and a light chain comprising a light chain variable region, wherein the light chain variable region comprises CDR1, CDR2 and CDR3 having the amino acid sequence of SEQ ID NO: 56, SEQ ID NO: 57, and SEQ ID NO: 58.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region comprises CDR1, CDR2 and CDR3 having the amino acid sequence of SEQ ID NO: 23, SEQ ID NO: 24, and SEQ ID NO: 25, and a light chain comprising a light chain variable region, wherein the light chain variable region comprises CDR1, CDR2 and CDR3 having the amino acid sequence of SEQ ID NO: 26, SEQ ID NO: 27, and SEQ ID NO: 28.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region comprises CDR1, CDR2 and CDR3 having the amino acid sequence of SEQ ID NO: 29, SEQ ID NO: 30, and SEQ ID NO: 31, and a light chain comprising a light chain variable region, wherein the light chain variable region comprises CDR1, CDR2 and CDR3 having the amino acid sequence of SEQ ID NO: 32, SEQ ID NO: 33, and SEQ ID NO: 34.

In some embodiments, the CDR described herein has one or two positions mutated. In some embodiments, the CDR comprising one or two mutated positions retain binding activity specific to misfolded SOD1 epitope having an amino acid sequence shown in SEQ ID NO: 142 or 144.

In some embodiments, the isolated antibody or binding fragment thereof binds to misfolded SOD1 epitope having an amino acid sequence shown in SEQ ID NO: 142 or 144, the antibody or binding fragment thereof comprises:
(i) a heavy chain comprises a heavy chain variable region comprising an amino acid sequence having at least 85%, 90%, 95%, 97%, 98%, 99%, 99.5%, 99.5% or 100% identity to the amino acid sequence of SEQ ID NO: 65, and a light chain comprises a light chain variable region comprising an amino acid sequence having at least 85%, 90%, 95%, 97%, 98%, 99%, 99.5%, 99.5% or 100% identity to the amino acid sequence of SEQ ID NO: 70;
(ii) a heavy chain comprises a heavy chain variable region comprising an amino acid sequence having at least 85%, 90%, 95%, 97%, 98%, 99%, 99.5%, 99.5% or 100% identity to the amino acid sequence of SEQ ID NO: 68, and a light chain comprises a light chain variable region comprising an amino acid sequence having at least 85%, 90%, 95%, 97%, 98%, 99%, 99.5%, 99.5% or 100% identity to the amino acid sequence of SEQ ID NO: 70;
(iii) a heavy chain comprises a heavy chain variable region comprising an amino acid sequence having at least 85%, 90%, 95%, 97%, 98%, 99%, 99.5%, 99.5% or 100% identity to the amino acid sequence of SEQ ID NO: 72, and a light chain comprises a light chain variable region comprising an amino acid sequence having at least 85%, 90%, 95%, 97%, 98%, 99%, 99.5%, 99.5% or 100% identity to the amino acid sequence of SEQ ID NO: 76;
(iv) a heavy chain comprises a heavy chain variable region comprising an amino acid sequence having at least 85%, 90%, 95%, 97%, 98%, 99%, 99.5%, 99.5% or 100% identity to the amino acid sequence of SEQ ID NO: 74, and a light chain comprises a light chain variable region comprising an amino acid sequence having at least 85%, 90%, 95%, 97%, 98%, 99%, 99.5%, 99.5% or 100% identity to the amino acid sequence of SEQ ID NO: 76;
(v) a heavy chain comprises a heavy chain variable region comprising an amino acid sequence having at least 85%, 90%, 95%, 97%, 98%, 99%, 99.5%, 99.5% or 100% identity to the amino acid sequence of SEQ ID NO: 78, and a light chain comprises a light chain variable region comprising an amino acid sequence having at least 85%, 90%, 95%, 97%, 98%, 99%, 99.5%, 99.5% or 100% identity to the amino acid sequence of SEQ ID NO: 82;
(vi) a heavy chain comprises a heavy chain variable region comprising an amino acid sequence having at least 85%, 90%, 95%, 97%, 98%, 99%, 99.5%, 99.5% or 100% identity to the amino acid sequence of SEQ ID NO: 80, and a light chain comprises a light chain variable region comprising an amino acid sequence having at least 85%, 90%, 95%, 97%, 98%, 99%, 99.5%, 99.5% or 100% identity to the amino acid sequence of SEQ ID NO: 82;
(vii) a heavy chain comprises a heavy chain variable region comprising an amino acid sequence having at least 85%, 90%, 95%, 97%, 98%, 99%, 99.5%, 99.5% or 100% identity to the amino acid sequence of SEQ ID NO: 84, and a light chain comprises a light chain variable region comprising an amino acid sequence having at least 85%, 90%, 95%, 97%, 98%, 99%, 99.5%, 99.5% or 100% identity to the amino acid sequence of SEQ ID NO: 86; or
(viii) a heavy chain comprises a heavy chain variable region comprising an amino acid sequence having at least 85%, 90%, 95%, 97%, 98%, 99%, 99.5%, 99.5% or 100% identity to the amino acid sequence of SEQ ID NO: 88, and a light chain comprises a light chain variable region comprising an amino acid sequence having at least 85%, 90%, 95%, 97%, 98%, 99%, 99.5%, 99.5% or 100% identity to the amino acid sequence of SEQ ID NO: 91.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region comprising an amino acid sequence having at least 85%, 90%, 95%, 97%, 98%, 99%, 99.5%, 99.5% or 100% identity to the amino acid sequence of SEQ ID NO: 65, and a light chain comprising a light chain variable region comprising an amino acid sequence having at least 85%, 90%, 95%, 97%, 98%, 99%, 99.5%, 99.5% or 100% identity to the amino acid sequence of SEQ ID NO: 70.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region comprising an amino acid sequence having at least 85%, 90%, 95%, 97%, 98%, 99%, 99.5%, 99.5% or 100% identity to the amino acid sequence of SEQ ID NO: 68, and a light chain comprising a light chain variable region comprising an amino acid sequence having at least 85%, 90%, 95%, 97%, 98%, 99%, 99.5%, 99.5% or 100% identity to the amino acid sequence of SEQ ID NO: 70.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region comprising an amino acid sequence having at least 85%, 90%, 95%, 97%, 98%, 99%, 99.5%, 99.5% or 100% identity to the amino acid sequence of SEQ ID NO: 72, and a light chain comprising a light chain variable region comprising an amino acid sequence having at least 85%, 90%, 95%, 97%, 98%, 99%, 99.5%, 99.5% or 100% identity to the amino acid sequence of SEQ ID NO: 76.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region comprising an amino acid sequence having at least 85%, 90%, 95%, 97%, 98%, 99%, 99.5%, 99.5% or 100% identity to the amino acid sequence of SEQ ID NO: 74, and a light chain comprising a light chain variable region comprising an amino acid sequence having at least 85%, 90%, 95%, 97%, 98%, 99%, 99.5%, 99.5% or 100% identity to the amino acid sequence of SEQ ID NO: 76.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region comprising an amino acid sequence having at least 85%, 90%, 95%, 97%, 98%, 99%, 99.5%, 99.5% or 100% identity to the amino acid sequence of SEQ ID NO: 78, and a light chain comprising a light chain variable region comprising an amino acid sequence having at least 85%, 90%, 95%, 97%, 98%, 99%, 99.5%, 99.5% or 100% identity to the amino acid sequence of SEQ ID NO: 82.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region comprising an amino acid sequence having at least 85%, 90%, 95%, 97%, 98%, 99%, 99.5%, 99.5% or 100% identity to the amino acid sequence of SEQ ID NO: 80, and a light chain comprising a light chain variable region comprising an amino acid sequence having at least 85%, 90%, 95%, 97%, 98%, 99%, 99.5%, 99.5% or 100% identity to the amino acid sequence of SEQ ID NO: 82.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region comprising an amino acid sequence having at least 85%, 90%, 95%, 97%, 98%, 99%, 99.5%, 99.5% or 100% identity to the amino acid sequence of SEQ ID NO: 84, and a light chain comprising a light chain variable region comprising an amino acid sequence having at least 85%, 90%, 95%, 97%, 98%, 99%, 99.5%, 99.5% or 100% identity to the amino acid sequence of SEQ ID NO: 86.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region comprising an amino acid sequence having at least 85%, 90%, 95%, 97%, 98%, 99%, 99.5%, 99.5% or 100% identity to the amino acid sequence of SEQ ID NO: 88, and a light chain comprising a light chain variable region comprising an amino acid sequence having at least 85%, 90%, 95%, 97%, 98%, 99%, 99.5%, 99.5% or 100% identity to the amino acid sequence of SEQ ID NO: 91.

In some embodiments, the percentage identity is outside of the CDRs described herein.

In some embodiments, the antibody binding fragment is a Fab, Fab′, F(ab′)2, scFv, dsFv, ds-scFv, dimer, minibody, diabody, or bispecific antibody binding fragment. In some embodiments, the antibody binding fragment is a scFv.

Also provided is an isolated antibody that binds to misfolded SOD1 epitope having an amino acid sequence shown in SEQ ID NO: 142 or 144, the antibody comprises:
(i) a heavy chain comprising an amino acid sequence having at least 85%, 90%, 95%, 97%, 98%, 99%, 99.5%, 99.5% or 100% identity to the amino acid sequence of SEQ ID NO: 109, and a light chain comprising an amino acid sequence having at least 85%, 90%, 95%, 97%, 98%, 99%, 99.5%, 99.5% or 100% identity to the amino acid sequence of SEQ ID NO: 114;
(ii) a heavy chain comprising an amino acid sequence having at least 85%, 90%, 95%, 97%, 98%, 99%, 99.5%, 99.5% or 100% identity to the amino acid sequence of SEQ ID NO: 112, and a light chain comprising an amino acid sequence having at least 85%, 90%, 95%, 97%, 98%, 99%, 99.5%, 99.5% or 100% identity to the amino acid sequence of SEQ ID NO: 114;
(iii) a heavy chain comprising an amino acid sequence having at least 85%, 90%, 95%, 97%, 98%, 99%, 99.5%, 99.5% or 100% identity to the amino acid sequence of SEQ ID NO: 116, and a light chain comprising an amino acid sequence having at least 85%, 90%, 95%, 97%, 98%, 99%, 99.5%, 99.5% or 100% identity to the amino acid sequence of SEQ ID NO: 120;
(iv) a heavy chain comprising an amino acid sequence having at least 85%, 90%, 95%, 97%, 98%, 99%, 99.5%, 99.5% or 100% identity to the amino acid sequence of SEQ ID NO: 118, and a light chain comprising an amino acid sequence having at least 85%, 90%, 95%, 97%, 98%, 99%, 99.5%, 99.5% or 100% identity to the amino acid sequence of SEQ ID NO: 120;
(v) a heavy chain comprising an amino acid sequence having at least 85%, 90%, 95%, 97%, 98%, 99%, 99.5%, 99.5% or 100% identity to the amino acid sequence of SEQ ID NO: 122, and a light chain comprising an amino acid sequence having at least 85%, 90%, 95%, 97%, 98%, 99%, 99.5%, 99.5% or 100% identity to the amino acid sequence of SEQ ID NO: 126;
(vi) a heavy chain comprising an amino acid sequence having at least 85%, 90%, 95%, 97%, 98%, 99%, 99.5%, 99.5% or 100% identity to the amino acid sequence of SEQ ID NO: 124, and a light chain comprising an amino acid sequence having at least 85%, 90%, 95%, 97%, 98%, 99%, 99.5%, 99.5% or 100% identity to the amino acid sequence of SEQ ID NO: 126;
(vii) a heavy chain comprising an amino acid sequence having at least 85%, 90%, 95%, 97%, 98%, 99%, 99.5%, 99.5% or 100% identity to the amino acid sequence of SEQ ID NO: 128, and a light chain comprising an amino acid sequence having at least 85%, 90%, 95%, 97%, 98%, 99%, 99.5%, 99.5% or 100% identity to the amino acid sequence of SEQ ID NO: 130; or
(viii) a heavy chain comprising an amino acid sequence having at least 85%, 90%, 95%, 97%, 98%, 99%, 99.5%, 99.5% or 100% identity to the amino acid sequence of SEQ ID NO: 132, and a light chain comprising an amino acid sequence having at least 85%, 90%, 95%, 97%, 98%, 99%, 99.5%, 99.5% or 100% identity to the amino acid sequence of SEQ ID NO: 135.

In some embodiments, the isolated antibody binds to misfolded SOD1 epitope having an amino acid sequence shown in SEQ ID NO: 142 or 144, the antibody comprises a heavy chain comprising an amino acid sequence having at least 85%, 90%, 95%, 97%, 98%, 99%, 99.5%, 99.5% or 100% identity to the amino acid sequence of SEQ ID NO: 109, and a light chain comprising an amino acid sequence having at least 85%, 90%, 95%, 97%, 98%, 99%, 99.5%, 99.5% or 100% identity to the amino acid sequence of SEQ ID NO: 114.

In some embodiments, the isolated antibody binds to misfolded SOD1 epitope having an amino acid sequence shown in SEQ ID NO: 142 or 144, the antibody comprises a heavy chain comprising an amino acid sequence having at least 85%, 90%, 95%, 97%, 98%, 99%, 99.5%, 99.5% or 100% identity to the amino acid sequence of SEQ ID NO: 112, and a light chain comprising an amino acid sequence having at least 85%, 90%, 95%, 97%, 98%, 99%, 99.5%, 99.5% or 100% identity to the amino acid sequence of SEQ ID NO: 114.

In some embodiments, the isolated antibody binds to misfolded SOD1 epitope having an amino acid sequence shown in SEQ ID NO: 142 or 144, the antibody comprises a heavy chain comprising an amino acid sequence having at least 85%, 90%, 95%, 97%, 98%, 99%, 99.5%, 99.5% or 100% identity to the amino acid sequence of SEQ ID NO: 116, and a light chain comprising an amino acid sequence having at least 85%, 90%, 95%, 97%, 98%, 99%, 99.5%, 99.5% or 100% identity to the amino acid sequence of SEQ ID NO: 120.

In some embodiments, the isolated antibody binds to misfolded SOD1 epitope having an amino acid sequence shown in SEQ ID NO: 142 or 144, the antibody comprises a heavy chain comprising an amino acid sequence having at least 85%, 90%, 95%, 97%, 98%, 99%, 99.5%, 99.5% or 100% identity to the amino acid sequence of SEQ ID NO: 118, and a light chain comprising an amino acid sequence having at least 85%, 90%, 95%, 97%, 98%, 99%, 99.5%, 99.5% or 100% identity to the amino acid sequence of SEQ ID NO: 120.

In some embodiments, the isolated antibody binds to misfolded SOD1 epitope having an amino acid sequence shown in SEQ ID NO: 142 or 144, the antibody comprises a heavy chain comprising an amino acid sequence having at least 85%, 90%, 95%, 97%, 98%, 99%, 99.5%, 99.5% or 100% identity to the amino acid sequence of SEQ ID NO: 122, and a light chain comprising an amino acid sequence having at least 85%, 90%, 95%, 97%, 98%, 99%, 99.5%, 99.5% or 100% identity to the amino acid sequence of SEQ ID NO: 126.

In some embodiments, the isolated antibody binds to misfolded SOD1 epitope having an amino acid sequence shown in SEQ ID NO: 142 or 144, the antibody comprises a heavy chain comprising an amino acid sequence having at least 85%, 90%, 95%, 97%, 98%, 99%, 99.5%, 99.5% or 100% identity to the amino acid sequence of SEQ ID NO: 124, and a light chain comprising an amino acid sequence having at least 85%, 90%, 95%, 97%, 98%, 99%, 99.5%, 99.5% or 100% identity to the amino acid sequence of SEQ ID NO: 126.

In some embodiments, the isolated antibody binds to misfolded SOD1 epitope having an amino acid sequence shown in SEQ ID NO: 142 or 144, the antibody comprises a heavy chain comprising an amino acid sequence having at least 85%, 90%, 95%, 97%, 98%, 99%, 99.5%, 99.5% or 100% identity to the amino acid sequence of SEQ ID NO: 128, and a light chain comprising an amino acid sequence having at least 85%, 90%, 95%, 97%, 98%, 99%, 99.5%, 99.5% or 100% identity to the amino acid sequence of SEQ ID NO: 130.

In some embodiments, the isolated antibody binds to misfolded SOD1 epitope having an amino acid sequence shown in SEQ ID NO: 142 or 144, the antibody comprises a heavy chain comprising an amino acid sequence having at least 85%, 90%, 95%, 97%, 98%, 99%, 99.5%, 99.5% or 100% identity to the amino acid sequence of SEQ ID NO: 132, and a light chain comprising an amino acid sequence having at least 85%, 90%, 95%, 97%, 98%, 99%, 99.5%, 99.5% or 100% identity to the amino acid sequence of SEQ ID NO: 135.

In some embodiments, the isolated antibody binds to misfolded SOD1 epitope having an amino acid sequence shown in SEQ ID NO: 142 or 144, the antibody comprises a heavy chain comprising an amino acid sequence having at least 85%, 90%, 95%, 97%, 98%, 99%, 99.5%, 99.5% or 100% identity to the amino acid sequence of SEQ ID NO: 93, and a light chain comprising an amino acid sequence having at least 85%, 90%, 95%, 97%, 98%, 99%, 99.5%, 99.5% or 100% identity to the amino acid sequence of SEQ ID NO: 95.

In some embodiments, the percentage identity is outside of the CDRs described herein.

### B. Modified Antibodies and Antibody Analogs

In some embodiments, the antibody or binding fragment thereof that binds to misfolded SOD1 contains one or more L-amino acids, D-amino acids, and/or non-standard amino acids. In various embodiments, the antibody or binding fragment thereof comprises amino acids, including carboxy- and/or amino-terminal amino acids in antibodies, or can be modified by PEGylation, methylation, amidation, acetylation, prenylation and/or substitution with other chemical groups that can change the antibody's or binding fragment thereof's circulating half-life without adversely affecting its activity. Examples of unconventional or un-natural amino acids include, but not limited to, citrulline, ornithine, norleucine, norvaline, 4-(E)-butenyl-4(R)-methyl-N-methylthreonine (MeBmt), N-methyl-leucine (MeLeu), aminoisobutyric acid, statine, and N-methyl-alanine (MeAla). Amino acids may participate in a disulfide bond. In some embodiments, the amino acid has the general structure H2N—C(H)(R)—COOH. In some embodiments, the amino acid is a naturally-occurring amino acid. In some embodiments, the amino acid is a synthetic or un-natural amino acid (e.g., α,α-disubstituted amino acids, N-alkyl amino acids); in some embodiments, the amino acid is a D-amino acid; in some embodiments, the amino acid is an L-amino acid. In some embodiments, the modified amino acids comprise a modification selected from the group consisting of methylation, amidation, acetylation, and/or substitution with other chemical groups. In some embodiments, the antibody or binding fragment thereof is modified by pegylation, acetylation, glycosylation, biotinylation, or prenylation.

The antibody or binding fragment thereof described herein can be fused to Fc domain and can also be a multimer, for example, to generate bispecific/biparatopic molecules. In some embodiments, the antibody or binding fragment thereof that binds to misfolded SOD1 is fused to a Fc domain. In some embodiments, the antibody or binding fragment thereof that binds to misfolded SOD1 is a multimer. In some embodiments, the antibody or binding fragment thereof that binds to misfolded SOD1 is fused to a Fc domain and is a multimer.

The antibody and binding fragment thereof are useful for diagnostic purposes, including in vivo imaging to identify endogenous sites of misfolded SOD1, and for sample testing to detect SOD1. The antibody and binding fragment thereof are also useful for therapeutic purposes to treat diseases in which misfolded SOD1 is implicated. In some embodiments, the antibody or binding fragment thereof described herein is for treating or diagnosing a medical condition, disease, or disorder mediated by a misfolded form of SOD1 in a subject.

For either purpose, the antibody or binding fragment can be conjugated to an appropriate agent, to form a conjugate. The activity bearing sequence of the antibody or binding fragment thereof is not appreciably impacted by the appropriate agent. For diagnostic purposes, appropriate agents are detectable labels that include radioisotopes or fluorescent markers for whole body imaging, and radioisotopes, enzymes, peptides, fluorescent labels and the like for sample testing. In these diagnostic approaches, the agent can serve as a label either directly, as such, or indirectly as an agent that will bind a desired label such as a labeled secondary antibody that binds the agent. For diagnostics, the detectable labels can be any of the various types used in the field of in vitro diagnostics, including particulate labels including biotin/streptavidin, metal sols such as colloidal gold, radioactive isotopes such as I¹²⁵ or Tc⁹⁹ presented for instance with a peptidic chelating agent of the N₂S₂, N₃S or N₄ type, chromophores including fluorescent markers such as FITC and PE, luminescent markers, phosphorescent markers and the like, as well as enzyme labels that convert a given substrate to a detectable marker, and polynucleotide tags that are revealed following amplification such as by polymerase chain reaction. Suitable enzyme labels include horseradish peroxidase, alkaline phosphatase and the like. For instance, the label can be the enzyme alkaline phosphatase, detected by measuring the presence or formation of chemiluminescence following conversion of 1,2 dioxetane substrates such as adamantyl methoxy phosphoryloxy phenyl dioxetane (AMPPD), disodium 3-(4-(methoxyspiro{1,2-dioxetane-3,2′-(5′-chloro)tricyclo{3.3.1.1 3,7}decan}-4-yl) phenyl phosphate (CSPD), as well as CDP and CDP-star® or other luminescent substrates well-known to those in the art, for example the chelates of suitable lanthanides such as Terbium(III) and Europium(III). The detection means is determined by the chosen label. Appearance of the label or its reaction products can be achieved using the naked eye, in the case where the label is particulate or chromatic and accumulates at appropriate levels, or using instruments such as a spectrophotometer, a luminometer, a fluorimeter, and the like, all in accordance with standard practice. In some embodiments, the antibody or binding fragment thereof further comprises an appropriate agent described herein. In some embodiments, the antibody or binding fragment thereof further comprises a detectable label described herein.

Imaging agents may be included in a composition or in additional compositions. Suitable imaging agents include commercially available agents used in positron emission tomography (PET), computer assisted tomography (CAT), single photon emission computerized tomography, x-ray, fluoroscopy, and magnetic resonance imaging (MRI).

Imaging agents useful with the antibody or binding fragment thereof to screen for endogenous sites for misfolded SOD1 include metals, radioactive isotopes and radioopaque agents (e.g., gallium, technetium, indium, strontium, iodine, barium, bromine and phosphorus-containing compounds), radiolucent agents, contrast agents, dyes (e.g., fluorescent dyes and chromophores) and enzymes that catalyze a colorimetric or fluorometric reaction. In general, such agents may be attached or entrapped using a variety of techniques as described above, and may be present in any orientation. See, e.g., U.S. Pat. Nos. 6,391,280; 7,875,454; 8,102,945; 10,086,073; and 10,449,261, which are expressly incorporated by reference herein.

Contrast agents according to the present disclosure are useful in the imaging modalities, such as X-ray contrast agents, light imaging probes, spin labels or radioactive units. Examples of suitable materials for use as contrast agents in MRI include the gadolinium chelates currently available, such as diethylene triamine pentaacetic acid (DTPA) and gadopentotate dimeglumine, as well as iron, magnesium, manganese, copper, and chromium. Examples of materials useful for CAT and x-rays include iodine-based materials, such as ionic monomers typified by diatrizoate and iothalamate, non-ionic monomers such as iopamidol, isohexol, and ioversol, non-ionic dimers, such as iotrol and iodixanol, and ionic dimers, for example, ioxagalte.

Agents for use with PET scan include N¹³ and fluorodeoxyglucose (FDG).

In some embodiments, the detectable label is a peptide label comprising three or more amino acid residues at the C-terminus, the N-terminus or both the C-terminus and the N-terminus. In some embodiments, the antibody or binding fragment thereof comprises one or more peptide labels. In some embodiments, the antibody or binding fragment thereof that binds to misfolded SOD1 further comprises one or more peptide labels comprising 3, 4, 5, 6, 7, 8, 9, 10, 11, 12, 13, 14, 15, 16, 17, 18, 19, 20, 21, 22, 23, 24, 25, 26, 27, 28, or 30 amino acid residues at the N-terminus. In some embodiments, the antibody or binding fragment thereof that binds to misfolded SOD1 further comprises one or more peptide labels comprising 3, 4, 5, 6, 7, 8, 9, 10, 11, 12, 13, 14, 15, 16, 17, 18, 19, 20, 21, 22, 23, 24, 25, 26, 27, 28, or 30 amino acid residues at the C-terminus. In some embodiments, the antibody or binding fragment thereof that binds to misfolded SOD1 further comprises one or more peptide labels comprising 3, 4, 5, 6, 7, 8, 9, 10, 11, 12, 13, 14, 15, 16, 17, 18, 19, 20, 21, 22, 23, 24, 25, 26, 27, 28, or 30 amino acid residues at both the C-terminus and the N-terminus.

### C. Misfold Specific Ubiquitin Ligase Fusion Proteins

The present disclosure also provides a fusion protein comprising the antibody or binding fragment thereof that binds to misfolded SOD1 and an E3 ligase or an active fragment thereof. In some embodiments, the fusion protein comprises the antibody or binding fragment thereof described herein and an E3 ligase or an active fragment thereof. The sequences of E3 ligases, truncated version, or an active fragment thereof described herein can have at least 85% identity and maintain ubiquitin ligase activity.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises:
(i) a heavy chain comprising a heavy chain variable region, wherein the heavy chain variable region comprises CDR1, CDR2, and CDR3 having the amino acid sequence of SEQ ID NO: 1, SEQ ID NO: 2, and SEQ ID NO: 3, and a light chain comprising a light chain variable region, wherein the light chain variable region comprises CDR1, CDR2, and CDR3 having the amino acid sequence of SEQ ID NO: 4, SEQ ID NO: 5, and SEQ ID NO: 6;
(ii) a heavy chain comprising a heavy chain variable region, wherein the heavy chain variable region comprises CDR1, CDR2, and CDR3 having the amino acid sequence of SEQ ID NO: 7, SEQ ID NO: 8, and SEQ ID NO: 9, and a light chain comprising a light chain variable region, wherein the light chain variable region comprises CDR1, CDR2 and CDR3 having the amino acid sequence of SEQ ID NO: 10, SEQ ID NO: 11 and SEQ ID NO: 12;
(iii) a heavy chain comprising a heavy chain variable region, wherein the heavy chain variable region comprises CDR1, CDR2 and CDR3 having the amino acid sequence of SEQ ID NO: 14, SEQ ID NO: 15, and SEQ ID NO: 16, and a light chain comprising a light chain variable region, wherein the light chain variable region comprises CDR1, CDR2 and CDR3 having the amino acid sequence of SEQ ID NO: 10, SEQ ID NO: 11, and SEQ ID NO: 12;
(iv) a heavy chain comprising a heavy chain variable region, wherein the heavy chain variable region comprises CDR1, CDR2, and CDR3 having the amino acid sequence of SEQ ID NO: 7, SEQ ID NO: 8, and SEQ ID NO: 9, and a light chain comprising a light chain variable region, wherein the light chain variable region comprises CDR1, CDR2 and CDR3 having the amino acid sequence of SEQ ID NO: 10, SEQ ID NO: 11 and SEQ ID NO: 13;
(v) a heavy chain comprising a heavy chain variable region, wherein the heavy chain variable region comprises CDR1, CDR2 and CDR3 having the amino acid sequence of SEQ ID NO: 14, SEQ ID NO: 15, and SEQ ID NO: 16, and a light chain comprising a light chain variable region, wherein the light chain variable region comprises CDR1, CDR2 and CDR3 having the amino acid sequence of SEQ ID NO: 10, SEQ ID NO: 11, and SEQ ID NO: 13;
(vi) a heavy chain comprising a heavy chain variable region, wherein the heavy chain variable region comprises CDR1, CDR2 and CDR3 having the amino acid sequence of SEQ ID NO: 53, SEQ ID NO: 54, and SEQ ID NO: 55, and a light chain comprising a light chain variable region, wherein the light chain variable region comprises CDR1, CDR2 and CDR3 having the amino acid sequence of SEQ ID NO: 56, SEQ ID NO: 57, and SEQ ID NO: 58;
(vii) a heavy chain comprising a heavy chain variable region, wherein the heavy chain variable region comprises CDR1, CDR2 and CDR3 having the amino acid sequence of SEQ ID NO: 23, SEQ ID NO: 24, and SEQ ID NO: 25, and a light chain comprising a light chain variable region, wherein the light chain variable region comprises CDR1, CDR2 and CDR3 having the amino acid sequence of SEQ ID NO: 26, SEQ ID NO: 27, and SEQ ID NO: 28; or
(viii) a heavy chain comprising a heavy chain variable region, wherein the heavy chain variable region comprises CDR1, CDR2 and CDR3 having the amino acid sequence of SEQ ID NO: 29, SEQ ID NO: 30, and SEQ ID NO: 31, and a light chain comprising a light chain variable region, wherein the light chain variable region comprises CDR1, CDR2 and CDR3 having the amino acid sequence of SEQ ID NO: 32, SEQ ID NO: 33, and SEQ ID NO: 34.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region comprises CDR1, CDR2, and CDR3 having the amino acid sequence of SEQ ID NO: 1, SEQ ID NO: 2, and SEQ ID NO: 3, and a light chain comprising a light chain variable region, wherein the light chain variable region comprises CDR1, CDR2, and CDR3 having the amino acid sequence of SEQ ID NO: 4, SEQ ID NO: 5, and SEQ ID NO: 6.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region comprises CDR1, CDR2, and CDR3 having the amino acid sequence of SEQ ID NO: 7, SEQ ID NO: 8, and SEQ ID NO: 9, and a light chain comprising a light chain variable region, wherein the light chain variable region comprises CDR1, CDR2 and CDR3 having the amino acid sequence of SEQ ID NO: 10, SEQ ID NO: 11 and SEQ ID NO: 12.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region comprises CDR1, CDR2 and CDR3 having the amino acid sequence of SEQ ID NO: 14, SEQ ID NO: 15, and SEQ ID NO: 16, and a light chain comprising a light chain variable region, wherein the light chain variable region comprises CDR1, CDR2 and CDR3 having the amino acid sequence of SEQ ID NO: 10, SEQ ID NO: 11, and SEQ ID NO: 12.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region comprises CDR1, CDR2, and CDR3 having the amino acid sequence of SEQ ID NO: 7, SEQ ID NO: 8, and SEQ ID NO: 9, and a light chain comprising a light chain variable region, wherein the light chain variable region comprises CDR1, CDR2 and CDR3 having the amino acid sequence of SEQ ID NO: 10, SEQ ID NO: 11 and SEQ ID NO: 13.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region comprises CDR1, CDR2 and CDR3 having the amino acid sequence of SEQ ID NO: 14, SEQ ID NO: 15, and SEQ ID NO: 16, and a light chain comprising a light chain variable region, wherein the light chain variable region comprises CDR1, CDR2 and CDR3 having the amino acid sequence of SEQ ID NO: 10, SEQ ID NO: 11, and SEQ ID NO: 13.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region comprises CDR1, CDR2 and CDR3 having the amino acid sequence of SEQ ID NO: 53, SEQ ID NO: 54, and SEQ ID NO: 55, and a light chain comprising a light chain variable region, wherein the light chain variable region comprises CDR1, CDR2 and CDR3 having the amino acid sequence of SEQ ID NO: 56, SEQ ID NO: 57, and SEQ ID NO: 58.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region comprises CDR1, CDR2 and CDR3 having the amino acid sequence of SEQ ID NO: 23, SEQ ID NO: 24, and SEQ ID NO: 25, and a light chain comprising a light chain variable region, wherein the light chain variable region comprises CDR1, CDR2 and CDR3 having the amino acid sequence of SEQ ID NO: 26, SEQ ID NO: 27, and SEQ ID NO: 28.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region comprises CDR1, CDR2 and CDR3 having the amino acid sequence of SEQ ID NO: 29, SEQ ID NO: 30, and SEQ ID NO: 31, and a light chain comprising a light chain variable region, wherein the light chain variable region comprises CDR1, CDR2 and CDR3 having the amino acid sequence of SEQ ID NO: 32, SEQ ID NO: 33, and SEQ ID NO: 34.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region comprises CDR1, CDR2 and CDR3 having the amino acid sequence of SEQ ID NO: 97, SEQ ID NO: 99, and SEQ ID NO: 101, and a light chain comprising a light chain variable region, wherein the light chain variable region comprises CDR1, CDR2 and CDR3 having the amino acid sequence of SEQ ID NO: 103, SEQ ID NO: 105, and SEQ ID NO: 107.

In some embodiments, the CDR described herein has one or two positions mutated. In some embodiments, the CDR comprising one or two mutated positions retain binding activity specific to misfolded SOD1 epitope having an amino acid sequence shown in SEQ ID NO: 142 or 144.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises:
(i) a heavy chain having a heavy chain variable region comprising an amino acid sequence having at least 85%, 90%, 95%, 97%, 98%, 99%, 99.5%, 99.5% or 100% identity to the amino acid sequence of SEQ ID NO: 65, and a light chain having a light chain variable region comprising an amino acid sequence having at least 85%, 90%, 95%, 97%, 98%, 99%, 99.5%, 99.5% or 100% identity to the amino acid sequence of SEQ ID NO: 70;
(ii) a heavy chain having a heavy chain variable region comprising an amino acid sequence having at least 85%, 90%, 95%, 97%, 98%, 99%, 99.5%, 99.5% or 100% identity to the amino acid sequence of SEQ ID NO: 68, and a light chain having a light chain variable region comprising an amino acid sequence having at least 85%, 90%, 95%, 97%, 98%, 99%, 99.5%, 99.5% or 100% identity to the amino acid sequence of SEQ ID NO: 70;
(iii) a heavy chain having a heavy chain variable region comprising an amino acid sequence having at least 85%, 90%, 95%, 97%, 98%, 99%, 99.5%, 99.5% or 100% identity to the amino acid sequence of SEQ ID NO: 72, and a light chain having a light chain variable region comprising an amino acid sequence having at least 85%, 90%, 95%, 97%, 98%, 99%, 99.5%, 99.5% or 100% identity to the amino acid sequence of SEQ ID NO: 76;
(iv) a heavy chain having a heavy chain variable region comprising an amino acid sequence having at least 85%, 90%, 95%, 97%, 98%, 99%, 99.5%, 99.5% or 100% identity to the amino acid sequence of SEQ ID NO: 74, and a light chain having a light chain variable region comprising an amino acid sequence having at least 85%, 90%, 95%, 97%, 98%, 99%, 99.5%, 99.5% or 100% identity to the amino acid sequence of SEQ ID NO: 76;
(v) a heavy chain having a heavy chain variable region comprising an amino acid sequence having at least 85%, 90%, 95%, 97%, 98%, 99%, 99.5%, 99.5% or 100% identity to the amino acid sequence of SEQ ID NO: 78, and a light chain having a light chain variable region comprising an amino acid sequence having at least 85%, 90%, 95%, 97%, 98%, 99%, 99.5%, 99.5% or 100% identity to the amino acid sequence of SEQ ID NO: 82;
(vi) a heavy chain having a heavy chain variable region comprising an amino acid sequence having at least 85%, 90%, 95%, 97%, 98%, 99%, 99.5%, 99.5% or 100% identity to the amino acid sequence of SEQ ID NO: 80, and a light chain having a light chain variable region comprising an amino acid sequence having at least 85%, 90%, 95%, 97%, 98%, 99%, 99.5%, 99.5% or 100% identity to the amino acid sequence of SEQ ID NO: 82;
(vii) a heavy chain having a heavy chain variable region comprising an amino acid sequence having at least 85%, 90%, 95%, 97%, 98%, 99%, 99.5%, 99.5% or 100% identity to the amino acid sequence of SEQ ID NO: 84, and a light chain having a light chain variable region comprising an amino acid sequence having at least 85%, 90%, 95%, 97%, 98%, 99%, 99.5%, 99.5% or 100% identity to the amino acid sequence of SEQ ID NO: 86; or
(viii) a heavy chain having a heavy chain variable region comprising an amino acid sequence having at least 85%, 90%, 95%, 97%, 98%, 99%, 99.5%, 99.5% or 100% identity to the amino acid sequence of SEQ ID NO: 88, and a light chain having a light chain variable region comprising an amino acid sequence having at least 85%, 90%, 95%, 97%, 98%, 99%, 99.5%, 99.5% or 100% identity to the amino acid sequence of SEQ ID NO: 91.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises a heavy chain having a heavy chain variable region comprising an amino acid sequence having at least 85%, 90%, 95%, 97%, 98%, 99%, 99.5%, 99.5% or 100% identity to the amino acid sequence of SEQ ID NO: 65, and a light chain having a light chain variable region comprising an amino acid sequence having at least 85%, 90%, 95%, 97%, 98%, 99%, 99.5%, 99.5% or 100% identity to the amino acid sequence of SEQ ID NO: 70.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises a heavy chain having a heavy chain variable region comprising an amino acid sequence having at least 85%, 90%, 95%, 97%, 98%, 99%, 99.5%, 99.5% or 100% identity to the amino acid sequence of SEQ ID NO: 68, and a light chain having a light chain variable region comprising an amino acid sequence having at least 85%, 90%, 95%, 97%, 98%, 99%, 99.5%, 99.5% or 100% identity to the amino acid sequence of SEQ ID NO: 70.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises a heavy chain having a heavy chain variable region comprising an amino acid sequence having at least 85%, 90%, 95%, 97%, 98%, 99%, 99.5%, 99.5% or 100% identity to the amino acid sequence of SEQ ID NO: 72, and a light chain having a light chain variable region comprising an amino acid sequence having at least 85%, 90%, 95%, 97%, 98%, 99%, 99.5%, 99.5% or 100% identity to the amino acid sequence of SEQ ID NO: 76.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises a heavy chain having a heavy chain variable region comprising an amino acid sequence having at least 85%, 90%, 95%, 97%, 98%, 99%, 99.5%, 99.5% or 100% identity to the amino acid sequence of SEQ ID NO: 74, and a light chain having a light chain variable region comprising an amino acid sequence having at least 85%, 90%, 95%, 97%, 98%, 99%, 99.5%, 99.5% or 100% identity to the amino acid sequence of SEQ ID NO: 76.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises a heavy chain having a heavy chain variable region comprising an amino acid sequence having at least 85%, 90%, 95%, 97%, 98%, 99%, 99.5%, 99.5% or 100% identity to the amino acid sequence of SEQ ID NO: 78, and a light chain having a light chain variable region comprising an amino acid sequence having at least 85%, 90%, 95%, 97%, 98%, 99%, 99.5%, 99.5% or 100% identity to the amino acid sequence of SEQ ID NO: 82.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises a heavy chain having a heavy chain variable region comprising an amino acid sequence having at least 85%, 90%, 95%, 97%, 98%, 99%, 99.5%, 99.5% or 100% identity to the amino acid sequence of SEQ ID NO: 80, and a light chain having a light chain variable region comprising an amino acid sequence having at least 85%, 90%, 95%, 97%, 98%, 99%, 99.5%, 99.5% or 100% identity to the amino acid sequence of SEQ ID NO: 82.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises a heavy chain having a heavy chain variable region comprising an amino acid sequence having at least 85%, 90%, 95%, 97%, 98%, 99%, 99.5%, 99.5% or 100% identity to the amino acid sequence of SEQ ID NO: 84, and a light chain having a light chain variable region comprising an amino acid sequence having at least 85%, 90%, 95%, 97%, 98%, 99%, 99.5%, 99.5% or 100% identity to the amino acid sequence of SEQ ID NO: 86.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises a heavy chain having a heavy chain variable region comprising an amino acid sequence having at least 85%, 90%, 95%, 97%, 98%, 99%, 99.5%, 99.5% or 100% identity to the amino acid sequence of SEQ ID NO: 88, and a light chain having a light chain variable region comprising an amino acid sequence having at least 85%, 90%, 95%, 97%, 98%, 99%, 99.5%, 99.5% or 100% identity to the amino acid sequence of SEQ ID NO: 91.

In some embodiments, the fusion protein comprises a heavy chain variable region, a first linker, a light chain variable region, a second linker, and an active E3 ligase fragment. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 65, a first linker, a light chain variable region comprising an amino acid sequence having at least 85%, 90%, 95%, 97%, 98%, 99%, 99.5%, 99.5% or 100% identity to the amino acid sequence of SEQ ID NO: 70, a second linker, and an active E3 ligase fragment comprising an amino acid sequence having at least 85%, 90%, 95%, 97%, 98%, 99%, 99.5%, 99.5% or 100% identity to the amino acid sequence of any one of SEQ ID NOs: 186-193, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 68, a first linker, a light chain variable region comprising an amino acid sequence having at least 85%, 90%, 95%, 97%, 98%, 99%, 99.5%, 99.5% or 100% identity to the amino acid sequence of SEQ ID NO: 70, a second linker, and an active E3 ligase fragment comprising an amino acid sequence having at least 85%, 90%, 95%, 97%, 98%, 99%, 99.5%, 99.5% or 100% identity to the amino acid sequence of any one of SEQ ID NOs: 186-193, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 72, a first linker, a light chain variable region comprising an amino acid sequence having at least 85%, 90%, 95%, 97%, 98%, 99%, 99.5%, 99.5% or 100% identity to the amino acid sequence of SEQ ID NO: 76, a second linker, and an active E3 ligase fragment comprising an amino acid sequence having at least 85%, 90%, 95%, 97%, 98%, 99%, 99.5%, 99.5% or 100% identity to the amino acid sequence of any one of SEQ ID NOs: 186-193, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 74, a first linker, a light chain variable region comprising an amino acid sequence having at least 85%, 90%, 95%, 97%, 98%, 99%, 99.5%, 99.5% or 100% identity to the amino acid sequence of SEQ ID NO: 76, a second linker, and an active E3 ligase fragment comprising an amino acid sequence having at least 85%, 90%, 95%, 97%, 98%, 99%, 99.5%, 99.5% or 100% identity to the amino acid sequence of any one of SEQ ID NOs: 186-193, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 78, a first linker, a light chain variable region comprising an amino acid sequence having at least 85%, 90%, 95%, 97%, 98%, 99%, 99.5%, 99.5% or 100% identity to the amino acid sequence of SEQ ID NO: 82, a second linker, and an active E3 ligase fragment comprising an amino acid sequence having at least 85%, 90%, 95%, 97%, 98%, 99%, 99.5%, 99.5% or 100% identity to the amino acid sequence of any one of SEQ ID NOs: 186-193, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 80, a first linker, a light chain variable region comprising an amino acid sequence having at least 85%, 90%, 95%, 97%, 98%, 99%, 99.5%, 99.5% or 100% identity to the amino acid sequence of SEQ ID NO: 82, a second linker, and an active E3 ligase fragment comprising an amino acid sequence having at least 85%, 90%, 95%, 97%, 98%, 99%, 99.5%, 99.5% or 100% identity to the amino acid sequence of any one of SEQ ID NOs: 186-193, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 84, a first linker, a light chain variable region comprising an amino acid sequence having at least 85%, 90%, 95%, 97%, 98%, 99%, 99.5%, 99.5% or 100% identity to the amino acid sequence of SEQ ID NO: 86, a second linker, and an active E3 ligase fragment comprising an amino acid sequence having at least 85%, 90%, 95%, 97%, 98%, 99%, 99.5%, 99.5% or 100% identity to the amino acid sequence of any one of SEQ ID NOs: 186-193, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 88, a first linker, a light chain variable region comprising an amino acid sequence having at least 85%, 90%, 95%, 97%, 98%, 99%, 99.5%, 99.5% or 100% identity to the amino acid sequence of SEQ ID NO: 91, a second linker, and an active E3 ligase fragment comprising an amino acid sequence having at least 85%, 90%, 95%, 97%, 98%, 99%, 99.5%, 99.5% or 100% identity to the amino acid sequence of any one of SEQ ID NOs: 186-193, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 65, a first linker, a light chain variable region comprising an amino acid sequence having at least 85%, 90%, 95%, 97%, 98%, 99%, 99.5%, 99.5% or 100% identity to the amino acid sequence of SEQ ID NO: 70, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 186, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 68, a first linker, a light chain variable region comprising an amino acid sequence having at least 85%, 90%, 95%, 97%, 98%, 99%, 99.5%, 99.5% or 100% identity to the amino acid sequence of SEQ ID NO: 70, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 186, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 72, a first linker, a light chain variable region comprising an amino acid sequence having at least 85%, 90%, 95%, 97%, 98%, 99%, 99.5%, 99.5% or 100% identity to the amino acid sequence of SEQ ID NO: 76, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 186, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 74, a first linker, a light chain variable region comprising an amino acid sequence having at least 85%, 90%, 95%, 97%, 98%, 99%, 99.5%, 99.5% or 100% identity to the amino acid sequence of SEQ ID NO: 76, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 186, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 78, a first linker, a light chain variable region comprising an amino acid sequence having at least 85%, 90%, 95%, 97%, 98%, 99%, 99.5%, 99.5% or 100% identity to the amino acid sequence of SEQ ID NO: 82, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 186, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 80, a first linker, a light chain variable region comprising an amino acid sequence having at least 85%, 90%, 95%, 97%, 98%, 99%, 99.5%, 99.5% or 100% identity to the amino acid sequence of SEQ ID NO: 82, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 186, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 84, a first linker, a light chain variable region comprising an amino acid sequence having at least 85%, 90%, 95%, 97%, 98%, 99%, 99.5%, 99.5% or 100% identity to the amino acid sequence of SEQ ID NO: 86, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 186, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 88, a first linker, a light chain variable region comprising an amino acid sequence having at least 85%, 90%, 95%, 97%, 98%, 99%, 99.5%, 99.5% or 100% identity to the amino acid sequence of SEQ ID NO: 91, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 186, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 65, a first linker, a light chain variable region comprising an amino acid sequence having at least 85%, 90%, 95%, 97%, 98%, 99%, 99.5%, 99.5% or 100% identity to the amino acid sequence of SEQ ID NO: 70, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 187. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 68, a first linker, a light chain variable region comprising an amino acid sequence having at least 85%, 90%, 95%, 97%, 98%, 99%, 99.5%, 99.5% or 100% identity to the amino acid sequence of SEQ ID NO: 70, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 187. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 72, a first linker, a light chain variable region comprising an amino acid sequence having at least 85%, 90%, 95%, 97%, 98%, 99%, 99.5%, 99.5% or 100% identity to the amino acid sequence of SEQ ID NO: 76, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 187. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 74, a first linker, a light chain variable region comprising an amino acid sequence having at least 85%, 90%, 95%, 97%, 98%, 99%, 99.5%, 99.5% or 100% identity to the amino acid sequence of SEQ ID NO: 76, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 187. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 78, a first linker, a light chain variable region comprising an amino acid sequence having at least 85%, 90%, 95%, 97%, 98%, 99%, 99.5%, 99.5% or 100% identity to the amino acid sequence of SEQ ID NO: 82, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 187. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 80, a first linker, a light chain variable region comprising an amino acid sequence having at least 85%, 90%, 95%, 97%, 98%, 99%, 99.5%, 99.5% or 100% identity to the amino acid sequence of SEQ ID NO: 82, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 187. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 84, a first linker, a light chain variable region comprising an amino acid sequence having at least 85%, 90%, 95%, 97%, 98%, 99%, 99.5%, 99.5% or 100% identity to the amino acid sequence of SEQ ID NO: 86, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 187. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 88, a first linker, a light chain variable region comprising an amino acid sequence having at least 85%, 90%, 95%, 97%, 98%, 99%, 99.5%, 99.5% or 100% identity to the amino acid sequence of SEQ ID NO: 91, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 187. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 65, a first linker, a light chain variable region comprising an amino acid sequence having at least 85%, 90%, 95%, 97%, 98%, 99%, 99.5%, 99.5% or 100% identity to the amino acid sequence of SEQ ID NO: 70, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 188. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 68, a first linker, a light chain variable region comprising an amino acid sequence having at least 85%, 90%, 95%, 97%, 98%, 99%, 99.5%, 99.5% or 100% identity to the amino acid sequence of SEQ ID NO: 70, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 188. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 72, a first linker, a light chain variable region comprising an amino acid sequence having at least 85%, 90%, 95%, 97%, 98%, 99%, 99.5%, 99.5% or 100% identity to the amino acid sequence of SEQ ID NO: 76, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 188. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 74, a first linker, a light chain variable region comprising an amino acid sequence having at least 85%, 90%, 95%, 97%, 98%, 99%, 99.5%, 99.5% or 100% identity to the amino acid sequence of SEQ ID NO: 76, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 188. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 78, a first linker, a light chain variable region comprising an amino acid sequence having at least 85%, 90%, 95%, 97%, 98%, 99%, 99.5%, 99.5% or 100% identity to the amino acid sequence of SEQ ID NO: 82, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 188. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 80, a first linker, a light chain variable region comprising an amino acid sequence having at least 85%, 90%, 95%, 97%, 98%, 99%, 99.5%, 99.5% or 100% identity to the amino acid sequence of SEQ ID NO: 82, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 188. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 84, a first linker, a light chain variable region comprising an amino acid sequence having at least 85%, 90%, 95%, 97%, 98%, 99%, 99.5%, 99.5% or 100% identity to the amino acid sequence of SEQ ID NO: 86, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 188. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 88, a first linker, a light chain variable region comprising an amino acid sequence having at least 85%, 90%, 95%, 97%, 98%, 99%, 99.5%, 99.5% or 100% identity to the amino acid sequence of SEQ ID NO: 91, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 188. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 65, a first linker, a light chain variable region comprising an amino acid sequence having at least 85%, 90%, 95%, 97%, 98%, 99%, 99.5%, 99.5% or 100% identity to the amino acid sequence of SEQ ID NO: 70, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 189. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 68, a first linker, a light chain variable region comprising an amino acid sequence having at least 85%, 90%, 95%, 97%, 98%, 99%, 99.5%, 99.5% or 100% identity to the amino acid sequence of SEQ ID NO: 70, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 189. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 72, a first linker, a light chain variable region comprising an amino acid sequence having at least 85%, 90%, 95%, 97%, 98%, 99%, 99.5%, 99.5% or 100% identity to the amino acid sequence of SEQ ID NO: 76, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 189. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 74, a first linker, a light chain variable region comprising an amino acid sequence having at least 85%, 90%, 95%, 97%, 98%, 99%, 99.5%, 99.5% or 100% identity to the amino acid sequence of SEQ ID NO: 76, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 189. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 78, a first linker, a light chain variable region comprising an amino acid sequence having at least 85%, 90%, 95%, 97%, 98%, 99%, 99.5%, 99.5% or 100% identity to the amino acid sequence of SEQ ID NO: 82, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 189. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 80, a first linker, a light chain variable region comprising an amino acid sequence having at least 85%, 90%, 95%, 97%, 98%, 99%, 99.5%, 99.5% or 100% identity to the amino acid sequence of SEQ ID NO: 82, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 189. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 84, a first linker, a light chain variable region comprising an amino acid sequence having at least 85%, 90%, 95%, 97%, 98%, 99%, 99.5%, 99.5% or 100% identity to the amino acid sequence of SEQ ID NO: 86, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 189. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 88, a first linker, a light chain variable region comprising an amino acid sequence having at least 85%, 90%, 95%, 97%, 98%, 99%, 99.5%, 99.5% or 100% identity to the amino acid sequence of SEQ ID NO: 91, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 189. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 65, a first linker, a light chain variable region comprising an amino acid sequence having at least 85%, 90%, 95%, 97%, 98%, 99%, 99.5%, 99.5% or 100% identity to the amino acid sequence of SEQ ID NO: 70, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 190. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 68, a first linker, a light chain variable region comprising an amino acid sequence having at least 85%, 90%, 95%, 97%, 98%, 99%, 99.5%, 99.5% or 100% identity to the amino acid sequence of SEQ ID NO: 70, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 190. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 72, a first linker, a light chain variable region comprising an amino acid sequence having at least 85%, 90%, 95%, 97%, 98%, 99%, 99.5%, 99.5% or 100% identity to the amino acid sequence of SEQ ID NO: 76, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 190. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 74, a first linker, a light chain variable region comprising an amino acid sequence having at least 85%, 90%, 95%, 97%, 98%, 99%, 99.5%, 99.5% or 100% identity to the amino acid sequence of SEQ ID NO: 76, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 190. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 78, a first linker, a light chain variable region comprising an amino acid sequence having at least 85%, 90%, 95%, 97%, 98%, 99%, 99.5%, 99.5% or 100% identity to the amino acid sequence of SEQ ID NO: 82, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 190. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 80, a first linker, a light chain variable region comprising an amino acid sequence having at least 85%, 90%, 95%, 97%, 98%, 99%, 99.5%, 99.5% or 100% identity to the amino acid sequence of SEQ ID NO: 82, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 190. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 84, a first linker, a light chain variable region comprising an amino acid sequence having at least 85%, 90%, 95%, 97%, 98%, 99%, 99.5%, 99.5% or 100% identity to the amino acid sequence of SEQ ID NO: 86, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 190. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 88, a first linker, a light chain variable region comprising an amino acid sequence having at least 85%, 90%, 95%, 97%, 98%, 99%, 99.5%, 99.5% or 100% identity to the amino acid sequence of SEQ ID NO: 91, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 190. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 65, a first linker, a light chain variable region comprising an amino acid sequence having at least 85%, 90%, 95%, 97%, 98%, 99%, 99.5%, 99.5% or 100% identity to the amino acid sequence of SEQ ID NO: 70, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 191. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 68, a first linker, a light chain variable region comprising an amino acid sequence having at least 85%, 90%, 95%, 97%, 98%, 99%, 99.5%, 99.5% or 100% identity to the amino acid sequence of SEQ ID NO: 70, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 191. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 72, a first linker, a light chain variable region comprising an amino acid sequence having at least 85%, 90%, 95%, 97%, 98%, 99%, 99.5%, 99.5% or 100% identity to the amino acid sequence of SEQ ID NO: 76, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 191. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 74, a first linker, a light chain variable region comprising an amino acid sequence having at least 85%, 90%, 95%, 97%, 98%, 99%, 99.5%, 99.5% or 100% identity to the amino acid sequence of SEQ ID NO: 76, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 191. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 78, a first linker, a light chain variable region comprising an amino acid sequence having at least 85%, 90%, 95%, 97%, 98%, 99%, 99.5%, 99.5% or 100% identity to the amino acid sequence of SEQ ID NO: 82, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 191. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 80, a first linker, a light chain variable region comprising an amino acid sequence having at least 85%, 90%, 95%, 97%, 98%, 99%, 99.5%, 99.5% or 100% identity to the amino acid sequence of SEQ ID NO: 82, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 191. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 84, a first linker, a light chain variable region comprising an amino acid sequence having at least 85%, 90%, 95%, 97%, 98%, 99%, 99.5%, 99.5% or 100% identity to the amino acid sequence of SEQ ID NO: 86, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 191. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 88, a first linker, a light chain variable region comprising an amino acid sequence having at least 85%, 90%, 95%, 97%, 98%, 99%, 99.5%, 99.5% or 100% identity to the amino acid sequence of SEQ ID NO: 91, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 191. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 65, a first linker, a light chain variable region comprising an amino acid sequence having at least 85%, 90%, 95%, 97%, 98%, 99%, 99.5%, 99.5% or 100% identity to the amino acid sequence of SEQ ID NO: 70, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 192. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 68, a first linker, a light chain variable region comprising an amino acid sequence having at least 85%, 90%, 95%, 97%, 98%, 99%, 99.5%, 99.5% or 100% identity to the amino acid sequence of SEQ ID NO: 70, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 192. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 72, a first linker, a light chain variable region comprising an amino acid sequence having at least 85%, 90%, 95%, 97%, 98%, 99%, 99.5%, 99.5% or 100% identity to the amino acid sequence of SEQ ID NO: 76, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 192. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 74, a first linker, a light chain variable region comprising an amino acid sequence having at least 85%, 90%, 95%, 97%, 98%, 99%, 99.5%, 99.5% or 100% identity to the amino acid sequence of SEQ ID NO: 76, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 192. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 78, a first linker, a light chain variable region comprising an amino acid sequence having at least 85%, 90%, 95%, 97%, 98%, 99%, 99.5%, 99.5% or 100% identity to the amino acid sequence of SEQ ID NO: 82, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 192. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 80, a first linker, a light chain variable region comprising an amino acid sequence having at least 85%, 90%, 95%, 97%, 98%, 99%, 99.5%, 99.5% or 100% identity to the amino acid sequence of SEQ ID NO: 82, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 192. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 84, a first linker, a light chain variable region comprising an amino acid sequence having at least 85%, 90%, 95%, 97%, 98%, 99%, 99.5%, 99.5% or 100% identity to the amino acid sequence of SEQ ID NO: 86, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 192. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 88, a first linker, a light chain variable region comprising an amino acid sequence having at least 85%, 90%, 95%, 97%, 98, 99%, 99.5%, 99.5% or 100% identity to the amino acid sequence of SEQ ID NO: 91, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 192. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 65, a first linker, a light chain variable region comprising an amino acid sequence having at least 85%, 90%, 95%, 97%, 98%, 99%, 99.5%, 99.5% or 100% identity to the amino acid sequence of SEQ ID NO: 70, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 193. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 68, a first linker, a light chain variable region comprising an amino acid sequence having at least 85%, 90%, 95%, 97%, 98%, 99%, 99.5%, 99.5% or 100% identity to the amino acid sequence of SEQ ID NO: 70, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 193. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 72, a first linker, a light chain variable region comprising an amino acid sequence having at least 85%, 90%, 95%, 97%, 98%, 99%, 99.5%, 99.5% or 100% identity to the amino acid sequence of SEQ ID NO: 76, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 193. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 74, a first linker, a light chain variable region comprising an amino acid sequence having at least 85%, 90%, 95%, 97%, 98%, 99%, 99.5%, 99.5% or 100% identity to the amino acid sequence of SEQ ID NO: 76, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 193. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 78, a first linker, a light chain variable region comprising an amino acid sequence having at least 85%, 90%, 95%, 97%, 98%, 99%, 99.5%, 99.5% or 100% identity to the amino acid sequence of SEQ ID NO: 82, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 193. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 80, a first linker, a light chain variable region comprising an amino acid sequence having at least 85%, 90%, 95%, 97%, 98%, 99%, 99.5%, 99.5% or 100% identity to the amino acid sequence of SEQ ID NO: 82, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 193. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 84, a first linker, a light chain variable region comprising an amino acid sequence having at least 85%, 90%, 95%, 97%, 98%, 99%, 99.5%, 99.5% or 100% identity to the amino acid sequence of SEQ ID NO: 86, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 193. In some embodiments, the fusion protein comprises a heavy chain variable region comprising an amino acid sequence having at least 85%, 90%, 95%, 97%, 98%, 99%, 99.5%, 99.5% or 100% identity to the amino acid sequence of SEQ ID NO: 88, a first linker, a light chain variable region comprising an amino acid sequence having at least 85%, 90%, 95%, 97%, 98%, 99%, 99.5%, 99.5% or 100% identity to the amino acid sequence of SEQ ID NO: 91, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 193. In some embodiments, the fusion protein comprises a heavy chain variable region comprising CDR1, CDR2, and CDR3 having the amino acid sequence of SEQ ID NO: 1, SEQ ID NO: 2, and SEQ ID NO: 3, a first linker, a light chain variable region comprising CDR1, CDR2, and CDR3 having the amino acid sequence of SEQ ID NO: 4, SEQ ID NO: 5, and SEQ ID NO: 6, a second linker, and an active E3 ligase fragment comprising an amino acid sequence having at least 85%, 90%, 95%, 97%, 98%, 99%, 99.5%, 99.5% or 100% identity to the amino acid sequence of any one of SEQ ID NOs: 186-193, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising CDR1, CDR2, and CDR3 having the amino acid sequence of SEQ ID NO: 7, SEQ ID NO: 8, and SEQ ID NO: 9, a first linker, a light chain variable region comprising CDR1, CDR2 and CDR3 having the amino acid sequence of SEQ ID NO: 10, SEQ ID NO: 11 and SEQ ID NO: 12, a second linker, and an active E3 ligase fragment comprising an amino acid sequence having at least 85%, 90%, 95%, 97%, 98%, 99%, 99.5%, 99.5% or 100% identity to the amino acid sequence of any one of SEQ ID NOs: 186-193, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising CDR1, CDR2 and CDR3 having the amino acid sequence of SEQ ID NO: 14, SEQ ID NO: 15, and SEQ ID NO: 16, a first linker, a light chain variable region comprising CDR1, CDR2 and CDR3 having the amino acid sequence of SEQ ID NO: 10, SEQ ID NO: 11, and SEQ ID NO: 12, a second linker, and an active E3 ligase fragment comprising an amino acid sequence having at least 85%, 90%, 95%, 97%, 98%, 99%, 99.5%, 99.5% or 100% identity to the amino acid sequence of any one of SEQ ID NOs: 186-193, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising CDR1, CDR2, and CDR3 having the amino acid sequence of SEQ ID NO: 7, SEQ ID NO: 8, and SEQ ID NO: 9, a first linker, a light chain variable region comprising CDR1, CDR2 and CDR3 having the amino acid sequence of SEQ ID NO: 10, SEQ ID NO: 11 and SEQ ID NO: 13, a second linker, and an active E3 ligase fragment comprising an amino acid sequence having at least 85%, 90%, 95%, 97%, 98%, 99%, 99.5%, 99.5% or 100% identity to the amino acid sequence of any one of SEQ ID NOs: 186-193, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a the heavy chain variable region comprising CDR1, CDR2 and CDR3 having the amino acid sequence of SEQ ID NO: 14, SEQ ID NO: 15, and SEQ ID NO: 16, a first linker, a light chain variable region comprising CDR1, CDR2 and CDR3 having the amino acid sequence of SEQ ID NO: 10, SEQ ID NO: 11, and SEQ ID NO: 13, a second linker, and an active E3 ligase fragment comprising an amino acid sequence having at least 85%, 90%, 95%, 97%, 98%, 99%, 99.5%, 99.5% or 100% identity to the amino acid sequence of any one of SEQ ID NOs: 186-193, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising CDR1, CDR2 and CDR3 having the amino acid sequence of SEQ ID NO: 53, SEQ ID NO: 54, and SEQ ID NO: 55, a first linker, a the light chain variable region comprising CDR1, CDR2 and CDR3 having the amino acid sequence of SEQ ID NO: 56, SEQ ID NO: 57, and SEQ ID NO: 58, a second linker, and an active E3 ligase fragment comprising an amino acid sequence having at least 85%, 90%, 95%, 97%, 98%, 99%, 99.5%, 99.5% or 100% identity to the amino acid sequence of any one of SEQ ID NOs: 186-193, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising CDR1, CDR2 and CDR3 having the amino acid sequence of SEQ ID NO: 23, SEQ ID NO: 24, and SEQ ID NO: 25, a first linker, a light chain variable region comprising CDR1, CDR2 and CDR3 having the amino acid sequence of SEQ ID NO: 26, SEQ ID NO: 27, and SEQ ID NO: 28, a second linker, and an active E3 ligase fragment comprising an amino acid sequence having at least 85%, 90%, 95%, 97%, 98%, 99%, 99.5%, 99.5% or 100% identity to the amino acid sequence of any one of SEQ ID NOs: 186-193, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising CDR1, CDR2 and CDR3 having the amino acid sequence of SEQ ID NO: 29, SEQ ID NO: 30, and SEQ ID NO: 31, a first linker, a light chain variable region comprising CDR1, CDR2 and CDR3 having the amino acid sequence of SEQ ID NO: 32, SEQ ID NO: 33, and SEQ ID NO: 34, a second linker, and an active E3 ligase fragment comprising an amino acid sequence having at least 85%, 90%, 95%, 97%, 98%, 99%, 99.5%, 99.5% or 100% identity to the amino acid sequence of any one of SEQ ID NOs: 186-193, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising CDR1, CDR2, and CDR3 having the amino acid sequence of SEQ ID NO: 1, SEQ ID NO: 2, and SEQ ID NO: 3, a first linker, a light chain variable region comprising CDR1, CDR2, and CDR3 having the amino acid sequence of SEQ ID NO: 4, SEQ ID NO: 5, and SEQ ID NO: 6, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 186, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising CDR1, CDR2, and CDR3 having the amino acid sequence of SEQ ID NO: 7, SEQ ID NO: 8, and SEQ ID NO: 9, a first linker, a light chain variable region comprising CDR1, CDR2 and CDR3 having the amino acid sequence of SEQ ID NO: 10, SEQ ID NO: 11 and SEQ ID NO: 12, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 186, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising CDR1, CDR2 and CDR3 having the amino acid sequence of SEQ ID NO: 14, SEQ ID NO: 15, and SEQ ID NO: 16, a first linker, a light chain variable region comprising CDR1, CDR2 and CDR3 having the amino acid sequence of SEQ ID NO: 10, SEQ ID NO: 11, and SEQ ID NO: 12, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 186, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising CDR1, CDR2, and CDR3 having the amino acid sequence of SEQ ID NO: 7, SEQ ID NO: 8, and SEQ ID NO: 9, a first linker, a light chain variable region comprising CDR1, CDR2 and CDR3 having the amino acid sequence of SEQ ID NO: 10, SEQ ID NO: 11 and SEQ ID NO: 13, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 186, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a the heavy chain variable region comprising CDR1, CDR2 and CDR3 having the amino acid sequence of SEQ ID NO: 14, SEQ ID NO: 15, and SEQ ID NO: 16, a first linker, a light chain variable region comprising CDR1, CDR2 and CDR3 having the amino acid sequence of SEQ ID NO: 10, SEQ ID NO: 11, and SEQ ID NO: 13, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 186, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising CDR1, CDR2 and CDR3 having the amino acid sequence of SEQ ID NO: 53, SEQ ID NO: 54, and SEQ ID NO: 55, a first linker, a the light chain variable region comprising CDR1, CDR2 and CDR3 having the amino acid sequence of SEQ ID NO: 56, SEQ ID NO: 57, and SEQ ID NO: 58, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 186, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising CDR1, CDR2 and CDR3 having the amino acid sequence of SEQ ID NO: 23, SEQ ID NO: 24, and SEQ ID NO: 25, a first linker, a light chain variable region comprising CDR1, CDR2 and CDR3 having the amino acid sequence of SEQ ID NO: 26, SEQ ID NO: 27, and SEQ ID NO: 28, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 186, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising CDR1, CDR2 and CDR3 having the amino acid sequence of SEQ ID NO: 29, SEQ ID NO: 30, and SEQ ID NO: 31, a first linker, a light chain variable region comprising CDR1, CDR2 and CDR3 having the amino acid sequence of SEQ ID NO: 32, SEQ ID NO: 33, and SEQ ID NO: 34, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 186, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the fusion protein comprises a heavy chain variable region comprising CDR1, CDR2, and CDR3 having the amino acid sequence of SEQ ID NO: 1, SEQ ID NO: 2, and SEQ ID NO: 3, a first linker, a light chain variable region comprising CDR1, CDR2, and CDR3 having the amino acid sequence of SEQ ID NO: 4, SEQ ID NO: 5, and SEQ ID NO: 6, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 187. In some embodiments, the fusion protein comprises a heavy chain variable region comprising CDR1, CDR2, and CDR3 having the amino acid sequence of SEQ ID NO: 7, SEQ ID NO: 8, and SEQ ID NO: 9, a first linker, a light chain variable region comprising CDR1, CDR2 and CDR3 having the amino acid sequence of SEQ ID NO: 10, SEQ ID NO: 11 and SEQ ID NO: 12, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 187. In some embodiments, the fusion protein comprises a heavy chain variable region comprising CDR1, CDR2 and CDR3 having the amino acid sequence of SEQ ID NO: 14, SEQ ID NO: 15, and SEQ ID NO: 16, a first linker, a light chain variable region comprising CDR1, CDR2 and CDR3 having the amino acid sequence of SEQ ID NO: 10, SEQ ID NO: 11, and SEQ ID NO: 12, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 187. In some embodiments, the fusion protein comprises a heavy chain variable region comprising CDR1, CDR2, and CDR3 having the amino acid sequence of SEQ ID NO: 7, SEQ ID NO: 8, and SEQ ID NO: 9, a first linker, a light chain variable region comprising CDR1, CDR2 and CDR3 having the amino acid sequence of SEQ ID NO: 10, SEQ ID NO: 11 and SEQ ID NO: 13, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 187. In some embodiments, the fusion protein comprises a the heavy chain variable region comprising CDR1, CDR2 and CDR3 having the amino acid sequence of SEQ ID NO: 14, SEQ ID NO: 15, and SEQ ID NO: 16, a first linker, a light chain variable region comprising CDR1, CDR2 and CDR3 having the amino acid sequence of SEQ ID NO: 10, SEQ ID NO: 11, and SEQ ID NO: 13, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 187. In some embodiments, the fusion protein comprises a heavy chain variable region comprising CDR1, CDR2 and CDR3 having the amino acid sequence of SEQ ID NO: 53, SEQ ID NO: 54, and SEQ ID NO: 55, a first linker, a the light chain variable region comprising CDR1, CDR2 and CDR3 having the amino acid sequence of SEQ ID NO: 56, SEQ ID NO: 57, and SEQ ID NO: 58, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 187. In some embodiments, the fusion protein comprises a heavy chain variable region comprising CDR1, CDR2 and CDR3 having the amino acid sequence of SEQ ID NO: 23, SEQ ID NO: 24, and SEQ ID NO: 25, a first linker, a light chain variable region comprising CDR1, CDR2 and CDR3 having the amino acid sequence of SEQ ID NO: 26, SEQ ID NO: 27, and SEQ ID NO: 28, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 187. In some embodiments, the fusion protein comprises a heavy chain variable region comprising CDR1, CDR2 and CDR3 having the amino acid sequence of SEQ ID NO: 29, SEQ ID NO: 30, and SEQ ID NO: 31, a first linker, a light chain variable region comprising CDR1, CDR2 and CDR3 having the amino acid sequence of SEQ ID NO: 32, SEQ ID NO: 33, and SEQ ID NO: 34, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 187. In some embodiments, the fusion protein comprises a heavy chain variable region comprising CDR1, CDR2, and CDR3 having the amino acid sequence of SEQ ID NO: 1, SEQ ID NO: 2, and SEQ ID NO: 3, a first linker, a light chain variable region comprising CDR1, CDR2, and CDR3 having the amino acid sequence of SEQ ID NO: 4, SEQ ID NO: 5, and SEQ ID NO: 6, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 188. In some embodiments, the fusion protein comprises a heavy chain variable region comprising CDR1, CDR2, and CDR3 having the amino acid sequence of SEQ ID NO: 7, SEQ ID NO: 8, and SEQ ID NO: 9, a first linker, a light chain variable region comprising CDR1, CDR2 and CDR3 having the amino acid sequence of SEQ ID NO: 10, SEQ ID NO: 11 and SEQ ID NO: 12, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 188. In some embodiments, the fusion protein comprises a heavy chain variable region comprising CDR1, CDR2 and CDR3 having the amino acid sequence of SEQ ID NO: 14, SEQ ID NO: 15, and SEQ ID NO: 16, a first linker, a light chain variable region comprising CDR1, CDR2 and CDR3 having the amino acid sequence of SEQ ID NO: 10, SEQ ID NO: 11, and SEQ ID NO: 12, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 188. In some embodiments, the fusion protein comprises a heavy chain variable region comprising CDR1, CDR2, and CDR3 having the amino acid sequence of SEQ ID NO: 7, SEQ ID NO: 8, and SEQ ID NO: 9, a first linker, a light chain variable region comprising CDR1, CDR2 and CDR3 having the amino acid sequence of SEQ ID NO: 10, SEQ ID NO: 11 and SEQ ID NO: 13, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 188. In some embodiments, the fusion protein comprises a the heavy chain variable region comprising CDR1, CDR2 and CDR3 having the amino acid sequence of SEQ ID NO: 14, SEQ ID NO: 15, and SEQ ID NO: 16, a first linker, a light chain variable region comprising CDR1, CDR2 and CDR3 having the amino acid sequence of SEQ ID NO: 10, SEQ ID NO: 11, and SEQ ID NO: 13, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 188. In some embodiments, the fusion protein comprises a heavy chain variable region comprising CDR1, CDR2 and CDR3 having the amino acid sequence of SEQ ID NO: 53, SEQ ID NO: 54, and SEQ ID NO: 55, a first linker, a the light chain variable region comprising CDR1, CDR2 and CDR3 having the amino acid sequence of SEQ ID NO: 56, SEQ ID NO: 57, and SEQ ID NO: 58, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 188. In some embodiments, the fusion protein comprises a heavy chain variable region comprising CDR1, CDR2 and CDR3 having the amino acid sequence of SEQ ID NO: 23, SEQ ID NO: 24, and SEQ ID NO: 25, a first linker, alight chain variable region comprising CDR1, CDR2 and CDR3 having the amino acid sequence of SEQ ID NO: 26, SEQ ID NO: 27, and SEQ ID NO: 28, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 188. In some embodiments, the fusion protein comprises a heavy chain variable region comprising CDR1, CDR2 and CDR3 having the amino acid sequence of SEQ ID NO: 29, SEQ ID NO: 30, and SEQ ID NO: 31, a first linker, a light chain variable region comprising CDR1, CDR2 and CDR3 having the amino acid sequence of SEQ ID NO: 32, SEQ ID NO: 33, and SEQ ID NO: 34, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 188. In some embodiments, the fusion protein comprises a heavy chain variable region comprising CDR1, CDR2, and CDR3 having the amino acid sequence of SEQ ID NO: 1, SEQ ID NO: 2, and SEQ ID NO: 3, a first linker, a light chain variable region comprising CDR1, CDR2, and CDR3 having the amino acid sequence of SEQ ID NO: 4, SEQ ID NO: 5, and SEQ ID NO: 6, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 189. In some embodiments, the fusion protein comprises a heavy chain variable region comprising CDR1, CDR2, and CDR3 having the amino acid sequence of SEQ ID NO: 7, SEQ ID NO: 8, and SEQ ID NO: 9, a first linker, a light chain variable region comprising CDR1, CDR2 and CDR3 having the amino acid sequence of SEQ ID NO: 10, SEQ ID NO: 11 and SEQ ID NO: 12, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 189. In some embodiments, the fusion protein comprises a heavy chain variable region comprising CDR1, CDR2 and CDR3 having the amino acid sequence of SEQ ID NO: 14, SEQ ID NO: 15, and SEQ ID NO: 16, a first linker, a light chain variable region comprising CDR1, CDR2 and CDR3 having the amino acid sequence of SEQ ID NO: 10, SEQ ID NO: 11, and SEQ ID NO: 12, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 189. In some embodiments, the fusion protein comprises a heavy chain variable region comprising CDR1, CDR2, and CDR3 having the amino acid sequence of SEQ ID NO: 7, SEQ ID NO: 8, and SEQ ID NO: 9, a first linker, a light chain variable region comprising CDR1, CDR2 and CDR3 having the amino acid sequence of SEQ ID NO: 10, SEQ ID NO: 11 and SEQ ID NO: 13, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 189. In some embodiments, the fusion protein comprises a the heavy chain variable region comprising CDR1, CDR2 and CDR3 having the amino acid sequence of SEQ ID NO: 14, SEQ ID NO: 15, and SEQ ID NO: 16, a first linker, a light chain variable region comprising CDR1, CDR2 and CDR3 having the amino acid sequence of SEQ ID NO: 10, SEQ ID NO: 11, and SEQ ID NO: 13, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 189. In some embodiments, the fusion protein comprises a heavy chain variable region comprising CDR1, CDR2 and CDR3 having the amino acid sequence of SEQ ID NO: 53, SEQ ID NO: 54, and SEQ ID NO: 55, a first linker, a the light chain variable region comprising CDR1, CDR2 and CDR3 having the amino acid sequence of SEQ ID NO: 56, SEQ ID NO: 57, and SEQ ID NO: 58, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 189. In some embodiments, the fusion protein comprises a heavy chain variable region comprising CDR1, CDR2 and CDR3 having the amino acid sequence of SEQ ID NO: 23, SEQ ID NO: 24, and SEQ ID NO: 25, a first linker, a light chain variable region comprising CDR1, CDR2 and CDR3 having the amino acid sequence of SEQ ID NO: 26, SEQ ID NO: 27, and SEQ ID NO: 28, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 189. In some embodiments, the fusion protein comprises a heavy chain variable region comprising CDR1, CDR2 and CDR3 having the amino acid sequence of SEQ ID NO: 29, SEQ ID NO: 30, and SEQ ID NO: 31, a first linker, a light chain variable region comprising CDR1, CDR2 and CDR3 having the amino acid sequence of SEQ ID NO: 32, SEQ ID NO: 33, and SEQ ID NO: 34, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 189. In some embodiments, the fusion protein comprises a heavy chain variable region comprising CDR1, CDR2, and CDR3 having the amino acid sequence of SEQ ID NO: 1, SEQ ID NO: 2, and SEQ ID NO: 3, a first linker, a light chain variable region comprising CDR1, CDR2, and CDR3 having the amino acid sequence of SEQ ID NO: 4, SEQ ID NO: 5, and SEQ ID NO: 6, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 190. In some embodiments, the fusion protein comprises a heavy chain variable region comprising CDR1, CDR2, and CDR3 having the amino acid sequence of SEQ ID NO: 7, SEQ ID NO: 8, and SEQ ID NO: 9, a first linker, a light chain variable region comprising CDR1, CDR2 and CDR3 having the amino acid sequence of SEQ ID NO: 10, SEQ ID NO: 11 and SEQ ID NO: 12, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 190. In some embodiments, the fusion protein comprises a heavy chain variable region comprising CDR1, CDR2 and CDR3 having the amino acid sequence of SEQ ID NO: 14, SEQ ID NO: 15, and SEQ ID NO: 16, a first linker, a light chain variable region comprising CDR1, CDR2 and CDR3 having the amino acid sequence of SEQ ID NO: 10, SEQ ID NO: 11, and SEQ ID NO: 12, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 190. In some embodiments, the fusion protein comprises a heavy chain variable region comprising CDR1, CDR2, and CDR3 having the amino acid sequence of SEQ ID NO: 7, SEQ ID NO: 8, and SEQ ID NO: 9, a first linker, a light chain variable region comprising CDR1, CDR2 and CDR3 having the amino acid sequence of SEQ ID NO: 10, SEQ ID NO: 11 and SEQ ID NO: 13, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 190. In some embodiments, the fusion protein comprises a the heavy chain variable region comprising CDR1, CDR2 and CDR3 having the amino acid sequence of SEQ ID NO: 14, SEQ ID NO: 15, and SEQ ID NO: 16, a first linker, a light chain variable region comprising CDR1, CDR2 and CDR3 having the amino acid sequence of SEQ ID NO: 10, SEQ ID NO: 11, and SEQ ID NO: 13, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 190. In some embodiments, the fusion protein comprises a heavy chain variable region comprising CDR1, CDR2 and CDR3 having the amino acid sequence of SEQ ID NO: 53, SEQ ID NO: 54, and SEQ ID NO: 55, a first linker, a the light chain variable region comprising CDR1, CDR2 and CDR3 having the amino acid sequence of SEQ ID NO: 56, SEQ ID NO: 57, and SEQ ID NO: 58, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 190. In some embodiments, the fusion protein comprises a heavy chain variable region comprising CDR1, CDR2 and CDR3 having the amino acid sequence of SEQ ID NO: 23, SEQ ID NO: 24, and SEQ ID NO: 25, a first linker, a light chain variable region comprising CDR1, CDR2 and CDR3 having the amino acid sequence of SEQ ID NO: 26, SEQ ID NO: 27, and SEQ ID NO: 28, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 190. In some embodiments, the fusion protein comprises a heavy chain variable region comprising CDR1, CDR2 and CDR3 having the amino acid sequence of SEQ ID NO: 29, SEQ ID NO: 30, and SEQ ID NO: 31, a first linker, a light chain variable region comprising CDR1, CDR2 and CDR3 having the amino acid sequence of SEQ ID NO: 32, SEQ ID NO: 33, and SEQ ID NO: 34, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 190. In some embodiments, the fusion protein comprises a heavy chain variable region comprising CDR1, CDR2, and CDR3 having the amino acid sequence of SEQ ID NO: 1, SEQ ID NO: 2, and SEQ ID NO: 3, a first linker, a light chain variable region comprising CDR1, CDR2, and CDR3 having the amino acid sequence of SEQ ID NO: 4, SEQ ID NO: 5, and SEQ ID NO: 6, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 191. In some embodiments, the fusion protein comprises a heavy chain variable region comprising CDR1, CDR2, and CDR3 having the amino acid sequence of SEQ ID NO: 7, SEQ ID NO: 8, and SEQ ID NO: 9, a first linker, a light chain variable region comprising CDR1, CDR2 and CDR3 having the amino acid sequence of SEQ ID NO: 10, SEQ ID NO: 11 and SEQ ID NO: 12, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 191. In some embodiments, the fusion protein comprises a heavy chain variable region comprising CDR1, CDR2 and CDR3 having the amino acid sequence of SEQ ID NO: 14, SEQ ID NO: 15, and SEQ ID NO: 16, a first linker, a light chain variable region comprising CDR1, CDR2 and CDR3 having the amino acid sequence of SEQ ID NO: 10, SEQ ID NO: 11, and SEQ ID NO: 12, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 191. In some embodiments, the fusion protein comprises a heavy chain variable region comprising CDR1, CDR2, and CDR3 having the amino acid sequence of SEQ ID NO: 7, SEQ ID NO: 8, and SEQ ID NO: 9, a first linker, a light chain variable region comprising CDR1, CDR2 and CDR3 having the amino acid sequence of SEQ ID NO: 10, SEQ ID NO: 11 and SEQ ID NO: 13, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 191. In some embodiments, the fusion protein comprises a the heavy chain variable region comprising CDR1, CDR2 and CDR3 having the amino acid sequence of SEQ ID NO: 14, SEQ ID NO: 15, and SEQ ID NO: 16, a first linker, a light chain variable region comprising CDR1, CDR2 and CDR3 having the amino acid sequence of SEQ ID NO: 10, SEQ ID NO: 11, and SEQ ID NO: 13, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 191. In some embodiments, the fusion protein comprises a heavy chain variable region comprising CDR1, CDR2 and CDR3 having the amino acid sequence of SEQ ID NO: 53, SEQ ID NO: 54, and SEQ ID NO: 55, a first linker, a the light chain variable region comprising CDR1, CDR2 and CDR3 having the amino acid sequence of SEQ ID NO: 56, SEQ ID NO: 57, and SEQ ID NO: 58, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 191. In some embodiments, the fusion protein comprises a heavy chain variable region comprising CDR1, CDR2 and CDR3 having the amino acid sequence of SEQ ID NO: 23, SEQ ID NO: 24, and SEQ ID NO: 25, a first linker, a light chain variable region comprising CDR1, CDR2 and CDR3 having the amino acid sequence of SEQ ID NO: 26, SEQ ID NO: 27, and SEQ ID NO: 28, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 191. In some embodiments, the fusion protein comprises a heavy chain variable region comprising CDR1, CDR2 and CDR3 having the amino acid sequence of SEQ ID NO: 29, SEQ ID NO: 30, and SEQ ID NO: 31, a first linker, a light chain variable region comprising CDR1, CDR2 and CDR3 having the amino acid sequence of SEQ ID NO: 32, SEQ ID NO: 33, and SEQ ID NO: 34, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 191. In some embodiments, the fusion protein comprises a heavy chain variable region comprising CDR1, CDR2, and CDR3 having the amino acid sequence of SEQ ID NO: 1, SEQ ID NO: 2, and SEQ ID NO: 3, a first linker, a light chain variable region comprising CDR1, CDR2, and CDR3 having the amino acid sequence of SEQ ID NO: 4, SEQ ID NO: 5, and SEQ ID NO: 6, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 192. In some embodiments, the fusion protein comprises a heavy chain variable region comprising CDR1, CDR2, and CDR3 having the amino acid sequence of SEQ ID NO: 7, SEQ ID NO: 8, and SEQ ID NO: 9, a first linker, a light chain variable region comprising CDR1, CDR2 and CDR3 having the amino acid sequence of SEQ ID NO: 10, SEQ ID NO: 11 and SEQ ID NO: 12, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 192. In some embodiments, the fusion protein comprises a heavy chain variable region comprising CDR1, CDR2 and CDR3 having the amino acid sequence of SEQ ID NO: 14, SEQ ID NO: 15, and SEQ ID NO: 16, a first linker, a light chain variable region comprising CDR1, CDR2 and CDR3 having the amino acid sequence of SEQ ID NO: 10, SEQ ID NO: 11, and SEQ ID NO: 12, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 192. In some embodiments, the fusion protein comprises a heavy chain variable region comprising CDR1, CDR2, and CDR3 having the amino acid sequence of SEQ ID NO: 7, SEQ ID NO: 8, and SEQ ID NO: 9, a first linker, a light chain variable region comprising CDR1, CDR2 and CDR3 having the amino acid sequence of SEQ ID NO: 10, SEQ ID NO: 11 and SEQ ID NO: 13, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 192. In some embodiments, the fusion protein comprises a the heavy chain variable region comprising CDR1, CDR2 and CDR3 having the amino acid sequence of SEQ ID NO: 14, SEQ ID NO: 15, and SEQ ID NO: 16, a first linker, a light chain variable region comprising CDR1, CDR2 and CDR3 having the amino acid sequence of SEQ ID NO: 10, SEQ ID NO: 11, and SEQ ID NO: 13, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 192. In some embodiments, the fusion protein comprises a heavy chain variable region comprising CDR1, CDR2 and CDR3 having the amino acid sequence of SEQ ID NO: 53, SEQ ID NO: 54, and SEQ ID NO: 55, a first linker, a the light chain variable region comprising CDR1, CDR2 and CDR3 having the amino acid sequence of SEQ ID NO: 56, SEQ ID NO: 57, and SEQ ID NO: 58, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 192. In some embodiments, the fusion protein comprises a heavy chain variable region comprising CDR1, CDR2 and CDR3 having the amino acid sequence of SEQ ID NO: 23, SEQ ID NO: 24, and SEQ ID NO: 25, a first linker, a light chain variable region comprising CDR1, CDR2 and CDR3 having the amino acid sequence of SEQ ID NO: 26, SEQ ID NO: 27, and SEQ ID NO: 28, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 192. In some embodiments, the fusion protein comprises a heavy chain variable region comprising CDR1, CDR2 and CDR3 having the amino acid sequence of SEQ ID NO: 29, SEQ ID NO: 30, and SEQ ID NO: 31, a first linker, a light chain variable region comprising CDR1, CDR2 and CDR3 having the amino acid sequence of SEQ ID NO: 32, SEQ ID NO: 33, and SEQ ID NO: 34, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 192. In some embodiments, the fusion protein comprises a heavy chain variable region comprising CDR1, CDR2, and CDR3 having the amino acid sequence of SEQ ID NO: 1, SEQ ID NO: 2, and SEQ ID NO: 3, a first linker, a light chain variable region comprising CDR1, CDR2, and CDR3 having the amino acid sequence of SEQ ID NO: 4, SEQ ID NO: 5, and SEQ ID NO: 6, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 193. In some embodiments, the fusion protein comprises a heavy chain variable region comprising CDR1, CDR2, and CDR3 having the amino acid sequence of SEQ ID NO: 7, SEQ ID NO: 8, and SEQ ID NO: 9, a first linker, a light chain variable region comprising CDR1, CDR2 and CDR3 having the amino acid sequence of SEQ ID NO: 10, SEQ ID NO: 11 and SEQ ID NO: 12, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 193. In some embodiments, the fusion protein comprises a heavy chain variable region comprising CDR1, CDR2 and CDR3 having the amino acid sequence of SEQ ID NO: 14, SEQ ID NO: 15, and SEQ ID NO: 16, a first linker, a light chain variable region comprising CDR1, CDR2 and CDR3 having the amino acid sequence of SEQ ID NO: 10, SEQ ID NO: 11, and SEQ ID NO: 12, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 193. In some embodiments, the fusion protein comprises a heavy chain variable region comprising CDR1, CDR2, and CDR3 having the amino acid sequence of SEQ ID NO: 7, SEQ ID NO: 8, and SEQ ID NO: 9, a first linker, a light chain variable region comprising CDR1, CDR2 and CDR3 having the amino acid sequence of SEQ ID NO: 10, SEQ ID NO: 11 and SEQ ID NO: 13, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 193. In some embodiments, the fusion protein comprises a the heavy chain variable region comprising CDR1, CDR2 and CDR3 having the amino acid sequence of SEQ ID NO: 14, SEQ ID NO: 15, and SEQ ID NO: 16, a first linker, a light chain variable region comprising CDR1, CDR2 and CDR3 having the amino acid sequence of SEQ ID NO: 10, SEQ ID NO: 11, and SEQ ID NO: 13, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 193. In some embodiments, the fusion protein comprises a heavy chain variable region comprising CDR1, CDR2 and CDR3 having the amino acid sequence of SEQ ID NO: 53, SEQ ID NO: 54, and SEQ ID NO: 55, a first linker, a the light chain variable region comprising CDR1, CDR2 and CDR3 having the amino acid sequence of SEQ ID NO: 56, SEQ ID NO: 57, and SEQ ID NO: 58, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 193. In some embodiments, the fusion protein comprises a heavy chain variable region comprising CDR1, CDR2 and CDR3 having the amino acid sequence of SEQ ID NO: 23, SEQ ID NO: 24, and SEQ ID NO: 25, a first linker, a light chain variable region comprising CDR1, CDR2 and CDR3 having the amino acid sequence of SEQ ID NO: 26, SEQ ID NO: 27, and SEQ ID NO: 28, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 193. In some embodiments, the fusion protein comprises a heavy chain variable region comprising CDR1, CDR2 and CDR3 having the amino acid sequence of SEQ ID NO: 29, SEQ ID NO: 30, and SEQ ID NO: 31, a first linker, a light chain variable region comprising CDR1, CDR2 and CDR3 having the amino acid sequence of SEQ ID NO: 32, SEQ ID NO: 33, and SEQ ID NO: 34, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 193. In some embodiments, the antibody or antigen-binding fragment thereof, or the fusion protein described herein further comprises a methionine or a signal peptide at the N-terminus. In some embodiments, a polypeptide comprising at least 85% identity to the amino acid sequence of SEQ ID NO: 65, 68, 72, 74, 78, 80, 84, 88, 162, 164, 166, 168, 170, 172, 174, or 176 further comprises a methionine or a signal peptide at the N-terminus. In some embodiments, the signal peptide is not at the N-terminus, for example it may be present in a fusion protein and may not function as a cleavage site. In other embodiments, the antibody or antigen binding fragment thereof or the fusion protein lacks any signal peptide. In some embodiments, the signal peptide comprises the amino acid sequence of SEQ ID NO: 224. In some embodiments, a polypeptide comprising at least 85% identity to the amino acid sequence of SEQ ID NO: 82, 86, 91, 166, 168, 170, 172, 174, or 176 further comprises a methionine or a signal peptide at the N-terminus. In some embodiments, the signal peptide comprises the amino acid sequence of SEQ ID NO: 225. In some embodiments, a polypeptide comprising at least 85% identity to the amino acid sequence of SEQ ID NO: 70, 162, or 164, further comprises a methionine or a signal peptide at the N-terminus. In some embodiments, the signal peptide comprises the amino acid sequence of SEQ ID NO: 226. In some embodiments, any antibody, antigen-binding fragment, fusion protein, or polypeptide described herein can be with or without a signal peptide and/or a methionine at the N-terminus. In some embodiments, any amino acid sequence in Tables 5, 6, or 12 can be with or without a signal peptide and/or a methionine at the N-terminus. In some embodiments, any antibody, antigen-binding fragment, fusion protein, or polypeptide described herein can be with or without a signal peptide, optionally SEQ ID NO: 224, 225, or 226. In some embodiments, the polypeptide comprising at least 85% identity to the amino acid sequence of SEQ ID NO: 166, 168, 170, 172, 174, or 176 can have a deletion of the amino acid sequence comprising SEQ ID NO: 225. In some embodiments, the polypeptide comprising at least 85% identity to the amino acid sequence of SEQ ID NO: 162 or 164 can have a deletion of the amino acid sequence comprising SEQ ID NO: 226. In some embodiments, a signal peptide can be used as a linker. In some embodiments, the first linker comprises an amino acid sequence GSGSG (SEQ ID NO: 222) or SEQ ID NO: 221. In some embodiments, the second linker comprises an amino acid sequence GSGSG (SEQ ID NO: 222) or SEQ ID NO: 221. In some embodiments, the fusion protein comprises a polypeptide having at least 85%, 90%, 95%, 97%, 98%, 99%, 99.5%, 99.5% or 100% identity to the amino acid sequence of any one of SEQ ID NOs: 162, 164, 166, 168, 170, 172, 174, and 176. In some embodiments, the fusion protein comprises a polypeptide having at least 85%, 90%, 95%, 97%, 98%, 99%, 99.5%, 99.5% or 100% identity to the amino acid sequence of any one of SEQ ID NOs: 162, 164, 166, 168, 170, 172, 174, and 176, and further comprises a 6-His tag or FLAG tag at the 5′ end or the 3′ end. In some embodiments, the fusion protein comprises a polypeptide having at least 85%, 90%, 95%, 97%, 98%, 99%, 99.5%, 99.5% or 100% identity to the amino acid sequence of any one of SEQ ID NOs: 162, 164, 166, 168, 170, 172, 174, and 176, and further comprises a 6-His tag at the 3′ end. The sequences described herein can have addition and/or deletion of tags such as 6-His tag or FLAG tag, and nucleic acid encoding same.

The E3 ligases that are useful as part of the fusion protein can be a member of E3 ligase families U-BOX, HECT, F-BOX, RBR, or RING. In some embodiments, the E3 ligase is a member of the U-BOX, HECT, F-BOX, RBR, or RING family of E3 ligases, or an active fragment thereof. In some embodiments, the member of the U-BOX family of ligases is CHIP, UBE4A, or UBOX5 (RNF37), or an active fragment thereof. In some embodiments, the member of the HECT family of ligases is NEDD4L or UBR5, or an active fragment thereof. In some embodiments, the member of the F-BOX family of ligases is BTrCP, or an active fragment thereof. In some embodiments, the member of the RBR family is Parkin, or an active fragment thereof. In some embodiments, the member of the RING family is RNF4 or an active fragment thereof. In some embodiments, E3 ligase comprises an amino acid sequence having at least 85%, 90%, 95%, 97%, 98%, 99%, 99.5%, 99.5% or 100% identity to the amino acid sequence of in any one of SEQ ID NOs: 145, 147, 149, 151, 153, 155, 157, and 159. In some embodiments, E3 ligase comprises an amino acid sequence having at least 85%, 90%, 95%, 97%, 98%, 99%, 99.5%, 99.5% or 100% identity to amino acids 128-303 of SEQ ID NO: 145. In some embodiments, E3 ligase comprises an amino acid sequence having at least 85%, 90%, 95%, 97%, 98%, 99%, 99.5%, 99.5% or 100% identity to amino acids 873-1066 of SEQ ID NO: 147. In some embodiments, E3 ligase comprises an amino acid sequence having at least 85%, 90%, 95%, 97%, 98%, 99%, 99.5%, 99.5% or 100% identity to amino acids 2-340 of SEQ ID NO: 149. In some embodiments, E3 ligase comprises an amino acid sequence having at least 85%, 90%, 95%, 97%, 98%, 99%, 99.5%, 99.5% or 100% identity to amino acids 616-975 of SEQ ID NO: 151. In some embodiments, E3 ligase comprises an amino acid sequence having at least 85%, 90%, 95%, 97%, 98%, 99%, 99.5%, 99.5% or 100% identity to amino acids 2455-2799 of SEQ ID NO: 153. In some embodiments, E3 ligase comprises an amino acid sequence having at least 85%, 90%, 95%, 97%, 98%, 99%, 99.5%, 99.5% or 100% identity to amino acids 1-261 of SEQ ID NO: 155. In some embodiments, E3 ligase comprises an amino acid sequence having at least 85%, 90%, 95%, 97%, 98%, 99%, 99.5%, 99.5% or 100% identity to amino acids 220-465 of SEQ ID NO: 157. In some embodiments, E3 ligase comprises an amino acid sequence having at least 85%, 90%, 95%, 97%, 98%, 99%, 99.5%, 99.5% or 100% identity to amino acids 76-190 of SEQ ID NO: 159. sequence of in any one of SEQ ID NOs: 145, 147, 149, 151, 153, 155, 157, and 159. In some embodiments, the active fragment of the E3 ligase comprises an amino acid sequence having at least 85%, 90%, 95%, 97%, 98%, 99%, 99.5%, 99.5% or 100% identity to the amino acid sequence of in any one of SEQ ID NOs: 186-193, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the active fragment of the E3 ligase comprises an amino acid sequence having at least 85%, 90%, 95%, 97%, 98%, 99%, 99.5%, 99.5% or 100% identity to the amino acid sequence of in any one of SEQ ID NOs: 186-193, or a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the active fragment of the E3 ligase comprises an amino acid sequence having at least 85%, 90%, 95%, 97%, 98%, 99%, 99.5%, 99.5% or 100% identity to the amino acid sequence of in any one of SEQ ID NOs: 186-193. In some embodiments, the active fragment of the E3 ligase comprises an amino acid sequence having at least 85%, 90%, 95%, 97%, 98%, 99%, 99.5%, 99.5% or 100% identity to the amino acid sequence of SEQ ID NO: 186. In some embodiments, an active E3 ligase comprises a truncated E3 ligase comprising an amino acid sequence having at least 85%, 90%, 95%, 97%, 98%, 99%, 99.5%, 99.5% or 100% identity to the amino acid sequence of SEQ ID NO: 223. In some embodiments, the active fragment of the E3 ligase comprises an amino acid sequence having at least 85%, 90%, 95%, 97%, 98%, 99%, 99.5%, 99.5% or 100% identity to the amino acid sequence of SEQ ID NO: 187. In some embodiments, the active fragment of the E3 ligase comprises an amino acid sequence having at least 85%, 90%, 95%, 97%, 98%, 99%, 99.5%, 99.5% or 100% identity to the amino acid sequence of SEQ ID NO: 188. In some embodiments, the active fragment of the E3 ligase comprises an amino acid sequence having at least 85%, 90%, 95%, 97%, 98%, 99%, 99.5%, 99.5% or 100% identity to the amino acid sequence of SEQ ID NO: 189. In some embodiments, the active fragment of the E3 ligase comprises an amino acid sequence having at least 85%, 90%, 95%, 97%, 98%, 99%, 99.5%, 99.5% or 100% identity to the amino acid sequence of SEQ ID NO: 190. In some embodiments, the active fragment of the E3 ligase comprises an amino acid sequence having at least 85%, 90%, 95%, 97%, 98%, 99%, 99.5%, 99.5% or 100% identity to the amino acid sequence of SEQ ID NO: 191. In some embodiments, the active fragment of the E3 ligase comprises an amino acid sequence having at least 85%, 90%, 95%, 97%, 98%, 99%, 99.5%, 99.5% or 100% identity to the amino acid sequence of SEQ ID NO: 192. In some embodiments, the active fragment of the E3 ligase comprises an amino acid sequence having at least 85%, 90%, 95%, 97%, 98%, 99%, 99.5%, 99.5% or 100% identity to the amino acid sequence of SEQ ID NO: 193. In some embodiments, the percentage identity is outside of the CDRs described herein.

### D. Nucleic Acids

Also provide is a nucleic acid encoding the antibody or binding fragment thereof, or fusion protein described herein. The nucleic acid sequences encoding the antibody or binding fragment thereof, or the fusion protein, may be expressed in a variety of host cells that secrete the expression product, including E. coli, other bacterial hosts, yeast, and various higher eukaryotic cells such as the COS, CHO and HeLa cells lines and myeloma cell lines. The recombinant protein gene will be operably linked to appropriate expression control sequences for each host. For E. coli this includes a promoter such as the T7, trp, or lambda promoters, a ribosome binding site and preferably a transcription termination signal. For eukaryotic cells, the control sequences will include a promoter and may include an enhancer derived from immunoglobulin genes, SV40, cytomegalovirus, etc., and a polyadenylation sequence, and may include splice donor and acceptor sequences.

The vectors of the disclosure can be transferred into the chosen host cell by well-known methods such as calcium chloride transformation for E. coli and calcium phosphate treatment or electroporation for mammalian cells. Cells transformed by the vectors can be selected by resistance to antibiotics conferred by genes contained on the vectors, such as the amp, gpt, neo and hyg genes. In addition, nucleic acids may be introduced into mammalian cells by viral vectors.

Once expressed and secreted, the antibody or binding fragment thereof, or fusion protein, can be purified according to standard procedures of the art, including ammonium sulfate precipitation, affinity columns, column chromatography, gel electrophoresis and the like (see, Scopes, R. K. Protein Purification: Principles and Practice. Springer Science & Business Media, 2013; Burgess, Richard R., and Murray P. Deutscher, eds. Guide to Protein Purification. Academic Press, 2009). Substantially pure compositions of at least about 90 to 95% homogeneity can be obtained, and 98 to 99% or more homogeneity can also be obtained for pharmaceutical uses. Once purified, partially or to homogeneity as desired, the antibody or binding fragment thereof, or fusion protein, may then be used therapeutically.

Accordingly, also provided is an isolated antibody or binding fragment thereof that binds to misfolded SOD1 epitope having an amino acid sequence shown in SEQ ID NO: 142 or 144, the antibody or binding fragment thereof comprises:
(i) a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 66 or 67; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 71;
(ii) a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 69; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 71;
(iii) a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 73; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 77;
(iv) a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 75; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 77;
(v) a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 79; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 83;
(vi) a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 81; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 83;
(vii) a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 85; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 87; or
(viii) a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 89 or 90; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 92.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 66 or 67; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 71.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 69; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 71.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 73; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 77.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 75; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 77.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 79; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 83.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 81; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 83.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 85; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 87.

In some embodiments, the isolated antibody or binding fragment thereof binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 89 or 90; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 92.

In some embodiments, the percentage identity is outside of the CDRs described herein.

Also provided is an isolated antibody that binds to misfolded SOD1 epitope having an amino acid sequence shown in SEQ ID NO: 142 or 144, the antibody comprises:
(i) a heavy chain encoded by nucleic acid sequence having at least 85%, 90%, 95%, 97%, 98%, 99%, 99.5%, 99.5% or 100% identity to the nucleic acid sequence of SEQ ID NO: 110 or 111, and a light chain encoded by nucleic acid sequence having at least 85%, 90%, 95%, 97%, 98%, 99%, 99.5%, 99.5% or 100% identity to the nucleic acid sequence of SEQ ID NO: 115;
(ii) a heavy chain encoded by nucleic acid sequence having at least 85%, 90%, 95%, 97%, 98%, 99%, 99.5%, 99.5% or 100% identity to the nucleic acid sequence of SEQ ID NO: 113, and a light chain encoded by nucleic acid sequence having at least 85%, 90%, 95%, 97%, 98%, 99%, 99.5%, 99.5% or 100% identity to the nucleic acid sequence of SEQ ID NO: 115;
(iii) a heavy chain encoded by nucleic acid sequence having at least 85%, 90%, 95%, 97%, 98%, 99%, 99.5%, 99.5% or 100% identity to the nucleic acid sequence of SEQ ID NO: 117, and a light chain encoded by nucleic acid sequence having at least 85%, 90%, 95%, 97%, 98%, 99%, 99.5%, 99.5% or 100% identity to the nucleic acid sequence of SEQ ID NO: 121;
(iv) a heavy chain encoded by nucleic acid sequence having at least 85%, 90%, 95%, 97%, 98%, 99%, 99.5%, 99.5% or 100% identity to the nucleic acid sequence of SEQ ID NO: 119, and a light chain encoded by nucleic acid sequence having at least 85%, 90%, 95%, 97%, 98%, 99%, 99.5%, 99.5% or 100% identity to the nucleic acid sequence of SEQ ID NO: 121;
(v) a heavy chain encoded by nucleic acid sequence having at least 85%, 90%, 95%, 97%, 98%, 99%, 99.5%, 99.5% or 100% identity to the nucleic acid sequence of SEQ ID NO: 123, and a light chain encoded by nucleic acid sequence having at least 85%, 90%, 95%, 97%, 98%, 99%, 99.5%, 99.5% or 100% identity to the nucleic acid sequence of SEQ ID NO: 127;
(vi) a heavy chain encoded by nucleic acid sequence having at least 85%, 90%, 95%, 97%, 98%, 99%, 99.5%, 99.5% or 100% identity to the nucleic acid sequence of SEQ ID NO: 125, and a light chain encoded by nucleic acid sequence having at least 85%, 90%, 95%, 97%, 98%, 99%, 99.5%, 99.5% or 100% identity to the nucleic acid sequence of SEQ ID NO: 127;
(vii) a heavy chain encoded by nucleic acid sequence having at least 85%, 90%, 95%, 97%, 98%, 99%, 99.5%, 99.5% or 100% identity to the nucleic acid sequence of SEQ ID NO: 129, and a light chain encoded by nucleic acid sequence having at least 85%, 90%, 95%, 97%, 98%, 99%, 99.5%, 99.5% or 100% identity to the nucleic acid sequence of SEQ ID NO: 131; or
(viii) a heavy chain encoded by nucleic acid sequence having at least 85%, 90%, 95%, 97%, 98%, 99%, 99.5%, 99.5% or 100% identity to the nucleic acid sequence of SEQ ID NO: 133 or 134, and a light chain encoded by nucleic acid sequence having at least 85%, 90%, 95%, 97%, 98%, 99%, 99.5%, 99.5% or 100% identity to the nucleic acid sequence of SEQ ID NO: 136.

In some embodiments, the isolated antibody that binds to misfolded SOD1 epitope having an amino acid sequence shown in SEQ ID NO: 142 or 144, the antibody comprises a heavy chain encoded by nucleic acid sequence having at least 85%, 90%, 95%, 97%, 98%, 99%, 99.5%, 99.5% or 100% identity to the nucleic acid sequence of SEQ ID NO: 110 or 111, and a light chain encoded by nucleic acid sequence having at least 85%, 90%, 95%, 97%, 98%, 99%, 99.5%, 99.5% or 100% identity to the nucleic acid sequence of SEQ ID NO: 115.

In some embodiments, the isolated antibody that binds to misfolded SOD1 epitope having an amino acid sequence shown in SEQ ID NO: 142 or 144, the antibody comprises a heavy chain encoded by nucleic acid sequence having at least 85%, 90%, 95%, 97%, 98%, 99%, 99.5%, 99.5% or 100% identity to the nucleic acid sequence of SEQ ID NO: 113, and a light chain encoded by nucleic acid sequence having at least 85%, 90%, 95%, 97%, 98%, 99%, 99.5%, 99.5% or 100% identity to the nucleic acid sequence of SEQ ID NO: 115.

In some embodiments, the isolated antibody that binds to misfolded SOD1 epitope having an amino acid sequence shown in SEQ ID NO: 142 or 144, the antibody comprises a heavy chain encoded by nucleic acid sequence having at least 85%, 90%, 95%, 97%, 98%, 99%, 99.5%, 99.5% or 100% identity to the nucleic acid sequence of SEQ ID NO: 117, and a light chain encoded by nucleic acid sequence having at least 85%, 90%, 95%, 97%, 98%, 99%, 99.5%, 99.5% or 100% identity to the nucleic acid sequence of SEQ ID NO: 121.

In some embodiments, the isolated antibody that binds to misfolded SOD1 epitope having an amino acid sequence shown in SEQ ID NO: 142 or 144, the antibody comprises a heavy chain encoded by nucleic acid sequence having at least 85%, 90%, 95%, 97%, 98%, 99%, 99.5%, 99.5% or 100% identity to the nucleic acid sequence of SEQ ID NO: 119, and a light chain encoded by nucleic acid sequence having at least 85%, 90%, 95%, 97%, 98%, 99%, 99.5%, 99.5% or 100% identity to the nucleic acid sequence of SEQ ID NO: 121.

In some embodiments, the isolated antibody that binds to misfolded SOD1 epitope having an amino acid sequence shown in SEQ ID NO: 142 or 144, the antibody comprises a heavy chain encoded by nucleic acid sequence having at least 85%, 90%, 95%, 97%, 98%, 99%, 99.5%, 99.5% or 100% identity to the nucleic acid sequence of SEQ ID NO: 123, and a light chain encoded by nucleic acid sequence having at least 85%, 90%, 95%, 97%, 98%, 99%, 99.5%, 99.5% or 100% identity to the nucleic acid sequence of SEQ ID NO: 127.

In some embodiments, the isolated antibody that binds to misfolded SOD1 epitope having an amino acid sequence shown in SEQ ID NO: 142 or 144, the antibody comprises a heavy chain encoded by nucleic acid sequence having at least 85%, 90%, 95%, 97%, 98%, 99%, 99.5%, 99.5% or 100% identity to the nucleic acid sequence of SEQ ID NO: 125, and a light chain encoded by nucleic acid sequence having at least 85%, 90%, 95%, 97%, 98%, 99%, 99.5%, 99.5% or 100% identity to the nucleic acid sequence of SEQ ID NO: 127.

In some embodiments, the isolated antibody that binds to misfolded SOD1 epitope having an amino acid sequence shown in SEQ ID NO: 142 or 144, the antibody comprises a heavy chain encoded by nucleic acid sequence having at least 85%, 90%, 95%, 97%, 98%, 99%, 99.5%, 99.5% or 100% identity to the nucleic acid sequence of SEQ ID NO: 129, and a light chain encoded by nucleic acid sequence having at least 85%, 90%, 95%, 97%, 98%, 99%, 99.5%, 99.5% or 100% identity to the nucleic acid sequence of SEQ ID NO: 131.

In some embodiments, the isolated antibody that binds to misfolded SOD1 epitope having an amino acid sequence shown in SEQ ID NO: 142 or 144, the antibody comprises a heavy chain encoded by nucleic acid sequence having at least 85%, 90%, 95%, 97%, 98%, 99%, 99.5%, 99.5% or 100% identity to the nucleic acid sequence of SEQ ID NO: 133 or 134, and a light chain encoded by nucleic acid sequence having at least 85%, 90%, 95%, 97%, 98%, 99%, 99.5%, 99.5% or 100% identity to the nucleic acid sequence of SEQ ID NO: 136.

Also provided is a fusion protein comprising an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises:
(i) a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 66 or 67; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 71;
(ii) a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 69; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 71;
(iii) a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 73; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 77;
(iv) a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 75; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 77;
(v) a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 79; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 83;
(vi) a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 81; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 83;
(vii) a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 85; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 87; or
(viii) a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 89 or 90; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 92.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 66 or 67; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 71.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 69; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 71.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 73; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 77.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 75; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 77.

In some embodiments, the fusion protein comprises an E3 ligase or an active fragment thereof, and an antibody or binding fragment thereof that binds to misfolded SOD1 epitope having an amino acid sequence shown in SEQ ID NO: 142 or 144, the antibody or binding fragment thereof comprises a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 79; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 83.

In some embodiments, the fusion protein comprises an E3 ligase or an active fragment thereof, and an antibody or binding fragment thereof that binds to misfolded SOD1 epitope having an amino acid sequence shown in SEQ ID NO: 142, the antibody or binding fragment thereof comprises a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 81; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 83.

In some embodiments, the fusion protein comprises an E3 ligase or an active fragment thereof, and an antibody or binding fragment thereof that binds to misfolded SOD1 epitope having an amino acid sequence shown in SEQ ID NO: 142, the antibody or binding fragment thereof comprises a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 85; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 87.

In some embodiments, the fusion protein comprises an E3 ligase or an active fragment thereof, and an antibody or binding fragment thereof that binds to misfolded SOD1 epitope having an amino acid sequence shown in SEQ ID NO: 142, the antibody or binding fragment thereof comprises a heavy chain comprising a heavy chain variable region, wherein the heavy chain variable region is encoded by a nucleic acid sequence having at least 85%, 90%, 95%, 97%, 98%, 99%, 99.5%, 99.5% or 100% identity to the nucleic acid sequence of SEQ ID NO: 89 or 90; and a light chain comprising a light chain variable region, wherein the light chain variable region is encoded by a nucleic acid sequence having at least 85%, 90%, 95%, 97%, 98%, 99%, 99.5%, 99.5% or 100% identity to the nucleic acid sequence of SEQ ID NO: 92.

In some embodiments, the active fragment of the E3 ligase comprises an amino acid sequence encoded by a nucleic acid sequence having at least 85%, 90%, 95%, 97%, 98%, 99%, 99.5%, 99.5% or 100% identity to the nucleic acid sequence of any one of SEQ ID NOs: 146, 148, 150, 152, 154, 156, 158, and 160. In some embodiments, the fusion protein comprises an amino acid encoded by a nucleic acid sequence having at least 85%, 90%, 95%, 97%, 98%, 99%, 99.5%, 99.5% or 100% identity to the nucleic acid sequence of any one of SEQ ID NOs: 161, 163, 165, 167, 169, 171, 173, 175, and 179-185.

In some embodiments, the percentage identity is outside of the CDRs described herein.

In some embodiments, the antibody or antigen-binding fragment thereof, or the fusion protein described herein comprises a sequence shown in Table 4 with the N-terminus methionine deleted. In some embodiments, the antibody or antigen-binding fragment thereof, or the fusion protein described herein comprises a sequence shown in Table 4 with first 15, 16, 17, 18, 29, 20, 21, 22, 23, 24, 25, 26, 27, 28, 29, or 30 amino acids deleted, and optionally substituted by a methionine. In some embodiments, a sequence shown in Table 4 further comprises a signal peptide at the N-terminus. In some embodiments, the antibody or antigen-binding fragment thereof, or the fusion protein described herein comprises a sequence shown in Table 5 with the first 15, 16, 17, 18, 29, 20, 21, 22, 23, 24, 25, 26, 27, 28, 29, or 30 amino acids deleted. In some embodiments, the antibody or antigen-binding fragment thereof, or the fusion protein described herein comprises a sequence shown in Table 5 with the first 15, 16, 17, 18, 29, 20, 21, 22, 23, 24, 25, 26, 27, 28, 29, or 30 amino acids deleted, and optionally substituted by a methionine.

In some embodiments, the polypeptide disclosed herein is encoded by a nucleic acid.

In some embodiments, there is also provided a vector comprising a nucleic acid encoding an antibody or binding fragment thereof, or a fusion protein comprising an E3 ligase or an active fragment thereof and an antibody or bind fragment thereof described herein. To express the antibody or fragment thereof, or the fusion protein, the nucleic acid is operatively linked to transcriptional and translational control sequences. Useful vectors include plasmids, retroviruses, cosmids, and the like. The vector and expression control sequences are chosen to be compatible with the expression host cell used. The nucleic acid encoding the heavy chain or variable region thereof and the nucleic acid encoding the light chain or variable region thereof, can be inserted into separate vectors. In some embodiments, nucleic acids encoding the heavy chain or variable region thereof and the nucleic acid encoding the light chain or variable region thereof are inserted into the same vector. The nucleic acids are inserted into the vector by standard methods known in the art, for example, ligation of complementary restriction sites on the antibody gene fragment and vector, or blunt end ligation if no restriction sites are present.

### III. Pharmaceutical Formulations and Medicaments

In another aspect, the antibody or binding fragment thereof, the fusion protein or the nucleic acid described herein, as well as variants and modifications thereof, are provided as a pharmaceutical composition for therapeutic use. In an embodiment, the pharmaceutical formulation comprises an isolated antibody or binding fragment thereof, or fusion protein described herein.

Representative delivery regimens include oral, parenteral (including subcutaneous, intramuscular and intravenous injection), rectal, buccal (including sublingual), transdermal, inhalation, ocular and intranasal. In one embodiment, delivery of compounds entails subcutaneous injection of a controlled-release injectable formulation. In some embodiments, compounds described herein are useful for subcutaneous, intranasal and inhalation administration.

The selection of the exact dose and composition and the most appropriate delivery regimen will be influenced by, inter alia, the pharmacological properties of the selected antibody or binding fragment thereof, fusion protein or nucleic acid, the nature and severity of the condition being treated, and the physical condition and mental acuity of the recipient. Additionally, the route of administration will result in differential amounts of absorbed material. Bioavailabilities for administration of compounds through different routes are particularly variable, with amounts from less than 1% to near 100% being seen. Typically, bioavailability from routes other than intravenous, intraperitoneal or subcutaneous injection are 50% or less.

The pharmaceutical compositions or formulations of the present disclosure can be formulated with a physiologically acceptable carrier or excipient to prepare a pharmaceutical composition. The carrier and composition can be sterile. The formulation should suit the mode of administration, for example intravenous or subcutaneous administration. Methods of formulating compositions are known in the art (see, e.g., Remington's Pharmaceuticals Sciences, 17th Edition, Mack Publishing Co., (Alfonso R. Gennaro, editor) (1989); herein incorporated by reference).

Suitable pharmaceutically acceptable carriers include, but not limited to, water, salt solutions (e.g., NaCl), saline, buffered saline, alcohols, glycerol, ethanol, gum arabic, vegetable oils, benzyl alcohols, polyethylene glycols, gelatin, carbohydrates such as lactose, amylose or starch, sugars such as mannitol, sucrose, or others, dextrose, magnesium stearate, talc, silicic acid, viscous paraffin, perfume oil, fatty acid esters, hydroxymethylcellulose, polyvinyl pyrolidone, etc., as well as combinations thereof. The pharmaceutical preparations can, if desired, be mixed with auxiliary agents (e.g., lubricants, preservatives, stabilizers, wetting agents, emulsifiers, salts for influencing osmotic pressure, buffers, coloring and/or aromatic substances and the like) which do not deleteriously react with the active compounds or interference with their activity. In an embodiment, a water-soluble carrier suitable for intravenous administration is used. Pharmaceutically acceptable salts retain the desired biological activity of the parent antibody or binding fragment thereof without toxic side effects.

The composition or medicament, if desired, can also contain minor amounts of wetting or emulsifying agents, or pH buffering agents. The composition can be a liquid solution, suspension, emulsion, sustained release formulation, or powder. The composition can also be formulated as a suppository, with traditional binders and carriers such as triglycerides.

The composition or medicament can be formulated in accordance with the routine procedures as a pharmaceutical composition adapted for administration to human beings. For example, in an embodiment, a composition for intravenous administration typically is a solution in sterile isotonic aqueous buffer. Where necessary, the composition may also include a solubilizing agent and a local anesthetic to ease pain at the site of the injection. Generally, the ingredients are supplied either separately or mixed together in unit dosage form, for example, as a dry lyophilized powder or water free concentrate in a hermetically sealed container such as an ampule or sachette indicating the quantity of active agent. Where the composition is to be administered by infusion, it can be dispensed with an infusion bottle containing sterile pharmaceutical grade water, saline or dextrose/water. Where the composition is administered by injection, an ampule of sterile water for injection or saline can be provided so that the ingredients may be mixed prior to administration.

In some embodiments, the pharmaceutical composition comprises a liquid carrier such as, but not limited to, water, saline, phosphate buffered saline, Ringer's solution, dextrose solution, serum-containing solutions, Hank's solution, other aqueous physiologically balanced solutions, oils, esters and glycols.

The antibody or binding fragment thereof, fusion protein or nucleic acid as described herein can be formulated as neutral or salt forms. As stated above, pharmaceutically acceptable salts include those formed with free amino groups such as those derived from hydrochloric, phosphoric, acetic, oxalic, tartaric acids, etc., and those formed with free carboxyl groups such as those derived from sodium, potassium, ammonium, calcium, ferric hydroxides, isopropylamine, triethylamine, 2-ethylamino ethanol, histidine, procaine, etc.

The pharmaceutical formulations of the present disclosure contain, as the binding agent, antibody or binding fragment thereof may be mixed with an excipient, diluted by an excipient or enclosed within a carrier, which can be in the form of a capsule, sachet, paper or other container, according to well-known methods and pharmaceutical compositions. The composition may be administered by any route suitable for antibody or binding fragment thereof, fusion protein, or nucleic acid administration, including parenteral, intravenous, subcutaneous, or intramuscular administration. Typically, the antibody or binding fragment thereof, fusion protein or nucleic acid is dissolved or suspended in a sterile injectable solution, at a concentration sufficient to provide the required dose in 0.5 to 2 ml or less. Pharmaceutical compositions of this disclosure suitable for parenteral administrations comprise one or more compounds of the disclosure in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, solutes which render the formulation isotonic with the blood of the intended recipient or suspending or thickening agents.

Injectable depot forms are made by forming microencapsulated matrices of the drug in biodegradable polymers such as polylactide-polyglycolide. Depending on the ratio of drug to polymer, and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues. The injectable materials can be sterilized for example, by filtration through a bacterial-retaining filter.

The pharmaceutical compositions may be presented in unit-dose or multi-dose sealed containers, for example, ampules and vials, and may be stored in a lyophilized condition requiring only the addition of the sterile liquid carrier, for example water for injection, immediately prior to use. Extemporaneous injection solutions and suspensions may be prepared from sterile powders, granules and tablets of the type described above.

### IV. Methods of Treatment and Uses

Methods of treatment and uses are contemplated for diseases and conditions mediated by misfolded SOD1, for example, amyotrophic lateral sclerosis (ALS), Alzheimer's disease or Parkinson's disease, or frontotemporal dementia. The misfolded SOD1 target represents an opportunity for antibody or binding fragment thereof, fusion protein or nucleic acid such as those described herein to be used to treat or prevent diseases and conditions mediated by misfolded SOD1.

Accordingly, in one embodiment there is provided, a method of treating a medical condition, disease, or disorder mediated by a misfolded form of SOD1 in a subject in need thereof, the method comprises administering to the subject an antibody or binding fragment thereof, a fusion protein, a nuclei acid, or a pharmaceutical composition described herein. In some embodiments, the medical condition, disease or disorder is a neurodegenerative condition, disease or disorder. In some embodiments, the neurodegenerative condition, disease or disorder is amyotrophic lateral sclerosis (ALS), Alzheimer's disease or Parkinson's disease or frontotemporal dementia. In some embodiments, the ALS is sporadic ALS. In some embodiments, the ALS is familial ALS. In some embodiments, the neurodegenerative condition, disease or disorder is Alzheimer's disease. In some embodiments, the neurodegenerative condition, disease or disorder is Parkinson's disease. In some embodiments, the neurodegenerative condition, disease or disorder is frontotemporal dementia. In some embodiments, the misfolded form of SOD1 comprises SOD1 monomer, a dimer comprising SOD1 monomers, or an aggregate comprising SOD1 monomers and/or dimers, or toxic trimers. In some embodiments, the SOD1 monomer comprises a mutant SOD1 monomer. In some embodiments, the mutant SOD1 monomer comprise one or more mutations selected from the group consisting of A4V, G93A, G85R, D90A, G127X, V148G, H46R, G37R, C6G, and E100G, and wherein X is truncation mutation. In some embodiments, the mutant SOD1 monomer comprise one or more mutations selected from Table 1.

TABLE 1
List of Detected Mutations in SOD1 in ALS, including FALS.

| Amino Acid | Mutation |
|---|---|
| 3 | K−> E (in ALS) |
| 4 | A −> S (in FALS) |
| 4 | A −> T (in FALS) |
| 4 | A −> V (in FALS) |
| 4 | A −> F (in ALS) |
| 6 | C −> G (in ALS) |
| 6 | C −> F (in FALS) |
| 6 | C −> S (in ALS) |
| 6 | C −>W (in ALS) |
| 7 | V −> E (in FALS) |
| 8 | L −> Q (in FALS) |
| 8 | L −> V (in FALS) |
| 10 | G −> V (in ALS) |
| 10 | G −> R (in ALS) |
| 11 | D −> Y |
| 12 | G −> R (in FALS) |
| 14 | V −> G (in FALS) |
| 14 | V −> M (in FALS) |
| 16 | G −> S (in ALS) |
| 16 | G −> A (in ALS) |
| 19 | N −>S (in ALS) |
| 20 | F −> C (in ALS) |
| 21 | E −> G (in FALS) |
| 21 | E −> K (in FALS) |
| 22 | Q −> R (in ALS) |
| 22 | Q −> L (in ALS) |
| 27 | G del GGACCA (in ALS) |
| 31 | V −> A (in ALS) |
| 37 | G −> R (in FALS) |
| 37 | G −> V (in ALS) |
| 38 | L −> R (in FALS) |
| 38 | L −> V (in FALS) |
| 41 | G −> D (in FALS) |
| 41 | G −> S (in FALS) |
| 43 | H −> R (in FALS) |
| 45 | F −> C (in FALS) |
| 45 | F −> S (in ALS) |
| 46 | H −> R (in FALS) |
| 48 | H −> Q (in FALS) |
| 48 | H −> R (in ALS) |
| 49 | E −> K (in FALS) |
| 54 | T −> R (in ALS) |
| 65 | N −> S (in FALS) |
| 66 | P −> S (in ALS) |
| 67 | L −> P (in ALS) |
| 67 | L −> R (in FALS) |
| 71 | H −> Y (in ALS) |
| 72 | G −> S (in FALS) |
| 72 | G −> C (in ALS) |
| 76 | D −> Y (in FALS) |
| 76 | D −> C (in ALS) |
| 80 | H −> A (in ALS) |
| 80 | H −> R (in ALS) |
| 84 | L −> F (in FALS) |
| 84 | L −> V (in FALS) |
| 85 | G −> R (in FALS) |
| 85 | G −> S (in ALS) |
| 86 | N −> K (in ALS) |
| 86 | N −> S (in FALS) |
| 87 | V −> A (in ALS) |
| 89 | A −> T (in ALS) |
| 89 | A −> V (in FALS) |
| 90 | D −> A (in FALS) |
| 90 | D −> V (in FALS) |
| 93 | G −> A (in FALS) |
| 93 | G −> S (in ALS) |
| 93 | G −> C (in FALS) |
| 93 | G −> D (in FALS) |
| 93 | G −> R (in FALS) |
| 93 | G −> V (in FALS) |
| 95 | A −> G (in ALS) |
| 96 | D −> N (in ALS) |
| 96 | D −> Mfs8 (in ALS) |
| 97 | V −> M (in ALS) |
| 99 | I −> V (in ALS) |
| 100 | E −> G (in FALS) |
| 100 | E −> K (in FALS) |
| 101 | D −> H (in ALS) |
| 101 | D −> Y (in ALS) |
| 101 | D −> G (in FALS). |
| 101 | D −> N (in FALS). |
| 104 | I −> F (in FALS). |
| 105 | S −> L (in FALS) |
| 105 | S del SL |
| 106 | L −> V (in FALS) |
| 106 | L −> F (in ALS) |
| 108 | G −> V (in FALS) |
| 109 | D −> Y (in ALS) |
| 111 | C −> Y (in ALS) |
| 112 | I −> M (in FALS) |
| 112 | I −> T (in FALS) |
| 113 | I −> T (in FALS) |
| 113 | I −> F (in ALS) |
| 114 | G −> A (in FALS) |
| 115 | R −> G (in FALS) |
| 118 | V −> L (in ALS) |
| 118 | V −> VFLQ (in FALS) |
| 121 | E −> G (in ALS) |
| 124 | D −> G (in ALS) |
| 124 | D −> V (in FALS) |
| 125 | D −> H (in FALS) |
| 126 | L −> Z (in ALS) |
| 126 | L −> S (in FALS) |
| 127 | G −> R (in ALS) |
| 127 | G −> X (in ALS) |
| 133 | Missing (in ALS) |
| 134 | S −> N (in FALS) |
| 134 | S −> T (in ALS) |
| 137 | T −> A (in ALS) |
| 137 | T −> R (in ALS) |
| 138 | G −> E (in ALS) |
| 139 | N −> K (in FALS) |
| 139 | N −> H (in ALS) |
| 140 | A −> A (FALS) |
| 140 | A −> G (in ALS) |
| 141 | G −> E (in ALS) |
| 141 | G −> X (in ALS) |
| 141 | G −> A (in ALS) |
| 144 | L −> F (in FALS) |
| 144 | L −> S (in FALS) |
| 145 | A −> T (in FALS) |
| 145 | A −> G (in ALS) |
| 146 | C −> X (in ALS) |
| 146 | C −> R (in FALS) |
| 147 | G −> R (ALS) |
| 147 | G −> S (in ALS) |
| 148 | V −> G (in FALS) |
| 148 | V −> I (in FALS) |
| 149 | I −> T (in FALS) |
| 151 | I −> T (in FALS) |

Also provided is use of an antibody or binding fragment thereof, a fusion protein, a nucleic acid, or a pharmaceutical composition described herein, for treating a medical condition, disease, or disorder mediated by a misfolded form of SOD1 in a subject in need thereof. In some embodiments, the medical condition, disease or disorder is a neurodegenerative condition, disease or disorder. In some embodiments, the neurodegenerative condition, disease or disorder is amyotrophic lateral sclerosis (ALS), Alzheimer's disease or Parkinson's disease, or frontotemporal dementia. In some embodiments, the ALS is sporadic ALS. In some embodiments, the ALS is familial ALS. In some embodiments, the neurodegenerative condition, disease or disorder is Alzheimer's disease. In some embodiments, the neurodegenerative condition, disease or disorder is Parkinson's disease. In some embodiments, the neurodegenerative condition, disease or disorder is frontotemporal dementia. In some embodiments, the misfolded form of SOD1 comprises SOD1 monomer, a dimer comprising SOD1 monomers, or an aggregate comprising SOD1 monomers and/or dimers, or toxic trimers. In some embodiments, the SOD1 monomer comprises a mutant SOD1 monomer. In some embodiments, the mutant SOD1 monomer comprise one or more mutations selected from the group consisting of A4V, G93A, G85R, D90A, G127X, V148G, H46R, G37R, C6G, and E100G, and wherein X is truncation mutation. In some embodiments, the mutant SOD1 monomer comprise one or more mutations selected from Table 1.

Further provided is use of an antibody or binding fragment thereof, a fusion protein, a nucleic acid, or a pharmaceutical composition described herein, for the manufacture of a medicament for treating a medical condition, disease, or disorder mediated by a misfolded form of SOD1 in a subject in need thereof. In some embodiments, the medical condition, disease or disorder is a neurodegenerative condition, disease or disorder. In some embodiments, the neurodegenerative condition, disease or disorder is amyotrophic lateral sclerosis (ALS), Alzheimer's disease or Parkinson's disease, or frontotemporal dementia. In some embodiments, the ALS is sporadic ALS. In some embodiments, the ALS is familial ALS. In some embodiments, the neurodegenerative condition, disease or disorder is Alzheimer's disease. In some embodiments, the neurodegenerative condition, disease or disorder is Parkinson's disease. In some embodiments, the neurodegenerative condition, disease or disorder is frontotemporal dementia. In some embodiments, the misfolded form of SOD1 comprises SOD1 monomer, a dimer comprising SOD1 monomers, or an aggregate comprising SOD1 monomers and/or dimers, and toxic trimers. In some embodiments, the SOD1 monomer comprises a mutant SOD1 monomer. In some embodiments, the mutant SOD1 monomer comprise one or more mutations selected from the group consisting of A4V, G93A, G85R, D90A, G127X, V148G, H46R, G37R, C6G, and E100G, and wherein X is truncation mutation. In some embodiments, the mutant SOD1 monomer comprise one or more mutations selected from Table 1.

Further provided is an antibody or binding fragment thereof, a fusion protein, a nucleic acid, or a pharmaceutical composition described herein, for use in treating or preventing a disease in a subject. In some embodiments, the medical condition, disease or disorder is a neurodegenerative condition, disease or disorder. In some embodiments, the neurodegenerative condition, disease or disorder is amyotrophic lateral sclerosis (ALS), Alzheimer's disease or Parkinson's disease, or frontotemporal dementia. In some embodiments, the ALS is sporadic ALS. In some embodiments, the ALS is familial ALS. In some embodiments, the neurodegenerative condition, disease or disorder is Alzheimer's disease. In some embodiments, the neurodegenerative condition, disease or disorder is Parkinson's disease. In some embodiments, the neurodegenerative condition, disease or disorder is frontotemporal dementia. In some embodiments, the misfolded form of SOD1 comprises SOD1 monomer, a dimer comprising SOD1 monomers, or an aggregate comprising SOD1 monomers and/or dimers, and toxic trimers. In some embodiments, the SOD1 monomer comprises a mutant SOD1 monomer. In some embodiments, the mutant SOD1 monomer comprise one or more mutations selected from the group consisting of A4V, G93A, G85R, D90A, G127X, V148G, H46R, G37R, C6G, and E100G, and wherein X is truncation mutation. In some embodiments, the mutant SOD1 monomer comprise one or more mutations selected from Table 1.

In some embodiments, the antibody or binding fragment thereof, the fusion protein, the nucleic acid, or pharmaceutical composition may be administered in combination with one or more additional therapeutic agents. Co-administration includes simultaneous administration in separate compositions (also referred to as concurrent administration), administration at different times in separate compositions, or administration in a composition in which both agents are present. In some embodiments, the additional therapeutic agent is an ALS therapeutic agent. In some embodiments, the ALS therapeutic agent is Tofersen (Qalsody), AMVX0035 (RELYVRIO), edaravone (Radicava™), riluzole (Rilutek), thickened riluzole (Tiglutik), riluzole oral film (Exservan™), or Nuedexta®. In some embodiments, the ALS therapeutic agent is an anti-sense oligonucleotide. In some embodiments, the ALS therapeutic agent is Tofersen.

### V. Routes of Administration

An antibody or binding fragment thereof, fusion protein, or pharmaceutical composition that binds to misfolded SOD1 as described herein may be administered by any appropriate route. In some embodiments, the antibody or binding fragment thereof, fusion protein, nucleic acid or the pharmaceutical composition is administered parenterally. In some embodiments, the parenteral administration is selected from intravenous, intradermal, inhalation, transdermal (topical), intraocular, intramuscular, subcutaneous, intramuscular, and/or transmucosal administration. In some embodiments, the antibody or binding fragment thereof, fusion protein, nucleic acid or the pharmaceutical composition as described herein is administered subcutaneously. As used herein, the term “subcutaneous tissue”, is defined as a layer of loose, irregular connective tissue immediately beneath the skin. For example, the subcutaneous administration may be performed by injecting a composition into areas including, but not limited to, thigh region, abdominal region, gluteal region, or scapular region. In some embodiments, the antibody or binding fragment thereof, fusion protein, nucleic acid or the pharmaceutical composition as described herein is administered intravenously. In other embodiments, an antibody or binding fragment thereof, fusion protein, or pharmaceutical composition that binds to misfolded SOD1 as described herein is administered by direct administration to a target tissue, the nervous system (e.g., direct injection into the brain; intraventricularly; intrathecally). Alternatively, an antibody or binding fragment thereof, fusion protein or pharmaceutical composition that binds to misfolded SOD1 as described herein (or a composition or medicament containing a misfolded SOD1 antibody or binding fragment thereof, fusion protein or nucleic acid as described herein) can be administered by inhalation, parenterally, intradermally, transdermally, or transmucosally (e.g., orally or nasally). More than one route can be used concurrently, if desired.

In some embodiments, an antibody or binding fragment thereof, fusion protein, or pharmaceutical composition that binds to misfolded SOD1 as described herein is administered orally. In some embodiments, the present disclosure provides solid dosage forms of antibody or fragment, fusion protein, or pharmaceutical composition thereof that binds to misfolded SOD1 as described herein for oral administration including (a) an antibody or binding fragment thereof, fusion protein, or pharmaceutical composition that binds to misfolded SOD1, (b) at least one pharmaceutically acceptable pH-lowering agent, (c) at least one absorption enhancer effective to promote bioavailability of the antibody or binding fragment thereof, fusion protein or pharmaceutical composition that binds to misfolded SOD1, and (d) a protective vehicle. In some embodiments, the solid dosage form is a capsule or tablet.

The disclosure also contemplates additional methods for administering the antibody or binding fragment thereof, fusion protein, nucleic acid, or pharmaceutical composition across the blood brain barrier such as those directed at transiently increasing the permeability of the blood brain barrier as described in U.S. Pat. No. 7,012,061 “Method for increasing the permeability of the blood brain barrier”, herein incorporated by reference.

A person skilled in the art will recognize the variety of suitable methods for administering the compounds of the invention directly to the brain or across the blood brain barrier and be able to modify these methods in order to safely administer the products of the invention.

### VI. Dosing and Formulation

An effective quantity of an antibody or binding fragment thereof, a fusion protein, or a pharmaceutical composition that binds to misfolded SOD1, or a nucleic acid that encodes said antibody or binding fragment thereof, a fusion protein, or a pharmaceutical composition, is employed in treatment. The dosage of antibodies or fragments thereof, fusion protein, nucleic acid or pharmaceutical composition used in accordance with the disclosure varies depending on the antibody or binding fragment thereof, a fusion protein, or a pharmaceutical composition, and the condition being treated.

The dosage form is optionally a liquid dosage form. The term “liquid dosage form” refers to non-solid dosage forms suitable for, but not limited to, parenteral, intravenous, subcutaneous, intramuscular, intracranial, intraventricular, intrathecal, intraorbital, ophthalmic, intracapsular, intraspinal, intracisternal, intraperitoneal, intranasal, aerosol or oral administration. Solutions of a compound of the invention can be prepared in water suitably mixed with a surfactant such as hydroxypropylcellulose. Dispersions can also be prepared in glycerol, liquid polyethylene glycols, DMSO and mixtures thereof with or without alcohol, and in oils. Under ordinary conditions of storage and use, these preparations contain a preservative to prevent the growth of microorganisms. A person skilled in the art would know how to prepare suitable formulations. Conventional procedures and ingredients for the selection and preparation of suitable formulations are described, for example, in Remington's Pharmaceutical Sciences (2003-20th edition) and in The United States Pharmacopeia: The National Formulary (USP 24 NF19) published in 1999. Formulations optionally contain excipients including, but not limited to, a buffering agent, an anti-oxidant, a stabilizer, a carrier, a diluent, and an agent for pH adjustment.

The pharmaceutical forms suitable for injectable use include sterile aqueous solutions or dispersion and sterile powders for the extemporaneous preparation of sterile injectable solutions or dispersions. In all cases the form must be sterile and must be fluid to the extent that easy syringeability exists.

A person skilled in the art would recognize that the dosage form and formulation chosen depends on characteristics of the composition. For example a person skilled in the art would know that a composition comprising an antibody may require a different formulation than a composition comprising a nucleic acid and would choose a formulation and dosage form suitable to the composition.

Additional factors that affect the effective dose of a formulation include the route of administration, the target site, the physiological state of the subject, the species of the subject, whether the treatment is prophylactic or therapeutic, and whether other medications are administered.

Antibodies, for example, anti-SOD1 antibodies such as a scFv or fusion protein comprising a scFv can be used by intravenous infusion. The therapeutic concentration of SOD1 scFv or scFv-E3 ligase fusion protein may be 1-10 micrograms per mL local concentration in the CNS. In the setting of a non-disrupted blood brain barrier (BBB) only 1/100 to 1/1000 of antibodies, for example IgG, penetrates the CNS. Thus, the concentration of therapeutic antibody in the peripheral circulation necessary to reach this concentration in the CNS would be on the order of 100 micrograms/ml to maximally 10 mg/ml, close to the pre-treatment level of antibody in human plasma. Considering the human blood volume to be about 5 liters, dosing of 50 grams would be the upper limit, which is similar to the dose of pooled polyclonal intravenous immunoglobulin (IVIG) used to treat many disorders. Considering the degradation of human antibody, for example IgG, requires 3-4 weeks, dosing once per 3 weeks should constitute an effective regimen. However, dosing of scFv or scFv-E3 ligase fusion protein could be higher or lower than the above calculation, depending on the specifics of the particular condition. For example, mild disruption of the BBB has been noted in ALS, which is presumably maximal in regions of greatest neuroinflammation, e.g., those regions in which the disease is most manifest, such as the anterior horn motor neurons, and the cortical motor neurons, as well as certain fiber tracts that are subserved by cortical motor neurons. Thus, it is possible that selective BBB disruption in regions of maximal disease will permit therapeutic efficacy for lower circulating concentrations of anti-misfolded SOD1 antibodies.

The antibodies or fusion protein described herein can be used for direct infusion into the CNS via the intraventricular route or by the intrathecal route. Examples of medical devices which are used for this purpose are manufactured by MedTronic. As the CSF recirculates several times daily, ongoing infusion is required, rather than a 3-4 week dosing regimen. The end concentration of 1-10 micrograms per mL will be achieved by infusion of as much as 5 mg per day in the 500 mL per day CSF.

Various embodiments may include differing dosing regimen. In some embodiments, the antibody or binding fragment thereof, the fusion protein, the nucleic acid, or the pharmaceutical composition that binds to misfolded SOD1 is administered via continuous infusion. In some embodiments, the continuous infusion is intravenous. In other embodiments, the continuous infusion is subcutaneous. Alternatively or additionally, in some embodiments, the antibody or binding fragment thereof, the fusion protein, the nucleic acid, or the pharmaceutical composition that binds to misfolded SOD1 is administered bimonthly, monthly, twice monthly, triweekly, biweekly, weekly, twice weekly, thrice weekly, daily, twice daily, or on another clinically desirable dosing schedule. The dosing regimen for a single subject need not be at a fixed interval, but can be varied over time, depending on the needs of the subject.

In one embodiment, the local dosage is administered at least once a day until a therapeutic result is achieved. The dosage can be administered twice a day, but more or less frequent dosing can be suitable. Once a therapeutic result is achieved, the antibody or binding fragment thereof, the fusion protein, the nucleic acid, or the pharmaceutical composition can be tapered or discontinued. Occasionally, side effects warrant discontinuation of therapy. An effective quantity of the antibody or binding fragment thereof, the fusion protein, the nucleic acid, or the pharmaceutical composition of interest is employed in treatment. The dose should be sufficient to ameliorate symptoms or signs of the disease treated without producing unacceptable toxicity to the patient.

The antibody or binding fragment thereof, or fusion protein described herein can be delivered by any method known to the skilled person, for example, delivery by using a recombinant viral vector. In some embodiments, the antibody or binding fragment thereof, or fusion protein described herein, is delivered by a recombinant viral vector. In some embodiments, the viral vector is an adenoviral vector, an adeno-associated vims (AAV) vector, a herpes simplex virus (HSV) vector a lentivirus vector, a retrovirus vector, an SV-40-type virus vector, or a vaccinia virus vector. In some embodiments, the viral vector is an AAV vector. In some embodiments, the AAV vector is an AAV9 vector. In some embodiments, the AAV vector has an engineered capsid that efficiently transduces the central and/or the peripheral nervous system. In some embodiments, the AAV is AAV-PHP.eB.

The recombinant viral vector can comprise regulatory sequences allowing expression of the encoded antibody or binding fragment thereof, or fusion protein, such as for example, a promoter, enhancer internal ribosome entry sites (IRES), and/or sequences encoding protein transduction domains (PTD). In some embodiments, the viral vector comprises a promoter region, operably linked to the coding sequence, to cause or improve expression of the antibody or binding fragment thereof, or fusion protein. In some embodiments, the promoter is ubiquitous, tissue-specific, strong, weak, regulated, or chimeric, to allow efficient and suitable production of the antibody or binding fragment thereof, or fusion protein. In some embodiments, the promoter is a cellular, viral, fungal, plant or synthetic promoter. In some embodiments, the promoter for use is functional in nervous and muscle cells. In some embodiments, the promoter is functional in motor neurons and glial cells. In some embodiments, the promoter is functional in neuron, glial, and skeletal muscle cells, for example the promoter is a neural specific promoter, a glial specific promoter or a skeletal muscle specific promoter, with for example non-substantial activation of operably linked sequences in other cell types. In some embodiments, the promoter is an RNA polymerase III-dependent promoter or an RNA polymerase II-dependent promoter. In some embodiments, the regulated promoter is a Tet on/off element-containing promoter, a rapamycin-inducible promoter, or a metallothionein promoter. In some embodiments, the motor neuron specific promoter is a Calcitonin Gene-Related Peptide (CGRP) promoter, a Choline Acetyl Transferase (ChAT) promoter, or a Homeobox 9 (HB9) promoter. In some embodiments, the neuron-specific promoter is a Neuron Specific Enolase (NSE) promoter, a Synapsin promoter, or a Neuron Specific Silencer Elements (NRSE) promoter. In some embodiments, the glial cells-specific promoter is a Glial Fibrillary Acidic Protein (GFAP) promoter. In some embodiments, the promoter is a CMV promoter, an RSV promoter, a SV40 promoter, or a Chicken beta actin/CMV (CBA) promoter. In some embodiments, the promoter is a phosphoglycerate kinase (PGK) promoter or an Elongation Factor 1alpha (EF1alpha) promoter. In some embodiments, the promoter is a prion promoter. In some embodiments, the promoter is CAG promoter.

The recombinant viral vector for delivering and expressing mUBLs can be delivered at a pharmaceutically acceptable dose by direct stereotaxic brain infusion, spinal cord injection, intravascular delivery, or intramuscular delivery. In some embodiments, the recombinant viral vector encoding a mUBL is administered by stereotaxic brain infusion or spinal cord injection. In some embodiments, the recombinant viral vector encoding a mUBL is administered intravascularly or intramuscularly.

### VII. Kits

In some embodiments, the present disclosure further provides kits or other articles of manufacture which contain an antibody or binding fragment thereof, a fusion protein, a nucleic acid, or a pharmaceutical composition that binds misfolded SOD1 described herein, as well as instructions for its reconstitution (if lyophilized) and/or use. Kits or other articles of manufacture may include a container, a syringe, vial and any other articles, devices or equipment useful in administration (e.g., subcutaneous, by inhalation). Suitable containers include, for example, bottles, vials, syringes (e.g., pre-filled syringes), ampules, cartridges, reservoirs, or lyo-jects. The container may be formed from a variety of materials such as glass or plastic. In some embodiments, the container is a pre-filled syringe. Suitable pre-filled syringes include, but not limited to, borosilicate glass syringes with baked silicone coating, borosilicate glass syringes with sprayed silicone, or plastic resin syringes without silicone.

Typically, the container may hold formulations and a label on, or associated with, the container that may indicate directions for reconstitution and/or use. For example, the label may indicate that the formulation is reconstituted to concentrations as described above. The label may further indicate that the formulation is useful or intended for, for example, subcutaneous administration. In some embodiments, the container may contain a single dose of a stable formulation containing an antibody or binding fragment thereof, a fusion protein, a nucleic acid, or a pharmaceutical composition that binds misfolded SOD1. In various embodiments, a single dose of the stable formulation is present in a volume of less than about 15 ml, about 10 ml, about 5.0 ml, about 4.0 ml, about 3.5 ml, about 3.0 ml, about 2.5 ml, about 2.0 ml, about 1.5 ml, about 1.0 ml, or about 0.5 ml. Alternatively, the container holding the formulation may be a multi-use vial, which allows for repeat administrations (e.g., from 2-6 administrations) of the formulation. Kits or other articles of manufacture may further include a second container comprising a suitable diluent (e.g., BWFI, saline, buffered saline). Upon mixing of the diluent and the formulation, the final polypeptide or nucleic acid concentration in the reconstituted formulation may be at least about 0.2 μg/ml (e.g., at least about 0.5 μg/ml, at least about 1 μg/ml, at least about 2 μg/ml, at least about 5 μg/ml, at least about 10 μg/ml, at least about 20 μg/ml, at least about 25 μg/ml, at least about 50 μg/ml, at least about 75 μg/ml, at least about 0.1 mg/ml, at least about 0.2 mg/ml, at least about 0.5 mg/ml, at least about 1 mg/ml, at least about 2 mg/ml, at least about 2.5 mg/ml, at least about 5 mg/ml, at least about 10 mg/ml, at least about 20 mg/ml, at least about 30 mg/ml, at least about 40 mg/ml, at least about 50 mg/ml, at least about 75 mg/ml, at least about 100 mg/ml). Kits or other articles of manufacture may further include other materials desirable from a commercial and user standpoint, including other buffers, diluents, filters, needles, syringes, and package inserts with instructions for use. In some embodiments, kits or other articles of manufacture may include an instruction for self-administration.

The following non-limiting Examples are illustrative of the present disclosure:

### EXAMPLES

### Example 1. Epitope Mapping of Mouse Antibodies 3H1 and 8D1

Peptide targets for misfolded aggregated SOD1 provide immunogens for the development of mouse monoclonal antibodies for biochemical characterization of epitope exposure were synthesized and characterized. The peptide DLGKGGNEESTKTG (SEQ ID NO: 137) was synthesized and conjugated to KLH (keyhole limpet hemocyanin). Since the sequence DLGKGGNEESTKTG (SEQ ID NO: 137) does not have an endogenous cysteine residue, it was conjugated with KLH using the sulfo-MBS method (Pierce reagent and method) for thiol group conjugation.

SOD1 epitope DLGKGGNEESTKTG (SEQ ID NO: 137) was further analysed to determine subportions (e.g. discrete epitopes) that are immunogenic. Immunogenicity analysis of SOD1 including antigenicity plotting is used to identify discrete epitopes. Isolated peptides corresponding to these discrete epitopes are synthesized and an immunogen comprising the isolated peptide corresponding to one or more of these discrete epitopes can be used to generate antibodies.

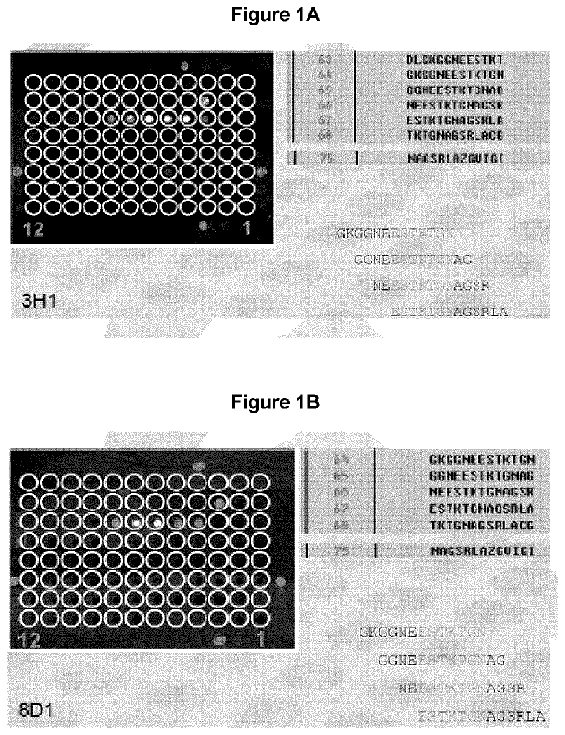

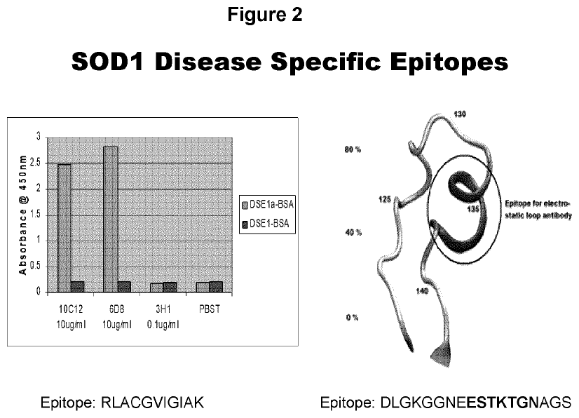

Epitope mapping experiments using isolated peptides were conducted to reveal the binding target of the mouse anti-misfolded SOD1 antibodies 3H1 and 8D1 The isolated peptides used includes DLGKGGNEESTKTG (SEQ ID NO: 137), GKGGNEESTKTGN (SEQ ID NO: 138), GGNEESTKTGNAG (SEQ ID NO: 139), NEESTKTGNAGSR (SEQ ID NO: 140), ESTKTGNAGSRLA (SEQ ID NO: 141), TKTGNAGSRLACG (SEQ ID NO: 194), and NAGSRLAZGVIGI (SEQ ID NO: 195). FIG. 1 shows the results of the epitope mapping experiment. The experiment shows that 3H1 and 8D1 are capable of binding to at least isolated peptides GKGGNEESTKTGN (SEQ ID NO: 138), GGNEESTKTGNAG (SEQ ID NO: 139), NEESTKTGNAGSR (SEQ ID NO: 140), and ESTKTGNAGSRLA (SEQ ID NO: 141) indicating a minimal epitope of ESTKTGN (SEQ ID NO: 144). This epitope ESTKTGN (SEQ ID NO: 144) is present in the electrostatic loop of the DLGKGGNEESTKTG (SEQ ID NO: 137) portion of SOD1 protein.

### Example 2. Sequences of 3H1

The mouse 3H1 antibody has been isolated and fully sequenced. RT-PCR was carried out using 5′ RACE and gene specific reverse primers which amplify the appropriate mouse immunoglobulin heavy chain (IgG2a) and light chain (kappa) variable region sequences. The specific bands were excised and cloned into pCR-Blunt JI-TOPO vector for sequencing, and the constructs were transformed into E. coli. At least 8 colonies of each chain were picked and PCR screened for the presence of amplified regions prior to sequencing and additional colonies were picked as necessary (see Table 2). Selected PCR positive clones were sequenced. DNA sequences were analyzed by BLAST and SnapGene to confirm homology to mouse antibody sequences.

TABLE 2
Summary of 3H1 PCR testing and DNA Sequencing

| | | Number of Sequences with Antibody ORF |
|---|---|---|
| Sample | Isotype | with the correct isotype |
| Mouse 3H1 | IgG2a | 8 |
| Mouse 3H1 | Kappa | 6 |

Analysis of DNA & Amino Acid Sequences: DNA sequences determined for 3H1 heavy chains and kappa chains are shown below in Table 4. The consensus DNA sequences and translated protein sequences of 3H1 are shown in Table 3. The ATG start codon and corresponding methionine amino acid is in italics, complementarity determining regions (CDRs) are bold according to IMGT/LIGM-DB. 3H1 produces mRNA containing sequences which code for IgG2a heavy chains and kappa. The consensus sequences for both heavy and light variable regions have been determined (see Table 3 for consensus sequences and Table 4 for raw sequences).

TABLE 3
Consensus DNA sequence and translated protein sequences
of the variable region of 3H1.

| Clone | | Consensus | |
|---|---|---|---|
| Names (For | | DNA | |
| Mouse Abs) | Chain | Sequence | Translated Protein Sequence |
| 3H1 | heavy chain | SEQ ID | YLGLNCVFIVFLLKGVQSEVKLEESGGGLVQPGGSMKLSC |
| | variable | NO: 93 | VASGSTFSNYWMNWVRQSPEKGLEWVAEIRLKSNTYAIHY |
| | region | | AESVKGRFTISRDNSKSSVYLQMNNLRAEDTGIYYCTGNA |
| | | | MDFWGQGTSVTVSS (SEQ ID NO: 94) |
| 3H1 | light chain | SEQ ID | KLPVRLLVLMFWIPASSSDVVMTQTPLSLPVSLGDQASIS |
| | variable | NO: 95 | CRSSQSLVYSNGNTYLHWYLQKPGQSPKLLIYKVSNRFSG |
| | region | | VPDRFSGSGSGTDFTLRISRVEAEDLGVYFCSQSTHVPPW |
| | | | TFGGGTKLEIK (SEQ ID NO: 96) |

Mouse 3H1 antibody has consensus heavy chain CDR sequences:
GSTFSNYW (SEQ ID NO: 97) corresponding to nucleic acid sequence GGATCCACTTTCAGTAACTACTGG (SEQ ID NO: 98);
VAEIRLKSNT (SEQ ID NO: 99) corresponding to nucleic acid sequence GTTGCTGAAATTAGATTGAAATCTAATACT (SEQ ID NO: 100); and
TGNADF (SEQ ID NO: 101) corresponding to nucleic acid sequence ACCGGCAATGCTATGGACTTC (SEQ ID NO: 102).

Mouse 3H1 antibody has consensus light chain CDR sequences:
QSLVYSNGNTY (SEQ ID NO: 103) corresponding to nucleic acid sequence CAGAGCCTTGTATATAGTAATGGAAACACCTAT (SEQ ID NO: 104);
KVS (SEQ ID NO: 105) corresponding to nucleic acid sequence AAAGTTTCC (SEQ ID NO: 106); and
SQSTHVPPWT (SEQ ID NO: 107) corresponding to nucleic acid sequence TCTCAAAGTACACATGTTCCTCCGTGGACG (SEQ ID NO: 108).

TABLE 4
Heavy and Light Chain Variable Raw Amino Acid and Nucleic Acid Sequences

| | |
|---|---|
| | Nucleic |
| | acid |
| Amino acid sequence | sequence |

3H1 IgG2A

| | | |
|---|---|---|
| 2A1 | MYLGLNCVFIVFLLKGVQSEVKLEESGGGLVQPGGSMKLSCVASGSTESNYWM | SEQ ID |
| | NWVRQSPEKGLEWVAEIRLKSNTYAIHYAESVKGRFTISRDNSKSSVYLQMNN | NO: 197 |
| | LRAEDTGIYYCTGNAMDFWGQGTSVTVSSAKTTA | |
| | (SEQ ID NO: 196) | |
| 2A2 or | MYLGLNCVFIVFLLKGVQSEVKLEESGGGLVQPGGSMKLSCVASGSTESNYWM | SEQ ID |
| 2A | NWVRQSPEKGLEWVAEIRLKSNTYAIHYAESVKGRFTISRDNSKSSVYLQMNN | NO: 199 |
| | LRAEDTGIYYCTGNAMDFWGQGTSVTVSSAKTTAPS (SEQ ID NO: 198) | |
| 2A3 | SEQ ID NO: 198 | SEQ ID |
| | | NO: 199 |
| 2A4 | MYLGLNCVFIVFLLKGVQSEVKLEESGGGLVQPGGSMKLSCVASGSTFSNYWM | SEQ ID |
| | NWVRQSPEKGLEWVAEIRLKSNTYAIHYAESVKGRFTISRDNSKSSVYLQMNN | NO: 201 |
| | LRAEDTGIYYCTGNAMDFWGQGT SVTVS SAKTTAPSVYPLAPVCGDTTGS SVT | |
| | LKGEFV* | |
| | (SEQ ID NO: 200) | |
| 2A5 | MYLGLNCVFIVFLLKGVQSEVKLEESGGGLVQPGGSMKLSCVASGSTFSNYWM | SEQ ID |
| | NWVRQSPEKGLEWVAEIRLKSNTYAIHYAESVKGRFTISRDNSKSSVYLQMNN | NO: 203 |
| | LRAEDTGIYYCTGNAMDFWGQGTSVTVSSAKTTAPSVYPLAPVCGDTTGSSVT | |
| | LGCLVKGYFPEPVTLTWNSGSLSSGVHTFPAVLQSDLYTLSSSVTVTSSTWPS | |
| | QSITCNVAHPASSTKVDKKIEQGRIRLNLQD* | |
| | (SEQ ID NO: 202) | |
| 2A6 | MYLGLNCVFIVFLLKGVQSEVKLEESGGGLVQPGGSMKLSCVASGSTESNYWM | SEQ ID |
| | NWVRQSPEKGLEWVAEIRLKSNTYAIHYAESVKGRFTISRDNSKSSVYLQMNN | NO: 205 |
| | LRAEDTGIYYCTGNAMDFWGQGT SVTVS SAKTTAP | |
| | (SEQ ID NO: 204) | |
| 2A7 | MYLGLNCVFIVFLLKGVQSEVKLEESGGGLVQPGGSMKLSCVASGSTFSNYWM | SEQ ID |
| | NWVRQSPEKGLEWVAEIRLKSNTYAIHYAESVKGRFTISRDNSKSSVYLQMNN | NO: 207 |
| | LRAEDTGIYYCTGNAMDFWGQGT SVTVS SAKTTAPSV | |
| | (SEQ ID NO: 206) | |
| 2A8 | (SEQ ID NO: 204) | SEQ ID |
| | | NO: 208 |

3H1 Kappa

| | | |
|---|---|---|
| K2 | MKLPVRLLVLMFWIPASSSDVVMTQTPLSLPVSLGDQASISCRSSQSLVYSNG | SEQ ID |
| | NTYLHWYLQKPGQSPKLLIYKVSNRFSGVPDRESGSGSGTDFTLRISRVEAED | NO: 210 |
| | LGVYFCSQSTHVPPWTFGGGTKLEIKRADAAP | |
| | (SEQ ID NO: 209) | |
| K3 | SEQ ID NO: 209 | SEQ ID |
| | | NO: 210 |
| K4 | SEQ ID NO: 209 | SEQ ID |
| | | NO: 210 |
| K5 | MKLPVRLLVLMFWIPASSSDVVMTQTPLSLPVSLGDQASISCRSSQSLVYSNG | SEQ ID |
| | NTYLHWYLQKPGQSPKLLIYKVSNRFSGVPDRESGSGSGTDETLRISRVEAED | NO: 212 |
| | LGVYFCSQSTHVPPWTFGGGTKLEIKRADAAPTR* (SEQ ID NO: 211) | |
| K6 | MKLPVRLLVLMFWIPASSSDVVMTQTPLSLPVSLGDQASISCRSSQSLVYSNG | SEQ ID |
| | NTYLHWYLQKPGQSPKLLIYKVSNRFSGVPDRESGSGSGTDFTLRISRVEAED | NO: 214 |
| | LGVYFCSQSTHVPPWTFGGGTKLEIKRADAAPT (SEQ ID NO: 213) | |
| K7 | MKLPVRLLVLMFWIPASSSDVVMTQTPLSLPVSLGDQASISCRSSQSLVYSNG | SEQ ID |
| | NTYLHWYLQKPGQSPKLLIYKVSNRFSGVPDRESGSGSGTDETLRISRVEAED | NO: 216 |
| | LGVYFCSQSTHVPPWTFGGGTKLEIKRADAAPTV* (SEQ ID NO: 215) | |

### Example 3A. Rabbit Anti-Misfolded SOD1 Antibodies

Further anti-misfolded SOD1 antibodies were raised in rabbits using isolated peptide NEESTKTGN (SEQ ID NO: 142). Their amino acid and corresponding nucleic acid sequences are listed in Table 5. The mature variable regions are underlined and separately shown in Table 6. The CDRs of these antibodies are shown in Table 7.

TABLE 5
Anti-SOD1 Rabbit Antibodies Amino Acid and Nucleic Acid Sequences

| | | Nucleic |
|---|---|---|
| | | Acid |
| Antibody | Amino Acid Sequence | Sequence |
| A-7-35- | METGLRWLLLVAVLKGVQCQSVEESGGRLVSPGGSLTLTCRVSGSDRRTYVMRW | SEQ ID |
| H16 | VRQAPGKGLEWIGTIRLDGSRNYATWAKGRFTISKSSSTTMELRMTSLTSEDTA | NO: 110 |
| | TYFCVRDDLWGPGTLVTVSSGQPKAPSVFPLAPCCGDTPSSTVTLGCLVKGYLP | |
| | EPVTVTWNSGTLTNGVRTFPSVRQSSGLYSLSSVVSVTSSSQPVTCNVAHPATN | |
| | TKVDKTVAPSTCSKPTCPPPELLGGPSVFIFPPKPKDTLMISRTPEVTCVVVDV | |
| | SQDDPEVQFTWYINNEQVRTARPPLREQQFNSTIRVVSTLPIAHQDWLRGKEFK | |
| | CKVHNKALPAPIEKTISKARGQPLEPKVYTMGPPREELSSRSVSLTCMINGFYP | |
| | SDISVEWEKNGKAEDNYKTTPAVLDSDGSYFLYSKLSVPTSEWQRGDVFTCSVM | |
| | HEALHNHYTQKSISRSPGK (SEQ ID NO: 109) | |
| A-7-35- | SEQ ID NO: 109 | SEQ ID |
| H17 | | NO: 111 |
| A-7-35- | METGLRWLLLVAVLKGVQCQSVEESGGRLVSPGGSLTLTCRVSGSDRRTYVMRW | SEQ ID |
| H18 | VRQAPGKGLEWIGTIRLDGSRNYATWAKGRFTISKSSSTTMELRMTSLTSEDTA | NO: 113 |
| | TYFCVRDDLGGPGTLVTVSSGQPKAPSVFPLAPCCGDTPSSTVTLGCLVKGYLP | |
| | EPVTVTWNSGTLTNGVRTFPSVRQSSGLYSLSSVVSVTSSSQPVTCNVAHPATN | |
| | TKVDKTVAPSTCSKPTCPPPELLGGPSVFIFPPKPKDTLMISRTPEVTCVVVDV | |
| | SQDDPEVQFTWYINNEQVRTARPPLREQQFNSTIRVVSTLPIAHQDWLRGKEFK | |
| | CKVHNKALPAPIEKTISKARGQPLEPKVYTMGPPREELSSRSVSLTCMINGFYP | |
| | SDISVEWEKNGKAEDNYKTTPAVLDSDGSYFLYSKLSVPTSEWQRGDVFTCSVM | |
| | HEALHNHYTQKSISRSPGK (SEQ ID NO: 112) | |
| A-7-35- | MDTRAPTQLLGLLLLWLPGARCADIVMTQTPASVEVAVGGTVTIKCQASQSISN | SEQ ID |
| L15 | LLAWYQQKPGQPPKLLIYKASTLESGVPSRFKGSGSGTQFALTISGVQCDDAAT | NO: 115 |
| | YYCQQGFDSTDVDNLFGGGTEVVVAGDPVAPTVLIFPPAADQVATGTVTIVCVA | |
| | NKYFPDVTVTWEVDGTTQTTGIENSKTPQNSADCTYNLSSTLTLTSTQYNSHKE | |
| | YTCKVTQGTTSVVQSLNRGDC (SEQ ID NO: 114) | |
| A-7-58- | METGLRWLLLVAVLKGVQCQSVEESGGRLVTPGTPLTLTCTVSGIDLSSSVMAW | SEQ ID |
| H1 | VRQAPGKGLEWIGIITNRGNRYYAKWAKGRFTISRTSTTVGLKITSPTTADTAT | NO: 117 |
| | YFCGRGSVWGPGTLVTVSSGQPKAPSVFPLAPCCGDTPSSTVTLGCLVKGYLPE | |
| | PVTVTWNSGTLTNGVRTFPSVRQSSGLYSLSSVVSVTSSSQPVTCNVAHPATNT | |
| | KVDKTVAPSTCSKPTCPPPELLGGPSVFIFPPKPKDTLMISRTPEVTCVVVDVS | |
| | QDDPEVQFTWYINNEQVRTARPPLREQQFNSTIRVVSTLPIAHQDWLRGKEFKC | |
| | KVHNKALPAPIEKTISKARGQPLEPKVYTMGPPREELSSRSVSLTCMINGFYPS | |
| | DISVEWEKNGKAEDNYKTTPAVLDSDGSYFLYSKLSVPTSEWQRGDVFTCSVMH | |
| | EALHNHYTQKSISRSPGK (SEQ ID NO: 116) | |
| A-7-58- | METGLRWLLLVAVLKGVQCQSVEESGGRLVTPGTPLTLTCTVSGIDLSSSVMVW | SEQ ID |
| H2 | VRQAPGKGLEWIGIITNRGNRYYAKWAKGRFTISRTSTTVGLKITSPTTADTAT | NO: 119 |
| | YFCGRGSVWGPGTLVTVSSGQPKAPSVFPLAPCCGDTPSSTVTLGCLVKGYLPE | |
| | PVTVTWNSGTLTNGVRTFPSVRQSSGLYSLSSVVSVTSSSQPVTCNVAHPATNT | |
| | KVDKTVAPSTCSKPTCPPPELLGGPSVFIFPPKPKDTLMISRTPEVTCVVVDVS | |
| | QDDPEVQFTWYINNEQVRTARPPLREQQENSTIRVVSTLPIAHQDWLRGKEFKC | |
| | KVHNKALPAPIEKTISKARGQPLEPKVYTMGPPREELSSRSVSLTCMINGFYPS | |
| | DISVEWEKNGKAEDNYKTTPAVLDSDGSYFLYSKLSVPTSEWQRGDVFTCSVMH | |
| | EALHNHYTQKSISRSPGK (SEQ ID NO: 118) | |
| A-7-58- | MDTRAPTQLLGLLLLWLPGATFAQVLTQTPASVSAAIGGTVTIKCQSSQSFLDD | SEQ ID |
| L1, L2 | NDLAWYQKKPGRPPKRLIKSASTLDSGVPSRFSGSGSGTQFTLTISDVQCADAA | NO: 121 |
| | TYYCLGDYDCSVGDCSAFGGGTEVVVKGDPVAPTVLIFPPAADQVATGTVTIVC | |
| | VANKYFPDVTVTWEVDGTTQTTGIENSKTPQNSADCTYNLSSTLTLTSTQYNSH | |
| | KEYTCKVTQGTTSVVQSENRGDC (SEQ ID NO: 120) | |
| A-7-63- | METGLRWLLLVAVLKGVQCQSVGESGGGLVTPGTPLTLTCTVSGFSLSSYGVSW | SEQ ID |
| H2 | VRQAPGKGLEYIGFISTRGNPYYASWAKGRFTISKTSTTVDLKITSPTTEDTAT | NO: 123 |
| | YFCAGATVVGDSIWGPGTLVTVSSGQPKAPSVFPLAPCCGDTPSSTVTLGCLVK | |
| | GYLPEPVTVTWNSGTLTNGVRTFPSVRQSSGLYSLSSVVSVTSSSQPVTCNVAH | |
| | PATNTKVDKTVAPSTCSKPTCPPPELLGGPSVFIFPPKPKDTLMISRTPEVTCV | |
| | VVDVSQDDPEVQFTWYINNEQVRTARPPLREQQFNSTIRVVSTLPIAHQDWLRG | |
| | KEFKCKVHNKALPAPIEKTISKARGQPLEPKVYTMGPPREELSSRSVSLTCMIN | |
| | GFYPSDISVEWEKNGKAEDNYKTTPAVLDSDGSYFLYSKLSVPTSEWQRGDVFT | |
| | CSVMHEALHNHYTQKSISRSPGK (SEQ ID NO: 122) | |
| A-7-63- | METGLRWLLLVAVLKGVQCQSVEESGGGLVTPGTPLTLTCTVSGFSLSSYGVSW | SEQ ID |
| H3 | VRQAPGKGLEYIGFISTRGNPYYASWAKGRFTISKTSTTVDLKITSPTTEDTAT | NO: 125 |
| | YFCAGATVVGDSIWGPGTLVTVSSGQPKAPSVFPLAPCCGDTPSSTVTLGCLVK | |
| | GYLPEPVTVTWNSGTLTNGVRTFPSVRQSSGLYSLSSVVSVTSSSQPVTCNVAH | |
| | PATNTKVDKTVAPSTCSKPTCPPPELLGGPSVFIFPPKPKDTLMISRTPEVTCV | |
| | VVDVSQDDPEVQFTWYINNEQVRTARPPLREQQFNSTIRVVSTLPIAHQDWLRG | |
| | KEFKCKVHNKALPAPIEKTISKARGQPLEPKVYTMGPPREELSSRSVSLTCMIN | |
| | GFYPSDISVEWEKNGKAEDNYKTTPAVLDSDGSYFLYSKLSVPTSEWQRGDVFT | |
| | CSVMHEALHNHYTQKSISRSPGK (SEQ ID NO: 124) | |
| A-7-63- | MDTRAPTQLLGLLLLWLPGATFAQVLTQTASSVSAAVGGTVTISCQSSQSVYKG | SEQ ID |
| L1 | YWQAWYQQKPGQPPKLLIYETSTLASGVPSRFSGSGSGTQFTLTISGVQCDDAA | NO: 127 |
| | TYYCAGGYSNNIYTFGGGTEVVVKGDPVAPTVLIFPPAADQVATGTVTIVCVAN | |
| | KYFPDVTVTWEVDGTTQTTGIENSKTPQNSADCTYNLSSTLTLTSTQYNSHKEY | |
| | TCKVTQGTTSVVQSFNRGDC (SEQ ID NO: 126) | |
| A-7-85- | METGLRWLLLVAVLKGVQCQSVEESGGRLVTPGTPLTLTCTVSGIDLSRSVMAW | SEQ ID |
| H3-2 | VRQAPGKGLEWIGIVTNRGNRYYANWAKGRFTISKTSTTVGLKIPSPTTADTAT | NO: 129 |
| | YFCGKGAVWGPGTLVTVSSGQPKAPSVFPLAPCCGDTPSSTVTLGCLVKGYLPE | |
| | PVTVTWNSGTLTNGVRTFPSVRQSSGLYSLSSVVSVTSSSQPVTCNVAHPATNT | |
| | KVDKTVAPSTCSKPTCPPPELLGGPSVFIFPPKPKDTLMISRTPEVTCVVVDVS | |
| | QDDPEVQFTWYINNEQVRTARPPLREQQFNSTIRVVSTLPIAHQDWLRGKEFKC | |
| | KVHNKALPAPIEKTISKARGQPLEPKVYTMGPPREELSSRSVSLTCMINGFYPS | |
| | DISVEWEKNGKAEDNYKTTPAVLDSDGSYFLYSKLSVPTSEWQRGDVFTCSVMH | |
| | EALHNHYTQKSISRSPGK (SEQ ID NO: 128) | |
| A-7-85- | MDTRAPTQLLGLLLLWLPGATFAQVLTQTPSSVSAAVGGTVTINCQSSQSFLDD | SEQ ID |
| L1-2 | NDLAWFQQKPGRPPKRLIKSASTLDSGVPSRFKGSGSGTQFTLTISDVQCNDAA | NO: 131 |
| | TYYCLGDYDCSVGDCSAFGGGTEVVVKGDPVAPTVLIFPPAADQVATGTVTIVC | |
| | VANKYFPDVTVTWEVDGTTQTTGIENSKTPQNSADCTYNLSSTLTLTSTQYNSH | |
| | KEYTCKVTQGTTSVVQSENRGDC (SEQ ID NO: 130) | |
| A-7-93- | METGLRWLLLVAVLKGVQCQSVEESGGRLVTPGTPLTLTCTVSGIDLRRSVMAW | SEQ ID |
| H1 | VRQAPGKGLEWIGIISDRGNTYYASWAKGRFTISRTSTTVALKMTSPTTADTAT | NO: 133 |
| | YFCGRGNVWGPGTLVTVSSGQPKAPSVFPLAPCCGDTPSSTVTLGCLVKGYLPE | |
| | PVTVTWNSGTLTNGVRTFPSVRQSSGLYSLSSVVSVTSSSQPVTCNVAHPATNT | |
| | KVDKTVAPSTCSKPTCPPPELLGGPSVFIFPPKPKDTLMISRTPEVTCVVVDVS | |
| | QDDPEVQFTWYINNEQVRTARPPLREQQFNSTIRVVSTLPIAHQDWLRGKEFKC | |
| | KVHNKALPAPIEKTISKARGQPLEPKVYTMGPPREELSSRSVSLTCMINGFYPS | |
| | DISVEWEKNGKAEDNYKTTPAVLDSDGSYFLYSKLSVPTSEWQRGDVFTCSVMH | |
| | EALHNHYTQKSISRSPGK (SEQ ID NO: 132) | |
| A-7-93- | SEQ ID NO: 132 | SEQ ID |
| H3 | | NO: 134 |
| A-7-93- | MDTRAPTQLLGLLLLWLPGATFAQVLTQTPSSVSAAVGGTVTINCQSSQRLYDD | SEQ ID |
| L1, L3 | NDCAWYQKKPGQPPKRLIHDASILDSGVSSRFSGSGSGTQFTLTISGVQCEDAA | NO: 136 |
| | TYYCLGDYDCSVADCSTFGGGTEVVVKGDPVAPTVLIFPPAADQVATGTVTIVC | |
| | VANKYFPDVTVTWEVDGTTQTTGIENSKTPQNSADCTYNLSSTLTLTSTQYNSH | |
| | KEYTCKVTQGTTSVVQSENRGDC (SEQ ID NO: 135) | |

TABLE 6
Amino Acid and Nucleic Acid Sequences of Variable Regions

| Variable | | Nucleic Acid |
|---|---|---|
| Region | Amino Acid Sequence | Sequence |
| A-35 H16 | QSVEESGGRLVSPGGSLTLTCRVSGSDRRTYVMRWVRQAPGKGLEWIGTI | SEQ ID |
| | RLDGSRNYATWAKGRFTISKSSSTTMELRMTSLTSEDTATYFCVRDDLWG | NO: 66 |
| | PGTLVTVSS (SEQ ID NO: 65) | |
| A-35 H17 | SEQ ID NO: 65 | SEQ ID |
| | | NO: 67 |
| A-35 H18 | QSVEESGGRLVSPGGSLTLTCRVSGSDRRTYVMRWVRQAPGKGLEWIGTI | SEQ ID |
| | RLDGSRNYATWAKGRFTISKSSSTTMELRMTSLTSEDTATYFCVRDDLGG | NO: 69 |
| | PGTLVTVSS (SEQ ID NO: 68) | |
| A-35 L15 | ADIVMTQTPASVEVAVGGTVTIKCQASQSISNLLAWYQQKPGQPPKLLIY | SEQ ID |
| | KASTLESGVPSRFKGSGSGTQFALTISGVQCDDAATYYCQQGFDSTDVDN | NO: 71 |
| | LFGGGTEVVVA (SEQ ID NO: 70) | |
| A-58 H1 | QSVEESGGRLVTPGTPLTLTCTVSGIDLSSSVMAWVRQAPGKGLEWIGII | SEQ ID |
| | TNRGNRYYAKWAKGRFTISRTSTTVGLKITSPTTADTATYFCGRGSVWGP | NO: 73 |
| | GTLVTVSS (SEQ ID NO: 72) | |
| A-58 H2 | QSVEESGGRLVTPGTPLTLTCTVSGIDLSSSVMVWVRQAPGKGLEWIGII | SEQ ID |
| | TNRGNRYYAKWAKGRFTISRTSTTVGLKITSPTTADTATYFCGRGSVWGP | NO: 75 |
| | GTLVTVSS (SEQ ID NO: 74) | |
| A-58 L1, | QVLTQTPASVSAAIGGTVTIKCQSSQSFLDDNDLAWYQKKPGRPPKRLIK | SEQ IL |
| L2 | SASTLDSGVPSRFSGSGSGTQFTLTISDVQCADAATYYCLGDYDCSVGDC | NO: 77 |
| | SAFGGGTEVVVK (SEQ ID NO: 76) | |
| A-63 H2 | QSVGESGGGLVTPGTPLTLTCTVSGFSLSSYGVSWVRQAPGKGLEYIGFI | SEQ ID |
| | STRGNPYYASWAKGRFTISKTSTTVDLKITSPTTEDTATYFCAGATVVGD | NO: 79 |
| | SIWGPGTLVTVSS (SEQ ID NO: 78) | |
| A-63 H3 | QSVEESGGGLVTPGTPLTLTCTVSGFSLSSYGVSWVRQAPGKGLEYIGFI | SEQ ID |
| | STRGNPYYASWAKGRFTISKTSTTVDLKITSPTTEDTATYFCAGATVVGD | NO: 81 |
| | SIWGPGTLVTVSS (SEQ ID NO: 80) | |
| A-63 L1 | QVLTQTASSVSAAVGGTVTISCQSSQSVYKGYWQAWYQQKPGQPPKLLIY | SEQ ID |
| | ETSTLASGVPSRFSGSGSGTQFTLTISGVQCDDAATYYCAGGYSNNIYTF | NO: 83 |
| | GGGTEVVVK (SEQ ID NO: 82) | |
| A-85 H3-2 | QSVEESGGRLVTPGTPLTLTCTVSGIDLSRSVMAWVRQAPGKGLEWIGIV | SEQ ID |
| | TNRGNRYYANWAKGRFTISKTSTTVGLKIPSPTTADTATYFCGKGAVWGP | NO: 85 |
| | GTLVTVSS (SEQ ID NO: 84) | |
| A-85 L1-2, | QVLTQTPSSVSAAVGGTVTINCQSSQSFLDDNDLAWFQQKPGRPPKRLIK | SEQ ID |
| L2-2 | SASTLDSGVPSRFKGSGSGTQFTLTISDVQCNDAATYYCLGDYDCSVGDC | NO: 87 |
| | SAFGGGTEVVVK (SEQ ID NO: 86) | |
| A-93 H1 | QSVEESGGRLVTPGTPLTLTCTVSGIDLRRSVMAWVRQAPGKGLEWIGII | SEQ ID |
| | SDRGNTYYASWAKGRFTISRTSTTVALKMTSPTTADTATYFCGRGNVWGP | NO: 89 |
| | GTLVTVSS (SEQ ID NO: 88) | |
| A-93 H3 | (SEQ ID NO: 88) | SEQ ID |
| | | NO: 90 |
| A-93 L1, | QVLTQTPSSVSAAVGGTVTINCQS SQRLYDDNDCAWYQKKPGQPPKRLIH | SEQ ID |
| L3 | DASILDSGVSSRFSGSGSGTQFTLTISGVQCEDAATYYCLGDYDCSVADC | NO: 92 |
| | STFGGGTEVVVK (SEQ ID NO: 91) | |

TABLE 7
Amino Acid Sequences of CDRs

| SEQ ID NO: | CDR | Sequence |
|---|---|---|
| 1 | A-7-35/H/CDR1 | GSDRRTYVMR |
| 2 | A-7-35/H/CDR2 | TIRLDGSRNY |
| 3 | A-7-35/H/CDR3 | DDL |
| 4 | A-7-35/L/CDR1 | QASQSISNLLA |
| 5 | A-7-35/L/CDR2 | KASTLES |
| 6 | A-7-35/L/CDR3 | QQGFDSTDVDN |
| 7 | A-7-58a/H/CDR1 | GIDLSSSVMA |
| 8 | A-7-58a/H/CDR2 | IITNRGNRYY |
| 9 | A-7-58a/H/CDR3 | GSV |
| 10 | A-7-58a or b/L1/CDR1 | QSSQSFLDDNDLA |
| 11 | A-7-58a or b/L1/CDR2 | SASTLDS |
| 12 | A-7-58a or b/L1/CDR3 | LGDYDCSVGDCS |
| 13 | A-7-58a or b/L2/CDR3 | LGDYDCSVGDCSA |
| 14 | A-7-58b/H/CDR1 | GIDLSSSVMV |
| 15 | A-7-58b/H/CDR2 | IITNRGNRYY |
| 16 | A-7-58b/H/CDR3 | GSV |
| 17 | A-7-63/H/CDR1 | GFSLSSYGVS |
| 18 | A-7-63/H/CDR2 | FISTRGNPYY |
| 19 | A-7-63/H/CDR3 | ATVVGDSI |
| 20 | A-7-63/L/CDR1 | QSSQSVYKGY |
| 21 | A-7-63/L/CDR2 | ETSTLAS |
| 22 | A-7-63/L/CDR3 | AGGYSNNIYT |
| 23 | A-7-85/H/CDR1 | GIDLSRSVMA |
| 24 | A-7-85/H/CDR2 | IVTNRGNRYY |
| 25 | A-7-85/H/CDR3 | GAV |
| 26 | A-7-85/L/CDR1 | QSSQSFLDDNDLA |
| 27 | A-7-85/L/CDR2 | SASTLDS |
| 28 | A-7-85/L/CDR3 | LGDYDCSVGDCSA |
| 29 | A-7-93/H/CDR1 | GIDLRRSVMA |
| 30 | A-7-93/H/CDR2 | IISDRGNTYY |
| 31 | A-7-93/H/CDR3 | GNV |
| 32 | A-7-93/L/CDR1 | QSSQRLYDDNDCA |
| 33 | A-7-93/L/CDR2 | DASILDS |
| 34 | A-7-93/L/CDR3 | LGDYDCSVADCST |
| 35 | A-7-63/H/CDR1 (CCG) | GFSLSSYGVS |
| 36 | A-7-63/H/CDR2 (CCG) | FISTRGNPYYAS |
| 37 | A-7-63/H/CDR3 (CCG) | AGATVVGDSI |
| 38 | A-7-63/L/CDR1 (CCG) | QSSQSVYKGYWQA |
| 39 | A-7-63/L/CDR2 (CCG) | ETSTLAS |
| 40 | A-7-63/L/CDR3 (CCG) | AGGYSNNIYT |
| 41 | A-7-63/H/CDR1 (PARATOME) | FSLSSYGVS |
| 42 | A-7-63/H/CDR2 (PARATOME) | YIGFISTRGNPYY |
| 43 | A-7-63/H/CDR3 (PARATOME) | GATVVGDSI |
| 44 | A-7-63/L/CDR1 (PARATOME) | QSVYKGYWQA |
| 45 | A-7-63/L/CDR2 (PARATOME) | LLIYETSTLAS |
| 46 | A-7-63/L/CDR3 (PARATOME) | GGYSNNIYT |
| 47 | A-7-63/H/CDR1 (KABAT) | YGVSWVR |
| 48 | A-7-63/H/CDR2 (KABAT) | FISTRGNPYYASWAKGR |
| 49 | A-7-63/H/CDR3 (KABAT) | GATVVGDSIW |
| 50 | A-7-63/L/CDR1 (KABAT) | SSQSVYKGYWQ |
| 51 | A-7-63/L/CDR2 (KABAT) | YETSTLA |
| 52 | A-7-63/L/CDR3 (KABAT) | CAGGYSNNI |
| 53 | A-7-63/H/CDR1 (CHOTHIA) | FSLSSYGV |
| 54 | A-7-63/H/CDR2 (CHOTHIA) | TRGNP |
| 55 | A-7-63/H/CDR3 (CHOTHIA) | GATVVGDSIW |
| 56 | A-7-63/L/CDR1 (CHOTHIA) | SSQSVYKGYWQ |
| 57 | A-7-63/L/CDR2 (CHOTHIA) | YETSTLA |
| 58 | A-7-63/L/CDR3 (CHOTHIA) | CAGGYSNNIY |
| 59 | A-7-63/H/CDR1 (AHO) | SGFSLSSYGVSWVR |
| 60 | A-7-63/H/CDR2 (AHO) | YYASWAKGRFTISKTSTTVDL |
| 61 | A-7-63/H/CDR3 (AHO) | LVTVSSGOPKAPSVFPLAPCCGDTPSSTVT |
| 62 | A-7-63/L/CDR1 (AHO) | SQSVYKGYWQAWYQQK |
| 63 | A-7-63/L/CDR2 (AHO) | GVPSRFSGSGSGTQFTL |
| 64 | A-7-63/L/CDR3 (AHO) | KGDPVAPTVLIFPPAADQVATGTVTIVCV |

### Example 3B. Generating Rabbit Anti-Misfolded SOD1 Antibodies Via Hybridomas

Three-month-old New Zealand rabbits are immunized with 100 gtg of soluble synthetic peptides NEESTKTGN (SEQ ID NO: 142) or ESTKTGN (SEQ ID NO: 144) administered with Freund's adjuvant under a protocol of five subcutaneous injections and two test bleeds per rabbit. Titers are monitored regularly during the immunization period. Positive immunoreactive rabbits are identified by ELISA and Western blot analysis and chosen for rabbit monoclonal preparation. To generate rabbit hybridomas, splenocytes from the immunized rabbits are isolated and fused with a rabbit hybridoma fusion partner. Hybridoma clones secreting the misfold-specific SOD1 antibody are selected through ELISA screens of hybridoma supernatants.

Clonal cell lines expressing rabbit IgG for the misfold-specific antigen are further screened by Western blot analysis and immunohistochemistry using a panel of cell lines expressing either wild-type SOD1, with or without amplification, or misfolded SOD1 with a linearized alpha-helical sequence from the electrostatic loop.

### Example 3C: Generating Humanized Rabbit Anti-Misfolded SOD1 Antibodies Via CDR Grafting

Rabbit monoclonal anti-misfolded SOD1 antibodies are produced as in Example 3B. Antibody structures and/or sequences are analyzed to determine the combined Kabat, IMGT, and Paratome complementary determining regions (CDR; Zhang et al., 2017). The identified CDRs from rabbit monoclonal anti-misfolded SOD1 antibodies are then humanized through grafting onto a suitable stable human Ig germline framework to produce an equivalent humanized scFv for each rabbit monoclonal antibody. Genes encoding DNA sequences of CDR grafted scFvs are synthesized by overlap extension PCR. The DNA constructs are then transformed into E. coli with expression plasmids. Three hours after induction, E. coli cells are harvested, inclusion bodies are isolated, and humanized scFv proteins are purified, then renatured by rapid dilution into refolding buffer.

Humanized scFvs are then characterized and compared to the original rabbit monoclonal anti-misfolded SOD1 antibodies through ELISA screens. The humanized scFvs are further screened by Western blot analysis and immunohistochemistry using a panel of cell lines expressing either wild-type SOD1, with or without amplification, or misfolded SOD1 with a linearized alpha-helical sequence from the electrostatic loop.

### Example 4A. Binding Analysis of Rabbit Anti-SOD1 Antibodies

Supernatants producing hybridoma cell cultures producing rabbit monoclonal antibodies are analyzed by indirect ELISA for antibody activity on plates coated with 1 μg/well of peptide or protein antigen. Table 8A summarized the results of the binding assay. As shown in the experiment, the two A-7-85 antibodies (7-85 H3-2/L1-2 and 7-85 H3-2/L2-2) show 2× more specificity than 3H1. Sequence analysis in Example 3A shows that 7-85 H3-2/L1-2 and 7-85 H3-2/L2-2 have identical sequence. The binding of anti-SOD1 antibodies were also characterized by ratio analyzes. By Peak agg/dim OD ratio method, all A-7 antibodies analyzed are more specific than 3H1. By ratio of EC₅₀ method, most A-7 antibodies are less specific than 3H1, however, EC₅₀ values for dimeric SOD1 were extrapolated as full curves are not available (except for A-7-63).

TABLE 8A
Binding Analysis of Rabbit anti-SOD1 Antibodies

| | | | | | | Ab conc |
|---|---|---|---|---|---|---|
| | | | | Ratio | | (ng/mL) |
| | | | | Agg/Dim | Peak | at peak |
| | | | | SOD1 | agg/dim | agg/dim |
| Antibody | Peptide | Agg SOD1 | Dim SOD1 | EC₅₀ | OD ratio | OD ratio |
| A-7-58 H1/L1 | 3.52 × 10⁻¹¹ | 3.77 × 10⁻¹¹ | 1.15 × 10⁻⁹ | 30.4 | 12.65 | 10.34 |
| A-7-58 H2/L2 | 3.13 × 10⁻¹¹ | 9.72 × 10⁻⁹ | Cannot be | n/a | Peak | n/a |
| | | | accurately | | ratio has | |
| | | | calculated | | not been | |
| | | | | | reached | |
| A-7-63 H2/L1 | 2.68 × 10⁻¹¹ | 2.39 × 10⁻¹¹ | 6.37 × 10⁻¹⁰ | 26.7 | 10.69 | 6.01 |
| A-7-63 H3/L1 | 1.63 × 10⁻¹¹ | 1.56 × 10⁻¹¹ | 5.13 × 10⁻¹⁰ | 32.8 | 11.61 | 5.11 |
| A-7-85 H3- | 3.10 × 10⁻¹¹ | 5.02 × 10⁻¹¹ | 6.41 × 10⁻⁹ | 127.7 | 14.06 | 27.13 |
| 2/L1-2 | | | | | | |
| A-7-85 H3- | 3.82 × 10⁻¹¹ | 5.82 × 10⁻¹¹ | 5.10 × 10⁻⁹ | 87.7 | 14.44 | 26.14 |
| 2/L2-2 | | | | | | |
| A-7-93 H1/L1 | 2.97 × 10⁻¹¹ | 7.57 × 10⁻¹¹ | 1.95 × 10⁻⁹ | 25.8 | 11.87 | 21.92 |
| A-7-93 H3/L3 | 3.28 × 10⁻¹¹ | 8.30 × 10⁻¹¹ | 1.88 × 10⁻⁹ | 22.6 | 11.30 | 23.83 |
| 3H1 | 1.49 × 10⁻¹⁰ | 1.50 × 10⁻⁹ | 7.90 × 10⁻⁸ | 52.6 | 9.76 | 617.43 |

### Example 4B. Generation of scFv

B lymphocytes produced from immunization of rabbit with the antigen NEESTKTGN was isolated and immortalized with myeloma cells. Hybridoma cell lines secreting monoclonal antibodies are then selected using limited dilution cloning. The rabbit antibodies have been isolated and sequenced. RT-PCR was carried out using 5′ RACE and gene specific reverse primers which amplify the appropriate rabbit immunoglobulin heavy chain and light chain variable region sequences. The specific bands were excised and cloned into pCR-Blunt II-TOPO vector for sequencing, and the constructs were transformed into E. coli. Colonies of each chain were picked and PCR screened for the presence of amplified regions prior to sequencing and additional colonies were picked as necessary. Selected PCR positive clones were sequenced. DNA sequences were analyzed by BLAST and SnapGene to confirm homology to rabbit antibody sequences. Once sequences have been confirmed, the heavy chain and light chain variable sequences were cloned in expression vector pcDNA3-R4-uAb for the generation of anti-SOD1 scFvs (e.g. A-7-35a, 35, 58a, 58b, 63a, 63b, 85, and 93).

### Example 5A. Targeting Misfolded SOD1 for Proteasomal Degradation Via SOD1-scFv-E3 Ligase Fusion Protein

### Methods and Materials

### Plasmids

Expression vector pcDNA3-R4-uAb containing a single chain variable fragment for Beta-galactosidase fused to a truncated E3 ligase CHIP (carboxyl terminus of Hsc70-interacting protein) were obtained from Addgene (Addgene plasmid 101800, deposited by Matthew De Lisa (Portnoff et al., 2014)). The mUbLs used in this study were generated by replacing the Beta-galactosidase scFv with anti-SOD1 scFvs described in Example 4B. Table 8B shows which mUbL number corresponding with which fusion protein comprising what particular scFv (see Table 12 for sequences).

TABLE 8B
mUbL Number, mUbL Name, and A-scFv

| mUbL Number | mUbL Name | A-scFv |
|---|---|---|
| 1 | 35a | 35a |
| 2 | 35b | 35b |
| 3 | 58a | 58a |
| 4 | 58b | 58b |
| 5 | 63a | 63a |
| 6 | 63b | 63b |
| 7 | 85 | 85 |
| 8 | 93 | 93 |

Vectors for the expression of C-terminally EGFP-tagged SOD1 variants SOD1^{WT}, SOD1^{A4V}, SOD1^{G93A}, SOD1^{G85R}, SOD1^{D90A}, SOD1^{G127X}, SOD1^{V148G}, SOD1^{H46R}, SOD1^{G37R}, SOD1^{C6G}, SOD1^{E100G} on a pEGFP-N1 backbone have been previously described (Turner et al 2005, Farrawell et al 2019).

### Cell Culture and Transfection

Human Embryonic Kidney (HEK293), Neuro2a (N2a) and neuroblastoma SHSY5Y cells were maintained in Dulbecco's Modified Eagle's Medium/Ham's Nutrient Mixture F12 (DMEM/F12 supplemented with 10% fetal bovine serum (FBS, Bovogen Biologicals, Australia)). Cells were maintained at 37° C. in a humidified incubator with 5% atmospheric CO₂. For confocal microscopy, cells were grown in 96 well optical bottom plates (Thermoscientific, Australia). Cells were grown in six well plates for cell lysate experiments. Cells were plated at ~25% confluency 24 h prior to transfection with TransIT-X2 transfection reagent (Mirus Bio, USA). Transfections were carried out according to manufacturer's instructions with 0.1 ug DNA per well for a 96-well plate, 0.5 ug DNA per well for 24-well plates, 2.5 ug DNA per well for 6 well plates. For co-transfections, the amount of DNA was divided equally between constructs.

### Immunofluorescence

HEK293 cells were plated into 96 well optical bottom plate (Thermofisher, USA) or coverslips and co-transfected with GFP-tagged SOD1A4V and different mUbLs. After 48 hours, cells were fixed for 20 min at room temperature (RT) with 4% paraformaldehyde (PFA) (Merck Millipore, USA) in phosphate-buffered saline (PBS). Cells were permeabilised in 0.11% Triton X-100 (TX-100) in PBS for 10 min before blocking for 1 h at RT with 5% FBS, 1% bovine serum albumin (BSA) 0.3% TX-100 in PBS. Cells were incubated with mouse primary antibodies against the Histidine tag (Ab18184, Abcam, UK; 1:1000 dilution) overnight at 4° C. followed by Alexa Fluor 647-conjugated Goat anti-mouse-IgG secondary antibody (ab150115, Abcam, UK; 1:1000 dilution) for 1 h at RT. All antibodies were diluted in blocking buffer and cells were washed with PBS between each incubation step.

### Measurement of Ubiquitin-Proteasome System Function

To determine the mode of action of mUbLs, HEK293 cells were co-transfected with SOD1-A4V-EGFP and mUbLs and treated overnight (~18 h) with 10 M of the proteasome inhibitor MG132.

### Fluorescence Measurements

The fluorescence of cells expressing SOD1 was monitored over 48 h in an Incucyte automated fluorescent microscope (Essen BioScience, USA) as described in McAlary et al. (2016). Images were acquired every 2 h and analysed using a processing definition trained to select GFP-positive cells. The number of GFP-positive cells were normalised to GFP-positive cells in the control for mUbL wells. To measure insoluble GFP aggregates, 48 h post transfection cells were treated with 0.03% saponin (Sigma, Germany) in PBS. After a 10 min incubation at room temperature, GFP signal was analysed using a processing definition trained to select GFP-positive cells. To measure soluble fluorescence after saponin treatment, 100 ul of media was transferred to a fresh 96 well plate and GFP soluble fluorescence was measured on POLARstar Omega plate reader (BMG, Germany). Settings included a 2×2 matrix well scan reading from the bottom optic with excitation at 485 nm and emission at 520 nm.

### High Throughput Fluorescence Measurements

A Thunder automated microscope (Leica, Germany) was used for plate-based image acquisition. HEK293 cells co-expressing SOD1 mutant EGFP-tagged constructs and mUbLs were co-transfected into a 96 well optical bottom plate. Each well was imaged in a 9×9 tile scan. No image overlapping was used in order to avoid duplicating cell counts in later analysis stages.

### Image Analysis

All images generated via automated microscopy underwent pre-processing quality control to omit out of focus images. Out of focus images were manually assessed by users and excluded from the data set. After quality control processing, images were processed in CellProfiler to segment cells within the range of 30-130 pixel units and measure intensity, granularity, size/shape, intensity distribution and texture.

### Cell Lysis

HEK293 cells grown in six-well plates and transfected with mUbLs were harvested 48 h post transfection with trypsin-EDTA (Gibco). Cells were washed with PBS before being resuspended in RIPA buffer [50 mM Tris-HCl pH 7.4, 1% (w/v) sodium deoxycholate, 150 mM NaCl, 1 mM EDTA, 1% TX-100, 0.1% SDS, 10 mM NEM, 1 mM sodium orthovanadate, Halt™ Protease Inhibitor Cocktail (Thermo Scientific)]. Protein concentration was determined with a DC assay.

### Protein Analysis

Cell lysates with a total protein concentration of 30 μg were mixed with 4× reducing SDS-PAGE sample buffer [200 mM Tris-HCl pH 6.8, 8% SDS (w/v), 40% glycerol (v/v), 50 mM EDTA, 0.08% bromophenol blue (w/v), 4% β-mercaptoethanol (v/v)]. and heated for 10 min at 70° C. before being loaded onto 4-20% Criterion™ TGX Stain-Free™ gels (BioRad, Australia). Gels were electrophoresed for 5 min at 100 V and then 1 h at 150 V. Following electrophoresis, total protein on the gel was quantified using a Criterion Stain Free™ Imager (BioRad, Australia) prior to transferring for immunoblotting. Proteins separated by SDS-PAGE were transferred onto methanol activated Amersham™ Hybond™ 0.2 μm PVDF membranes (GE Healthcare, USA) at 100 V for 1 h at 4° C. using 1× transfer buffer (25 mM Tris-base 20% methanol (v/v) 192 mM glycine). Membranes were imaged with a stain-free imager post-transfer to confirm transfer and measure total protein. The membrane was blocked in 5% skim milk powder in Tris-buffered saline with 0.2% (v/v) Tween-20 (TBST) for 1 h at RT before probing with primary antibody overnight at 4° C. The following day membranes were washed three times with TBST over 30 min before incubating with secondary antibody for 1 h at RT. The membrane was visualised with chemiluminescent substrate (Thermo Scientific) on the Amersham Imager 6600RGB. Analysis and quantification were performed using ImageJ (version 1.53c). Membranes were then stripped with sodium azide for 2 h at RT if needed. The following primary antibodies were utilised for immunoblotting: mouse anti-FLAG (F1804 and F3165, Sigma, Germany; 1:2500), rabbit anti-SOD1 (Ab13498, Abcam, UK; 1:5000), rabbit anti-GFP (ab290, Abcam, UK; 1:10000) rabbit anti-GAPDH (G8795, Sigma, Germany; 1:50000), mouse anti-GAPDH (G9545, Sigma, Germany; 1:50000). Secondary antibodies goat anti-mouse-IgG conjugated to HRP (P044701-2, Dako Agilent; 1:5000) or goat anti-rabbit-HRP (P044801-2, Dako Agilent; 1:5000) were used as needed.

### Co-Immunoprecipitation

Dynabead co-immunoprecipitation kit (Thermofisher, USA) was used for the detection of SOD1 binding by mUbLs according to manufacturer instructions. Briefly, HEK293 cells were grown in 6 well plates, co-transfected with 250 ng each of mUbL and SOD1 A4V-GFP. 48 h post transfection, cells were lysed with extraction IP buffer. The cell lysates were mixed with 1.5 mg of magnetic dynabeads coupled to anti-His tag antibodies (ab18184, Abcam) for 30 min at 4° C. After washing, bound proteins were eluted and boiled in 4× Laemmeli sample buffer for analysis by immunoblotting.

### Results and Discussion

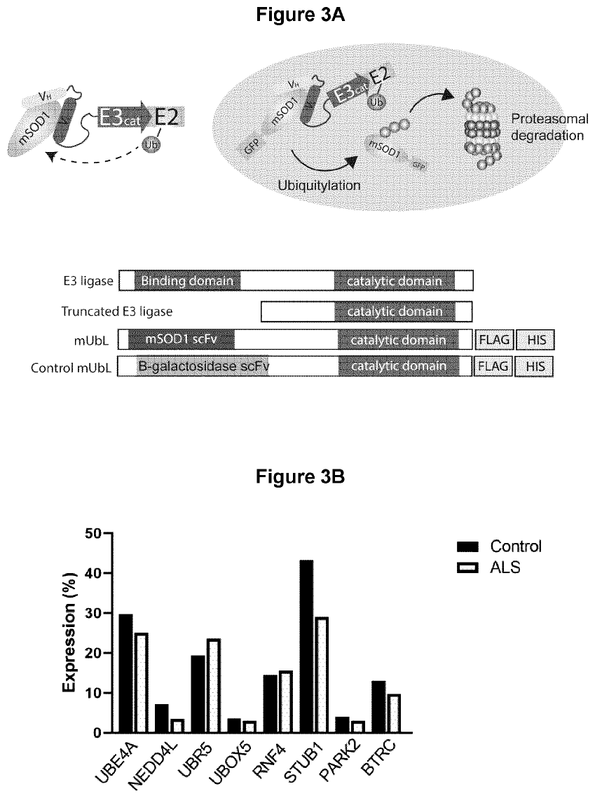

FIG. 3 shows the design of misfold specific Ubiquitin Ligase fusion proteins (mUbL). The heavy and light chain variable fragments for 8 different scFv clones (labelled mUbL1-8; see Table 9) specific for misfolded SOD1 were fused via a flexible Glycine-Serine linker to a truncated E3 ligase (FIG. 3A). See Table 6 for the amino acid and nucleic acid sequences of the heavy chain and light chain variable fragments present in each clone. The constructs include a Flag and His tag for detection. Initially, the E3 ligase carboxy-terminus of Hsc70 interacting protein (CHIP) was selected for mUbL construction due to its high expression levels and location in both human and mouse cells lines (see Table 10A). mUbLs were designed to bind specifically to misfolded forms of SOD1 and thus bringing an E3 ubiquitin ligase in proximity of the misfolded SOD1 to result in the ubiquitination and proteasomal degradation of misfolded SOD1. A construct containing an scFv for $-galactosidase fused to truncated CHIP was used as a control. FIG. 3B shows gene expression data from human postmortem anterior horn. E3 ligases have been shown to be part of a wide cohort of proteins which show differential expression in ventral horn spinal cord tissue from ALS donors relative to control samples (D'Erchia et al., 2017).

TABLE 9
The heavy and light variable fragments
in the 8 different scFv clones.

| mUbL Number | mUbL Name | Variable Regions |
|---|---|---|
| 1 | 35a | A-35 L15 + A-35 H17 |
| 2 | 35b | A-35 L15 + A-35 H18 |
| 3 | 58a | A-58 L1 + A-58 H1 |
| 4 | 58b | A-58 L1 + A-58 H2 |
| 5 | 63a | A-63 L1 + A-63 H2 |
| 6 | 63b | A-63 L1 + A-63 H3 |
| 7 | 85 | A-85 H3-2 + A-85 L1-2 |
| 8 | 93 | A-93 H1 + A-93 L1 |

TABLE 10A
Expression levels and cellular location of ligases that can be used in mUbL.

| | Human | | | | | |
|---|---|---|---|---|---|---|
| | Cerebral cortex | | Spinal | Mouse | | |
| | Expression | | cord | Cerebral | HEK293 | |
| | in neuronal | Expression | Expression | cortex | Expression | |
| | cells | (NX) | (NX) | pTPM | (NX) | Location |
| CHIP | Medium | 53.3 | 34.5 | 138.3 | 67.6 | Nucleo-plasm |
| | | | | | | and cytosol |
| UBE4A | Medium | 36.6 | 25.5 | 30.9 | 7.6 | Localised to |
| | | | | | | nuclear speckles |
| NEDD4L | Medium | 24.7 | 3.2 | 22.1 | 4.7 | Unknown |
| UBR5 | No data | 32.8 | 17.5 | 18.9 | 8.6 | Nucleo-plasm |
| | | | | | | and cytosol |
| RNF4 | Low | 50.3 | 23.1 | 64.6 | 12.8 | Nucleo-plasm |
| | | | | | | and nuclear |
| | | | | | | bodies |
| UBOX5 | Not | 16.3 | 7.9 | 7.5 | 2.9 | Nucleo-plasm |
| | detected | | | | | |
| BTrCP | Medium | 28.0 | 10.2 | 55.4 | 8.9 | Nucleo-plasm, |
| | | | | | | cytosol, plasma |
| | | | | | | membrane |
| Parkin | High | 13.4 | 5.9 | 3.4 | 0.6 | Nuclear speckles |
| | | | | | | and cytosol |

Data in Table 10A is sourced from proteinatlas.org. mRNA expression data is given an NX or normalised expression score by combining data from three transcriptome datasets; the human protein atlas (HPA), the Genotype-Tissue Expression Portal (GTEx) and CAGE data generated by the Fantom5 consortium.

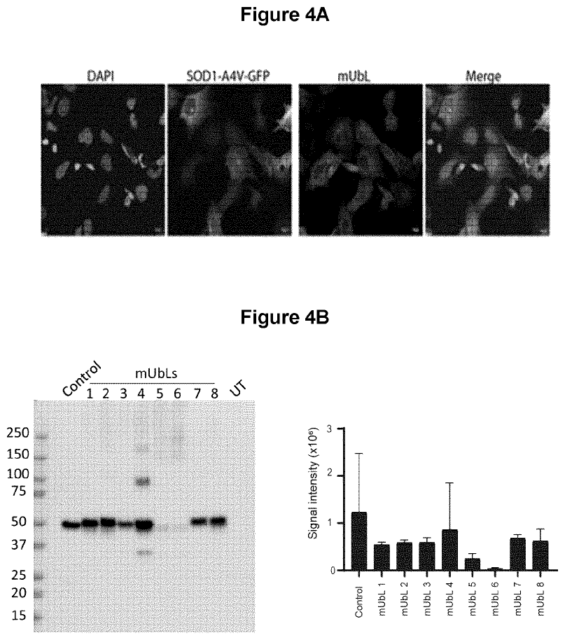

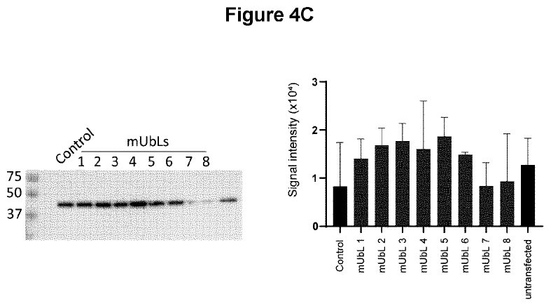

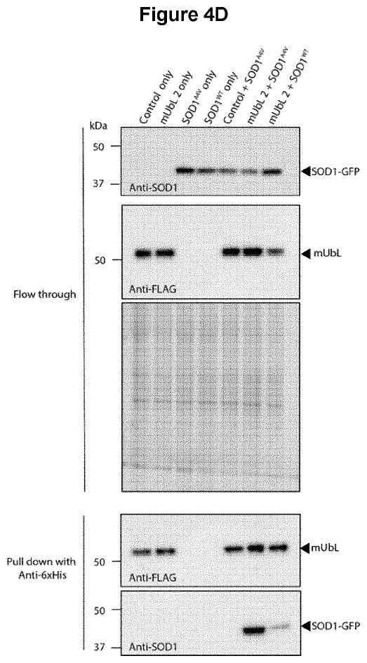

FIG. 4 shows that mUbLs are expressed in the nucleus and cytoplasm of HEK293 cells and interact with misfolded SOD1-A4V. HEK293 cells transfected with mUbL and SOD1-A4V-GFP were fixed, permeabilized and stained for the mUbL C terminal His tag using anti-His antibodies 48hpost-transfection (FIG. 4A; scale bars 10 uM). HEK293 cells were transfected with mUbLs and lysed with RIPA buffer 48 h post transfection. Immunoblotting with anti-FLAG antibodies showed that the mUbLs were present in the soluble fraction (FIG. 4B; Control is beta-galactosidase scFv, and “UT” is untransfected). Significance was determined using one-way ANOVA with Dunnett's multiple comparisons test with comparisons made against HEK293 cells transfected with the mUbLs and control, and no difference was detected amongst the mUbLs, and between the mUbLs and the control. FIG. 4C is a Western blot showing that mUbLs do not degrade endogenous SOD1. Error bars represent SD of the mean of 2 separate experiments and the whole experiment was repeated. Significance was determined using one-way ANOVA with Dunnett's multiple comparisons test with comparisons made against untransfected HEK293 cells. No significant difference was detected amongst the mUBLs and between the mUbLs and the control. Next, magnetic beads were coupled to anti-His antibodies and then mixed with lysates containing different combinations of mUbL 2 or the control construct and SOD1-EGFP. The flow through fractions were probed with anti-SOD1 and anti-FLAG antibodies which confirmed SOD1 and mUbL in the lysates (FIG. 4D). Elution fractions showed mUbL 2 could strongly bind SOD1^{A4V}-EGFP and weakly bind SOD1^{WT}-EGFP. mUbL 5 also binds to SOD1A4v. Blot is representative of 3 biological replicates.

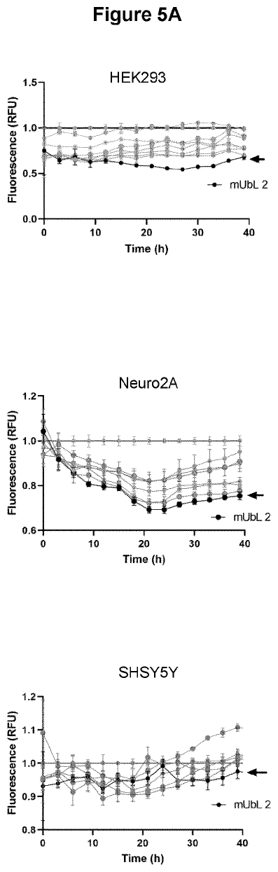

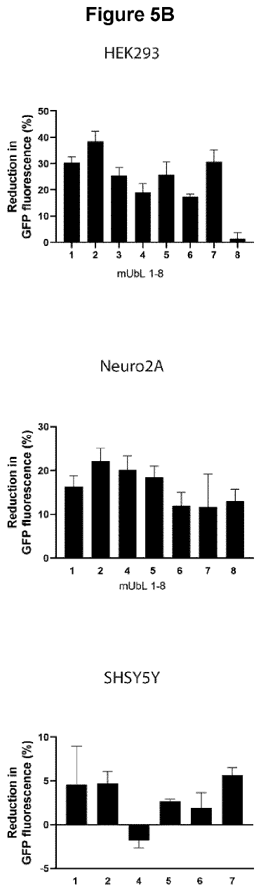

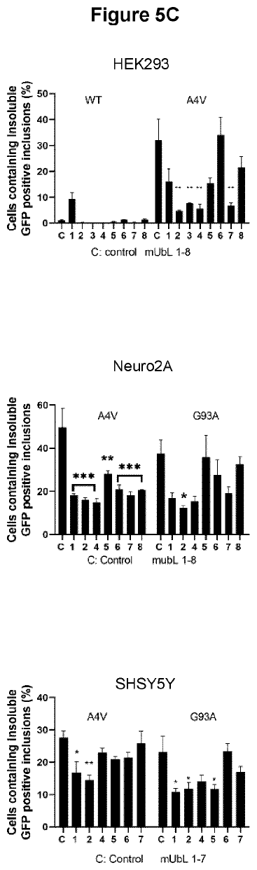

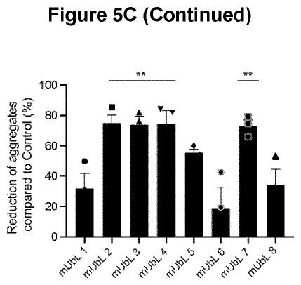

FIG. 5 shows that mUbLs reduce SOD1-A4V-GFP fluorescence in HEK293, Neuro2a and SHSY5Y cell lines. Cells were co-transfected with either SOD1-WT-GFP, SOD1-A4V-GFP or SOD1-G93A-GFP and individual mUbLs, and fluorescence was measured every 3 h for 48 h then cells were treated with saponin to induce pores in the membrane, so that soluble proteins can diffuse out of the cells, but insoluble aggregates remained trapped and were counted. Total fluorescence measurements normalised to SOD1-A4V-GFP over 48 h showed all mUbLs were able to reduce the amount of SOD1-A4V-GFP (FIG. 5A). Area under the curve calculations show the reduction in fluorescence over time relative to cells co-transfected with control mUbL and SOD1-A4V-GFP. mUbL 2 was consistently effective across all 3 cell lines with reductions in fluorescence ranging from 5% in SHSY5Y cells to 40% in HEK293 cells (FIG. 5B). In cells co-transfected with SOD1^{G93A}-EGFP, four of the mUbLs could significantly reduce the number of insoluble aggregates compared to cells transfected with the control construct (FIG. 5C, last panel). mUbL 2 was also the most effective at reducing the number of insoluble fluorescent aggregates present in transfected cells by maintaining SOD1 at levels where it could diffuse out through saponin induced pores in the cell membrane (FIG. 5C). Statistical significance was determined against cells transfected with the control mUbL p<0.01, one way ANOVA with Dunnett's multiple comparisons Graphs are representative from at least 3 experiments in each cell line.

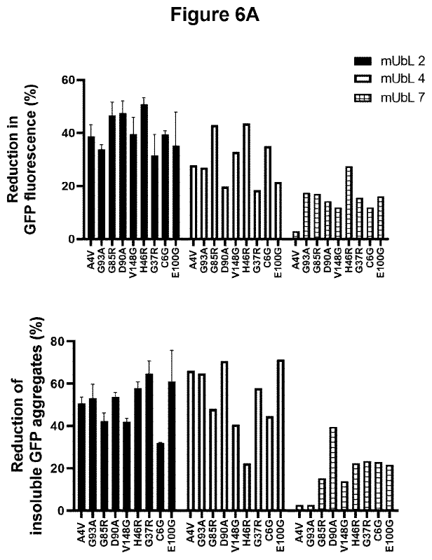

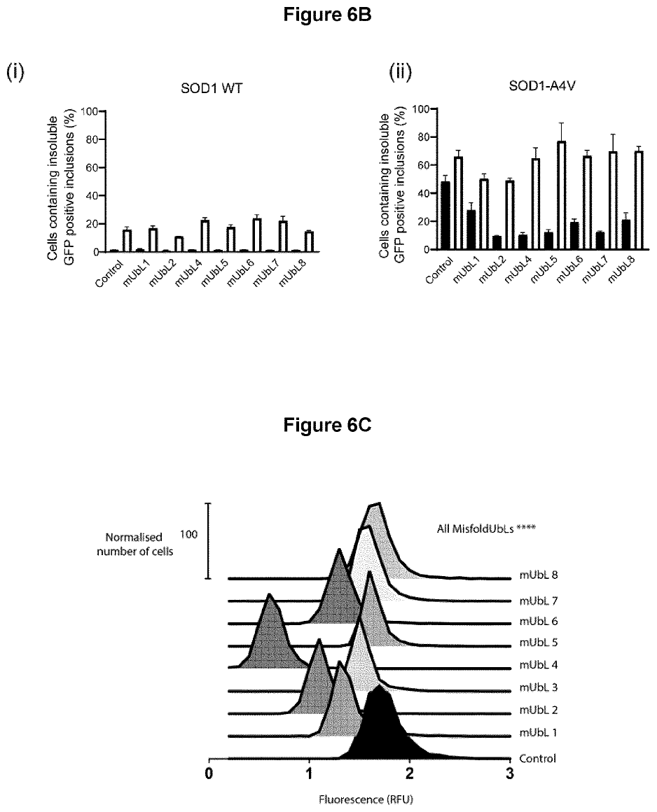

FIG. 6 shows that mUbLs can reduce misfolded SOD1 levels across a range of SOD1 mutations. HEK293 cells co-transfected with A4V, G93A, G85R, D90A, G127X, V148G, H46R, G37R, C6G, or E100G were examined for (i) total SOD1-GFP level across a 48-h time course and (ii) SOD1-GFP insoluble aggregates (FIG. 6A). For comparison purposes, the % reduction in the number of cells with aggregates relative to cells co-transfected with the control mUbL is presented. The bars represent percent reduction from controls. mUbL action is prevented when the proteasome is inhibited by treatment with MG132 (FIG. 6B and Table 10B). The proportion of cells with aggregates decreased on transfection with mUbLs (black bars) but this protective action was abolished when cells were incubated with MG132 (white bars). Next, automated microscopy was used to compare the effect of mUbLs on SOD1^{A4V}-EGFP fluorescence. HEK293 cells were co-transfected with mUbLs and SOD1^{A4V}-EGFP, fixed permeabilized and stained with anti-His antibodies. Plates were imaged, and fluorescence intensity analysed using CellProfiler. FIG. 6C shows frequency distribution graph showing all mUbLs could reduce SOD1^{A4V}-EGFP fluorescence compared to the control mUbL (shaded in black) with mUbL 4 and 2 showing the greatest reduction (one-way ANOVA with Dunnett's multiple comparisons test, p<0.0001).

TABLE 10B
Effectiveness of 35b-CHIP with different SOD1
mutants (compared to control construct

| | Reduction of insoluble | Reduction of total |
|---|---|---|
| Mutant | aggregates (%) | mutant-SOD1-EGFP (%) |
| G37R | 65 | 32 |
| E100G | 61 | 35 |
| H46R | 58 | 51 |
| D90A | 54 | 48 |
| G93A | 53 | 34 |
| A4V | 51 | 39 |
| G85R | 42 | 47 |
| C6G | 32 | 40 |

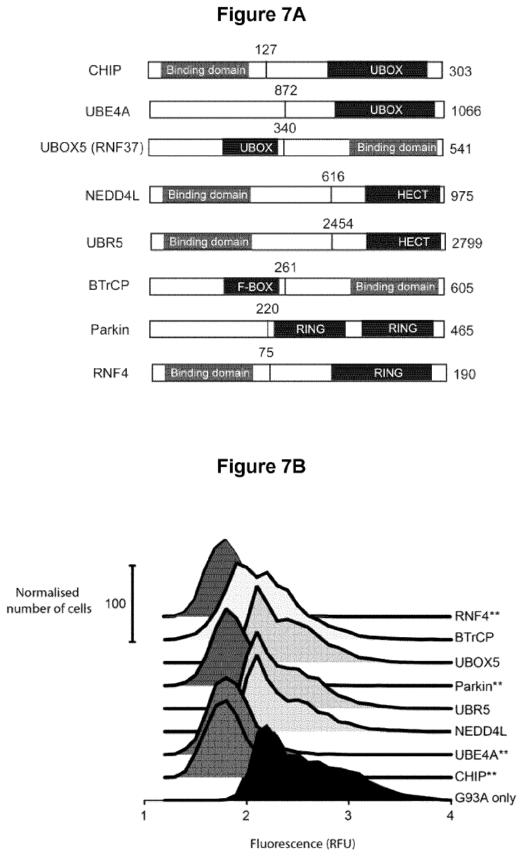

FIG. 7 shows the design of E3 ligase panel. A panel of ligases was selected and truncations designed (FIG. 7A). The panel included representatives from 5 families of ligases; U-BOX (CHIP, UBE4A, and UBOX5/RNF37), HECT (NEDD4L and UBR5), F-BOX (BtrCP), RBR (Parkin), and RING (RNF4; See Table 11). Some of the truncations were based on previous work by Ottis et al 2017 and Ibrahim et al 2020, herein incorporated by reference. In each case, the binding domain was removed from the E3 ligase to create a truncated ligase, and the scFv from mUbL 2 was fused to the truncated ligase to create a fusion protein (see Table 12), e.g. 35bX-ΔCHIP-HIS-FLAG, 35bX-ΔUBE4A-HIS-FLAG, HIS-FLAGtag-ΔUBOX5-35bX, HIS-FLAGtag-35bX-ΔNEDD4L, HIS-FLAGtag-35bX-ΔUBR5, HIS-FLAGtag-ΔBTrCP-35bX, 35bX-ΔParkin-HIS-FLAG, and 35bX-ΔRNF4-HIS-FLAG. Next, automated microscopy was used to compare the effect of the mUbL ligase panel on SOD1^{G93A}-EGFP fluorescence. HEK293 cells were co-transfected with one of the mUbL ligase panel and SOD1^{G93A}-EGFP. After 48 h cells were fixed, permeabilised and stained with anti-His antibodies. Plates were imaged, and fluorescence intensity analysed using CellProfiler. FIG. 7B shows normalised frequency distribution graph showing that when the expression of the mUbL ligase was controlled, CHIP, UBE4A, Parkin and RNF4 could significantly reduce SOD1^{G93A}-EGFP fluorescence compared to cells transfected with SOD1^{G93A}-EGFP alone (shaded in black). (Kruskall-Wallis one way ANOVA, n=35955, p<0.0001). Generally, the greater the expression, the greater the reduction in SOD1-G93A-GFP fluorescence, with CHIP in the high expression, high efficacy category.

Table 11. Sequences for E3 ligase panel.

TABLE 11
Sequences for E3 ligase panel.

| | | | Truncated |
|---|---|---|---|
| | | | Nucleic |
| | E3 | | Acid |
| Family | Ligase | Full Amino Acid Sequence | Sequence |
| U-BOX | CHIP | MKGKEEKEGGARLGAGGGSPEKSPSAQELKEQGNRLFVGRKYPEAA | SEQ ID |
| | | ACYGRAITRNPLVAVYYTNRALCYLKMQQHEQALADCRRALELDGQ | NO: 146 |
| | | SVKAHFFLGQCQLEMESYDEAIANLQRAYSLAKEQRLNFGDDIPSA | encoding |
| | | LRIAKKKRWNSIEERRIHQESELHSYLSRLIAAERERELEECQRNH | for |
| | | EGDEDDSHVRAQQACIEAKHDKYMADMDELFSQVDEKRKKRDIPDY | amino |
| | | LCGKISFELMREPCITPSGITYDRKDIEEHLQRVGHFDPVTRSPLT | acids |
| | | QEQLIPNLAMKEVIDAFISENGWVEDY (SEQ ID NO: 145) | 128-303 |
| U-BOX | UBE4A | MTDQENNNNISSNPFAALFGSLADAKQFAAIQKEQLKQQSDELPAS | SEQ ID |
| | | PDDSDNSVSESLDEFDYSVAEISRSFRSQQEICEQLNINHMIQRIF | NO: 148 |
| | | LITLDNSDPSLKSGNGIPSRCVYLEEMAVELEDQDWLDMSNVEQAL | encoding |
| | | FARLLLQDPGNHLINMTSSTTLNLSADRDAGERHIFCYLYSCFQRA | for |
| | | KEEITKVPENLLPFAVQCRNLTVSNTRTVLLTPEIYVDQNIHEQLV | amino |
| | | DLMLEAIQGAHFEDVTEFLEEVIEALILDEEVRTFPEVMIPVEDIL | acids |
| | | LGRIKDLELCQILLYAYLDILLYFTRQKDMAKVEVEYIQPKDPTNG | 873- |
| | | QMYQKTLLGVILSISCLLKTPGVVENHGYFLNPSRSSPQEIKVQEA | 1066 |
| | | NIHQFMAQFHEKIYQMLKNLLQLSPETKHCILSWLGNCLHANAGRT | |
| | | KIWANQMPEIFFQMYASDAFFLNLGAALLKLCQPFCKPRSSRLLTF | |
| | | NPTYCALKELNDEERKIKNVHMRGLDKETCLIPAVQEPKFPQNYNL | |
| | | VTENLALTEYTLYLGFHRLHDQMVKINQNLHRLQVAWRDAQQSSSP | |
| | | AADNLREQFERLMTIYLSTKTAMTEPQMLQNCLNLQVSMAVLLVQL | |
| | | AIGNEGSQPIELTFPLPDGYSSLAYVPEFFADNLGDFLIFLRRFAD | |
| | | DILETSADSLEHVLHFITIFTGSIERMKNPHLRAKLAEVLEAVMPH | |
| | | LDQTPNPLVSSVFHRKRVFCNFQYAPQLAEALIKVFVDIEFTGDPH | |
| | | QFEQKENYRRPMYPILRYMWGTDTYRESIKDLADYASKNLEAMNPP | |
| | | LFLRFLNLLMNDAIFLLDEAIQYLSKIKIQQIEKDRGEWDSLTPEA | |
| | | RREKEAGLQMFGQLARFHNIMSNETIGTLAFLTSEIKSLFVHPFLA | |
| | | ERIISMLNYFLQHLVGPKMGALKVKDFSEFDFKPQQLVSDICTIYL | |
| | | NLGDEENFCATVPKDGRSYSPTLFAQTVRVLKKINKPGNMIMAFSN | |
| | | LAERIKSLADLQQQEEETYADACDEFLDPIMSTLMCDPVVLPSSRV | |
| | | TVDRSTIARHLLSDQTDPFNRSPLTMDQIRPNTELKEKIQRWLAER | |
| | | KQQKEQLE (SEQ ID NO: 147) | |
| U-BOX | UBOX5 | MVINLCLPQFRPRIHCNKISADGYEVENLISEDLTKRSHGERTEYF | SEQ ID |
| | (RNF37) | IKPPVYVTVSFPENVEICRINIDLTAGGGQNVTGLEMYTSASSSRV | NO: 150 |
| | | SWNTPQCRTLGPAEPSVPDKEAFTLVGKVLLKNQSQVVFSHRGFKA | encoding |
| | | RPPFGAMEATLPSPAVVAQELWNKGALSLSHVAHLRICITHVTGGG | for |
| | | IPCIKRLEVWGQPAKTCSQEVIDSILLVTSENLPQDVALQAPALPM | amino |
| | | ESDCDPGDQPESQQAPSSLQKLAEIIQDVPEEFLDPITLEIMPCPM | acids |
| | | LLPSGKVIDQSTLEKCNRSEATWGRVPSDPFTGVAFTPHSQPLPHP | 2-340 |
| | | SLKARIDHFLLQHSIPGCHLLGRAQTALAVIPSSIVLPSQKRKIEQ | |
| | | AEHVPDSNFGVNASCFSATSPLVLPTTSEHTAKKMKATNEPSLTHM | |
| | | DCSTGPLSHEQKLSQSLEIALASTLGSMPSFTARLTRGQLQHLGTR | |
| | | GSNTSWRPGTGSEQPGSILGPECASCKRVFSPYFKKEPVYQLPCGH | |
| | | LLCRPCLGEKQRSLPMTCTACQRPVASQDVLRVHF (SEQ ID | |
| | | NO: 149) | |
| HECT | NEDD4 | MATGLGEPVYGLSEDEGESRILRVKVVSGIDLAKKDIFGASDPYVK | SEQ ID |
| | | LSLYVADENRELALVQTKTIKKTLNPKWNEEFYFRVNPSNHRLLFE | NO: 152 |
| | | VEDENRLTRDDFLGQVDVPLSHLPTEDPTMERPYTFKDFLLRPRSH | encoding |
| | | KSRVKGFLRLKMAYMPKNGGQDEENSDQRDDMEHGWEVVDSNDSAS | amino |
| | | QHQEELPPPPLPPGWEEKVDNLGRTYYVNHNNRTTQWHRPSLMDVS | acids |
| | | SESDNNIRQINQEAAHRRFRSRRHISEDLEPEPSEGGDVPEPWETI | 616-975 |
| | | SEEVNIAGDSLGLALPPPPASPGSRTSPQELSEELSRRLQITPDSN | |
| | | GEQFSSLIQREPSSRLRSCSVTDAVAEQGHLPPPSAPAGRARSSTV | |
| | | TGGEEPTPSVAYVHTTPGLPSGWEERKDAKGRTYYVNHNNRTTTWT | |
| | | RPIMQLAEDGASGSATNSNNHLIEPQIRRPRSLSSPTVTLSAPLEG | |
| | | AKDSPVRRAVKDTLSNPQSPQPSPYNSPKPQHKVTQSFLPPGWEMR | |
| | | IAPNGRPFFIDHNTKTTTWEDPRLKFPVHMRSKTSLNPNDLGPLPP | |
| | | GWEERIHLDGRTFYIDHNSKITQWEDPRLQNPAITGPAVPYSREFK | |
| | | QKYDYFRKKLKKPADIPNRFEMKLHRNNIFEESYRRIMSVKRPDVL | |
| | | KARLWIEFESEKGLDYGGVAREWFFLLSKEMENPYYGLFEYSATDN | |
| | | YTLQINPNSGLCNEDHLSYFTFIGRVAGLAVFHGKLLDGFFIRPFY | |
| | | KMMLGKQITLNDMESVDSEYYNSLKWILENDPTELDLMFCIDEENF | |
| | | GQTYQVDLKPNGSEIMVTNENKREYIDLVIQWRFVNRVQKQMNAFL | |
| | | EGFTELLPIDLIKIFDENELELLMCGLGDVDVNDWRQHSIYKNGYC | |
| | | PNHPVIQWFWKAVLLMDAEKRIRLLQFVTGTSRVPMNGFAELYGSN | |
| | | GPQLFTIEQWGSPEKLPRAHTCFNRLDLPPYETFEDLREKLLMAVE | |
| | | NAQGFEGVD (SEQ ID NO: 151) | |
| HECT | UBR5 | MTSIHFVVHPLPGTEDQLNDRLREVSEKLNKYNLNSHPPLNVLEQA | SEQ ID |
| | | TIKQCVVGPNHAAFLLEDGRVCRIGFSVQPDRLELGKPDNNDGSKL | NO: 154 |
| | | NSNSGAGRTSRPGRTSDSPWFLSGSETLGRLAGNTLGSRWSSGVGG | encoding |
| | | SGGGSSGRSSAGARDSRRQTRVIRTGRDRGSGLLGSQPQPVIPASV | for |
| | | IPEELISQAQVVLQGKSRSVIIRELQRTNLDVNLAVNNLLSRDDED | amino |
| | | GDDGDDTASESYLPGEDLMSLLDADIHSAHPSVIIDADAMFSEDIS | acids |
| | | YFGYPSFRRSSLSRLGSSRVLLLPLERDSELLRERESVLRLRERRW | 2455- |
| | | LDGASFDNERGSTSKEGEPNLDKKNTPVQSPVSLGEDLQWWPDKDG | 2799 |
| | | TKFICIGALYSELLAVSSKGELYQWKWSESEPYRNAQNPSLHHPRA | |
| | | TFLGLTNEKIVLLSANSIRATVATENNKVATWVDETLSSVASKLEH | |
| | | TAQTYSELQGERIVSLHCCALYTCAQLENSLYWWGVVPFSQRKKML | |
| | | EKARAKNKKPKSSAGISSMPNITVGTQVCLRNNPLYHAGAVAFSIS | |
| | | AGIPKVGVLMESVWNMNDSCRFQLRSPESLKNMEKASKTTEAKPES | |
| | | KQEPVKTEMGPPPSPASTCSDASSIASSASMPYKRRRSTPAPKEEE | |
| | | KVNEEQWSLREVVFVEDVKNVPVGKVLKVDGAYVAVKFPGTSSNTN | |
| | | CQNSSGPDADPSSLLQDCRLLRIDELQVVKTGGTPKVPDCFQRTPK | |
| | | KLCIPEKTEILAVNVDSKGVHAVLKTGNWVRYCIFDLATGKAEQEN | |
| | | NFPTSSIAFLGQNERNVAIFTAGQESPIILRDGNGTIYPMAKDCMG | |
| | | GIRDPDWLDLPPISSLGMGVHSLINLPANSTIKKKAAVIIMAVEKQ | |
| | | TLMQHILRCDYEACRQYLMNLEQAVVLEQNLQMLQTFISHRCDGNR | |
| | | NILHACVSVCFPTSNKETKEEEEAERSERNTFAERLSAVEAIANAI | |
| | | SVVSSNGPGNRAGSSSSRSLRLREMMRRSLRAAGLGRHEAGASSSD | |
| | | HQDPVSPPIAPPSWVPDPPAMDPDGDIDFILAPAVGSLTTAATGTG | |
| | | QGPSTSTIPGPSTEPSVVESKDRKANAHFILKLLCDSVVLQPYLRE | |
| | | LLSAKDARGMTPFMSAVSGRAYPAAITILETAQKIAKAEISSSEKE | |
| | | EDVFMGMVCPSGTNPDDSPLYVLCCNDTCSFTWTGAEHINQDIFEC | |
| | | RTCGLLESLCCCTECARVCHKGHDCKLKRTSPTAYCDCWEKCKCKT | |
| | | LIAGQKSARLDLLYRLLTATNLVTLPNSRGEHLLLFLVQTVARQTV | |
| | | EHCQYRPPRIREDRNRKTASPEDSDMPDHDLEPPRFAQLALERVLQ | |
| | | DWNALKSMIMFGSQENKDPLSASSRIGHLLPEEQVYLNQQSGTIRL | |
| | | DCFTHCLIVKCTADILLLDTLLGTLVKELQNKYTPGRREEAIAVTM | |
| | | RFLRSVARVFVILSVEMASSKKKNNFIPQPIGKCKRVFQALLPYAV | |
| | | EELCNVAESLIVPVRMGIARPTAPFTLASTSIDAMQGSEELFSVEP | |
| | | LPPRPSSDQSSSSSQSQSSYIIRNPQQRRISQSQPVRGRDEEQDDI | |
| | | VSADVEEVEVVEGVAGEEDHHDEQEEHGEENAEAEGQHDEHDEDGS | |
| | | DMELDLLAAAETESDSESNHSNQDNASGRRSVVTAATAGSEAGASS | |
| | | VPAFFSEDDSQSNDSSDSDSSSSQSDDIEQETFMLDEPLERTTNSS | |
| | | HANGAAQAPRSMQWAVRNTQHQRAASTAPSSTSTPAASSAGLIYID | |
| | | PSNLRRSGTISTSAAAAAAALEASNASSYLTSASSLARAYSIVIRQ | |
| | | ISDLMGLIPKYNHLVYSQIPAAVKLTYQDAVNLQNYVEEKLIPTWN | |
| | | WMVSIMDSTEAQLRYGSALASAGDPGHPNHPLHASQNSARRERMTA | |
| | | REEASLRTLEGRRRATLLSARQGMMSARGDELNYALSLMRSHNDEH | |
| | | SDVLPVLDVCSLKHVAYVFQALIYWIKAMNQQTTLDTPQLERKRTR | |
| | | ELLELGIDNEDSEHENDDDTNQSATLNDKDDDSLPAETGQNHPFFR | |
| | | RSDSMTFLGCIPPNPFEVPLAEAIPLADQPHLLQPNARKEDLFGRP | |
| | | SQGLYSSSASSGKCLMEVTVDRNCLEVLPTKMSYAANLKNVMNMQN | |
| | | RQKKEGEEQPVLPEETESSKPGPSAHDLAAQLKSSLLAEIGLTESE | |
| | | GPPLTSFRPQCSFMGMVISHDMLLGRWRLSLELFGRVFMEDVGAEP | |
| | | GSILTELGGFEVKESKFRREMEKLRNQQSRDLSLEVDRDRDLLIQQ | |
| | | TMRQLNNHFGRRCATTPMAVHRVKVTFKDEPGEGSGVARSFYTAIA | |
| | | QAFLSNEKLPNLECIQNANKGTHTSLMQRLRNRGERDREREREREM | |
| | | RRSSGLRAGSRRDRDRDFRRQLSIDTRPFRPASEGNPSDDPEPLPA | |
| | | HRQALGERLYPRVQAMQPAFASKITGMLLELSPAQLLLLLASEDSL | |
| | | RARVDEAMELIIAHGRENGADSILDLGLVDSSEKVQQENRKRHGSS | |
| | | RSVVDMDLDDTDDGDDNAPLFYQPGKRGFYTPRPGKNTEARLNCER | |
| | | NIGRILGLCLLQNELCPITLNRHVIKVLLGRKVNWHDFAFFDPVMY | |
| | | ESLRQLILASQSSDADAVFSAMDLAFAIDLCKEEGGGQVELIPNGV | |
| | | NIPVTPQNVYEYVRKYAEHRMLVVAEQPLHAMRKGLLDVLPKNSLE | |
| | | DLTAEDFRLLVNGCGEVNVQMLISFTSENDESGENAEKLLQFKRWF | |
| | | WSIVEKMSMTERQDLVYFWTSSPSLPASEEGFQPMPSITIRPPDDQ | |
| | | HLPTANTCISRLYVPLYSSKQILKQKLLLAIKTKNFGFV (SEQ | |
| | | ID NO: 153) | |
| F-BOX | BTrCP | MDPAEAVLQEKALKFMCSMPRSLWLGCSSLADSMPSLRCLYNPGTG | SEQ ID |
| | | ALTAFQNSSEREDCNNGEPPRKIIPEKNSLRQTYNSCARLCLNQET | NO: 156 |
| | | VCLASTAMKTENCVAKTKLANGTSSMIVPKQRKLSASYEKEKELCV | encoding |
| | | KYFEQWSESDQVEFVEHLISQMCHYQHGHINSYLKPMLQRDFITAL | for |
| | | PARGLDHIAENILSYLDAKSLCAAELVCKEWYRVTSDGMLWKKLIE | amino |
| | | RMVRTDSLWRGLAERRGWGQYLFKNKPPDGNAPPNSFYRALYPKII | acids |
| | | QDIETIESNWRCGRHSLQRIHCRSETSKGVYCLQYDDQKIVSGLRD | 1-261 |
| | | NTIKIWDKNTLECKRILTGHTGSVLCLQYDERVIITGSSDSTVRVW | |
| | | DVNTGEMLNTLIHHCEAVLHLRFNNGMMVTCSKDRSIAVWDMASPT | |
| | | DITLRRVLVGHRAAVNVVDFDDKYIVSASGDRTIKVWNTSTCEFVR | |
| | | TLNGHKRGIACLQYRDRLVVSGSSDNTIRLWDIECGACLRVLEGHE | |
| | | ELVRCIRFDNKRIVSGAYDGKIKVWDLVAALDPRAPAGTLCLRTLV | |
| | | EHSGRVFRLQFDEFQIVSSSHDDTILIWDFLNDPAAQAEPPRSPSR | |
| | | TYTYISR (SEQ ID NO: 155) | |
| RBR | Parkin | MIVFVRFNSSHGFPVEVDSDTSIFQLKEVVAKRQGVPADQLRVIFA | SEQ ID |
| | | GKELRNDWTVQNCDLDQQSIVHIVQRPWRKGQEMNATGGDDPRNAA | NO: 158 |
| | | GGCEREPQSLTRVDLSSSVLPGDSVGLAVILHTDSRKDSPPAGSPA | encoding |
| | | GRSIYNSFYVYCKGPCQRVQPGKLRVQCSTCRQATLTLTQGPSCWD | for |
| | | DVLIPNRMSGECQSPHCPGTSAEFFFKCGAHPTSDKETSVALHLIA | amino |
| | | TNSRNITCITCTDVRSPVLVFQCNSRHVICLDCFHLYCVTRLNDRQ | acids |
| | | FVHDPQLGYSLPCVAGCPNSLIKELHHFRILGEEQYNRYQQYGAEE | 220-465 |
| | | CVLQMGGVLCPRPGCGAGLLPEPDQRKVTCEGGNGLGCGFAFCREC | |
| | | KEAYHEGECSAVFEASGTTTQAYRVDERAAEQARWEAASKETIKKT | |
| | | TKPCPRCHVPVEKNGGCMHMKCPQPQCRLEWCWNCGCEWNRVCMGD | |
| | | HWFDV (SEQ ID NO: 157) | |
| RING | RNF4 | MSTRKRRGGAINSRQAQKRTREATSTPEISLEAEPIELVETAGDEI | SEQ ID |
| | | VDLTCESLEPVVVDLTHNDSVVIVDERRRPRRNARRLPQDHADSCV | NO: 160 |
| | | VSSDDEELSRDRDVYVTTHTPRNARDEGATGLRPSGTVSCPICMDG | encoding |
| | | YSEIVQNGRLIVSTECGHVFCSQCLRDSLKNANTCPTCRKKINHKR | for |
| | | YHPIYI(SEQ ID NO: 159) | amino |
| | | | acids |
| | | | 76-190 |

TABLE 12
Sequences of mUbLs and ligase domains.

| mUbLs | Sequence |
|---|---|
| mUbL-35a nucleic | SEQ ID NO: 161 |
| acids | |
| mUbL-35a amino | QSVEESGGRLVSPGGSLTLTCRVSGSDRRTYVMRWVRQAPGKGLEWIGTIRLDGSRN |
| acids (fusion of 35a | YATWAKGRFTISKSSSTTMELRMTSLTSEDTATYFCVRDDLWGPGTLVTVSSGGGGS |
| and truncated CHIP; | GGGGSGGGGSMDTRAPTQLLGLLLLWLPGARCADIVMTQTPASVEVAVGGTVTIKCQ |
| without signal | ASQSISNLLAWYQQKPGQPPKLLIYKASTLESGVPSRFKGSGSGTQFALTISGVQCD |
| peptide and tags) | DAATYYCQQGFDSTDVDNLFGGGTEVVVAGSGSGRLNFGDDIPSALRIAKKKRWNSI |
| | EERRIHQESELHSYLSRLIAAERERELEECQRNHEGDEDDSHVRAQQACIEAKHDKY |
| | MADMDELFSQVDEKRKKRDIPDYLCGKISFELMREPCITPSGITYDRKDIEEHLQRV |
| | GHFDPVTRSPLTQEQLIPNLAMKEVIDAFISENGWVEDYVDGA ( SEQ ID NO: |
| | 162) |
| mUbL-35b nucleic | SEQ ID NO: 163 |
| acids (35bX-ACHIP- | |
| HIS-FLAG) | |
| mUbL-35b amino | QSVEESGGRLVSPGGSLTLTCRVSGSDRRTYVMRWVRQAPGKGLEWIGTIRLDGSRN |
| acids (fusion of 35b | YATWAKGRFTISKSSSTTMELRMTSLTSEDTATYFCVRDDLGGPGTLVTVSSGGGGS |
| and truncated CHIP; | GGGGSGGGGSMDTRAPTQLLGLLLLWLPGARCADIVMTQTPASVEVAVGGTVTIKCQ |
| without signal | ASQSISNLLAWYQQKPGQPPKLLIYKASTLESGVPSRFKGSGSGTQFALTISGVQCD |
| peptide and tags) | DAATYYCQQGFDSTDVDNLFGGGTEVVVAGSGSGRLNFGDDIPSALRIAKKKRWNSI |
| | EERRIHQESELHSYLSRLIAAERERELEECQRNHEGDEDDSHVRAQQACIEAKHDKY |
| | MADMDELFSQVDEKRKKRDIPDYLCGKISFELMREPCITPSGITYDRKDIEEHLQRV |
| | GHFDPVTRSPLTQEQLIPNLAMKEVIDAFISENGWVEDYVDGA ( SEQ ID NO: |
| | 164) |
| mUbL-58a nucleic | SEQ ID NO: 165 |
| acid | |
| mUbL-58a amino | QSVEESGGRLVTPGTPLTLTCTVSGIDLSSSVMAWVRQAPGKGLEWIGIITNRGNRY |
| acids (fusion of 58a | YAKWAKGRFTISRTSTTVGLKITSPTTADTATYFCGRGSVWGPGTLVTVSSGGGGSG |
| and truncated CHIP; | GGGSGGGGSMDTRAPTQLLGLLLLWLPGATFAQVLTQTPASVSAAIGGTVTIKCQSS |
| without signal | QSFLDDNDLAWYQKKPGRPPKRLIKSASTLDSGVPSRFSGSGSGTQFTLTISDVQCA |
| peptide and tags) | DAATYYCLGDYDCSVGDCSAFGGGTEVVVKGSGSGRLNFGDDIPSALRIAKKKRWNS |
| | IEERRIHQESELHSYLSRLIAAERERELEECQRNHEGDEDDSHVRAQQACIEAKHDK |
| | YMADMDELFSQVDEKRKKRDIPDYLCGKISFELMREPCITPSGITYDRKDIEEHLQR |
| | VGHFDPVTRSPLTQEQLIPNLAMKEVIDAFISENGWVEDYVDGA (SEQ ID NO: |
| | 166) |
| mUbL-58b nucleic | SEQ ID NO: 167 |
| acids | |
| mUbL-58b amino | QSVEESGGRLVTPGTPLTLTCTVSGIDLSSSVMVWVRQAPGKGLEWIGIITNRGNRY |
| acids (fusion of 58b | YAKWAKGRFTISRTSTTVGLKITSPTTADTATYFCGRGSVWGPGTLVTVSSGGGGSG |
| and truncated CHIP; | GGGSGGGGSMDTRAPTQLLGLLLLWLPGATFAQVLTQTPASVSAAIGGTVTIKCQSS |
| without signal | QSFLDDNDLAWYQKKPGRPPKRLIKSASTLDSGVPSRFSGSGSGTQFTLTISDVQCA |
| peptide and tags) | DAATYYCLGDYDCSVGDCSAFGGGTEVVVKGSGSGRLNFGDDIPSALRIAKKKRWNS |
| | IEERRIHQESELHSYLSRLIAAERERELEECQRNHEGDEDDSHVRAQQACIEAKHDK |
| | YMADMDELFSQVDEKRKKRDIPDYLCGKISFELMREPCITPSGITYDRKDIEEHLQR |
| | VGHFDPVTRSPLTQEQLIPNLAMKEVIDAFISENGWVEDYVDGA (SEQ ID NO: |
| | 168) |
| mUbL-63a nucleic | SEQ ID NO: 169 |
| acids | |
| mUbL-63a amino | QSVGESGGGLVTPGTPLTLTCTVSGFSLSSYGVSWVRQAPGKGLEYIGFISTRGNPY |
| acids (fusion of 63a | YASWAKGRFTISKTSTTVDLKITSPTTEDTATYFCAGATVVGDSIWGPGTLVTVSSG |
| and truncated CHIP; | GGGSGGGGSGGGGSMDTRAPTQLLGLLLLWLPGATFAQVLTQTASSVSAAVGGTVTI |
| without signal | SCQSSQSVYKGYWQAWYQQKPGQPPKLLIYETSTLASGVPSRFSGSGSGTQFTLTIS |
| peptide and tags) | GVQCDDAATYYCAGGYSNNIYTFGGGTEVVVKGSGSGRLNFGDDIPSALRIAKKKRW |
| | NSIEERRIHQESELHSYLSRLIAAERERELEECQRNHEGDEDDSHVRAQQACIEAKH |
| | DKYMADMDELFSQVDEKRKKRDIPDYLCGKISFELMREPCITPSGITYDRKDIEEHL |
| | QRVGHFDPVTRSPLTQEQLIPNLAMKEVIDAFISENGWVEDYVDGA (SEQ ID NO: |
| | 170) |
| mUbL-63b nucleic | SEQ ID NO: 171 |
| acids | |
| mUbL-63b amino | QSVEESGGGLVTPGTPLTLTCTVSGFSLSSYGVSWVRQAPGKGLEYIGFISTRGNPY |
| acids (fusion of 63b | YASWAKGRFTISKTSTTVDLKITSPTTEDTATYFCAGATVVGDSIWGPGTLVTVSSG |
| and truncated CHIP; | GGGSGGGGSGGGGSMDTRAPTQLLGLLLLWLPGATFAQVLTQTASSVSAAVGGTVTI |
| without signal | SCQSSQSVYKGYWQAWYQQKPGQPPKLLIYETSTLASGVPSRFSGSGSGTQFTLTIS |
| peptide and tags) | GVQCDDAATYYCAGGYSNNIYTFGGGTEVVVKGSGSGRLNFGDDIPSALRIAKKKRW |
| | NSIEERRIHQESELHSYLSRLIAAERERELEECQRNHEGDEDDSHVRAQQACIEAKH |
| | DKYMADMDELFSQVDEKRKKRDIPDYLCGKISFELMREPCITPSGITYDRKDIEEHL |
| | QRVGHFDPVTRSPLTQEQLIPNLAMKEVIDAFISENGWVEDYVDGA (SEQ ID NO: |
| | 172) |
| mUbL-85 nucleic | SEQ ID NO: 173 |
| acids | |
| mUbL-85 amino | QSVEESGGRLVTPGTPLTLTCTVSGIDLSRSVMAWVRQAPGKGLEWIGIVINRGNRY |
| acids (fusion of 85 | YANWAKGRFTISKTSTTVGLKIPSPTTADTATYFCGKGAVWGPGTLVTVSSGGGGSG |
| and truncated CHIP; | GGGSGGGGSMDTRAPTQLLGLLLLWLPGATFAQVLTQTPSSVSAAVGGTVTINCQSS |
| without signal | QSFLDDNDLAWFQQKPGRPPKRLIKSASTLDSGVPSRFKGSGSGTQFTLTISDVQCN |
| peptide and tags) | DAATYYCLGDYDCSVGDCSAFGGGTEVVVKGSGSGRLNFGDDIPSALRIAKKKRWNS |
| | IEERRIHQESELHSYLSRLIAAERERELEECQRNHEGDEDDSHVRAQQACIEAKHDK |
| | YMADMDELFSQVDEKRKKRDIPDYLCGKISFELMREPCITPSGITYDRKDIEEHLQR |
| | VGHFDPVTRSPLTQEQLIPNLAMKEVIDAFISENGWVEDYVDGA (SEQ ID NO: |
| | 174) |
| mUbL-93 nucleic | SEQ ID NO: 175 |
| acids | |
| mUbL-93 amino | QSVEESGGRLVTPGTPLTLTCTVSGIDLRRSVMAWVRQAPGKGLEWIGIISDRGNTY |
| acids (fusion of 93 | YASWAKGRFTISRTSTTVALKMTSPTTADTATYFCGRGNVWGPGTLVTVSSGGGGSG |
| and truncated CHIP; | GGGSGGGGSMDTRAPTQLLGLLLLWLPGATFAQVLTQTPSSVSAAVGGTVTINCQSS |
| without signal | QRLYDDNDCAWYQKKPGQPPKRLIHDASILDSGVSSRFSGSGSGTQFTLTISGVQCE |
| peptide and tags) | DAATYYCLGDYDCSVADCSTFGGGTEVVVKGSGSGRLNFGDDIPSALRIAKKKRWNS |
| | IEERRIHQESELHSYLSRLIAAERERELEECQRNHEGDEDDSHVRAQQACIEAKHDK |
| | YMADMDELFSQVDEKRKKRDIPDYLCGKISFELMREPCITPSGITYDRKDIEEHLQR |
| | VGHFDPVTRSPLTQEQLIPNLAMKEVIDAFISENGWVEDYVDGA (SEQ ID NO: |
| | 176) |
| Control nucleic acids | SEQ ID NO: 177 |
| (beta-gal with no | |
| scFv) | |
| Control amino acids | MAEVQLVESGGSLVKPGGSLRLSCAASGFTFSNYSMNWVRQAPGKGLEWISSISGSS |
| (beta-gal with no | RYIYYADFVKGRFTISRDNATNSLYLQMNSLRAEDTAVYYCVRSSITIFGGGMDVWG |
| scFv) | RGTLVTVSSGGGGSGGGGSGGGGSQSVLTQPASVSGSPGQSITISCAGTSSDVGGYN |
| | YVSWYQQHPGKAPKLMIYEDSKRPSGVSNRFSGSKSGNTASLTISGLQAEDEADYYC |
| | SSYTTRSTRVFGGGTKLAVLGAAAEFGSGSGRLNFGDDIPSALRIAKKKRWNSIEER |
| | RIHQESELHSYLSRLIAAERERELEECQRNHEGDEDDSHVRAQQACIEAKHDKYMAD |
| | MDELFSQVDEKRKKRDIPDYLCGKISFELMREPCITPSGITYDRKDIEEHLQRVGHF |
| | DPVTRSPLTQEQLIPNLAMKEVIDAFISENGWVEDYVDGA (SEQ ID NO: 178) |
| 35bX-AUBE4A- | SEQ ID NO: 179 |
| HIS-FLAG | |
| HIS-FLAGtag- | SEQ ID NO: 180 |
| AUBOX5-35bX | |
| HIS-FLAGtag- | SEQ ID NO: 181 |
| 35bX-ANEDD4L | |
| HIS-FLAGtag- | SEQ ID NO: 182 |
| 35bX-AUBR5 | |
| HIS-FLAGtag- | SEQ ID NO: 183 |
| ABTrCP-35bX | |
| 35bX-AParkin-HIS- | SEQ ID NO: 184 |
| FLAG | |
| 35bX-ARNF4-HIS- | SEQ ID NO: 185 |
| FLAG | |

(ligase)

| | |
|---|---|
| Ubox (ligase) | DIPDYLCGKISFELMREPCITPSGITYDRKDIEEHLQRVGHFDPVTRSPLTQEQLIP |
| domain of CHIP | NLAMKEVIDAFISENGWV (SEQ ID NO: 186) |
| Ubox (ligase) | ACDEFLDPIMSTLMCDPVVLPSSRVTVDRSTIARHLLSDQTDPFNRSPLTMDQIRPN |
| domain of UBE4A | TELKEKIQRWLAERKQQ (SEQ ID NO: 187) |
| Ubox (ligase) | DVPEEFLDPITLEIMPCPMLLPSGKVIDQSTLEKCNRSEATWGRVPSDPFTGVAFTP |
| domain of UBOX5 | HSQPLPHPSLKARIDHFLLQHSIP (SEQ ID NO: 188) |
| (RNF37) | |
| HECT (ligase) | RPDVLKARLWIEFESEKGLDYGGVAREWFFLLSKEMFNPYYGLFEYSATDNYTLQIN |
| domain of NEDD4L | PNSGLCNEDHLSYFTFIGRVAGLAVFHGKLLDGFFIRPFYKMMLGKQITLNDMESVD |
| | SEYYNSLKWILENDPTELDLMFCIDEENFGQTYQVDLKPNGSEIMVINENKREYIDL |
| | VIQWRFVNRVQKQMNAFLEGFTELLPIDLIKIFDENELELLMCGLGDVDVNDWRQHS |
| | IYKNGYCPNHPVIQWFWKAVLLMDAEKRIRLLQFVTGTSRVPMNGFAELYGSNGPQL |
| | FTIEQWGSPEKLPRAHTCENRLDLPPYETFEDLREKLLMAVENAQGFEGV ( SEQ |
| | ID NO: 189) |
| HECT (ligase) | LDLGLVDSSEKVQQENRKRHGSSRSVVDMDLDDTDDGDDNAPLFYQPGKRGFYTPRP |
| domain of UBR5 | GKNTEARLNCFRNIGRILGLCLLQNELCPITLNRHVIKVLLGRKVNWHDFAFFDPVM |
| | YESLRQLILASQSSDADAVESAMDLAFAIDLCKEEGGGQVELIPNGVNIPVTPQNVY |
| | EYVRKYAEHRMLVVAEQPLHAMRKGLLDVLPKNSLEDLTAEDFRLLVNGCGEVNVQM |
| | LISFTSENDESGENAEKLLQFKRWFWSIVEKMSMTERQDLVYFWTSSPSLPASEEGF |
| | QPMPSITIRPPDDQHLPTANTCISRLYVPLYSSKQILKQKLLLAIKTKNFGFV |
| | (SEQ ID NO: 190) |
| F-BOX (ligase) | DHIAENILSYLDAKSLCAAELVCKEWYRVTSDGMLWKKL (SEQ ID NO: 191) |
| domain of BTrCP | |
| RBR (ligase) domain | CITCTDVRSPVLVFQCNSRHVICLDCFHLYCVTRLNDRQFVHDPQLGYSLPCVAGCP |
| of Parkin | NSLIKELHHFRILGEEQYNRYQQYGAEECVLQMGGVLCPRPGCGAGLLPEPDQRKVT |
| | CEGGNGLGCGFAFCRECKEAYHEGECSAVFEASGTTTQAYRVDERAAEQARWEAASK |
| | ETIKKTTKPCPRCHVPVEKNGGCMHMKCPQPQCRLEWCWNC (SEQ ID NO: 192) |
| RING (ligase) | PICMDGYSEIVQNGRLIVSTECGHVFCSQCLRDSLKNANTCPTCR (SEQ ID NO: |
| domain of RNF4 | 193) |
| Truncated CHIP | RLNFGDDIPSALRIAKKKRWNSIEERRIHQESELHSYLSRLIAAERERELEECQRNH |
| including the coiled | EGDEDDSHVRAQQACIEAKHDKYMADMDELFSQVDEKRKKRDIPDYLCGKISFELMR |
| coil region and the | EPCITPSGITYDRKDIEEHLQRVGHFDPVTRSPLTQEQLIPNLAMKEVIDAFISENG |
| catalytic ligase Ubox | WVEDYVDGA (SEQ ID NO: 223) |
| region | |

TABLE 13
Signal Peptides

| | |
|---|---|
| Signal peptide 1 | METGLRWLLLVAVLKGVQC |
| | (SEQ ID NO: 224) |
| Signal peptide 2 | MDTRAPTQLLGLLLLWLPGATFA |
| | (SEQ ID NO: 225) |
| Signal peptide 3 | MDTRAPTQLLGLLLLWLPGARC |
| | (SEQ ID NO: 226) |

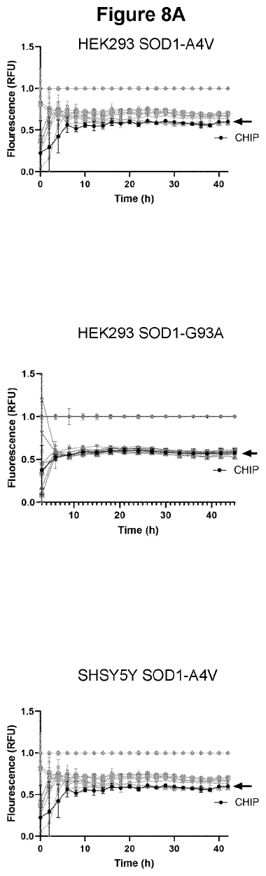

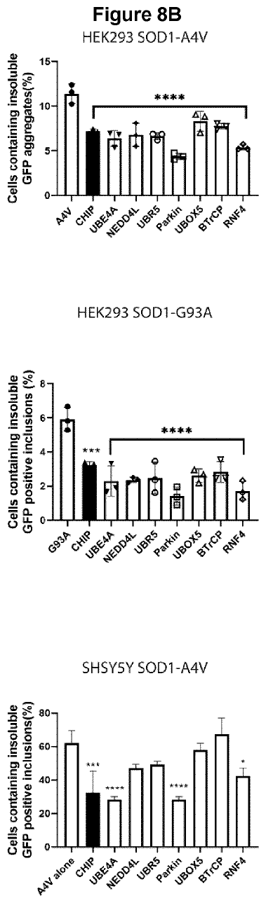

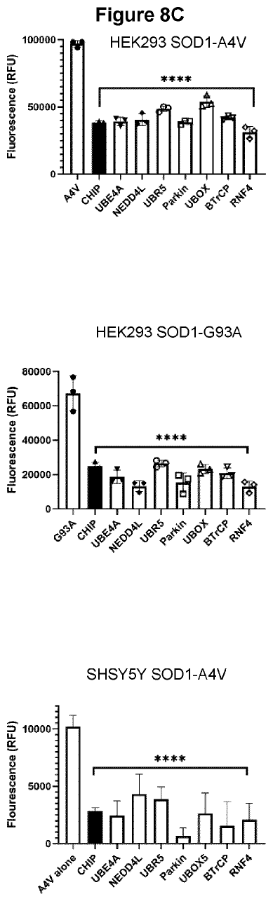

FIG. 8 shows that the E3 ligase fusion panel can reduce SOD1-A4V and G93A-GFP fluorescence in HEK293 and SHSY5Y cells. In HEK293 cells, all ligases were able to reduce SOD1-A4V and G93A total fluorescence over a 48 h time course with very little difference between the effectiveness of the ligases (FIG. 8A). Graphs are representative from at least 3 experiments. Data from SHSY5Y cells support this finding with a reduction in total fluorescence over time (one replicate). In addition, the fusion E3 ligases were effective at reducing the number of insoluble fluorescent aggregates present in transfected cells after saponin treatment with Parkin showing significant reduction in A4V transfected cells and both Parkin and RNF4 showing significant reduction in G93A transfected cells (FIG. 81B). Statistical significance was determined against cells transfected with SOD1-A4V or G93A alone using one way ANOVA with Dunnett's multiple comparisons (p<0.0001=****, p<0.001=***, p<0.01=** p<0.05=*) SHSY5Y transfected cells also showed a reduction in the number of SOD1-A4V insoluble aggregates (one replicate). Further, the level of soluble fluorescence in the media after saponin treatment showed reductions in SOD1-GFP by all ligases with NEDD4L, Parkin, and RNF4 showing significance in G93A transfected cells (FIG. 8C). CHIP is shown in solid black for comparison in all graphs.

### Example 5B. Using mUbL In Vivo in a SOD1^{G93A} Mouse Model

### Methods

### Animal Study

All research was approved by the Animal Ethics Committee (AE21/10) of the University of Wollongong (Wollongong, Australia) and complied with the National Health and Medical Research Institute, Australian Code of Practice for the Care and Use of Animals for Scientific Purposes.

Generating the mUbL Transgenic Mouse Line

The generation of transgenic mice harbouring the mUbL gene was as previously described (Delerue & Ittner 2017). Briefly, cDNA for the mUbL (35b fused to CHIP, i.e. was incorporated into an expression cassette plasmid including inverted terminal repeats (ITR), the synapsin promoter, the woodchuck hepatitis virus post-transcriptional regulatory element (WPRE), and the bovine growth hormone polyadenylation sequence (BGH poly A). Purified Cas9 mRNA, purified guide RNA designed to target the ROSA26 safe harbour region and the mUbL plasmid were injected into the cytoplasm of isolated mouse oocytes. Injected oocytes were implanted in wild-type C57BL/6 female mice with immediate mating. Gene insertion was confirmed using PCR-based analysis of progeny. After identification of a successful founder (F0) mouse (B6. Cg-Tg(hsyn-mUbL)Yer/j), inheritance was confirmed by breeding the F0 with wild-type C57BL/6 to produce F1 offspring.

Genotyping of mUbL Mice

Genotyping of mUbL mice at GEM was done by PCR amplification of genomic DNA obtained from tail biopsies using two primer sets; 5′-AGTCACCATCAAGTGCCAGG-3′ (SEQ ID NO: 217) and 3′-TCATCACCCTCGTGGTTTCG-5′ (SEQ ID NO: 218) and 5′-ATTACGTCGACGGAGCAGAC-3′ (SEQ ID NO: 219) and 3′-AAGGAAGGTCCGCTGGATTG-5′ (SEQ ID NO: 220).

Cross Breeding mUbL Mice with SOD1G93A Mice

Female mUbL mice were bred in harem with male mice hemizygous for the human SOD1G93A transgene maintained on a C57BL/6 background (B6-Tg (SOD1-G93A)1Gur/j). All mice were housed in individually ventilated cages (IVC) (Type Mouse Version 1; Airlaw, Smithfield, Australia; air change: 90-120 times per hour averaged; passive exhaust ventilation system).

### Mouse Study Conditions and Information

Mice were housed at the University of Wollongong in IVC cages (Greenline GM500, Techniplast, Australia) under a 12:12 h light-dark cycle (illuminated from 0700 to 1900 h). Mice were caged with littermates where attainable, with 2-4 females or 1-4 males per cage. IVC cages included a layering of Bed-O'Cobs™ corn cob bedding (Techniplast, Australia), tissues, Bed r'Nest™ (Techniplast, Australia), a plastic house and a PVC tunnel. Food and water were available ad libitum. When mice reached 100 days old or became symptomatic (clinical score of 2) and unable to access water or food easily, water-soaked food pellets were placed on the cage floor and longer sippers placed on water bottles.

### Body Weight and ALS Neurological Score

Mice were weighed three times a week at the same time of day to prevent diurnal variations. They were also assessed for neurological score three times a week using the criteria outlined by the ALS Therapy Development Institute (TDI). Scoring and weighing of mice commenced at 50 days old and was performed by observers blinded to genotype.

### Rotarod Performance

Locomotor function of mice was assessed weekly, beginning at 50 days old, using a five-lane accelerating rotarod (RotaRod Advanced, TSE Systems, Hesse, Germany). Mice were habituated to the rotarod in the three days immediately prior to commencing testing. Habituation sessions consisted of three acclimatisation sessions, with the first session run at a continuous 4 rotations per minute (rpm) for 180 s. The second and third habituation sessions were performed at an inclining speed of 4-20 rpm over a 180 s period. During the testing phase, the rotation speed of the rotarod was accelerated from 4 to 20 rpm over a 180 s period with the time taken to fail the task (latency to fall) recorded for each mouse. Mice were given two independent runs with a 30-60 s rest between runs. The average time and the maximum time each mouse was able to remain on the rod was recorded and included in the data analysis. To control for odour cues, the apparatus was cleaned with 70% ethanol after each trial.

### End Stage

End-stage was attained when a mouse reached an ALS score of 4 (inability to right itself within 10 s after being placed on either side), or 20% weight loss compared to their initial pre-symptomatic disease maximum body weight at postnatal day 50. Once mice reached end stage, mice were euthanised by asphyxiation using a slow-filled carbon dioxide inhalation technique.

### Post-Mortem Analysis

### Protein Extraction

PBS-perfused brains and spinal cords were dissected, and the lumbar portion excised using spinal cord enlargement to identify the region. Tissue was snap-frozen in liquid N2 and stored at −80° C. until required. Detergent-soluble proteins were extracted by homogenising tissue in ice-cold PBS containing 0.01% Triton-X-100 containing 100× Halt protease inhibitor cocktail using a micro pestle. A ratio of 5× extraction reagent per weight of tissue was utilised. Homogenates were sonicated for 3×5 s bursts at 50% amplitude, incubated on ice for 20 min and cleared (20,000×g at 4° C. for 30 min). To extract detergent-insoluble proteins, the resulting pellet was resuspended in 8M Urea buffer at 2×weight of original tissue and incubated on ice for 10 min. Insoluble material was cleared (20,000×g for 30 min). Protein concentration was determined using the Bio-Rad DC assay (Hercules, CA) for soluble fractions and the nanodrop for insoluble fractions. Protein samples were stored at −80° C. until required.

mUbL Protein Detection

Soluble protein with a total protein concentration of 20 g was mixed with 4× reducing SDS-PAGE sample buffer (200 mM Tris-HCl pH 6.8, 8% SDS (w/v), 40% glycerol (v/v), 50 mM EDTA, 0.08% bromophenol blue (w/v), 4% β-mercaptoethanol (v/v)) and heated for 5 min at 95° C. before being loaded onto 4-20% Criterion™ TGX Stain-Free™ gels (BioRad, Australia). Gels were electrophoresed for 1 h at 160 V. Following electrophoresis, total protein on the gel was quantified using a Criterion Stain Free™ Imager (BioRad, Australia) prior to transferring for immunoblotting. Proteins separated by SDS-PAGE were transferred onto methanol activated Amersham™ Hybond™ 0.2 μm PVDF membranes (GE Healthcare, USA) at 100 V for 1 h at 4° C. using 1× transfer buffer (25 mM Tris-base 20% methanol (v/v) 192 mM glycine). Membranes were imaged with a stain-free imager post-transfer to confirm transfer and measure total protein. The membrane was blocked in 5% skim milk powder in Tris-buffered saline with 0.2% (v/v) Tween-20 (TBST) for 1 h at RT before probing with primary antibody overnight at 4° C. The following day membranes were washed three times with TBST over 30 min before incubating with secondary antibody for 1 h at RT. The membrane was visualised with chemiluminescent substrate (Thermo Scientific) on the Amersham Imager 6600RGB. Analysis and quantification was performed using ImageJ.

### Results and Discussion

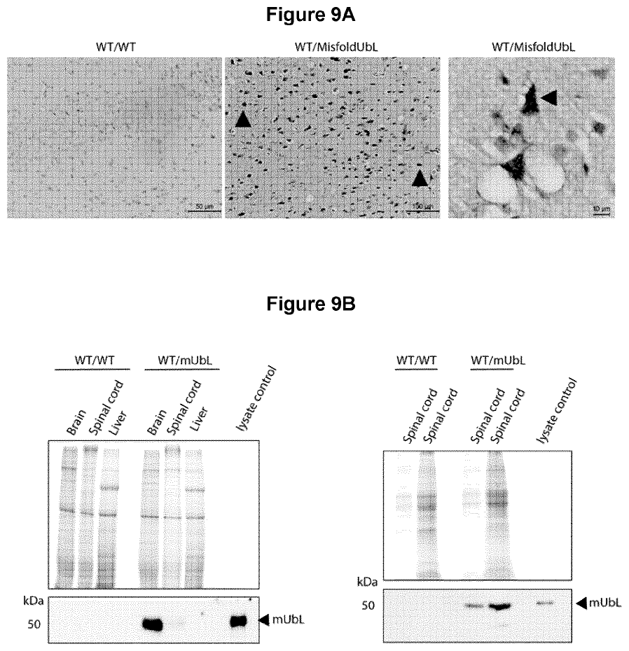

FIG. 9A shows expression of mUbLs in neuronal cells in brain tissue. Brain sections (5 uM) from WT/WT or WT/mUbL mice were fixed, paraffin embedded, and labelled with anti-FLAG antibody. Tissue was stained with DAB and counterstained in haematoxylin. Representative images are shown with scale bars. mUbL indicated with black arrows. FIG. 9B shows expression of mUbLs in brain and spinal cord but not liver in WT/mUbL mice. Left panel of Brain, spinal cord and liver from WT/WT or WT/mUbL mice were homogenised separated by SDS-PAGE and blotted with anti-HIS (FIG. 9B, left panel). Cell lysate from transient transfection of mUbL used as positive control. Spinal cord samples from different WT/WT or WT/mUbL mice were homogenised separated by SDS-PAGE and blotted with anti-HIS (FIG. 9B, right panel). Representative images from n>3 replicates are shown.

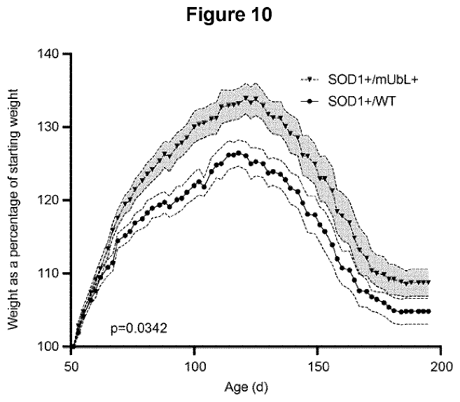

FIG. 10 shows mUbL transgene attenuates and delays weight loss in the early symptomatic phase of ALS in male but not female SOD1^{G93A} mice. Male mice showed a significant difference in body weight with SOD1^{G93A}/mUbL mice having an average weight gain of 120.8% and SOD1^{G93A}/WT mice 115.3%. *P<0.0342 two way ANOVA with Tukey's multiple comparisons test. No significant difference in weight loss was found between SOD1^{G93A}/mUbL and SOD1^{G93A}/WT female mice. Data represent mean±SEM, n=10-12 mice per gender per genotype.

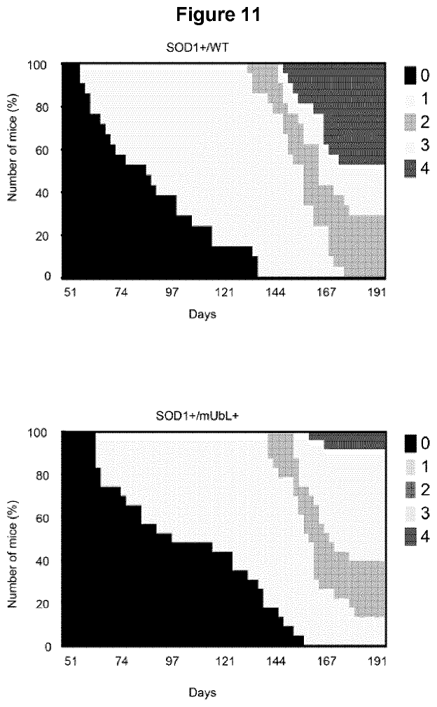

FIG. 11 shows the mUbL transgene delays disease progression in SOD1^{G93A} mice. SOD1^{G93A}/mUbL mice progressed more slowly through the stages of disease as indicated by neurological score.

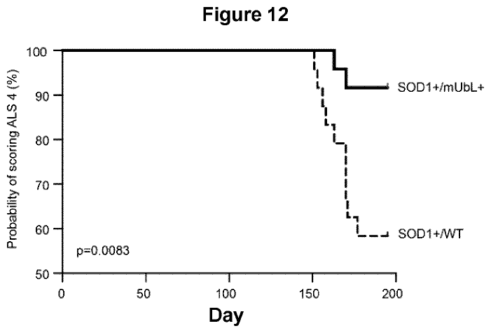

FIG. 12 shows the mUbL transgene attenuated clinical phenotype at end stage of the disease with only 9% of SOD1^{G93A}/mUbL reaching a neurological score of 4 compared to 48% of SOD1^{G93A}/WT mice.

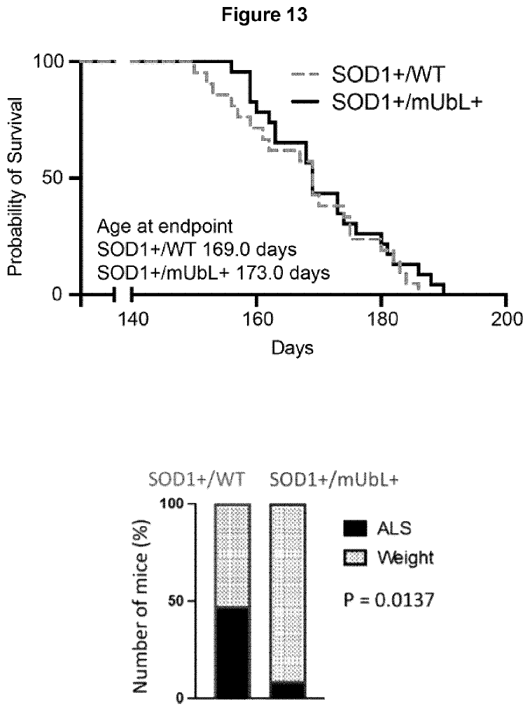

FIG. 13 shows mUbL affecting phenotype of SOD1^{G93A} at endpoint. Pre-defined criteria mandated that mice were to be euthanized once they either reached 20% loss in body weight compared to their pre-symptomatic maximum body weight, or they showed inability to right themselves within 10 s after being placed on either side (a neurological score of 4). The present data shows that mUbL improves phenotype of SOD1^{G93A} at endpoint. Although there was no difference in survival between SOD1^{G93A}/mUbL and SOD1^{G93A}/WT mice at the endpoint, this masked the fact only 9% SOD1^{G93A}/mUbL mice reached the end point because of complete limb paralysis, i.e. inability to right themselves within 10 s after being placed on either side. The 43.5% of the SOD1^{G93A}/mUbL were euthanized because of weight loss, but otherwise have delayed disease onset (i.e. a neurological score of 1 or 2). The high probability of significant delay in disease onset by mUbL was a remarkable and surprising finding given that ALS is known to be a very difficult disease to treat.

Some incremental advances have been made in the treatment of ALS. Tofersen, also known as BIIB067, is an antisense oligonucleotide (ASO) that targets SOD1 mRNA to prevent the translation of SOD1 protein. Tofersen, and all ASOs cannot, differentiate between wild type and mutant SOD1 protein. A phase III clinical trial of Tofersen showed a reduction of SOD1 protein in the CSF of 29-40% (Miller et al., 2022 N Engl J Med). Despite not meeting clinical endpoints, Tofersen was granted approval by the FDA under the accelerated approval pathway. The decision was based on a reduction in plasma neurofilament light (NfL), which serves as a blood-based biomarker of axonal (nerve) injury and neurodegeneration. Here, although the promoter use for mUbL expression is brain and neuronal specific, an attenuation of ALS phenotype was observed. Based on this, if promoter for spinal cord and/or glial cells were used, further improvement can be expected. Indeed, the present inventors have shown that mUbLs are a promising treatment modality for ALS. A combination therapy, for example, by utilizing a mUbL and an ASO such as Tofersen, can also be useful.

### Example 5C. AAV Delivery of mUbL

AAV constructs expressing mUbL described herein. The mUbL 35b-CHIP is cloned into the multiple cloning site of an AAV Vector (pAAV-CAG-EGFP) under the CAG promoter for expression across neuronal, glial and skeletal muscle (see Ittner et al 2016; Jackson et al 2016 for promoter). AAV vectors are packaged into AAV-pHP.eB (see Chan et al 2017 for AAV) and injected into the tail vein of SOD1^{G93A} mice at PN0. Mice are then monitored until endpoint with weight and neurological score measured three times a week. Motor control and strength are assessed weekly by rotarod and grip strength tests. If efficacy is observed, then the AAV is delivered at PN90 and a similar study performed. mUbLs are useful for ALS treatment in viral delivery system such as AAV.

### Example 6. Administration to ALS Subjects: Antibody or Binding Fragment Thereof

An antibody or binding fragment thereof directed against misfolded SOD1 epitope of SEQ ID NO: 144 is administered to human ALS subjects. The antibody or binding fragment thereof is administered to the subjects at 6, 8, 12, 16 and 20 weeks.

Subjects are monitored for indications of slowing or arrest of SOD aggregation and disease progression. Autoimmune manifestations are also monitored.

Subjects are monitored regularly for adverse effects such as signs of pain and distress that might be a result of the immunizations. Autoimmune manifestations are monitored.

Administration of antibody or binding fragment thereof directed against misfolded SOD1 epitope of SEQ ID NO: 144 slows the progression of ALS. Slowing or arrest of SOD1 aggregation or abrogation of disease progression occurs for therapeutically active antibodies.

### Example 7. Administration to ALS Subjects: MUbLs

mUbLs (fusion protein comprising scFv and E3 ligase) directed against misfolded SOD1 epitope of SEQ ID NO: 144 are administered to human ALS subjects. Subjects are monitored for indications of slowing or arrest of SOD aggregation and disease progression. Administration of mUbLs directed to misfolded SOD1 epitope of SEQ ID NO: 144 slows the progression of ALS disease in subjects. Slowing or arrest of SOD1 aggregation or abrogation of disease progression occurs for therapeutically active antibodies.

mUbLs directed against misfolded SOD1 epitope of SEQ ID NO: 144 are administered to human ALS subjects. A pharmaceutical composition comprising 1-140 grams (up to 2 grams/kilo) of the mUbLs is administered by intravenous infusion to produce a local concentration that ranges from 1 to 10 micrograms per ml in the CNS. In one regimen, the formulation comprises a mUbLs directed against misfolded SOD1 epitope of SEQ ID NO: 144. In another regimen, the formulation comprises two or more mUbLs. The dosing regimen will vary on the physiological condition of the subjects and the response of the subject to treatment. In one dosing regimen, the dosing is once every 3 or 4 weeks. In other regimens, dosing is once per week, twice per week, three times per week, or once per 2 weeks.

### Example 8. Intraventricular or Intrathecal Administration of mUbLs to ALS Subjects

mUbLs (fusion protein comprising scFv and E3 ligase) directed against misfolded SOD1 epitope of SEQ ID NO: 144 are directly administered into the CNS of ALS subjects by intraventricular or intrathecal infusion using an infusion pump such as the infusion pumps produced by MedTronics (Minneapolis, MN, USA). ALS subjects are infused with 0.5 to 5 mg per day of mUbL to obtain an end concentration of 1-10 micrograms per ml in the CNS infused at a maximal rate of 1 ml/h. In one regimen, the formulation comprises a mUbL directed against SEQ ID NO: 144. In another regimen, the formulation comprises two or more mUbLs.

When mUbLs are administered to ALS subjects by intrathecal injection an equal volume of cerebrospinal fluid is withdrawn through the same needle used for the injection to avoid an increase in pressure due to the injection volume. Subjects are given a dose that ranges from 1% to 10% of the corresponding systemic dose. Subjects receive a single dose of the mUbLs formulation. Alternatively, subjects receive multiple doses of the mUbLs formulation. In one regimen, the formulation comprises a mUbL directed against misfolded SOD1 epitope of SEQ ID NO: 144. In another regimen, the formulation comprises two or more mUbLs. In alternate regimens, dosing is once per week, twice per week, three times per week, once every two weeks, once every three weeks or once every month. In another regimen, the dosing varies depending on the physiological condition of the subject and the response of the subject to the treatment.

Subjects are monitored for indications of slowing or arrest of SOD aggregation and disease progression. Administration of a mUbL directed against misfolded SOD1 epitope of SEQ ID NO: 144 slows the progression of ALS disease in subjects. Slowing or arrest of SOD1 aggregation or abrogation of disease progression occurs for therapeutically active mUbL.

### Example 9. Administration to ALS Subjects: Antibody or Binding Fragment Thereof

An antibody or binding fragment thereof directed against misfolded SOD1 epitope of SEQ ID NO: 144 are administered to human ALS subjects. The antibody or binding fragment thereof is administered to the subjects at 6, 8, 12, 16 and 20 weeks.

Subjects are monitored for indications of slowing or arrest of SOD aggregation and disease progression. Autoimmune manifestations are also monitored.

Subjects are monitored regularly for adverse effects such as signs of pain and distress that might be a result of the immunizations. Autoimmune manifestations are monitored.

Administration of antibody or binding fragment thereof directed against misfolded SOD1 epitope of SEQ ID NO: 144 slows the progression of ALS. Slowing or arrest of SOD1 aggregation or abrogation of disease progression occurs for therapeutically active antibodies.

### Example 10. Administration to ALS Subjects: MUbLs

mUbLs (fusion protein comprising scFv and E3 ligase) directed against misfolded SOD1 epitope of SEQ ID NO: 144 are administered to human ALS subjects. Subjects are monitored for indications of slowing or arrest of SOD aggregation and disease progression. Administration of mUbLs directed to misfolded SOD1 epitope of SEQ ID NO: 144 slows the progression of ALS disease in subjects. Slowing or arrest of SOD1 aggregation or abrogation of disease progression occurs for therapeutically active mUbLs.

mUbLs directed against misfolded SOD1 epitope of SEQ ID NO: 144 are administered to human ALS subjects. A pharmaceutical composition comprising 1-140 grams (up to 2 grams/kilo) of the mUbLs is administered by intravenous infusion to produce a local concentration that ranges from 1 to 10 micrograms per ml in the CNS. In one regimen, the formulation comprises a mUbLs directed against misfolded SOD1 epitope of SEQ ID NO: 144. In another regimen, the formulation comprises two or more mUbLs. The dosing regimen will vary on the physiological condition of the subjects and the response of the subject to treatment. In one dosing regimen, the dosing is once every 3 or 4 weeks. In other regimens, dosing is once per week, twice per week, three times per week, or once per 2 weeks.

### Example 11. Intraventricular or Intrathecal Administration of mUbLs to ALS Subjects

mUbLs (fusion protein comprising scFv and E3 ligase) directed against misfolded SOD1 epitope of SEQ ID NO: 144 are directly administered into the CNS of ALS subjects by intraventricular or intrathecal infusion using an infusion pump such as the infusion pumps produced by MedTronics (Minneapolis, MN, USA). ALS subjects are infused with 0.5 to 5 mg per day of mUbL to obtain an end concentration of 1-10 micrograms per ml in the CNS infused at a maximal rate of 1 ml/h. In one regimen, the formulation comprises a mUbL directed against SEQ ID NO: 144. In another regimen, the formulation comprises two or more mUbLs.

When mUbLs are administered to ALS subjects by intrathecal injection an equal volume of cerebrospinal fluid is withdrawn through the same needle used for the injection to avoid an increase in pressure due to the injection volume. Subjects are given a dose that ranges from 1% to 10% of the corresponding systemic dose. Subjects receive a single dose of the mUbLs formulation. Alternatively, subjects receive multiple doses of the mUbLs formulation. In one regimen, the formulation comprises a mUbL directed against misfolded SOD1 epitope of SEQ ID NO: 144. In another regimen, the formulation comprises two or more mUbLs. In alternate regimens, dosing is once per week, twice per week, three times per week, once every two weeks, once every three weeks or once every month. In another regimen, the dosing varies depending on the physiological condition of the subject and the response of the subject to the treatment.

Subjects are monitored for indications of slowing or arrest of SOD aggregation and disease progression. Administration of a mUbL directed against misfolded SOD1 epitope of SEQ ID NO: 144 slows the progression of ALS disease in subjects. Slowing or arrest of SOD1 aggregation or abrogation of disease progression occurs for therapeutically active mUbL.

### Example 12. Administration to Alzheimer's Disease Subjects: Antibody or Binding Fragment Thereof

An antibody or binding fragment thereof directed against misfolded SOD1 epitope of SEQ ID NO: 144 are administered to human Alzheimer's disease subjects. The antibody or binding fragment thereof is administered to the subjects at 6, 8, 12, 16 and 20 weeks.

Subjects are monitored for indications of slowing or arrest of SOD aggregation and disease progression. Autoimmune manifestations are also monitored.

Subjects are monitored regularly for adverse effects such as signs of pain and distress that might be a result of the immunizations. Autoimmune manifestations are monitored.

Administration of antibody or binding fragment thereof directed against misfolded SOD1 epitope of SEQ ID NO: 144 slows the progression of Alzheimer's disease. Slowing or arrest of SOD1 aggregation or abrogation of disease progression occurs for therapeutically active antibodies.

### Example 13. Administration to Alzheimer's Disease Subjects: MUbLs

mUbLs (fusion protein comprising scFv and E3 ligase) directed against misfolded SOD1 epitope of SEQ ID NO: 144 are administered to human Alzheimer's disease subjects. Subjects are monitored for indications of slowing or arrest of SOD aggregation and disease progression. Administration of mUbLs directed to misfolded SOD1 epitope of SEQ ID NO: 144 slows the progression of Alzheimer's disease in subjects. Slowing or arrest of SOD1 aggregation or abrogation of disease progression occurs for therapeutically active mUbLs.

mUbLs directed against misfolded SOD1 epitope of SEQ ID NO: 144 are administered to human Alzheimer's disease subjects. A pharmaceutical composition comprising 1-140 grams (up to 2 grams/kilo) of the mUbLs is administered by intravenous infusion to produce a local concentration that ranges from 1 to 10 micrograms per ml in the CNS. In one regimen, the formulation comprises a mUbLs directed against misfolded SOD1 epitope of SEQ ID NO: 144. In another regimen, the formulation comprises two or more mUbLs. The dosing regimen will vary on the physiological condition of the subjects and the response of the subject to treatment. In one dosing regimen, the dosing is once every 3 or 4 weeks. In other regimens, dosing is once per week, twice per week, three times per week, or once per 2 weeks.

### Example 14. Intraventricular or Intrathecal Administration of mUbLs to Alzheimer's Disease Subjects

mUbLs (fusion protein comprising scFv and E3 ligase) directed against misfolded SOD1 epitope of SEQ ID NO: 144 are directly administered into the CNS of Alzheimer's disease subjects by intraventricular or intrathecal infusion using an infusion pump such as the infusion pumps produced by MedTronics (Minneapolis, MN, USA). Alzheimer's disease subjects are infused with 0.5 to 5 mg per day of mUbL to obtain an end concentration of 1-10 micrograms per ml in the CNS infused at a maximal rate of 1 ml/h. In one regimen, the formulation comprises a mUbL directed against SEQ ID NO: 144. In another regimen, the formulation comprises two or more mUbLs.

When mUbLs are administered to Alzheimer's disease subjects by intrathecal injection an equal volume of cerebrospinal fluid is withdrawn through the same needle used for the injection to avoid an increase in pressure due to the injection volume. Subjects are given a dose that ranges from 1% to 10% of the corresponding systemic dose. Subjects receive a single dose of the mUbLs formulation. Alternatively, subjects receive multiple doses of the mUbLs formulation. In one regimen, the formulation comprises a mUbL directed against misfolded SOD1 epitope of SEQ ID NO: 144. In another regimen, the formulation comprises two or more mUbLs. In alternate regimens, dosing is once per week, twice per week, three times per week, once every two weeks, once every three weeks or once every month. In another regimen, the dosing varies depending on the physiological condition of the subject and the response of the subject to the treatment.

Subjects are monitored for indications of slowing or arrest of SOD aggregation and disease progression. Administration of a mUbL directed against misfolded SOD1 epitope of SEQ ID NO: 144 slows the progression of Alzheimer's disease in subjects. Slowing or arrest of SOD1 aggregation or abrogation of disease progression occurs for therapeutically active mUbL.

### Example 15. Administration to Parkinson's Disease Subjects: Antibody or Binding Fragment Thereof

An antibody or binding fragment thereof directed against misfolded SOD1 epitope of SEQ ID NO: 144 are administered to human Parkinson's disease subjects. The antibody or binding fragment thereof is administered to the subjects at 6, 8, 12, 16 and 20 weeks.

Subjects are monitored for indications of slowing or arrest of SOD aggregation and disease progression. Autoimmune manifestations are also monitored.

Subjects are monitored regularly for adverse effects such as signs of pain and distress that might be a result of the immunizations. Autoimmune manifestations are monitored.

Administration of antibody or binding fragment thereof directed against misfolded SOD1 epitope of SEQ ID NO: 144 slows the progression of Parkinson's disease. Slowing or arrest of SOD1 aggregation or abrogation of disease progression occurs for therapeutically active antibodies.

### Example 16. Administration to Parkinson's Disease Subjects: MUbLs

mUbLs (fusion protein comprising scFv and E3 ligase) directed against misfolded SOD1 epitope of SEQ ID NO: 144 are administered to human Parkinson's disease subjects. Subjects are monitored for indications of slowing or arrest of SOD aggregation and disease progression. Administration of mUbLs directed to misfolded SOD1 epitope of SEQ ID NO: 144 slows the progression of Parkinson's disease in subjects. Slowing or arrest of SOD1 aggregation or abrogation of disease progression occurs for therapeutically active mUbLs.

mUbLs directed against misfolded SOD1 epitope of SEQ ID NO: 144 are administered to human Parkinson's disease subjects. A pharmaceutical composition comprising 1-140 grams (up to 2 grams/kilo) of the mUbLs is administered by intravenous infusion to produce a local concentration that ranges from 1 to 10 micrograms per ml in the CNS. In one regimen, the formulation comprises a mUbLs directed against misfolded SOD1 epitope of SEQ ID NO: 144. In another regimen, the formulation comprises two or more mUbLs. The dosing regimen will vary on the physiological condition of the subjects and the response of the subject to treatment. In one dosing regimen, the dosing is once every 3 or 4 weeks. In other regimens, dosing is once per week, twice per week, three times per week, or once per 2 weeks.

### Example 17. Intraventricular or Intrathecal Administration of mUbLs to Parkinson's Disease Subjects

mUbLs (fusion protein comprising scFv and E3 ligase) directed against misfolded SOD1 epitope of SEQ ID NO: 144 are directly administered into the CNS of Parkinson's disease subjects by intraventricular or intrathecal infusion using an infusion pump such as the infusion pumps produced by MedTronics (Minneapolis, MN, USA). Parkinson's disease subjects are infused with 0.5 to 5 mg per day of mUbL to obtain an end concentration of 1-10 micrograms per ml in the CNS infused at a maximal rate of 1 ml/h. In one regimen, the formulation comprises a mUbL directed against SEQ ID NO: 144. In another regimen, the formulation comprises two or more mUbLs.

When mUbLs are administered to Parkinson's disease subjects by intrathecal injection an equal volume of cerebrospinal fluid is withdrawn through the same needle used for the injection to avoid an increase in pressure due to the injection volume. Subjects are given a dose that ranges from 1% to 10% of the corresponding systemic dose. Subjects receive a single dose of the mUbLs formulation. Alternatively, subjects receive multiple doses of the mUbLs formulation. In one regimen, the formulation comprises a mUbL directed against misfolded SOD1 epitope of SEQ ID NO: 144. In another regimen, the formulation comprises two or more mUbLs. In alternate regimens, dosing is once per week, twice per week, three times per week, once every two weeks, once every three weeks or once every month. In another regimen, the dosing varies depending on the physiological condition of the subject and the response of the subject to the treatment.

Subjects are monitored for indications of slowing or arrest of SOD aggregation and disease progression. Administration of a mUbL directed against misfolded SOD1 epitope of SEQ ID NO: 144 slows the progression of Parkinson's disease in subjects. Slowing or arrest of SOD1 aggregation or abrogation of disease progression occurs for therapeutically active mUbL.

### Example 18. Administration to Frontotemporal Dementia Subjects: Antibody or Binding Fragment Thereof

An antibody or binding fragment thereof directed against misfolded SOD1 epitope of SEQ ID NO: 144 are administered to human frontotemporal dementia subjects. The antibody or binding fragment thereof is administered to the subjects at 6, 8, 12, 16 and 20 weeks.

Subjects are monitored for indications of slowing or arrest of SOD aggregation and disease progression. Autoimmune manifestations are also monitored.

Subjects are monitored regularly for adverse effects such as signs of pain and distress that might be a result of the immunizations. Autoimmune manifestations are monitored.

Administration of antibody or binding fragment thereof directed against misfolded SOD1 epitope of SEQ ID NO: 144 slows the progression of frontotemporal dementia. Slowing or arrest of SOD1 aggregation or abrogation of disease progression occurs for therapeutically active antibodies.

### Example 19. Administration to Frontotemporal Dementia Subjects: MUbLs

mUbLs (fusion protein comprising scFv and E3 ligase) directed against misfolded SOD1 epitope of SEQ ID NO: 144 are administered to human frontotemporal dementia subjects. Subjects are monitored for indications of slowing or arrest of SOD aggregation and disease progression. Administration of mUbLs directed to misfolded SOD1 epitope of SEQ ID NO: 144 slows the progression of frontotemporal dementia in subjects. Slowing or arrest of SOD1 aggregation or abrogation of disease progression occurs for therapeutically active mUbLs.

mUbLs directed against misfolded SOD1 epitope of SEQ ID NO: 144 are administered to human frontotemporal dementia subjects. A pharmaceutical composition comprising 1-140 grams (up to 2 grams/kilo) of the mUbLs is administered by intravenous infusion to produce a local concentration that ranges from 1 to 10 micrograms per ml in the CNS. In one regimen, the formulation comprises a mUbLs directed against misfolded SOD1 epitope of SEQ ID NO: 144. In another regimen, the formulation comprises two or more mUbLs. The dosing regimen will vary on the physiological condition of the subjects and the response of the subject to treatment. In one dosing regimen, the dosing is once every 3 or 4 weeks. In other regimens, dosing is once per week, twice per week, three times per week, or once per 2 weeks.

### Example 20. Intraventricular or Intrathecal Administration of mUbLs to Frontotemporal Dementia Subjects

mUbLs (fusion protein comprising scFv and E3 ligase) directed against misfolded SOD1 epitope of SEQ ID NO: 144 are directly administered into the CNS of frontotemporal dementia subjects by intraventricular or intrathecal infusion using an infusion pump such as the infusion pumps produced by MedTronics (Minneapolis, MN, USA). Frontotemporal dementia subjects are infused with 0.5 to 5 mg per day of mUbL to obtain an end concentration of 1-10 micrograms per ml in the CNS infused at a maximal rate of 1 ml/h. In one regimen, the formulation comprises a mUbL directed against SEQ ID NO: 144. In another regimen, the formulation comprises two or more mUbLs.

When mUbLs are administered to frontotemporal dementia subjects by intrathecal injection an equal volume of cerebrospinal fluid is withdrawn through the same needle used for the injection to avoid an increase in pressure due to the injection volume. Subjects are given a dose that ranges from 1% to 10% of the corresponding systemic dose. Subjects receive a single dose of the mUbLs formulation. Alternatively, subjects receive multiple doses of the mUbLs formulation. In one regimen, the formulation comprises a mUbL directed against misfolded SOD1 epitope of SEQ ID NO: 144. In another regimen, the formulation comprises two or more mUbLs. In alternate regimens, dosing is once per week, twice per week, three times per week, once every two weeks, once every three weeks or once every month. In another regimen, the dosing varies depending on the physiological condition of the subject and the response of the subject to the treatment.

Subjects are monitored for indications of slowing or arrest of SOD aggregation and disease progression. Administration of a mUbL directed against misfolded SOD1 epitope of SEQ ID NO: 144 slows the progression of frontotemporal dementia in subjects. Slowing or arrest of SOD1 aggregation or abrogation of disease progression occurs for therapeutically active mUbL.

### Example 21. AAV Delivery of mUbLs to ALS Subjects

mUbLs (fusion protein comprising scFv and E3 ligase) directed against misfolded SOD1 epitope of SEQ ID NO: 144 are packaged for delivery in a recombinant adeno-associated virus (rAAV) vector, such as AAV9, comprising a prion promoter for CNS gene expression. The recombinant AAV encoding mUBLs (rAAV-mUBLs) are delivered at a pharmaceutically acceptable dose by direct stereotaxic brain infusion, spinal cord injection, intravascular delivery, or intramuscular delivery, to human ALS subjects.

Subjects receive a single dose of the rAAV-mUbLs formulation. Alternatively, subjects receive multiple doses of the rAAV-mUbLs formulation. In one regimen, the formulation comprises a rAAV expressing one mUbL directed against misfolded SOD1 epitope of SEQ ID NO: 144. In another regimen, the formulation comprises an rAAV expressing two or more mUbLs. In alternate regimens, dosing is once per week, twice per week, three times per week, once every two weeks, once every three weeks or once every month. In another regimen, the dosing varies depending on the physiological condition of the subject and the response of the subject to the treatment.

Subjects are monitored for indications of slowing or arrest of SOD aggregation and disease progression. Administration of rAAV-mUbL directed against misfolded SOD1 epitope of SEQ ID NO: 144 slows the progression of ALS in subjects. Slowing or arrest of SOD1 aggregation or abrogation of disease progression occurs for rAAV-mUbL expressing therapeutically active mUbL.

### Example 22. AAV Delivery of mUbLs to Alzheimer's Disease Subjects

mUbLs (fusion protein comprising scFv and E3 ligase) directed against misfolded SOD1 epitope of SEQ ID NO: 144 are packaged for delivery in a recombinant adeno-associated virus (rAAV) vector, such as AAV9, comprising a prion promoter for CNS gene expression. The recombinant AAV encoding mUBLs (rAAV-mUBLs) are delivered at a pharmaceutically acceptable dose by direct stereotaxic brain infusion, spinal cord injection, intravascular delivery, or intramuscular delivery, to human Alzheimer's Disease subjects.

Subjects receive a single dose of the rAAV-mUbLs formulation. Alternatively, subjects receive multiple doses of the rAAV-mUbLs formulation. In one regimen, the formulation comprises a rAAV expressing one mUbL directed against misfolded SOD1 epitope of SEQ ID NO: 144. In another regimen, the formulation comprises an rAAV expressing two or more mUbLs. In alternate regimens, dosing is once per week, twice per week, three times per week, once every two weeks, once every three weeks or once every month. In another regimen, the dosing varies depending on the physiological condition of the subject and the response of the subject to the treatment.

Subjects are monitored for indications of slowing or arrest of SOD aggregation and disease progression. Administration of rAAV-mUbL directed against misfolded SOD1 epitope of SEQ ID NO: 144 slows the progression of Alzheimer's Disease in subjects. Slowing or arrest of SOD1 aggregation or abrogation of disease progression occurs for rAAV-mUbL expressing therapeutically active mUbL.

### Example 23. AAV Delivery of mUbLs to Parkinson's Disease Subjects

mUbLs (fusion protein comprising scFv and E3 ligase) directed against misfolded SOD1 epitope of SEQ ID NO: 144 are packaged for delivery in a recombinant adeno-associated virus (rAAV) vector, such as AAV9, comprising a prion promoter for CNS gene expression. The recombinant AAV encoding mUBLs (rAAV-mUBLs) are delivered at a pharmaceutically acceptable dose by direct stereotaxic brain infusion, spinal cord injection, intravascular delivery, or intramuscular delivery, to human Parkinson's Disease subjects.

Subjects receive a single dose of the rAAV-mUbLs formulation. Alternatively, subjects receive multiple doses of the rAAV-mUbLs formulation. In one regimen, the formulation comprises a rAAV expressing one mUbL directed against misfolded SOD1 epitope of SEQ ID NO: 144. In another regimen, the formulation comprises an rAAV expressing two or more mUbLs. In alternate regimens, dosing is once per week, twice per week, three times per week, once every two weeks, once every three weeks or once every month. In another regimen, the dosing varies depending on the physiological condition of the subject and the response of the subject to the treatment.

Subjects are monitored for indications of slowing or arrest of SOD aggregation and disease progression. Administration of rAAV-mUbL directed against misfolded SOD1 epitope of SEQ ID NO: 144 slows the progression of Parkinson's Disease in subjects. Slowing or arrest of SOD1 aggregation or abrogation of disease progression occurs for rAAV-mUbL expressing therapeutically active mUbL.

### Example 24. AAV Delivery of mUbLs to Frontotemporal Dementia Subjects

mUbLs (fusion protein comprising scFv and E3 ligase) directed against misfolded SOD1 epitope of SEQ ID NO: 144 are packaged for delivery in a recombinant adeno-associated virus (rAAV) vector, such as AAV9, comprising a prion promoter for CNS gene expression. The recombinant AAV encoding mUBLs (rAAV-mUBLs) are delivered at a pharmaceutically acceptable dose by direct stereotaxic brain infusion, spinal cord injection, intravascular delivery, or intramuscular delivery, to human frontotemporal dementia subjects.

Subjects receive a single dose of the rAAV-mUbLs formulation. Alternatively, subjects receive multiple doses of the rAAV-mUbLs formulation. In one regimen, the formulation comprises a rAAV expressing one mUbL directed against misfolded SOD1 epitope of SEQ ID NO: 144. In another regimen, the formulation comprises an rAAV expressing two or more mUbLs. In alternate regimens, dosing is once per week, twice per week, three times per week, once every two weeks, once every three weeks or once every month. In another regimen, the dosing varies depending on the physiological condition of the subject and the response of the subject to the treatment.

Subjects are monitored for indications of slowing or arrest of SOD aggregation and disease progression. Administration of rAAV-mUbL directed against misfolded SOD1 epitope of SEQ ID NO: 144 slows the progression of frontotemporal dementia in subjects. Slowing or arrest of SOD1 aggregation or abrogation of disease progression occurs for rAAV-mUbL expressing therapeutically active mUbL.

While the present disclosure has been described with reference to what are presently considered to be the preferred example, it is to be understood that the disclosure is not limited to the disclosed example. To the contrary, the disclosure is intended to cover various modifications and equivalent arrangements included within the spirit and scope of the appended claims. It will be appreciated how various changes and modifications may be made without departing from the disclosure.

All publications, patents or patent applications mentioned in this specification are herein incorporated by reference to the same extent as if each independent documentation is specifically and individually indicated to be incorporated by reference.

### CITATIONS FOR REFERENCES REFERRED TO IN THE SPECIFICATION

Albitar, M., ed. Monoclonal Antibodies: Methods and Protocols. Vol. 378. Springer Science & Business Media, 2007.
Ausubel M et al., Current Protocols in Molecular Biology, Wiley Interscience, New York, 2001.
Borras, L., et al. “Generic approach for the generation of stable humanized single-chain Fv fragments from rabbit monoclonal antibodies.” Journal of Biological Chemistry 285.12 (2010): 9054-9066.
Burgess, R. R. and Deutscher, M. P. eds. Guide to Protein Purification. Academic Press, 2009.
Chan, K. Y., et al. “Engineered AAVs for efficient noninvasive gene delivery to the central and peripheral nervous systems.” Nat Neurosci. 20:8 (2017):1172-1179. Epub 2017 June 26.
Choi, J., et al. “Oxidative modifications and aggregation of Cu,Zn-superoxide dismutase associated with Alzheimer and Parkinson diseases”. Journal of Biological Chemistry; 280:11 (2005) 11648-11655.
Chothia, C., and Lesk, A. M. “Canonical structures for the hypervariable regions of immunoglobulins.” Journal of Molecular Biology 196:4 (1987) 901-917.
Chothia, C. et al. “Structural repertoire of the human VH segments.” Journal of Molecular Biology 227:3 (1992): 799-817.
Cole et al. “Monoclonal Antibodies in Cancer Therapy.” (1985) Allen R. Bliss, Inc., pages 77-96.
D'Erchia, A. M., et al. “Massive transcriptome sequencing of human spinal cord tissues provides new insights into motor neuron degeneration in ALS.” Scientific reports 7:1 (2017) 1-20.
Deleme F., and Ittner L. M. “Generation of Genetically Modified Mice through the Microinjection of Oocytes. J Vis Exp. (2017) June 15(124):55765.
Elam, J. S., et al. “Amyloid-like filaments and water-filled nanotubes formed by SOD1 mutant proteins linked to familial ALS.” Nature Structural & Molecular Biology 10.6 (2003) 461-467.
Farrawell N. E. et al. “CuATSM Protects Against the In Vitro Cytotoxicity of Wild-Type-Like Copper-Zinc Superoxide Dismutase Mutants but not Mutants That Dismpt Metal Binding.” ACS Chemical Neuroscience 10:3 (2019) 1555-1564.
Honegger, A. and Pluckthun A. “Yet another numbering scheme for immunoglobulin variable domains: an automatic modeling and analysis tool.” Journal of Molecular Biology 309:3 (2001) 657-670.
Huse, W, et al. “Generation of a large combinatorial library of the immunoglobulin repertoire in phage lambda.” Science 246:4935 (1989) 1275-1282.
Huston, J. S. et al, “Protein engineering of single-chain Fv analogs and fusion proteins.” Methods in enzymology. Vol. 203. Academic Press (1991) 46-88.
Ibrahim, A. F. M., et al. “Antibody RING-mediated destruction of endogenous proteins.” Molecular Cell 79.1 (2020) 155-166. doi.org/10.1016/j.molcel.2020.04.032
Jackson K. L., et al., “Better Targeting, Better Efficiency for Wide-Scale Neuronal Transduction with the Synapsin Promoter and AAV-PHP.B.” Front Mol Neurosci. (2016) November 4; 9:116. doi: 10.3389/fnmol.2016.00116. Erratum in: Front Mol Neurosci. (2016) December 22; 9:154. PMID: 27867348; PMCID: PMC5095393.
Johnson, S., and Bird, R. E. “Construction of single-chain Fv derivatives of monoclonal antibodies and their production in Escherichia coli” Methods in enzymology. Vol. 203. Academic Press (1991) 88-98.
Kabat, E. A. et al., “Sequences of Proteins of Immunological Interest”, 5^{th} Ed.” NIH 1 (1991) 688-961.
Kashmiri, S. V. S., et al. “SDR grafting—a new approach to antibody humanization.” Methods 36.1 (2005): 25-34.
Köhler, G. and Milstein, C. “Continuous cultures of fused cells secreting antibody of predefined specificity.” Nature 256.5517 (1975): 495-497.
Kolaskar, A. S. and Tongaonkar, P. C. “A semi-empirical method for prediction of antigenic determinants on protein antigens.” FEBS letters 276:1-2 (1990) 172-174.
Kozbor, D, and Roder, J, “The production of monoclonal antibodies from human lymphocytes.” Immunology Today 4:3 (1983) 72-79.
Kunik, V. et al. “Paratome: an online tool for systematic identification of antigen-binding regions in antibodies based on sequence or structure.” Nucleic Acids Research 40:W1 (2012) W521-W524.
Kuroiwa, Y., et al. “Cloned transchromosomic calves producing human immunoglobulin.” Nature biotechnology 20:9 (2002): 889-894.
Lefranc, M.-P., et al. “IMGT, the international ImMunoGeneTics database.” Nucleic Acids Research 27:1 (1999) 209-212.
MacCallum, R. M., et al. “Antibody-antigen interactions: contact analysis and binding site topography.” Journal of Molecular Biology 262:5 (1996) 732-745.
Maier, J. K. X. and Labute P. “Assessment of fully automated antibody homology modeling protocols in molecular operating environment.” Proteins: Structure, Function, and Bioinformatics 82:8 (2014) 1599-1610.
Mathis, S., et al. “Genetics of amyotrophic lateral sclerosis: A review.” Journal of the neurological sciences 399 (2019) 217-22.
Martin, A. C. et al. “Modeling antibody hypervariable loops: a combined algorithm.” Proceedings of the National Academy of Sciences 86:23 (1989) 9268-9272.
McAlary L. et al. “Susceptibility of Mutant SOD1 to Form a Destabilized Monomer Predicts Cellular Aggregation and Toxicity but Not In vitro Aggregation Propensity.” Frontiers in Neuroscience 10:499 (2016). doi.org/10.3389/fnins.2016.00499
Mernaugh R. and Mernaugh G., In: Molecular Methods in Plant Pathology, Singh and Singh (Eds.), CRC Press, FL (1995) 343-358.
Miller T. M. et al. “VALOR and OLE Working Group. Trial of Antisense Oligonucleotide Tofersen for SOD1 ALS.” N Engl J Med. (2022) September 22; 387(12):1099-1110.
Olsson L. and Kaplan H. S. “Human-human monoclonal antibody-producing hybridomas: Technical aspects.” Methods in Enzymol, 92 (1982) 3-16.
Ottis, P., et al. “Assessing different E3 ligases for small molecule induced protein ubiquitination and degradation.” ACS chemical biology 12.10 (2017): 2570-2578. DOI: 10.1021/acschembio.7b00485
Portnoff, A. D., et al. “Ubiquibodies, synthetic E3 ubiquitin ligases endowed with unnatural substrate specificity for targeted protein silencing.” Journal of Biological Chemistry 289:11 (2014) 7844-7855. doi.org/10.1074/jbc.M113.544825
Rader, C., et al. “The rabbit antibody repertoire as a novel source for the generation of therapeutic human antibodies.” Journal of Biological Chemistry 275.18 (2000): 13668-13676. Sambrook J. F. and Russell D. W., Molecular Cloning: A Laboratory Manual, 3rd edition, Cold Spring Harbor Laboratory Press, N.Y., 2001.
Scopes, R. K. Protein Purification: Principles and Practice. Springer Science & Business Media, 2013.
Steinberger, P., et al. “Generation and characterization of a recombinant human CCR5-specific antibody: a phage display approach for rabbit antibody humanization.” Journal of Biological Chemistry 275.46 (2000): 36073-36078.
Teng et al. “Construction and Testing of Mouse—Human Heteromyelomas for Human Monoclonal Antibody Production.” PNAS 80:12 (1983) 7308-7312.
Turner B. J., et al. “Impaired Extracellular Secretion of Mutant Superoxide Dismutase 1 Associates with Neurotoxicity in Familial Amyotrophic Lateral Sclerosis.” Journal of Neuroscience 25:1108-117. doi.org/10.1523/JNEUROSCI.4253-04.2005
Yu, Y., et al. “A humanized anti-VEGF rabbit monoclonal antibody inhibits angiogenesis and blocks tumor growth in xenograft models.” PloS one 5.2 (2010): e9072.
Zhang, Y.-F. and Ho, H. “Humanization of rabbit monoclonal antibodies via grafting combined Kabat/IMGT/Paratome complementarity-determining regions: Rationale and examples.” MAbs. Vol. 9. No. 3. Taylor & Francis, 2017.

We claim:

1. An isolated antibody or binding fragment thereof that binds to misfolded superoxide dismutase 1 (SOD1) epitope having an amino acid sequence shown in SEQ ID NO: 144, the antibody or binding fragment thereof comprises:
(i) a heavy chain comprising a heavy chain variable region, wherein the heavy chain variable region comprises CDR1, CDR2, and CDR3 having the amino acid sequence of SEQ ID NO: 1, SEQ ID NO: 2, and SEQ ID NO: 3, and a light chain comprising a light chain variable region, wherein the light chain variable region comprises CDR1, CDR2, and CDR3 having the amino acid sequence of SEQ ID NO: 4, SEQ ID NO: 5, and SEQ ID NO: 6;
(ii) a heavy chain comprising a heavy chain variable region, wherein the heavy chain variable region comprises CDR1, CDR2, and CDR3 having the amino acid sequence of SEQ ID NO: 7, SEQ ID NO: 8, and SEQ ID NO: 9, and a light chain comprising a light chain variable region, wherein the light chain variable region comprises CDR1, CDR2 and CDR3 having the amino acid sequence of SEQ ID NO: 10, SEQ ID NO: 11 and SEQ ID NO: 12;
(iii) a heavy chain comprising a heavy chain variable region, wherein the heavy chain variable region comprises CDR1, CDR2 and CDR3 having the amino acid sequence of SEQ ID NO: 14, SEQ ID NO: 15, and SEQ ID NO: 16, and a light chain comprising a light chain variable region, wherein the light chain variable region comprises CDR1, CDR2 and CDR3 having the amino acid sequence of SEQ ID NO: 10, SEQ ID NO: 11, and SEQ ID NO: 12;
(iv) a heavy chain comprising a heavy chain variable region, wherein the heavy chain variable region comprises CDR1, CDR2, and CDR3 having the amino acid sequence of SEQ ID NO: 7, SEQ ID NO: 8, and SEQ ID NO: 9, and a light chain comprising a light chain variable region, wherein the light chain variable region comprises CDR1, CDR2 and CDR3 having the amino acid sequence of SEQ ID NO: 10, SEQ ID NO: 11 and SEQ ID NO: 13;
(v) a heavy chain comprising a heavy chain variable region, wherein the heavy chain variable region comprises CDR1, CDR2 and CDR3 having the amino acid sequence of SEQ ID NO: 14, SEQ ID NO: 15, and SEQ ID NO: 16, and a light chain comprising a light chain variable region, wherein the light chain variable region comprises CDR1, CDR2 and CDR3 having the amino acid sequence of SEQ ID NO: 10, SEQ ID NO: 11, and SEQ ID NO: 13;
(vi) a heavy chain comprising a heavy chain variable region, wherein the heavy chain variable region comprises CDR1, CDR2 and CDR3 having the amino acid sequence of SEQ ID NO: 53, SEQ ID NO: 54, and SEQ ID NO: 55, and a light chain comprising a light chain variable region, wherein the light chain variable region comprises CDR1, CDR2 and CDR3 having the amino acid sequence of SEQ ID NO: 56, SEQ ID NO: 57, and SEQ ID NO: 58;
(vii) a heavy chain comprising a heavy chain variable region, wherein the heavy chain variable region comprises CDR1, CDR2 and CDR3 having the amino acid sequence of SEQ ID NO: 23, SEQ ID NO: 24, and SEQ ID NO: 25, and a light chain comprising a light chain variable region, wherein the light chain variable region comprises CDR1, CDR2 and CDR3 having the amino acid sequence of SEQ ID NO: 26, SEQ ID NO: 27, and SEQ ID NO: 28; or
(viii) a heavy chain comprising a heavy chain variable region, wherein the heavy chain variable region comprises CDR1, CDR2 and CDR3 having the amino acid sequence of SEQ ID NO: 29, SEQ ID NO: 30, and SEQ ID NO: 31, and a light chain comprising a light chain variable region, wherein the light chain variable region comprises CDR1, CDR2 and CDR3 having the amino acid sequence of SEQ ID NO: 32, SEQ ID NO: 33, and SEQ ID NO: 34.

2. The antibody or binding fragment thereof according to claim 1, wherein
(i) the heavy chain comprises the heavy chain variable region comprising an amino acid sequence having at least 85%, 90%, 95%, 97%, 98%, 99%, 99.5%, 99.5% or 100% identity to the amino acid sequence of SEQ ID NO: 65, and the light chain comprises the light chain variable region comprising an amino acid sequence having at least 85%, 90%, 95%, 97%, 98%, 99%, 99.5%, 99.5% or 100% identity to the amino acid sequence of SEQ ID NO: 70;
(ii) the heavy chain comprises the heavy chain variable region comprising an amino acid sequence having at least 85%, 90%, 95%, 97%, 98%, 99%, 99.5%, 99.5% or 100% identity to the amino acid sequence of SEQ ID NO: 68, and the light chain comprises the light chain variable region comprising an amino acid sequence having at least 85%, 90%, 95%, 97%, 98%, 99%, 99.5%, 99.5% or 100% identity to the amino acid sequence of SEQ ID NO: 70;
(iii) the heavy chain comprises the heavy chain variable region comprising an amino acid sequence having at least 85%, 90%, 95%, 97%, 98%, 99%, 99.5%, 99.5% or 100% identity to the amino acid sequence of SEQ ID NO: 72, and the light chain comprises the light chain variable region comprising an amino acid sequence having at least 85%, 90%, 95%, 97%, 98%, 99%, 99.5%, 99.5% or 100% identity to the amino acid sequence of SEQ ID NO: 76;
(iv) the heavy chain comprises the heavy chain variable region comprising an amino acid sequence having at least 85%, 90%, 95%, 97%, 98%, 99%, 99.5%, 99.5% or 100% identity to the amino acid sequence of SEQ ID NO: 74, and the light chain comprises the light chain variable region comprising an amino acid sequence having at least 85%, 90%, 95%, 97%, 98%, 99%, 99.5%, 99.5% or 100% identity to the amino acid sequence of SEQ ID NO: 76;
(v) the heavy chain comprises the heavy chain variable region comprising an amino acid sequence having at least 85%, 90%, 95%, 97%, 98%, 99%, 99.5%, 99.5% or 100% identity to the amino acid sequence of SEQ ID NO: 78, and the light chain comprises the light chain variable region comprising an amino acid sequence having at least 85%, 90%, 95%, 97%, 98%, 99%, 99.5%, 99.5% or 100% identity to the amino acid sequence of SEQ ID NO: 82;
(vi) the heavy chain comprises the heavy chain variable region comprising an amino acid sequence having at least 85%, 90%, 95%, 97%, 98%, 99%, 99.5%, 99.5% or 100% identity to the amino acid sequence of SEQ ID NO: 80, and the light chain comprises the light chain variable region comprising an amino acid sequence having at least 85%, 90%, 95%, 97%, 98%, 99%, 99.5%, 99.5% or 100% identity to the amino acid sequence of SEQ ID NO: 82;
(vii) the heavy chain comprises the heavy chain variable region comprising an amino acid sequence having at least 85%, 90%, 95%, 97%, 98%, 99%, 99.5%, 99.5% or 100% identity to the amino acid sequence of SEQ ID NO: 84, and the light chain comprises the light chain variable region comprising an amino acid sequence having at least 85%, 90%, 95%, 97%, 98%, 99%, 99.5%, 99.5% or 100% identity to the amino acid sequence of SEQ ID NO: 86; or
(viii) the heavy chain comprises the heavy chain variable region comprising an amino acid sequence having at least 85%, 90%, 95%, 97%, 98%, 99%, 99.5%, 99.5% or 100% identity to the amino acid sequence of SEQ ID NO: 88, and the light chain comprises the light chain variable region comprising an amino acid sequence having at least 85%, 90%, 95%, 97%, 98%, 99%, 99.5%, 99.5% or 100% identity to the amino acid sequence of SEQ ID NO: 91.

3. The antibody or binding fragment thereof according to claim 1, wherein the binding fragment is a Fab, Fab′, F(ab′)2, scFv, dsFv, ds-scFv, dimer, minibody, diabody, or bispecific antibody binding fragment.

4. The antibody or binding fragment thereof according to claim 1, wherein the binding fragment is a scFv; and/or wherein the antibody or binding fragment thereof comprises one or more amino acids selected from the group consisting of D-amino acids, modified amino acids, amino acid analogs or combinations thereof; and/or wherein the antibody or binding fragment thereof is modified by pegylation, acetylation, glycosylation, biotinylation, or Prenylation.

5. (canceled)

6. The antibody or binding fragment thereof according to claim 5, wherein the modified amino acids comprise a modification selected from the group consisting of methylation, amidation, acetylation, and/or substitution with other chemical groups.

7. (canceled)

8. A fusion protein comprising the antibody or binding fragment thereof according to claim 1 and i) an E3 ligase or active fragment thereof, ii) an Fc domain or iii) the E3 ligase or active fragment thereof and a Fc domain.

9. The fusion protein according to claim 8, wherein the E3 ligase is CHIP, UBE4A, NEDD4L, UBR5, RNF4, UBOX5, BTrCP, or Parkin, or an active fragment thereof.

10. The fusion protein of claim 8, comprising a heavy chain variable region comprising CDR1, CDR2, and CDR3 having the amino acid sequence of SEQ ID NO: 1, SEQ ID NO: 2, and SEQ ID NO: 3, a first linker, a light chain variable region comprising CDR1, CDR2, and CDR3 having the amino acid sequence of SEQ ID NO: 4, SEQ ID NO: 5, and SEQ ID NO: 6, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 186, or a truncated E3 ligase comprising an amino acid sequence having at least 85%, 90%, 95%, 97%, 98%, 99%, 99.5%, 99.5% or 100% identity to the amino acid sequence of SEQ ID NO: 223.

11. The fusion protein of claim 10, comprising a heavy chain variable region comprising an amino acid sequence having at least 85%, 90%, 95%, 97%, 98%, 99%, 99.5%, 99.5% or 100% identity to the amino acid sequence of SEQ ID NO: 68, a first linker, a light chain variable region comprising an amino acid sequence having at least 85%, 90%, 95%, 97%, 98%, 99%, 99.5%, 99.5% or 100% identity to the amino acid sequence of SEQ ID NO: 70, a second linker, and an active E3 ligase fragment comprising an amino acid sequence having at least 85%, 90%, 95%, 97%, 98%, 99%, 99.5%, 99.5% or 100% identity to the amino acid sequence of SEQ ID NO: 186, or a truncated E3 ligase comprising an amino acid sequence having at least 85%, 90%, 95%, 97%, 98%, 99%, 99.5%, 99.5% or 100% identity to the amino acid sequence of SEQ ID NO: 223.

12. The fusion protein of claim 10, formulated for viral delivery into a subject.

13. The fusion protein of claim 12, wherein the fusion protein is expressed and delivered by AAV.

14. A nucleic acid molecule encoding the antibody or binding fragment thereof according to any one of claim 1, or a fusion protein comprising the antibody or binding fragment thereof and an E3 ligase or active fragment thereof.

15. A vector comprising the nucleic acid of claim 14.

16. A pharmaceutical composition comprising the antibody or antibody thereof according to of any one of claim 1, a fusion protein comprising the antibody and an E3 ligase or active fragment thereof, a nuclei acid encoding the antibody or the fusion protein, or a vector comprising the nucleic acid molecule, and at least one pharmaceutical carrier.

17. A method for treating a medical condition, disease, or disorder mediated by a misfolded form of SOD1 in a subject in need thereof, the method comprises administering to the subject the antibody or binding fragment thereof according to claim 1, a fusion protein comprising the antibody and an E3 ligase or active fragment thereof, a nucleic acid encoding the antibody or the fusion protein, a vector comprising the nucleic acid molecule, or a pharmaceutical composition comprising the antibody, the fusion protein, the nucleic acid molecule, the vector or the pharmaceutical composition.

18. The method according to claim 17, wherein the medical condition, disease or disorder is a neurodegenerative condition, disease or disorder.

19. The method according to claim 18, wherein the neurodegenerative condition, disease or disorder is amyotrophic lateral sclerosis (ALS), Alzheimer's disease, Parkinson's disease, or frontotemporal dementia.

20. The method according to claim 19, wherein the ALS is sporadic ALS or familial ALS.

21.-24. (canceled)

25. The method according to claim 17, wherein the misfolded form of SOD1 comprises SOD1 monomer, a dimer comprising mutant SOD1 monomers, or an aggregate comprising SOD1 monomers and/or dimers, or toxic trimers.

26. The method according to claim 25, wherein the SOD1 monomer comprises a mutant SOD1 monomer.

27. The method according to claim 26, wherein the mutant SOD1 monomer comprises one or more mutations selected from the group consisting of A4V, G93A, G85R, D90A, G127X, V148G, H46R, G37R, C6G, and E100G, and wherein X is truncation mutation.

28. (canceled)


SEQUENCE LISTING (226 sequences)

SEQ ID NO: 1 - protein, 10 aa, Oryctolagus cuniculus
GSDRRTYVMR

SEQ ID NO: 2 - protein, 10 aa, Oryctolagus cuniculus
TIRLDGSRNY

SEQ ID NO: 3 - skipped

SEQ ID NO: 4 - protein, 11 aa, Oryctolagus cuniculus
QASQSISNLLA

SEQ ID NO: 5 - protein, 7 aa, Oryctolagus cuniculus
KASTLES

SEQ ID NO: 6 - protein, 11 aa, Oryctolagus cuniculus
QQGFDSTDVDN

SEQ ID NO: 7 - protein, 10 aa, Oryctolagus cuniculus
GIDLSSSVMA

SEQ ID NO: 8 - protein, 10 aa, Oryctolagus cuniculus
IITNRGNRYY

SEQ ID NO: 9 - skipped

SEQ ID NO: 10 - protein, 13 aa, Oryctolagus cuniculus
QSSQSFLDDNDLA

SEQ ID NO: 11 - protein, 7 aa, Oryctolagus cuniculus
SASTLDS

SEQ ID NO: 12 - protein, 12 aa, Oryctolagus cuniculus
LGDYDCSVGDCS

SEQ ID NO: 13 - protein, 13 aa, Oryctolagus cuniculus
LGDYDCSVGDCSA

SEQ ID NO: 14 - protein, 10 aa, Oryctolagus cuniculus
GIDLSSSVMV

SEQ ID NO: 15 - protein, 10 aa, Oryctolagus cuniculus
IITNRGNRYY

SEQ ID NO: 16 - skipped

SEQ ID NO: 17 - protein, 10 aa, Oryctolagus cuniculus
GFSLSSYGVS

SEQ ID NO: 18 - protein, 10 aa, Oryctolagus cuniculus
FISTRGNPYY

SEQ ID NO: 19 - protein, 8 aa, Oryctolagus cuniculus
ATVVGDSI

SEQ ID NO: 20 - protein, 10 aa, Oryctolagus cuniculus
QSSQSVYKGY

SEQ ID NO: 21 - protein, 7 aa, Oryctolagus cuniculus
ETSTLAS

SEQ ID NO: 22 - protein, 10 aa, Oryctolagus cuniculus
AGGYSNNIYT

SEQ ID NO: 23 - protein, 10 aa, Oryctolagus cuniculus
GIDLSRSVMA

SEQ ID NO: 24 - protein, 10 aa, Oryctolagus cuniculus
IVTNRGNRYY

SEQ ID NO: 25 - skipped

SEQ ID NO: 26 - protein, 13 aa, Oryctolagus cuniculus
QSSQSFLDDNDLA

SEQ ID NO: 27 - protein, 7 aa, Oryctolagus cuniculus
SASTLDS

SEQ ID NO: 28 - protein, 13 aa, Oryctolagus cuniculus
LGDYDCSVGDCSA

SEQ ID NO: 29 - protein, 10 aa, Oryctolagus cuniculus
GIDLRRSVMA

SEQ ID NO: 30 - protein, 10 aa, Oryctolagus cuniculus
IISDRGNTYY

SEQ ID NO: 31 - skipped

SEQ ID NO: 32 - protein, 13 aa, Oryctolagus cuniculus
QSSQRLYDDNDCA

SEQ ID NO: 33 - protein, 7 aa, Oryctolagus cuniculus
DASILDS

SEQ ID NO: 34 - protein, 13 aa, Oryctolagus cuniculus
LGDYDCSVADCST

SEQ ID NO: 35 - protein, 10 aa, Oryctolagus cuniculus
GFSLSSYGVS

SEQ ID NO: 36 - protein, 12 aa, Oryctolagus cuniculus
FISTRGNPYYAS

SEQ ID NO: 37 - protein, 10 aa, Oryctolagus cuniculus
AGATVVGDSI

SEQ ID NO: 38 - protein, 13 aa, Oryctolagus cuniculus
QSSQSVYKGYWQA

SEQ ID NO: 39 - protein, 7 aa, Oryctolagus cuniculus
ETSTLAS

SEQ ID NO: 40 - protein, 10 aa, Oryctolagus cuniculus
AGGYSNNIYT

SEQ ID NO: 41 - protein, 9 aa, Oryctolagus cuniculus
FSLSSYGVS

SEQ ID NO: 42 - protein, 13 aa, Oryctolagus cuniculus
YIGFISTRGNPYY

SEQ ID NO: 43 - protein, 9 aa, Oryctolagus cuniculus
GATVVGDSI

SEQ ID NO: 44 - protein, 10 aa, Oryctolagus cuniculus
QSVYKGYWQA

SEQ ID NO: 45 - protein, 11 aa, Oryctolagus cuniculus
LLIYETSTLAS

SEQ ID NO: 46 - protein, 9 aa, Oryctolagus cuniculus
GGYSNNIYT

SEQ ID NO: 47 - protein, 7 aa, Oryctolagus cuniculus
YGVSWVR

SEQ ID NO: 48 - protein, 17 aa, Oryctolagus cuniculus
FISTRGNPYYASWAKGR

SEQ ID NO: 49 - protein, 10 aa, Oryctolagus cuniculus
GATVVGDSIW

SEQ ID NO: 50 - protein, 11 aa, Oryctolagus cuniculus
SSQSVYKGYWQ

SEQ ID NO: 51 - protein, 7 aa, Oryctolagus cuniculus
YETSTLA

SEQ ID NO: 52 - protein, 9 aa, Oryctolagus cuniculus
CAGGYSNNI

SEQ ID NO: 53 - protein, 8 aa, Oryctolagus cuniculus
FSLSSYGV

SEQ ID NO: 54 - protein, 5 aa, Oryctolagus cuniculus
TRGNP

SEQ ID NO: 55 - protein, 10 aa, Oryctolagus cuniculus
GATVVGDSIW

SEQ ID NO: 56 - protein, 11 aa, Oryctolagus cuniculus
SSQSVYKGYWQ

SEQ ID NO: 57 - protein, 7 aa, Oryctolagus cuniculus
YETSTLA

SEQ ID NO: 58 - protein, 10 aa, Oryctolagus cuniculus
CAGGYSNNIY

SEQ ID NO: 59 - protein, 14 aa, Oryctolagus cuniculus
SGFSLSSYGVSWVR

SEQ ID NO: 60 - protein, 21 aa, Oryctolagus cuniculus
YYASWAKGRFTISKTSTTVDL

SEQ ID NO: 61 - protein, 30 aa, Oryctolagus cuniculus
LVTVSSGQPKAPSVFPLAPCCGDTPSSTVT

SEQ ID NO: 62 - protein, 16 aa, Oryctolagus cuniculus
SQSVYKGYWQAWYQQK

SEQ ID NO: 63 - protein, 17 aa, Oryctolagus cuniculus
GVPSRFSGSGSGTQFTL

SEQ ID NO: 64 - protein, 29 aa, Oryctolagus cuniculus
KGDPVAPTVLIFPPAADQVATGTVTIVCV

SEQ ID NO: 65 - protein, 109 aa, Oryctolagus cuniculus
QSVEESGGRLVSPGGSLTLTCRVSGSDRRTYVMRWVRQAPGKGLEWIGTIRLDGSRNYATWAKGRFTISKSSSTTMELRMTSLTSEDTATYFCVRDDLWGPGTLVTVSS

SEQ ID NO: 66 - DNA, 351 bp, Oryctolagus cuniculus
  source 1..351: mol_type = genomic DNA
atggagactgggctgcgctggcttctcctggtcgctgtgctcaaaggtgtccagtgtcagtcggtggaggagtccgggggtcgcctggtgtcgcctggaggatccctgacactcacctgccgtgtttctggaagcgaccgcaggacctatgtaatgcgctgggtccgccaggctccggggaaggggctggaatggatcggaaccattagacttgatggtagtagaaactatgcgacctgggcgaaaggccgtttcaccatctccaagtcctcgtcgaccacgatggagctgagaatgaccagtctgacaagcgaggacacggccacttatttctgtgtcagagacgacttg

SEQ ID NO: 67 - DNA, 384 bp, Oryctolagus cuniculus
  source 1..384: mol_type = genomic DNA
atggagactgggctgcgctggcttctcctggtcgctgtgctcaaaggtgtccagtgtcagtcggtggaggagtccgggggtcgcctggtgtcgcctggaggatccctgacactcacctgccgtgtttctggaagcgaccgcaggacctatgtaatgcgctgggtccgccaggctccggggaaggggctggaatggatcggaaccattagacttgatggtagtagaaactatgcgacctgggcgaaaggccgtttcaccatctccaagtcctcgtcgaccacgatggagctgaggatgaccagtctgacaagcgaggacacggccacttatttctgtgtcagagacgacttgtggggcccaggcaccctggtcaccgtctcctca

SEQ ID NO: 68 - protein, 109 aa, Oryctolagus cuniculus
QSVEESGGRLVSPGGSLTLTCRVSGSDRRTYVMRWVRQAPGKGLEWIGTIRLDGSRNYATWAKGRFTISKSSSTTMELRMTSLTSEDTATYFCVRDDLGGPGTLVTVSS

SEQ ID NO: 69 - DNA, 384 bp, Oryctolagus cuniculus
  source 1..384: mol_type = genomic DNA
atggagactgggctgcgctggcttctcctggtcgctgtgctcaaaggtgtccagtgtcagtcggtggaggagtccgggggtcgcctggtgtcgcctggaggatccctgacactcacctgccgtgtttctggaagcgaccgcaggacctatgtaatgcgctgggtccgccaggctccggggaaggggctggaatggatcggaaccattagacttgatggtagtagaaactatgcgacctgggcgaaaggccgtttcaccatctccaagtcctcgtcgaccacgatggagctgagaatgaccagtctgacaagcgaggacacggccacttatttctgtgtcagagacgacttggggggcccaggcaccctggtcaccgtctcctca

SEQ ID NO: 70 - protein, 111 aa, Oryctolagus cuniculus
ADIVMTQTPASVEVAVGGTVTIKCQASQSISNLLAWYQQKPGQPPKLLIYKASTLESGVPSRFKGSGSGTQFALTISGVQCDDAATYYCQQGFDSTDVDNLFGGGTEVVVA

SEQ ID NO: 71 - DNA, 399 bp, Oryctolagus cuniculus
  source 1..399: mol_type = genomic DNA
atggacacgagggcccccactcagctgctggggctcctgctgctctggctcccaggtgccagatgtgctgacatcgtgatgacccagactccagcctctgtggaggtagctgtgggaggcacagtcaccatcaagtgccaggccagtcagagcattagcaacctcttagcctggtatcagcagaaaccaggacagcctcccaagctcctgatctacaaggcttccactctggaatctggggtcccatcgcggttcaaaggcagtggatctgggacacagttcgctctcaccatcagcggcgtgcagtgtgacgatgctgccacttactactgtcaacagggtttcgatagtactgatgttgacaatcttttcggcggagggaccgaagtggtggtcgca

SEQ ID NO: 72 - protein, 108 aa, Oryctolagus cuniculus
QSVEESGGRLVTPGTPLTLTCTVSGIDLSSSVMAWVRQAPGKGLEWIGIITNRGNRYYAKWAKGRFTISRTSTTVGLKITSPTTADTATYFCGRGSVWGPGTLVTVSS

SEQ ID NO: 73 - DNA, 348 bp, Oryctolagus cuniculus
  source 1..348: mol_type = genomic DNA
atggagactgggctgcgctggcttctcctggtcgctgtgctcaaaggtgtccagtgtcagtcggtggaggagtccgggggtcgcctggtcacgcctgggacacccctgacactcacctgcacagtctctggaatcgacctcagtagcagtgtaatggcctgggtccgccaggctccagggaaggggctggaatggatcggaatcattactaatcgtggtaatagatactacgcgaagtgggcgaaaggccgattcaccatctccagaacctcgaccacggtgggtctgaagatcaccagcccgacaaccgcggacacggccacctatttctgtggcagaggtagcgtc

SEQ ID NO: 74 - protein, 108 aa, Oryctolagus cuniculus
QSVEESGGRLVTPGTPLTLTCTVSGIDLSSSVMVWVRQAPGKGLEWIGIITNRGNRYYAKWAKGRFTISRTSTTVGLKITSPTTADTATYFCGRGSVWGPGTLVTVSS

SEQ ID NO: 75 - DNA, 348 bp, Oryctolagus cuniculus
  source 1..348: mol_type = genomic DNA
atggagactgggctgcgctggcttctcctggtcgctgtgctcaaaggtgtccagtgtcagtcggtggaggagtccgggggtcgcctggtcacgcctgggacacccctgacactcacctgcacagtctctggaatcgacctcagtagcagtgtaatggtctgggtccgccaggctccagggaaggggctggaatggatcggaatcattactaatcgtggtaatagatactacgcgaagtgggcgaaaggccgattcaccatctccagaacctcgaccacggtgggtctgaagatcaccagtccgacaaccgcggacacggccacctatttctgtggcagaggtagcgtc

SEQ ID NO: 76 - protein, 112 aa, Oryctolagus cuniculus
QVLTQTPASVSAAIGGTVTIKCQSSQSFLDDNDLAWYQKKPGRPPKRLIKSASTLDSGVPSRFSGSGSGTQFTLTISDVQCADAATYYCLGDYDCSVGDCSAFGGGTEVVVK

SEQ ID NO: 77 - DNA, 405 bp, Oryctolagus cuniculus
  source 1..405: mol_type = genomic DNA
atggacacgagggcccccactcagctgctggggctcctgctgctctggctcccaggtgccacatttgcccaagtgctgacccagactccagcctcggtgtctgcggctattggaggcacagttaccatcaagtgccagtccagtcagagttttcttgatgacaatgacttagcctggtatcagaagaaaccagggcggcctcccaagcgcctgatcaaatctgcatcgactctagattctggggtcccatcacggttcagtggcagtggatctgggacacagttcactctcaccatcagcgacgtgcagtgtgccgatgctgccacttattactgtctaggcgattatgattgtagtgttggtgattgtagtgctttcggcggagggaccgaagtggtggtcaag

SEQ ID NO: 78 - protein, 113 aa, Oryctolagus cuniculus
QSVGESGGGLVTPGTPLTLTCTVSGFSLSSYGVSWVRQAPGKGLEYIGFISTRGNPYYASWAKGRFTISKTSTTVDLKITSPTTEDTATYFCAGATVVGDSIWGPGTLVTVSS

SEQ ID NO: 79 - DNA, 363 bp, Oryctolagus cuniculus
  source 1..363: mol_type = genomic DNA
atggagactgggctgcgctggcttctcctggtcgctgtgctcaaaggtgtccagtgtcagtcggtgggggagtccgggggaggcctggtcacgcctgggacacccctgacactcacctgcacagtctctggattctccctcagtagctatggagtgagctgggtccgccaggctccagggaaggggctggaatacatcggattcattagcactcgtggtaacccatactacgcgagctgggcgaaaggccgattcaccatctccaaaacctcgaccacggtggatctgaaaatcaccagtccgacaaccgaggacacggccacctatttctgtgccggggcaactgttgttggtgatagtatt

SEQ ID NO: 80 - protein, 113 aa, Oryctolagus cuniculus
QSVEESGGGLVTPGTPLTLTCTVSGFSLSSYGVSWVRQAPGKGLEYIGFISTRGNPYYASWAKGRFTISKTSTTVDLKITSPTTEDTATYFCAGATVVGDSIWGPGTLVTVSS

SEQ ID NO: 81 - DNA, 363 bp, Oryctolagus cuniculus
  source 1..363: mol_type = genomic DNA
atggagactgggctgcgctggcttctcctggtcgctgtgctcaaaggtgtccagtgtcagtcggtggaggagtccgggggaggcctggtcacgcctgggacacccctgacactcacctgcacagtctctggattctccctcagtagctatggagtgagctgggtccgccaggctccagggaaggggctggaatacatcggattcattagcactcgtggtaacccatactacgcgagctgggcgaaaggccgattcaccatctccaaaacctcgaccacggtggatctgaaaatcaccagtccgacaaccgaggacacggccacctatttctgtgccggggcaactgttgttggtgatagtatt

SEQ ID NO: 82 - protein, 109 aa, Oryctolagus cuniculus
QVLTQTASSVSAAVGGTVTISCQSSQSVYKGYWQAWYQQKPGQPPKLLIYETSTLASGVPSRFSGSGSGTQFTLTISGVQCDDAATYYCAGGYSNNIYTFGGGTEVVVK

SEQ ID NO: 83 - DNA, 396 bp, Oryctolagus cuniculus
  source 1..396: mol_type = genomic DNA
atggacacgagggcccccactcagctgctggggctcctgctgctctggctcccaggtgccacatttgcgcaagtgctgacccagactgcatcgtccgtgtctgcagctgtgggaggcacagtcaccatcagttgccagtccagtcagagtgtttataagggctactggcaagcctggtatcagcagaaaccagggcagcctcccaagctcctgatctacgaaacatccactctggcatctggggtcccatcgcggttcagcggcagtggatctgggacacagttcactctcaccatcagcggcgtgcagtgtgacgatgctgccacttactactgtgcaggcggttatagtaataatatttatactttcggcggagggaccgaggtggtggtcaag

SEQ ID NO: 84 - protein, 108 aa, Oryctolagus cuniculus
QSVEESGGRLVTPGTPLTLTCTVSGIDLSRSVMAWVRQAPGKGLEWIGIVTNRGNRYYANWAKGRFTISKTSTTVGLKIPSPTTADTATYFCGKGAVWGPGTLVTVSS

SEQ ID NO: 85 - DNA, 348 bp, Oryctolagus cuniculus
  source 1..348: mol_type = genomic DNA
atggagactgggctgcgctggcttctcctggtcgctgtgctcaaaggtgtccagtgtcagtcggtggaggagtccgggggtcgcctggtcacgcctgggacacccctgacactcacctgcacagtctctggaatcgacctcagtagaagtgtgatggcctgggtccgccaggctccagggaaggggctggaatggatcggaatcgttactaatcgtggtaatagatactacgctaactgggcgaaaggccgattcaccatctccaaaacctcgaccacggtgggtctgaagatccccagtccgacaaccgcggacacggccacctatttctgtggcaaaggtgccgtc

SEQ ID NO: 86 - protein, 112 aa, Oryctolagus cuniculus
QVLTQTPSSVSAAVGGTVTINCQSSQSFLDDNDLAWFQQKPGRPPKRLIKSASTLDSGVPSRFKGSGSGTQFTLTISDVQCNDAATYYCLGDYDCSVGDCSAFGGGTEVVVK

SEQ ID NO: 87 - DNA, 405 bp, Oryctolagus cuniculus
  source 1..405: mol_type = genomic DNA
atggacacgagggcccccactcagctgctggggctcctgctgctctggctcccaggtgccacatttgcccaagtgctgacccagactccatcctccgtgtctgcagctgttggaggcacagtcaccatcaattgccagtccagtcagagttttcttgatgacaacgacttagcctggtttcagcagaaaccagggcggcctcccaagcgcctgatcaaatctgcatcgactctagattctggggtcccatcgcggttcaaaggcagtggatctgggacacagttcactctcaccatcagcgacgtgcagtgtaacgatgctgccacttattactgtctaggcgattatgattgtagtgttggtgattgtagtgctttcggcggagggaccgaagtggtggtcaag

SEQ ID NO: 88 - protein, 108 aa, Oryctolagus cuniculus
QSVEESGGRLVTPGTPLTLTCTVSGIDLRRSVMAWVRQAPGKGLEWIGIISDRGNTYYASWAKGRFTISRTSTTVALKMTSPTTADTATYFCGRGNVWGPGTLVTVSS

SEQ ID NO: 89 - DNA, 348 bp, Oryctolagus cuniculus
  source 1..348: mol_type = genomic DNA
atggagactgggctgcgctggcttctcctggtcgcggtgctcaaaggtgtccagtgtcagtcggtggaggagtccgggggtcgcctggtcacgcctgggacacccctgacactcacctgcacagtctctggaatcgacctcagacgcagtgtaatggcctgggtccgccaggctccagggaaggggctggaatggatcggaatcattagtgatcgtggtaacacatactacgcgagctgggcgaaaggccgattcaccatctccagaacctcgaccacggtggctctgaaaatgaccagtccgacaaccgcggacacggccacctatttttgtggcagaggtaatgtc

SEQ ID NO: 90 - DNA, 381 bp, Oryctolagus cuniculus
  source 1..381: mol_type = genomic DNA
atggagactgggctgcgctggcttctcctggtcgctgtgctcaaaggtgtccagtgtcagtcggtggaggagtccgggggtcgcctggtcacgcctgggacacccctgacactcacctgcacagtctctggaatcgacctcagacgcagtgtaatggcctgggtccgccaggctccagggaaggggctggaatggatcggaatcattagtgatcgtggtaacacatactacgcgagctgggcgaaaggccgattcaccatctccagaacctcgaccacggtggctctgaaaatgaccagtccgacaaccgcggacacggccacctatttttgtggcagaggtaatgtctggggcccaggcaccctggtcaccgtctcctca

SEQ ID NO: 91 - protein, 112 aa, Oryctolagus cuniculus
QVLTQTPSSVSAAVGGTVTINCQSSQRLYDDNDCAWYQKKPGQPPKRLIHDASILDSGVSSRFSGSGSGTQFTLTISGVQCEDAATYYCLGDYDCSVADCSTFGGGTEVVVK

SEQ ID NO: 92 - DNA, 405 bp, Oryctolagus cuniculus
  source 1..405: mol_type = genomic DNA
atggacacgagggcccccactcagctgctggggctcctgctgctctggctcccaggtgccacatttgcccaagtgctgacccagactccatcctccgtgtctgcagctgtgggaggcacagtcaccatcaattgccagtccagtcagcggctctatgatgacaacgactgtgcctggtatcagaagaaaccagggcagcctcccaagcgcctgatccatgatgcatccattctagattctggggtctcatcgcggttcagtggcagtggatctgggacacagttcactctcaccatcagcggcgtgcagtgtgaagatgctgccacttactactgtctaggcgattatgattgtagtgttgctgattgtagtactttcggcggagggaccgaggtggtggtcaaa

SEQ ID NO: 93 - DNA, 402 bp, Mus musculus
  source 1..402: mol_type = genomic DNA
tacttgggactgaactgtgtattcatagtttttctcttaaaaggtgtccagagtgaagtgaagcttgaggagtctggaggaggcttggtgcaacctggaggatccatgaaactctcctgtgttgcctctggatccactttcagtaactactggatgaactgggtccgccagtctccagagaaggggcttgagtgggttgctgaaattagattgaaatctaatacttatgcaatacattatgcggagtctgtgaaagggaggttcaccatctcaagagataattccaaaagtagtgtctacctgcaaatgaacaacttaagagctgaagacactggcatttattactgtaccggcaatgctatggacttctggggtcaaggaacctcagtcaccgtctcctca

SEQ ID NO: 94 - protein, 134 aa, Mus musculus
YLGLNCVFIVFLLKGVQSEVKLEESGGGLVQPGGSMKLSCVASGSTFSNYWMNWVRQSPEKGLEWVAEIRLKSNTYAIHYAESVKGRFTISRDNSKSSVYLQMNNLRAEDTGIYYCTGNAMDFWGQGTSVTVSS

SEQ ID NO: 95 - DNA, 393 bp, Mus musculus
  source 1..393: mol_type = genomic DNA
aagttgcctgttaggctgttggtgctgatgttctggattcctgcttccagcagtgatgttgtgatgacccaaactccactctccctgcctgtcagtcttggagatcaagcctccatctcttgcagatctagtcagagccttgtatatagtaatggaaacacctatttacattggtacctgcagaagccaggccagtctccaaagctcctgatctacaaagtttccaaccgattttctggggtcccagacaggttcagtggcagtggatcagggacagatttcacactcaggatcagcagagtggaggctgaggatctgggagtttatttctgctctcaaagtacacatgttcctccgtggacgttcggtggaggcaccaagctggaaatcaaa

SEQ ID NO: 96 - protein, 131 aa, Mus musculus
KLPVRLLVLMFWIPASSSDVVMTQTPLSLPVSLGDQASISCRSSQSLVYSNGNTYLHWYLQKPGQSPKLLIYKVSNRFSGVPDRFSGSGSGTDFTLRISRVEAEDLGVYFCSQSTHVPPWTFGGGTKLEIK

SEQ ID NO: 97 - protein, 8 aa, Mus musculus
GSTFSNYW

SEQ ID NO: 98 - DNA, 24 bp, Mus musculus
  source 1..24: mol_type = genomic DNA
ggatccactttcagtaactactgg

SEQ ID NO: 99 - protein, 10 aa, Mus musculus
VAEIRLKSNT

SEQ ID NO: 100 - DNA, 30 bp, Mus musculus
  source 1..30: mol_type = genomic DNA
gttgctgaaattagattgaaatctaatact

SEQ ID NO: 101 - protein, 7 aa, Mus musculus
TGNAMDF

SEQ ID NO: 102 - DNA, 21 bp, Mus musculus
  source 1..21: mol_type = genomic DNA
accggcaatgctatggacttc

SEQ ID NO: 103 - protein, 11 aa, Mus musculus
QSLVYSNGNTY

SEQ ID NO: 104 - DNA, 33 bp, Mus musculus
  source 1..33: mol_type = genomic DNA
cagagccttgtatatagtaatggaaacacctat

SEQ ID NO: 105-106 - skipped (2 entries)

SEQ ID NO: 107 - protein, 10 aa, Mus musculus
SQSTHVPPWT

SEQ ID NO: 108 - DNA, 30 bp, Mus musculus
  source 1..30: mol_type = genomic DNA
tctcaaagtacacatgttcctccgtggacg

SEQ ID NO: 109 - protein, 451 aa, Oryctolagus cuniculus
METGLRWLLLVAVLKGVQCQSVEESGGRLVSPGGSLTLTCRVSGSDRRTYVMRWVRQAPGKGLEWIGTIRLDGSRNYATWAKGRFTISKSSSTTMELRMTSLTSEDTATYFCVRDDLWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 110 - DNA, 1356 bp, Oryctolagus cuniculus
  source 1..1356: mol_type = genomic DNA
atggagactgggctgcgctggcttctcctggtcgctgtgctcaaaggtgtccagtgtcagtcggtggaggagtccgggggtcgcctggtgtcgcctggaggatccctgacactcacctgccgtgtttctggaagcgaccgcaggacctatgtaatgcgctgggtccgccaggctccggggaaggggctggaatggatcggaaccattagacttgatggtagtagaaactatgcgacctgggcgaaaggccgtttcaccatctccaagtcctcgtcgaccacgatggagctgagaatgaccagtctgacaagcgaggacacggccacttatttctgtgtcagagacgacttgtggggcccaggcaccctggtcaccgtctcctcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 111 - DNA, 1353 bp, Oryctolagus cuniculus
  source 1..1353: mol_type = genomic DNA
gagactgggctgcgctggcttctcctggtcgctgtgctcaaaggtgtccagtgtcagtcggtggaggagtccgggggtcgcctggtgtcgcctggaggatccctgacactcacctgccgtgtttctggaagcgaccgcaggacctatgtaatgcgctgggtccgccaggctccggggaaggggctggaatggatcggaaccattagacttgatggtagtagaaactatgcgacctgggcgaaaggccgtttcaccatctccaagtcctcgtcgaccacgatggagctgaggatgaccagtctgacaagcgaggacacggccacttatttctgtgtcagagacgacttgtggggcccaggcaccctggtcaccgtctcctcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 112 - protein, 451 aa, Oryctolagus cuniculus
METGLRWLLLVAVLKGVQCQSVEESGGRLVSPGGSLTLTCRVSGSDRRTYVMRWVRQAPGKGLEWIGTIRLDGSRNYATWAKGRFTISKSSSTTMELRMTSLTSEDTATYFCVRDDLGGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 113 - DNA, 1356 bp, Oryctolagus cuniculus
  source 1..1356: mol_type = genomic DNA
atggagactgggctgcgctggcttctcctggtcgctgtgctcaaaggtgtccagtgtcagtcggtggaggagtccgggggtcgcctggtgtcgcctggaggatccctgacactcacctgccgtgtttctggaagcgaccgcaggacctatgtaatgcgctgggtccgccaggctccggggaaggggctggaatggatcggaaccattagacttgatggtagtagaaactatgcgacctgggcgaaaggccgtttcaccatctccaagtcctcgtcgaccacgatggagctgagaatgaccagtctgacaagcgaggacacggccacttatttctgtgtcagagacgacttggggggcccaggcaccctggtcaccgtctcctcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 114 - protein, 237 aa, Oryctolagus cuniculus
MDTRAPTQLLGLLLLWLPGARCADIVMTQTPASVEVAVGGTVTIKCQASQSISNLLAWYQQKPGQPPKLLIYKASTLESGVPSRFKGSGSGTQFALTISGVQCDDAATYYCQQGFDSTDVDNLFGGGTEVVVAGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSLNRGDC

SEQ ID NO: 115 - DNA, 714 bp, Oryctolagus cuniculus
  source 1..714: mol_type = genomic DNA
atggacacgagggcccccactcagctgctggggctcctgctgctctggctcccaggtgccagatgtgctgacatcgtgatgacccagactccagcctctgtggaggtagctgtgggaggcacagtcaccatcaagtgccaggccagtcagagcattagcaacctcttagcctggtatcagcagaaaccaggacagcctcccaagctcctgatctacaaggcttccactctggaatctggggtcccatcgcggttcaaaggcagtggatctgggacacagttcgctctcaccatcagcggcgtgcagtgtgacgatgctgccacttactactgtcaacagggtttcgatagtactgatgttgacaatcttttcggcggagggaccgaagtggtggtcgcaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcctcaataggggtgactgttag

SEQ ID NO: 116 - protein, 450 aa, Oryctolagus cuniculus
METGLRWLLLVAVLKGVQCQSVEESGGRLVTPGTPLTLTCTVSGIDLSSSVMAWVRQAPGKGLEWIGIITNRGNRYYAKWAKGRFTISRTSTTVGLKITSPTTADTATYFCGRGSVWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 117 - DNA, 1353 bp, Oryctolagus cuniculus
  source 1..1353: mol_type = genomic DNA
atggagactgggctgcgctggcttctcctggtcgctgtgctcaaaggtgtccagtgtcagtcggtggaggagtccgggggtcgcctggtcacgcctgggacacccctgacactcacctgcacagtctctggaatcgacctcagtagcagtgtaatggcctgggtccgccaggctccagggaaggggctggaatggatcggaatcattactaatcgtggtaatagatactacgcgaagtgggcgaaaggccgattcaccatctccagaacctcgaccacggtgggtctgaagatcaccagcccgacaaccgcggacacggccacctatttctgtggcagaggtagcgtctggggcccaggcaccctggtcaccgtctcctcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 118 - protein, 450 aa, Oryctolagus cuniculus
METGLRWLLLVAVLKGVQCQSVEESGGRLVTPGTPLTLTCTVSGIDLSSSVMVWVRQAPGKGLEWIGIITNRGNRYYAKWAKGRFTISRTSTTVGLKITSPTTADTATYFCGRGSVWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 119 - DNA, 1353 bp, Oryctolagus cuniculus
  source 1..1353: mol_type = genomic DNA
atggagactgggctgcgctggcttctcctggtcgctgtgctcaaaggtgtccagtgtcagtcggtggaggagtccgggggtcgcctggtcacgcctgggacacccctgacactcacctgcacagtctctggaatcgacctcagtagcagtgtaatggtctgggtccgccaggctccagggaaggggctggaatggatcggaatcattactaatcgtggtaatagatactacgcgaagtgggcgaaaggccgattcaccatctccagaacctcgaccacggtgggtctgaagatcaccagtccgacaaccgcggacacggccacctatttctgtggcagaggtagcgtctggggcccaggcaccctggtcaccgtctcctcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 120 - protein, 239 aa, Oryctolagus cuniculus
MDTRAPTQLLGLLLLWLPGATFAQVLTQTPASVSAAIGGTVTIKCQSSQSFLDDNDLAWYQKKPGRPPKRLIKSASTLDSGVPSRFSGSGSGTQFTLTISDVQCADAATYYCLGDYDCSVGDCSAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 121 - DNA, 720 bp, Oryctolagus cuniculus
  source 1..720: mol_type = genomic DNA
atggacacgagggcccccactcagctgctggggctcctgctgctctggctcccaggtgccacatttgcccaagtgctgacccagactccagcctcggtgtctgcggctattggaggcacagttaccatcaagtgccagtccagtcagagttttcttgatgacaatgacttagcctggtatcagaagaaaccagggcggcctcccaagcgcctgatcaaatctgcatcgactctagattctggggtcccatcacggttcagtggcagtggatctgggacacagttcactctcaccatcagcgacgtgcagtgtgccgatgctgccacttattactgtctaggcgattatgattgtagtgttggtgattgtagtgctttcggcggagggaccgaagtggtggtcaagggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 122 - protein, 455 aa, Oryctolagus cuniculus
METGLRWLLLVAVLKGVQCQSVGESGGGLVTPGTPLTLTCTVSGFSLSSYGVSWVRQAPGKGLEYIGFISTRGNPYYASWAKGRFTISKTSTTVDLKITSPTTEDTATYFCAGATVVGDSIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 123 - DNA, 1368 bp, Oryctolagus cuniculus
  source 1..1368: mol_type = genomic DNA
atggagactgggctgcgctggcttctcctggtcgctgtgctcaaaggtgtccagtgtcagtcggtgggggagtccgggggaggcctggtcacgcctgggacacccctgacactcacctgcacagtctctggattctccctcagtagctatggagtgagctgggtccgccaggctccagggaaggggctggaatacatcggattcattagcactcgtggtaacccatactacgcgagctgggcgaaaggccgattcaccatctccaaaacctcgaccacggtggatctgaaaatcaccagtccgacaaccgaggacacggccacctatttctgtgccggggcaactgttgttggtgatagtatttggggcccaggcaccctggtcaccgtctcctcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 124 - protein, 455 aa, Oryctolagus cuniculus
METGLRWLLLVAVLKGVQCQSVEESGGGLVTPGTPLTLTCTVSGFSLSSYGVSWVRQAPGKGLEYIGFISTRGNPYYASWAKGRFTISKTSTTVDLKITSPTTEDTATYFCAGATVVGDSIWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 125 - DNA, 1368 bp, Oryctolagus cuniculus
  source 1..1368: mol_type = genomic DNA
atggagactgggctgcgctggcttctcctggtcgctgtgctcaaaggtgtccagtgtcagtcggtggaggagtccgggggaggcctggtcacgcctgggacacccctgacactcacctgcacagtctctggattctccctcagtagctatggagtgagctgggtccgccaggctccagggaaggggctggaatacatcggattcattagcactcgtggtaacccatactacgcgagctgggcgaaaggccgattcaccatctccaaaacctcgaccacggtggatctgaaaatcaccagtccgacaaccgaggacacggccacctatttctgtgccggggcaactgttgttggtgatagtatttggggcccaggcaccctggtcaccgtctcctcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 126 - protein, 236 aa, Oryctolagus cuniculus
MDTRAPTQLLGLLLLWLPGATFAQVLTQTASSVSAAVGGTVTISCQSSQSVYKGYWQAWYQQKPGQPPKLLIYETSTLASGVPSRFSGSGSGTQFTLTISGVQCDDAATYYCAGGYSNNIYTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 127 - DNA, 711 bp, Oryctolagus cuniculus
  source 1..711: mol_type = genomic DNA
atggacacgagggcccccactcagctgctggggctcctgctgctctggctcccaggtgccacatttgcgcaagtgctgacccagactgcatcgtccgtgtctgcagctgtgggaggcacagtcaccatcagttgccagtccagtcagagtgtttataagggctactggcaagcctggtatcagcagaaaccagggcagcctcccaagctcctgatctacgaaacatccactctggcatctggggtcccatcgcggttcagcggcagtggatctgggacacagttcactctcaccatcagcggcgtgcagtgtgacgatgctgccacttactactgtgcaggcggttatagtaataatatttatactttcggcggagggaccgaggtggtggtcaagggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 128 - protein, 450 aa, Oryctolagus cuniculus
METGLRWLLLVAVLKGVQCQSVEESGGRLVTPGTPLTLTCTVSGIDLSRSVMAWVRQAPGKGLEWIGIVTNRGNRYYANWAKGRFTISKTSTTVGLKIPSPTTADTATYFCGKGAVWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 129 - DNA, 1353 bp, Oryctolagus cuniculus
  source 1..1353: mol_type = genomic DNA
atggagactgggctgcgctggcttctcctggtcgctgtgctcaaaggtgtccagtgtcagtcggtggaggagtccgggggtcgcctggtcacgcctgggacacccctgacactcacctgcacagtctctggaatcgacctcagtagaagtgtgatggcctgggtccgccaggctccagggaaggggctggaatggatcggaatcgttactaatcgtggtaatagatactacgctaactgggcgaaaggccgattcaccatctccaaaacctcgaccacggtgggtctgaagatccccagtccgacaaccgcggacacggccacctatttctgtggcaaaggtgccgtctggggcccaggcaccctggtcaccgtctcctcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 130 - protein, 239 aa, Oryctolagus cuniculus
MDTRAPTQLLGLLLLWLPGATFAQVLTQTPSSVSAAVGGTVTINCQSSQSFLDDNDLAWFQQKPGRPPKRLIKSASTLDSGVPSRFKGSGSGTQFTLTISDVQCNDAATYYCLGDYDCSVGDCSAFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 131 - DNA, 720 bp, Oryctolagus cuniculus
  source 1..720: mol_type = genomic DNA
atggacacgagggcccccactcagctgctggggctcctgctgctctggctcccaggtgccacatttgcccaagtgctgacccagactccatcctccgtgtctgcagctgttggaggcacagtcaccatcaattgccagtccagtcagagttttcttgatgacaacgacttagcctggtttcagcagaaaccagggcggcctcccaagcgcctgatcaaatctgcatcgactctagattctggggtcccatcgcggttcaaaggcagtggatctgggacacagttcactctcaccatcagcgacgtgcagtgtaacgatgctgccacttattactgtctaggcgattatgattgtagtgttggtgattgtagtgctttcggcggagggaccgaagtggtggtcaagggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 132 - protein, 450 aa, Oryctolagus cuniculus
METGLRWLLLVAVLKGVQCQSVEESGGRLVTPGTPLTLTCTVSGIDLRRSVMAWVRQAPGKGLEWIGIISDRGNTYYASWAKGRFTISRTSTTVALKMTSPTTADTATYFCGRGNVWGPGTLVTVSSGQPKAPSVFPLAPCCGDTPSSTVTLGCLVKGYLPEPVTVTWNSGTLTNGVRTFPSVRQSSGLYSLSSVVSVTSSSQPVTCNVAHPATNTKVDKTVAPSTCSKPTCPPPELLGGPSVFIFPPKPKDTLMISRTPEVTCVVVDVSQDDPEVQFTWYINNEQVRTARPPLREQQFNSTIRVVSTLPIAHQDWLRGKEFKCKVHNKALPAPIEKTISKARGQPLEPKVYTMGPPREELSSRSVSLTCMINGFYPSDISVEWEKNGKAEDNYKTTPAVLDSDGSYFLYSKLSVPTSEWQRGDVFTCSVMHEALHNHYTQKSISRSPGK

SEQ ID NO: 133 - DNA, 1353 bp, Oryctolagus cuniculus
  source 1..1353: mol_type = genomic DNA
atggagactgggctgcgctggcttctcctggtcgcggtgctcaaaggtgtccagtgtcagtcggtggaggagtccgggggtcgcctggtcacgcctgggacacccctgacactcacctgcacagtctctggaatcgacctcagacgcagtgtaatggcctgggtccgccaggctccagggaaggggctggaatggatcggaatcattagtgatcgtggtaacacatactacgcgagctgggcgaaaggccgattcaccatctccagaacctcgaccacggtggctctgaaaatgaccagtccgacaaccgcggacacggccacctatttttgtggcagaggtaatgtctggggcccaggcaccctggtcaccgtctcctcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 134 - DNA, 1353 bp, Oryctolagus cuniculus
  source 1..1353: mol_type = genomic DNA
atggagactgggctgcgctggcttctcctggtcgctgtgctcaaaggtgtccagtgtcagtcggtggaggagtccgggggtcgcctggtcacgcctgggacacccctgacactcacctgcacagtctctggaatcgacctcagacgcagtgtaatggcctgggtccgccaggctccagggaaggggctggaatggatcggaatcattagtgatcgtggtaacacatactacgcgagctgggcgaaaggccgattcaccatctccagaacctcgaccacggtggctctgaaaatgaccagtccgacaaccgcggacacggccacctatttttgtggcagaggtaatgtctggggcccaggcaccctggtcaccgtctcctcagggcaacctaaggctccatcagtcttcccactggccccctgctgcggggacacacccagctccacggtgaccctgggctgcctggtcaaagggtacctcccggagccagtgaccgtgacctggaactcgggcaccctcaccaatggggtacgcaccttcccgtccgtccggcagtcctcaggcctctactcgctgagcagcgtggtgagcgtgacctcaagcagccagcccgtcacctgcaacgtggcccacccagccaccaacaccaaagtggacaagaccgttgcgccctcgacatgcagcaagcccacgtgcccaccccctgaactcctggggggaccgtctgtcttcatcttccccccaaaacccaaggacaccctcatgatctcacgcacccccgaggtcacatgcgtggtggtggacgtgagccaggatgaccccgaggtgcagttcacatggtacataaacaacgagcaggtgcgcaccgcccggccgccgctacgggagcagcagttcaacagcacgatccgcgtggtcagcaccctccccatcgcgcaccaggactggctgaggggcaaggagttcaagtgcaaagtccacaacaaggcactcccggcccccatcgagaaaaccatctccaaagccagagggcagcccctggagccgaaggtctacaccatgggccctccccgggaggagctgagcagcaggtcggtcagcctgacctgcatgatcaacggcttctacccttccgacatctcggtggagtgggagaagaacgggaaggcagaggacaactacaagaccacgccggccgtgctggacagcgacggctcctacttcctctacagcaagctctcagtgcccacgagtgagtggcagcggggcgacgtcttcacctgctccgtgatgcacgaggccttgcacaaccactacacgcagaagtccatctcccgctctccgggtaaatga

SEQ ID NO: 135 - protein, 239 aa, Oryctolagus cuniculus
MDTRAPTQLLGLLLLWLPGATFAQVLTQTPSSVSAAVGGTVTINCQSSQRLYDDNDCAWYQKKPGQPPKRLIHDASILDSGVSSRFSGSGSGTQFTLTISGVQCEDAATYYCLGDYDCSVADCSTFGGGTEVVVKGDPVAPTVLIFPPAADQVATGTVTIVCVANKYFPDVTVTWEVDGTTQTTGIENSKTPQNSADCTYNLSSTLTLTSTQYNSHKEYTCKVTQGTTSVVQSFNRGDC

SEQ ID NO: 136 - DNA, 720 bp, Oryctolagus cuniculus
  source 1..720: mol_type = genomic DNA
atggacacgagggcccccactcagctgctggggctcctgctgctctggctcccaggtgccacatttgcccaagtgctgacccagactccatcctccgtgtctgcagctgtgggaggcacagtcaccatcaattgccagtccagtcagcggctctatgatgacaacgactgtgcctggtatcagaagaaaccagggcagcctcccaagcgcctgatccatgatgcatccattctagattctggggtctcatcgcggttcagtggcagtggatctgggacacagttcactctcaccatcagcggcgtgcagtgtgaagatgctgccacttactactgtctaggcgattatgattgtagtgttgctgattgtagtactttcggcggagggaccgaggtggtggtcaaaggtgatccagttgcacctactgtcctcatcttcccaccagctgctgatcaggtggcaactggaacagtcaccatcgtgtgtgtggcgaataaatactttcccgatgtcaccgtcacctgggaggtggatggcaccacccaaacaactggcatcgagaacagtaaaacaccgcagaattctgcagattgtacctacaacctcagcagcactctgacactgaccagcacacagtacaacagccacaaagagtacacctgcaaggtgacccagggcacgacctcagtcgtccagagcttcaataggggtgactgttag

SEQ ID NO: 137 - protein, 14 aa, Homo sapiens
DLGKGGNEESTKTG

SEQ ID NO: 138 - protein, 13 aa, Homo sapiens
GKGGNEESTKTGN

SEQ ID NO: 139 - protein, 13 aa, Homo sapiens
GGNEESTKTGNAG

SEQ ID NO: 140 - protein, 13 aa, Homo sapiens
NEESTKTGNAGSR

SEQ ID NO: 141 - protein, 13 aa, Homo sapiens
ESTKTGNAGSRLA

SEQ ID NO: 142 - protein, 9 aa, Homo sapiens
NEESTKTGN

SEQ ID NO: 143 - skipped

SEQ ID NO: 144 - protein, 7 aa, Homo sapiens
ESTKTGN

SEQ ID NO: 145 - protein, 303 aa, Homo sapiens
MKGKEEKEGGARLGAGGGSPEKSPSAQELKEQGNRLFVGRKYPEAAACYGRAITRNPLVAVYYTNRALCYLKMQQHEQALADCRRALELDGQSVKAHFFLGQCQLEMESYDEAIANLQRAYSLAKEQRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDY

SEQ ID NO: 146 - DNA, 540 bp, Homo sapiens
  source 1..540: mol_type = genomic DNA
cggctgaacttcggggacgacatccccagcgctcttcgaatcgcgaagaagaagcgctggaacagcattgaggagcggcgcatccaccaggagagcgagctgcactcctacctctccaggctcattgccgcggagcgtgagagggagctggaagagtgccagcgaaaccacgagggtgatgaggacgacagccacgtccgggcccagcaggcctgcattgaggccaagcacgacaagtacatggcggacatggacgagcttttttctcaggtggatgagaagaggaagaagcgagacatccccgactacctgtgtggcaagatcagctttgagctgatgcgggagccgtgcatcacgcccagtggcatcacctacgaccgcaaggacatcgaggagcacctgcagcgtgtgggtcattttgaccccgtgacccggagccccctgacccaggaacagctcatccccaacttggctatgaaggaggttattgacgcattcatctctgagaatggctgggtggaggattacgtcgacggagca

SEQ ID NO: 147 - protein, 1066 aa, Homo sapiens
MTDQENNNNISSNPFAALFGSLADAKQFAAIQKEQLKQQSDELPASPDDSDNSVSESLDEFDYSVAEISRSFRSQQEICEQLNINHMIQRIFLITLDNSDPSLKSGNGIPSRCVYLEEMAVELEDQDWLDMSNVEQALFARLLLQDPGNHLINMTSSTTLNLSADRDAGERHIFCYLYSCFQRAKEEITKVPENLLPFAVQCRNLTVSNTRTVLLTPEIYVDQNIHEQLVDLMLEAIQGAHFEDVTEFLEEVIEALILDEEVRTFPEVMIPVFDILLGRIKDLELCQILLYAYLDILLYFTRQKDMAKVFVEYIQPKDPTNGQMYQKTLLGVILSISCLLKTPGVVENHGYFLNPSRSSPQEIKVQEANIHQFMAQFHEKIYQMLKNLLQLSPETKHCILSWLGNCLHANAGRTKIWANQMPEIFFQMYASDAFFLNLGAALLKLCQPFCKPRSSRLLTFNPTYCALKELNDEERKIKNVHMRGLDKETCLIPAVQEPKFPQNYNLVTENLALTEYTLYLGFHRLHDQMVKINQNLHRLQVAWRDAQQSSSPAADNLREQFERLMTIYLSTKTAMTEPQMLQNCLNLQVSMAVLLVQLAIGNEGSQPIELTFPLPDGYSSLAYVPEFFADNLGDFLIFLRRFADDILETSADSLEHVLHFITIFTGSIERMKNPHLRAKLAEVLEAVMPHLDQTPNPLVSSVFHRKRVFCNFQYAPQLAEALIKVFVDIEFTGDPHQFEQKFNYRRPMYPILRYMWGTDTYRESIKDLADYASKNLEAMNPPLFLRFLNLLMNDAIFLLDEAIQYLSKIKIQQIEKDRGEWDSLTPEARREKEAGLQMFGQLARFHNIMSNETIGTLAFLTSEIKSLFVHPFLAERIISMLNYFLQHLVGPKMGALKVKDFSEFDFKPQQLVSDICTIYLNLGDEENFCATVPKDGRSYSPTLFAQTVRVLKKINKPGNMIMAFSNLAERIKSLADLQQQEEETYADACDEFLDPIMSTLMCDPVVLPSSRVTVDRSTIARHLLSDQTDPFNRSPLTMDQIRPNTELKEKIQRWLAERKQQKEQLE

SEQ ID NO: 148 - DNA, 582 bp, Homo sapiens
  source 1..582: mol_type = genomic DNA
ctggccgagaggatcatcagcatgctgaactacttcctgcagcacctggtgggccccaagatgggcgccctgaaggtgaaggacttcagcgagttcgacttcaagccccagcagctggtgagcgacatctgcaccatctacctgaacctgggcgacgaggagaacttctgcgccaccgtgcccaaggacggcaggagctacagccccaccctgttcgcccagaccgtgagggtgctgaagaagatcaacaagcccggcaacatgatcatggccttcagcaacctggccgagaggatcaagagcctggccgacctgcagcagcaggaggaggagacctacgccgacgcctgcgacgagttcctggaccccatcatgagcaccctgatgtgcgaccccgtggtgctgcccagcagcagggtgaccgtggacaggagcaccatcgccaggcacctgctgagcgaccagaccgaccccttcaacaggagccccctgaccatggaccagatcaggcccaacaccgagctgaaggagaagatccagaggtggctggccgagaggaagcagcagaaggagcagctggag

SEQ ID NO: 149 - protein, 541 aa, Homo sapiens
MVINLCLPQFRPRIHCNKISADGYEVENLISEDLTKRSHGFRTEYFIKPPVYVTVSFPFNVEICRINIDLTAGGGQNVTGLEMYTSASSSRVSWNTPQCRTLGPAEPSVPDKEAFTLVGKVLLKNQSQVVFSHRGFKARPPFGAMEATLPSPAVVAQELWNKGALSLSHVAHLRICITHVTGGGIPCIKRLEVWGQPAKTCSQEVIDSILLVTSENLPQDVALQAPALPMESDCDPGDQPESQQAPSSLQKLAEIIQDVPEEFLDPITLEIMPCPMLLPSGKVIDQSTLEKCNRSEATWGRVPSDPFTGVAFTPHSQPLPHPSLKARIDHFLLQHSIPGCHLLGRAQTALAVIPSSIVLPSQKRKIEQAEHVPDSNFGVNASCFSATSPLVLPTTSEHTAKKMKATNEPSLTHMDCSTGPLSHEQKLSQSLEIALASTLGSMPSFTARLTRGQLQHLGTRGSNTSWRPGTGSEQPGSILGPECASCKRVFSPYFKKEPVYQLPCGHLLCRPCLGEKQRSLPMTCTACQRPVASQDVLRVHF

SEQ ID NO: 150 - DNA, 1017 bp, Homo sapiens
  source 1..1017: mol_type = genomic DNA
gtgatcaacctgtgcctgccccagttcaggcccaggatccactgcaacaagatcagcgccgacggctacgaggtggagaacctgatcagcgaggacctgaccaagaggagccacggcttcaggaccgagtacttcatcaagccccccgtgtacgtgaccgtgagcttccccttcaacgtggagatctgcaggatcaacatcgacctgaccgccggcggcggccagaacgtgaccggcctggagatgtacaccagcgccagcagcagcagggtgagctggaacaccccccagtgcaggaccctgggccccgccgagcccagcgtgcccgacaaggaggccttcaccctggtgggcaaggtgctgctgaagaaccagagccaggtggtgttcagccacaggggcttcaaggccaggccccccttcggcgccatggaggccaccctgcccagccccgccgtggtggcccaggagctgtggaacaagggcgccctgagcctgagccacgtggcccacctgaggatctgcatcacccacgtgaccggcggcggcatcccctgcatcaagaggctggaggtgtggggccagcccgccaagacctgcagccaggaggtgatcgacagcatcctgctggtgaccagcgagaacctgccccaggacgtggccctgcaggcccccgccctgcccatggagagcgactgcgaccccggcgaccagcccgagagccagcaggcccccagcagcctgcagaagctggccgagatcatccaggacgtgcccgaggagttcctggaccccatcaccctggagatcatgccctgccccatgctgctgcccagcggcaaggtgatcgaccagagcaccctggagaagtgcaacaggagcgaggccacctggggcagggtgcccagcgaccccttcaccggcgtggccttcaccccccacagccagcccctgccccaccccagcctgaaggccaggatcgaccacttcctgctgcagcacagcatccccggctgc

SEQ ID NO: 151 - protein, 975 aa, Homo sapiens
MATGLGEPVYGLSEDEGESRILRVKVVSGIDLAKKDIFGASDPYVKLSLYVADENRELALVQTKTIKKTLNPKWNEEFYFRVNPSNHRLLFEVFDENRLTRDDFLGQVDVPLSHLPTEDPTMERPYTFKDFLLRPRSHKSRVKGFLRLKMAYMPKNGGQDEENSDQRDDMEHGWEVVDSNDSASQHQEELPPPPLPPGWEEKVDNLGRTYYVNHNNRTTQWHRPSLMDVSSESDNNIRQINQEAAHRRFRSRRHISEDLEPEPSEGGDVPEPWETISEEVNIAGDSLGLALPPPPASPGSRTSPQELSEELSRRLQITPDSNGEQFSSLIQREPSSRLRSCSVTDAVAEQGHLPPPSAPAGRARSSTVTGGEEPTPSVAYVHTTPGLPSGWEERKDAKGRTYYVNHNNRTTTWTRPIMQLAEDGASGSATNSNNHLIEPQIRRPRSLSSPTVTLSAPLEGAKDSPVRRAVKDTLSNPQSPQPSPYNSPKPQHKVTQSFLPPGWEMRIAPNGRPFFIDHNTKTTTWEDPRLKFPVHMRSKTSLNPNDLGPLPPGWEERIHLDGRTFYIDHNSKITQWEDPRLQNPAITGPAVPYSREFKQKYDYFRKKLKKPADIPNRFEMKLHRNNIFEESYRRIMSVKRPDVLKARLWIEFESEKGLDYGGVAREWFFLLSKEMFNPYYGLFEYSATDNYTLQINPNSGLCNEDHLSYFTFIGRVAGLAVFHGKLLDGFFIRPFYKMMLGKQITLNDMESVDSEYYNSLKWILENDPTELDLMFCIDEENFGQTYQVDLKPNGSEIMVTNENKREYIDLVIQWRFVNRVQKQMNAFLEGFTELLPIDLIKIFDENELELLMCGLGDVDVNDWRQHSIYKNGYCPNHPVIQWFWKAVLLMDAEKRIRLLQFVTGTSRVPMNGFAELYGSNGPQLFTIEQWGSPEKLPRAHTCFNRLDLPPYETFEDLREKLLMAVENAQGFEGVD

SEQ ID NO: 152 - DNA, 1080 bp, Homo sapiens
  source 1..1080: mol_type = genomic DNA
aacaggttcgagatgaagctgcacaggaacaacatcttcgaggagagctacaggaggatcatgagcgtgaagaggcccgacgtgctgaaggccaggctgtggatcgagttcgagagcgagaagggcctggactacggcggcgtggccagggagtggttcttcctgctgagcaaggagatgttcaacccctactacggcctgttcgagtacagcgccaccgacaactacaccctgcagatcaaccccaacagcggcctgtgcaacgaggaccacctgagctacttcaccttcatcggcagggtggccggcctggccgtgttccacggcaagctgctggacggcttcttcatcaggcccttctacaagatgatgctgggcaagcagatcaccctgaacgacatggagagcgtggacagcgagtactacaacagcctgaagtggatcctggagaacgaccccaccgagctggacctgatgttctgcatcgacgaggagaacttcggccagacctaccaggtggacctgaagcccaacggcagcgagatcatggtgaccaacgagaacaagagggagtacatcgacctggtgatccagtggaggttcgtgaacagggtgcagaagcagatgaacgccttcctggagggcttcaccgagctgctgcccatcgacctgatcaagatcttcgacgagaacgagctggagctgctgatgtgcggcctgggcgacgtggacgtgaacgactggaggcagcacagcatctacaagaacggctactgccccaaccaccccgtgatccagtggttctggaaggccgtgctgctgatggacgccgagaagaggatcaggctgctgcagttcgtgaccggcaccagcagggtgcccatgaacggcttcgccgagctgtacggcagcaacggcccccagctgttcaccatcgagcagtggggcagccccgagaagctgcccagggcccacacctgcttcaacaggctggacctgcccccctacgagaccttcgaggacctgagggagaagctgctgatggccgtggagaacgcccagggcttcgagggcgtggac

SEQ ID NO: 153 - protein, 2799 aa, Homo sapiens
MTSIHFVVHPLPGTEDQLNDRLREVSEKLNKYNLNSHPPLNVLEQATIKQCVVGPNHAAFLLEDGRVCRIGFSVQPDRLELGKPDNNDGSKLNSNSGAGRTSRPGRTSDSPWFLSGSETLGRLAGNTLGSRWSSGVGGSGGGSSGRSSAGARDSRRQTRVIRTGRDRGSGLLGSQPQPVIPASVIPEELISQAQVVLQGKSRSVIIRELQRTNLDVNLAVNNLLSRDDEDGDDGDDTASESYLPGEDLMSLLDADIHSAHPSVIIDADAMFSEDISYFGYPSFRRSSLSRLGSSRVLLLPLERDSELLRERESVLRLRERRWLDGASFDNERGSTSKEGEPNLDKKNTPVQSPVSLGEDLQWWPDKDGTKFICIGALYSELLAVSSKGELYQWKWSESEPYRNAQNPSLHHPRATFLGLTNEKIVLLSANSIRATVATENNKVATWVDETLSSVASKLEHTAQTYSELQGERIVSLHCCALYTCAQLENSLYWWGVVPFSQRKKMLEKARAKNKKPKSSAGISSMPNITVGTQVCLRNNPLYHAGAVAFSISAGIPKVGVLMESVWNMNDSCRFQLRSPESLKNMEKASKTTEAKPESKQEPVKTEMGPPPSPASTCSDASSIASSASMPYKRRRSTPAPKEEEKVNEEQWSLREVVFVEDVKNVPVGKVLKVDGAYVAVKFPGTSSNTNCQNSSGPDADPSSLLQDCRLLRIDELQVVKTGGTPKVPDCFQRTPKKLCIPEKTEILAVNVDSKGVHAVLKTGNWVRYCIFDLATGKAEQENNFPTSSIAFLGQNERNVAIFTAGQESPIILRDGNGTIYPMAKDCMGGIRDPDWLDLPPISSLGMGVHSLINLPANSTIKKKAAVIIMAVEKQTLMQHILRCDYEACRQYLMNLEQAVVLEQNLQMLQTFISHRCDGNRNILHACVSVCFPTSNKETKEEEEAERSERNTFAERLSAVEAIANAISVVSSNGPGNRAGSSSSRSLRLREMMRRSLRAAGLGRHEAGASSSDHQDPVSPPIAPPSWVPDPPAMDPDGDIDFILAPAVGSLTTAATGTGQGPSTSTIPGPSTEPSVVESKDRKANAHFILKLLCDSVVLQPYLRELLSAKDARGMTPFMSAVSGRAYPAAITILETAQKIAKAEISSSEKEEDVFMGMVCPSGTNPDDSPLYVLCCNDTCSFTWTGAEHINQDIFECRTCGLLESLCCCTECARVCHKGHDCKLKRTSPTAYCDCWEKCKCKTLIAGQKSARLDLLYRLLTATNLVTLPNSRGEHLLLFLVQTVARQTVEHCQYRPPRIREDRNRKTASPEDSDMPDHDLEPPRFAQLALERVLQDWNALKSMIMFGSQENKDPLSASSRIGHLLPEEQVYLNQQSGTIRLDCFTHCLIVKCTADILLLDTLLGTLVKELQNKYTPGRREEAIAVTMRFLRSVARVFVILSVEMASSKKKNNFIPQPIGKCKRVFQALLPYAVEELCNVAESLIVPVRMGIARPTAPFTLASTSIDAMQGSEELFSVEPLPPRPSSDQSSSSSQSQSSYIIRNPQQRRISQSQPVRGRDEEQDDIVSADVEEVEVVEGVAGEEDHHDEQEEHGEENAEAEGQHDEHDEDGSDMELDLLAAAETESDSESNHSNQDNASGRRSVVTAATAGSEAGASSVPAFFSEDDSQSNDSSDSDSSSSQSDDIEQETFMLDEPLERTTNSSHANGAAQAPRSMQWAVRNTQHQRAASTAPSSTSTPAASSAGLIYIDPSNLRRSGTISTSAAAAAAALEASNASSYLTSASSLARAYSIVIRQISDLMGLIPKYNHLVYSQIPAAVKLTYQDAVNLQNYVEEKLIPTWNWMVSIMDSTEAQLRYGSALASAGDPGHPNHPLHASQNSARRERMTAREEASLRTLEGRRRATLLSARQGMMSARGDFLNYALSLMRSHNDEHSDVLPVLDVCSLKHVAYVFQALIYWIKAMNQQTTLDTPQLERKRTRELLELGIDNEDSEHENDDDTNQSATLNDKDDDSLPAETGQNHPFFRRSDSMTFLGCIPPNPFEVPLAEAIPLADQPHLLQPNARKEDLFGRPSQGLYSSSASSGKCLMEVTVDRNCLEVLPTKMSYAANLKNVMNMQNRQKKEGEEQPVLPEETESSKPGPSAHDLAAQLKSSLLAEIGLTESEGPPLTSFRPQCSFMGMVISHDMLLGRWRLSLELFGRVFMEDVGAEPGSILTELGGFEVKESKFRREMEKLRNQQSRDLSLEVDRDRDLLIQQTMRQLNNHFGRRCATTPMAVHRVKVTFKDEPGEGSGVARSFYTAIAQAFLSNEKLPNLECIQNANKGTHTSLMQRLRNRGERDREREREREMRRSSGLRAGSRRDRDRDFRRQLSIDTRPFRPASEGNPSDDPEPLPAHRQALGERLYPRVQAMQPAFASKITGMLLELSPAQLLLLLASEDSLRARVDEAMELIIAHGRENGADSILDLGLVDSSEKVQQENRKRHGSSRSVVDMDLDDTDDGDDNAPLFYQPGKRGFYTPRPGKNTEARLNCFRNIGRILGLCLLQNELCPITLNRHVIKVLLGRKVNWHDFAFFDPVMYESLRQLILASQSSDADAVFSAMDLAFAIDLCKEEGGGQVELIPNGVNIPVTPQNVYEYVRKYAEHRMLVVAEQPLHAMRKGLLDVLPKNSLEDLTAEDFRLLVNGCGEVNVQMLISFTSFNDESGENAEKLLQFKRWFWSIVEKMSMTERQDLVYFWTSSPSLPASEEGFQPMPSITIRPPDDQHLPTANTCISRLYVPLYSSKQILKQKLLLAIKTKNFGFV

SEQ ID NO: 154 - DNA, 1035 bp, Homo sapiens
  source 1..1035: mol_type = genomic DNA
gagaacggcgccgacagcatcctggacctgggcctggtggacagcagcgagaaggtgcagcaggagaacaggaagaggcacggcagcagcaggagcgtggtggacatggacctggacgacaccgacgacggcgacgacaacgcccccctgttctaccagcccggcaagaggggcttctacacccccaggcccggcaagaacaccgaggccaggctgaactgcttcaggaacatcggcaggatcctgggcctgtgcctgctgcagaacgagctgtgccccatcaccctgaacaggcacgtgatcaaggtgctgctgggcaggaaggtgaactggcacgacttcgccttcttcgaccccgtgatgtacgagagcctgaggcagctgatcctggccagccagagcagcgacgccgacgccgtgttcagcgccatggacctggccttcgccatcgacctgtgcaaggaggagggcggcggccaggtggagctgatccccaacggcgtgaacatccccgtgaccccccagaacgtgtacgagtacgtgaggaagtacgccgagcacaggatgctggtggtggccgagcagcccctgcacgccatgaggaagggcctgctggacgtgctgcccaagaacagcctggaggacctgaccgccgaggacttcaggctgctggtgaacggctgcggcgaggtgaacgtgcagatgctgatcagcttcaccagcttcaacgacgagagcggcgagaacgccgagaagctgctgcagttcaagaggtggttctggagcatcgtggagaagatgagcatgaccgagaggcaggacctggtgtacttctggaccagcagccccagcctgcccgccagcgaggagggcttccagcccatgcccagcatcaccatcaggccccccgacgaccagcacctgcccaccgccaacacctgcatcagcaggctgtacgtgcccctgtacagcagcaagcagatcctgaagcagaagctgctgctggccatcaagaccaagaacttcggcttcgtg

SEQ ID NO: 155 - protein, 605 aa, Homo sapiens
MDPAEAVLQEKALKFMCSMPRSLWLGCSSLADSMPSLRCLYNPGTGALTAFQNSSEREDCNNGEPPRKIIPEKNSLRQTYNSCARLCLNQETVCLASTAMKTENCVAKTKLANGTSSMIVPKQRKLSASYEKEKELCVKYFEQWSESDQVEFVEHLISQMCHYQHGHINSYLKPMLQRDFITALPARGLDHIAENILSYLDAKSLCAAELVCKEWYRVTSDGMLWKKLIERMVRTDSLWRGLAERRGWGQYLFKNKPPDGNAPPNSFYRALYPKIIQDIETIESNWRCGRHSLQRIHCRSETSKGVYCLQYDDQKIVSGLRDNTIKIWDKNTLECKRILTGHTGSVLCLQYDERVIITGSSDSTVRVWDVNTGEMLNTLIHHCEAVLHLRFNNGMMVTCSKDRSIAVWDMASPTDITLRRVLVGHRAAVNVVDFDDKYIVSASGDRTIKVWNTSTCEFVRTLNGHKRGIACLQYRDRLVVSGSSDNTIRLWDIECGACLRVLEGHEELVRCIRFDNKRIVSGAYDGKIKVWDLVAALDPRAPAGTLCLRTLVEHSGRVFRLQFDEFQIVSSSHDDTILIWDFLNDPAAQAEPPRSPSRTYTYISR

SEQ ID NO: 156 - DNA, 783 bp, Homo sapiens
  source 1..783: mol_type = genomic DNA
atggaccccgccgaggccgtgctgcaggagaaggccctgaagttcatgtgcagcatgcccaggagcctgtggctgggctgcagcagcctggccgacagcatgcccagcctgaggtgcctgtacaaccccggcaccggcgccctgaccgccttccagaacagcagcgagagggaggactgcaacaacggcgagccccccaggaagatcatccccgagaagaacagcctgaggcagacctacaacagctgcgccaggctgtgcctgaaccaggagaccgtgtgcctggccagcaccgccatgaagaccgagaactgcgtggccaagaccaagctggccaacggcaccagcagcatgatcgtgcccaagcagaggaagctgagcgccagctacgagaaggagaaggagctgtgcgtgaagtacttcgagcagtggagcgagagcgaccaggtggagttcgtggagcacctgatcagccagatgtgccactaccagcacggccacatcaacagctacctgaagcccatgctgcagagggacttcatcaccgccctgcccgccaggggcctggaccacatcgccgagaacatcctgagctacctggacgccaagagcctgtgcgccgccgagctggtgtgcaaggagtggtacagggtgaccagcgacggcatgctgtggaagaagctgatcgagaggatggtgaggaccgacagcctgtggaggggcctggccgagaggaggggctggggccagtacctgttcaagaacaagccccccgacggcaac

SEQ ID NO: 157 - protein, 465 aa, Homo sapiens
MIVFVRFNSSHGFPVEVDSDTSIFQLKEVVAKRQGVPADQLRVIFAGKELRNDWTVQNCDLDQQSIVHIVQRPWRKGQEMNATGGDDPRNAAGGCEREPQSLTRVDLSSSVLPGDSVGLAVILHTDSRKDSPPAGSPAGRSIYNSFYVYCKGPCQRVQPGKLRVQCSTCRQATLTLTQGPSCWDDVLIPNRMSGECQSPHCPGTSAEFFFKCGAHPTSDKETSVALHLIATNSRNITCITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNCGCEWNRVCMGDHWFDV

SEQ ID NO: 158 - DNA, 738 bp, Homo sapiens
  source 1..738: mol_type = genomic DNA
aaggagaccagcgtggccctgcacctgatcgccaccaacagcaggaacatcacctgcatcacctgcaccgacgtgaggagccccgtgctggtgttccagtgcaacagcaggcacgtgatctgcctggactgcttccacctgtactgcgtgaccaggctgaacgacaggcagttcgtgcacgacccccagctgggctacagcctgccctgcgtggccggctgccccaacagcctgatcaaggagctgcaccacttcaggatcctgggcgaggagcagtacaacaggtaccagcagtacggcgccgaggagtgcgtgctgcagatgggcggcgtgctgtgccccaggcccggctgcggcgccggcctgctgcccgagcccgaccagaggaaggtgacctgcgagggcggcaacggcctgggctgcggcttcgccttctgcagggagtgcaaggaggcctaccacgagggcgagtgcagcgccgtgttcgaggccagcggcaccaccacccaggcctacagggtggacgagagggccgccgagcaggccaggtgggaggccgccagcaaggagaccatcaagaagaccaccaagccctgccccaggtgccacgtgcccgtggagaagaacggcggctgcatgcacatgaagtgcccccagccccagtgcaggctggagtggtgctggaactgcggctgcgagtggaacagggtgtgcatgggcgaccactggttcgacgtg

SEQ ID NO: 159 - protein, 190 aa, Homo sapiens
MSTRKRRGGAINSRQAQKRTREATSTPEISLEAEPIELVETAGDEIVDLTCESLEPVVVDLTHNDSVVIVDERRRPRRNARRLPQDHADSCVVSSDDEELSRDRDVYVTTHTPRNARDEGATGLRPSGTVSCPICMDGYSEIVQNGRLIVSTECGHVFCSQCLRDSLKNANTCPTCRKKINHKRYHPIYI

SEQ ID NO: 160 - DNA, 345 bp, Homo sapiens
  source 1..345: mol_type = genomic DNA
cccaggaggaacgccaggaggctgccccaggaccacgccgacagctgcgtggtgagcagcgacgacgaggagctgagcagggacagggacgtgtacgtgaccacccacacccccaggaacgccagggacgagggcgccaccggcctgaggcccagcggcaccgtgagctgccccatctgcatggacggctacagcgagatcgtgcagaacggcaggctgatcgtgagcaccgagtgcggccacgtgttctgcagccagtgcctgagggacagcctgaagaacgccaacacctgccccacctgcaggaagaagatcaaccacaagaggtaccaccccatctacatc

SEQ ID NO: 161 - DNA, 1413 bp, Homo sapiens
  source 1..1413: mol_type = genomic DNA
atggagactgggctgcgctggcttctcctggtcgctgtgctcaaaggtgtccagtgtcagtcggtggaggagtccgggggtcgcctggtgtcgcctggaggatccctgacactcacctgccgtgtttctggaagcgaccgcaggacctatgtaatgcgctgggtccgccaggctccggggaaggggctggaatggatcggaaccattagacttgatggtagtagaaactatgcgacctgggcgaaaggccgtttcaccatctccaagtcctcgtcgaccacgatggagctgagaatgaccagtctgacaagcgaggacacggccacttatttctgtgtcagagacgacttgtggggcccaggcaccctggtcaccgtctcctcaggtggaggcggttcaggcggaggtggatctggcggtggcggatcgatggacacgagggcccccactcagctgctggggctcctgctgctctggctcccaggtgccagatgtgctgacatcgtgatgacccagactccagcctctgtggaggtagctgtgggaggcacagtcaccatcaagtgccaggccagtcagagcattagcaacctcttagcctggtatcagcagaaaccaggacagcctcccaagctcctgatctacaaggcttccactctggaatctggggtcccatcgcggttcaaaggcagtggatctgggacacagttcgctctcaccatcagcggcgtgcagtgtgacgatgctgccacttactactgtcaacagggtttcgatagtactgatgttgacaatcttttcggcggagggaccgaagtggtggtcgcaggcagcggttctggccggctgaacttcggggacgacatccccagcgctcttcgaatcgcgaagaagaagcgctggaacagcattgaggagcggcgcatccaccaggagagcgagctgcactcctacctctccaggctcattgccgcggagcgtgagagggagctggaagagtgccagcgaaaccacgagggtgatgaggacgacagccacgtccgggcccagcaggcctgcattgaggccaagcacgacaagtacatggcggacatggacgagcttttttctcaggtggatgagaagaggaagaagcgagacatccccgactacctgtgtggcaagatcagctttgagctgatgcgggagccgtgcatcacgcccagtggcatcacctacgaccgcaaggacatcgaggagcacctgcagcgtgtgggtcattttgaccccgtgacccggagccccctgacccaggaacagctcatccccaacttggctatgaaggaggttattgacgcattcatctctgagaatggctgggtggaggattacgtcgacggagcagactacaaggacgatgacgacaagggataa

SEQ ID NO: 162 - protein, 442 aa, Homo sapiens
QSVEESGGRLVSPGGSLTLTCRVSGSDRRTYVMRWVRQAPGKGLEWIGTIRLDGSRNYATWAKGRFTISKSSSTTMELRMTSLTSEDTATYFCVRDDLWGPGTLVTVSSGGGGSGGGGSGGGGSMDTRAPTQLLGLLLLWLPGARCADIVMTQTPASVEVAVGGTVTIKCQASQSISNLLAWYQQKPGQPPKLLIYKASTLESGVPSRFKGSGSGTQFALTISGVQCDDAATYYCQQGFDSTDVDNLFGGGTEVVVAGSGSGRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDYVDGA

SEQ ID NO: 163 - DNA, 1413 bp, Homo sapiens
  source 1..1413: mol_type = genomic DNA
atggagactgggctgcgctggcttctcctggtcgctgtgctcaaaggtgtccagtgtcagtcggtggaggagtccgggggtcgcctggtgtcgcctggaggatccctgacactcacctgccgtgtttctggaagcgaccgcaggacctatgtaatgcgctgggtccgccaggctccggggaaggggctggaatggatcggaaccattagacttgatggtagtagaaactatgcgacctgggcgaaaggccgtttcaccatctccaagtcctcgtcgaccacgatggagctgagaatgaccagtctgacaagcgaggacacggccacttatttctgtgtcagagacgacttggggggcccaggcaccctggtcaccgtctcctcaggtggaggcggttcaggcggaggtggatctggcggtggcggatcgatggacacgagggcccccactcagctgctggggctcctgctgctctggctcccaggtgccagatgtgctgacatcgtgatgacccagactccagcctctgtggaggtagctgtgggaggcacagtcaccatcaagtgccaggccagtcagagcattagcaacctcttagcctggtatcagcagaaaccaggacagcctcccaagctcctgatctacaaggcttccactctggaatctggggtcccatcgcggttcaaaggcagtggatctgggacacagttcgctctcaccatcagcggcgtgcagtgtgacgatgctgccacttactactgtcaacagggtttcgatagtactgatgttgacaatcttttcggcggagggaccgaagtggtggtcgcaggcagcggttctggccggctgaacttcggggacgacatccccagcgctcttcgaatcgcgaagaagaagcgctggaacagcattgaggagcggcgcatccaccaggagagcgagctgcactcctacctctccaggctcattgccgcggagcgtgagagggagctggaagagtgccagcgaaaccacgagggtgatgaggacgacagccacgtccgggcccagcaggcctgcattgaggccaagcacgacaagtacatggcggacatggacgagcttttttctcaggtggatgagaagaggaagaagcgagacatccccgactacctgtgtggcaagatcagctttgagctgatgcgggagccgtgcatcacgcccagtggcatcacctacgaccgcaaggacatcgaggagcacctgcagcgtgtgggtcattttgaccccgtgacccggagccccctgacccaggaacagctcatccccaacttggctatgaaggaggttattgacgcattcatctctgagaatggctgggtggaggattacgtcgacggagcagactacaaggacgatgacgacaagggataa

SEQ ID NO: 164 - protein, 442 aa, Homo sapiens
QSVEESGGRLVSPGGSLTLTCRVSGSDRRTYVMRWVRQAPGKGLEWIGTIRLDGSRNYATWAKGRFTISKSSSTTMELRMTSLTSEDTATYFCVRDDLGGPGTLVTVSSGGGGSGGGGSGGGGSMDTRAPTQLLGLLLLWLPGARCADIVMTQTPASVEVAVGGTVTIKCQASQSISNLLAWYQQKPGQPPKLLIYKASTLESGVPSRFKGSGSGTQFALTISGVQCDDAATYYCQQGFDSTDVDNLFGGGTEVVVAGSGSGRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDYVDGA

SEQ ID NO: 165 - DNA, 1416 bp, Homo sapiens
  source 1..1416: mol_type = genomic DNA
atggagactgggctgcgctggcttctcctggtcgctgtgctcaaaggtgtccagtgtcagtcggtggaggagtccgggggtcgcctggtcacgcctgggacacccctgacactcacctgcacagtctctggaatcgacctcagtagcagtgtaatggcctgggtccgccaggctccagggaaggggctggaatggatcggaatcattactaatcgtggtaatagatactacgcgaagtgggcgaaaggccgattcaccatctccagaacctcgaccacggtgggtctgaagatcaccagcccgacaaccgcggacacggccacctatttctgtggcagaggtagcgtctggggcccaggcaccctggtcaccgtctcctcaggtggaggcggttcaggcggaggtggatctggcggtggcggatcgatggacacgagggcccccactcagctgctggggctcctgctgctctggctcccaggtgccacatttgcccaagtgctgacccagactccagcctcggtgtctgcggctattggaggcacagttaccatcaagtgccagtccagtcagagttttcttgatgacaatgacttagcctggtatcagaagaaaccagggcggcctcccaagcgcctgatcaaatctgcatcgactctagattctggggtcccatcacggttcagtggcagtggatctgggacacagttcactctcaccatcagcgacgtgcagtgtgccgatgctgccacttattactgtctaggcgattatgattgtagtgttggtgattgtagtgctttcggcggagggaccgaagtggtggtcaagggcagcggttctggccggctgaacttcggggacgacatccccagcgctcttcgaatcgcgaagaagaagcgctggaacagcattgaggagcggcgcatccaccaggagagcgagctgcactcctacctctccaggctcattgccgcggagcgtgagagggagctggaagagtgccagcgaaaccacgagggtgatgaggacgacagccacgtccgggcccagcaggcctgcattgaggccaagcacgacaagtacatggcggacatggacgagcttttttctcaggtggatgagaagaggaagaagcgagacatccccgactacctgtgtggcaagatcagctttgagctgatgcgggagccgtgcatcacgcccagtggcatcacctacgaccgcaaggacatcgaggagcacctgcagcgtgtgggtcattttgaccccgtgacccggagccccctgacccaggaacagctcatccccaacttggctatgaaggaggttattgacgcattcatctctgagaatggctgggtggaggattacgtcgacggagcagactacaaggacgatgacgacaagggataa

SEQ ID NO: 166 - protein, 443 aa, Homo sapiens
QSVEESGGRLVTPGTPLTLTCTVSGIDLSSSVMAWVRQAPGKGLEWIGIITNRGNRYYAKWAKGRFTISRTSTTVGLKITSPTTADTATYFCGRGSVWGPGTLVTVSSGGGGSGGGGSGGGGSMDTRAPTQLLGLLLLWLPGATFAQVLTQTPASVSAAIGGTVTIKCQSSQSFLDDNDLAWYQKKPGRPPKRLIKSASTLDSGVPSRFSGSGSGTQFTLTISDVQCADAATYYCLGDYDCSVGDCSAFGGGTEVVVKGSGSGRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDYVDGA

SEQ ID NO: 167 - DNA, 1416 bp, Homo sapiens
  source 1..1416: mol_type = genomic DNA
atggagactgggctgcgctggcttctcctggtcgctgtgctcaaaggtgtccagtgtcagtcggtggaggagtccgggggtcgcctggtcacgcctgggacacccctgacactcacctgcacagtctctggaatcgacctcagtagcagtgtaatggtctgggtccgccaggctccagggaaggggctggaatggatcggaatcattactaatcgtggtaatagatactacgcgaagtgggcgaaaggccgattcaccatctccagaacctcgaccacggtgggtctgaagatcaccagtccgacaaccgcggacacggccacctatttctgtggcagaggtagcgtctggggcccaggcaccctggtcaccgtctcctcaggtggaggcggttcaggcggaggtggatctggcggtggcggatcgatggacacgagggcccccactcagctgctggggctcctgctgctctggctcccaggtgccacatttgcccaagtgctgacccagactccagcctcggtgtctgcggctattggaggcacagttaccatcaagtgccagtccagtcagagttttcttgatgacaatgacttagcctggtatcagaagaaaccagggcggcctcccaagcgcctgatcaaatctgcatcgactctagattctggggtcccatcacggttcagtggcagtggatctgggacacagttcactctcaccatcagcgacgtgcagtgtgccgatgctgccacttattactgtctaggcgattatgattgtagtgttggtgattgtagtgctttcggcggagggaccgaagtggtggtcaagggcagcggttctggccggctgaacttcggggacgacatccccagcgctcttcgaatcgcgaagaagaagcgctggaacagcattgaggagcggcgcatccaccaggagagcgagctgcactcctacctctccaggctcattgccgcggagcgtgagagggagctggaagagtgccagcgaaaccacgagggtgatgaggacgacagccacgtccgggcccagcaggcctgcattgaggccaagcacgacaagtacatggcggacatggacgagcttttttctcaggtggatgagaagaggaagaagcgagacatccccgactacctgtgtggcaagatcagctttgagctgatgcgggagccgtgcatcacgcccagtggcatcacctacgaccgcaaggacatcgaggagcacctgcagcgtgtgggtcattttgaccccgtgacccggagccccctgacccaggaacagctcatccccaacttggctatgaaggaggttattgacgcattcatctctgagaatggctgggtggaggattacgtcgacggagcagactacaaggacgatgacgacaagggataa

SEQ ID NO: 168 - protein, 443 aa, Homo sapiens
QSVEESGGRLVTPGTPLTLTCTVSGIDLSSSVMVWVRQAPGKGLEWIGIITNRGNRYYAKWAKGRFTISRTSTTVGLKITSPTTADTATYFCGRGSVWGPGTLVTVSSGGGGSGGGGSGGGGSMDTRAPTQLLGLLLLWLPGATFAQVLTQTPASVSAAIGGTVTIKCQSSQSFLDDNDLAWYQKKPGRPPKRLIKSASTLDSGVPSRFSGSGSGTQFTLTISDVQCADAATYYCLGDYDCSVGDCSAFGGGTEVVVKGSGSGRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDYVDGA

SEQ ID NO: 169 - DNA, 1422 bp, Homo sapiens
  source 1..1422: mol_type = genomic DNA
atggagactgggctgcgctggcttctcctggtcgctgtgctcaaaggtgtccagtgtcagtcggtgggggagtccgggggaggcctggtcacgcctgggacacccctgacactcacctgcacagtctctggattctccctcagtagctatggagtgagctgggtccgccaggctccagggaaggggctggaatacatcggattcattagcactcgtggtaacccatactacgcgagctgggcgaaaggccgattcaccatctccaaaacctcgaccacggtggatctgaaaatcaccagtccgacaaccgaggacacggccacctatttctgtgccggggcaactgttgttggtgatagtatttggggcccaggcaccctggtcaccgtctcctcaggtggaggcggttcaggcggaggtggatctggcggtggcggatcgatggacacgagggcccccactcagctgctggggctcctgctgctctggctcccaggtgccacatttgcgcaagtgctgacccagactgcatcgtccgtgtctgcagctgtgggaggcacagtcaccatcagttgccagtccagtcagagtgtttataagggctactggcaagcctggtatcagcagaaaccagggcagcctcccaagctcctgatctacgaaacatccactctggcatctggggtcccatcgcggttcagcggcagtggatctgggacacagttcactctcaccatcagcggcgtgcagtgtgacgatgctgccacttactactgtgcaggcggttatagtaataatatttatactttcggcggagggaccgaggtggtggtcaagggcagcggttctggccggctgaacttcggggacgacatccccagcgctcttcgaatcgcgaagaagaagcgctggaacagcattgaggagcggcgcatccaccaggagagcgagctgcactcctacctctccaggctcattgccgcggagcgtgagagggagctggaagagtgccagcgaaaccacgagggtgatgaggacgacagccacgtccgggcccagcaggcctgcattgaggccaagcacgacaagtacatggcggacatggacgagcttttttctcaggtggatgagaagaggaagaagcgagacatccccgactacctgtgtggcaagatcagctttgagctgatgcgggagccgtgcatcacgcccagtggcatcacctacgaccgcaaggacatcgaggagcacctgcagcgtgtgggtcattttgaccccgtgacccggagccccctgacccaggaacagctcatccccaacttggctatgaaggaggttattgacgcattcatctctgagaatggctgggtggaggattacgtcgacggagcagactacaaggacgatgacgacaagggataa

SEQ ID NO: 170 - protein, 445 aa, Homo sapiens
QSVGESGGGLVTPGTPLTLTCTVSGFSLSSYGVSWVRQAPGKGLEYIGFISTRGNPYYASWAKGRFTISKTSTTVDLKITSPTTEDTATYFCAGATVVGDSIWGPGTLVTVSSGGGGSGGGGSGGGGSMDTRAPTQLLGLLLLWLPGATFAQVLTQTASSVSAAVGGTVTISCQSSQSVYKGYWQAWYQQKPGQPPKLLIYETSTLASGVPSRFSGSGSGTQFTLTISGVQCDDAATYYCAGGYSNNIYTFGGGTEVVVKGSGSGRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDYVDGA

SEQ ID NO: 171 - DNA, 1422 bp, Homo sapiens
  source 1..1422: mol_type = genomic DNA
atggagactgggctgcgctggcttctcctggtcgctgtgctcaaaggtgtccagtgtcagtcggtggaggagtccgggggaggcctggtcacgcctgggacacccctgacactcacctgcacagtctctggattctccctcagtagctatggagtgagctgggtccgccaggctccagggaaggggctggaatacatcggattcattagcactcgtggtaacccatactacgcgagctgggcgaaaggccgattcaccatctccaaaacctcgaccacggtggatctgaaaatcaccagtccgacaaccgaggacacggccacctatttctgtgccggggcaactgttgttggtgatagtatttggggcccaggcaccctggtcaccgtctcctcaggtggaggcggttcaggcggaggtggatctggcggtggcggatcgatggacacgagggcccccactcagctgctggggctcctgctgctctggctcccaggtgccacatttgcgcaagtgctgacccagactgcatcgtccgtgtctgcagctgtgggaggcacagtcaccatcagttgccagtccagtcagagtgtttataagggctactggcaagcctggtatcagcagaaaccagggcagcctcccaagctcctgatctacgaaacatccactctggcatctggggtcccatcgcggttcagcggcagtggatctgggacacagttcactctcaccatcagcggcgtgcagtgtgacgatgctgccacttactactgtgcaggcggttatagtaataatatttatactttcggcggagggaccgaggtggtggtcaagggcagcggttctggccggctgaacttcggggacgacatccccagcgctcttcgaatcgcgaagaagaagcgctggaacagcattgaggagcggcgcatccaccaggagagcgagctgcactcctacctctccaggctcattgccgcggagcgtgagagggagctggaagagtgccagcgaaaccacgagggtgatgaggacgacagccacgtccgggcccagcaggcctgcattgaggccaagcacgacaagtacatggcggacatggacgagcttttttctcaggtggatgagaagaggaagaagcgagacatccccgactacctgtgtggcaagatcagctttgagctgatgcgggagccgtgcatcacgcccagtggcatcacctacgaccgcaaggacatcgaggagcacctgcagcgtgtgggtcattttgaccccgtgacccggagccccctgacccaggaacagctcatccccaacttggctatgaaggaggttattgacgcattcatctctgagaatggctgggtggaggattacgtcgacggagcagactacaaggacgatgacgacaagggataa

SEQ ID NO: 172 - protein, 445 aa, Homo sapiens
QSVEESGGGLVTPGTPLTLTCTVSGFSLSSYGVSWVRQAPGKGLEYIGFISTRGNPYYASWAKGRFTISKTSTTVDLKITSPTTEDTATYFCAGATVVGDSIWGPGTLVTVSSGGGGSGGGGSGGGGSMDTRAPTQLLGLLLLWLPGATFAQVLTQTASSVSAAVGGTVTISCQSSQSVYKGYWQAWYQQKPGQPPKLLIYETSTLASGVPSRFSGSGSGTQFTLTISGVQCDDAATYYCAGGYSNNIYTFGGGTEVVVKGSGSGRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDYVDGA

SEQ ID NO: 173 - DNA, 1416 bp, Homo sapiens
  source 1..1416: mol_type = genomic DNA
atggagactgggctgcgctggcttctcctggtcgctgtgctcaaaggtgtccagtgtcagtcggtggaggagtccgggggtcgcctggtcacgcctgggacacccctgacactcacctgcacagtctctggaatcgacctcagtagaagtgtgatggcctgggtccgccaggctccagggaaggggctggaatggatcggaatcgttactaatcgtggtaatagatactacgctaactgggcgaaaggccgattcaccatctccaaaacctcgaccacggtgggtctgaagatccccagtccgacaaccgcggacacggccacctatttctgtggcaaaggtgccgtctggggcccaggcaccctggtcaccgtctcctcaggtggaggcggttcaggcggaggtggatctggcggtggcggatcgatggacacgagggcccccactcagctgctggggctcctgctgctctggctcccaggtgccacatttgcccaagtgctgacccagactccatcctccgtgtctgcagctgttggaggcacagtcaccatcaattgccagtccagtcagagttttcttgatgacaacgacttagcctggtttcagcagaaaccagggcggcctcccaagcgcctgatcaaatctgcatcgactctagattctggggtcccatcgcggttcaaaggcagtggatctgggacacagttcactctcaccatcagcgacgtgcagtgtaacgatgctgccacttattactgtctaggcgattatgattgtagtgttggtgattgtagtgctttcggcggagggaccgaagtggtggtcaagggcagcggttctggccggctgaacttcggggacgacatccccagcgctcttcgaatcgcgaagaagaagcgctggaacagcattgaggagcggcgcatccaccaggagagcgagctgcactcctacctctccaggctcattgccgcggagcgtgagagggagctggaagagtgccagcgaaaccacgagggtgatgaggacgacagccacgtccgggcccagcaggcctgcattgaggccaagcacgacaagtacatggcggacatggacgagcttttttctcaggtggatgagaagaggaagaagcgagacatccccgactacctgtgtggcaagatcagctttgagctgatgcgggagccgtgcatcacgcccagtggcatcacctacgaccgcaaggacatcgaggagcacctgcagcgtgtgggtcattttgaccccgtgacccggagccccctgacccaggaacagctcatccccaacttggctatgaaggaggttattgacgcattcatctctgagaatggctgggtggaggattacgtcgacggagcagactacaaggacgatgacgacaagggataa

SEQ ID NO: 174 - protein, 443 aa, Homo sapiens
QSVEESGGRLVTPGTPLTLTCTVSGIDLSRSVMAWVRQAPGKGLEWIGIVTNRGNRYYANWAKGRFTISKTSTTVGLKIPSPTTADTATYFCGKGAVWGPGTLVTVSSGGGGSGGGGSGGGGSMDTRAPTQLLGLLLLWLPGATFAQVLTQTPSSVSAAVGGTVTINCQSSQSFLDDNDLAWFQQKPGRPPKRLIKSASTLDSGVPSRFKGSGSGTQFTLTISDVQCNDAATYYCLGDYDCSVGDCSAFGGGTEVVVKGSGSGRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDYVDGA

SEQ ID NO: 175 - DNA, 1416 bp, Homo sp.
  source 1..1416: mol_type = genomic DNA
atggagactgggctgcgctggcttctcctggtcgcggtgctcaaaggtgtccagtgtcagtcggtggaggagtccgggggtcgcctggtcacgcctgggacacccctgacactcacctgcacagtctctggaatcgacctcagacgcagtgtaatggcctgggtccgccaggctccagggaaggggctggaatggatcggaatcattagtgatcgtggtaacacatactacgcgagctgggcgaaaggccgattcaccatctccagaacctcgaccacggtggctctgaaaatgaccagtccgacaaccgcggacacggccacctatttttgtggcagaggtaatgtctggggcccaggcaccctggtcaccgtctcctcaggtggaggcggttcaggcggaggtggatctggcggtggcggatcgatggacacgagggcccccactcagctgctggggctcctgctgctctggctcccaggtgccacatttgcccaagtgctgacccagactccatcctccgtgtctgcagctgtgggaggcacagtcaccatcaattgccagtccagtcagcggctctatgatgacaacgactgtgcctggtatcagaagaaaccagggcagcctcccaagcgcctgatccatgatgcatccattctagattctggggtctcatcgcggttcagtggcagtggatctgggacacagttcactctcaccatcagcggcgtgcagtgtgaagatgctgccacttactactgtctaggcgattatgattgtagtgttgctgattgtagtactttcggcggagggaccgaggtggtggtcaaaggcagcggttctggccggctgaacttcggggacgacatccccagcgctcttcgaatcgcgaagaagaagcgctggaacagcattgaggagcggcgcatccaccaggagagcgagctgcactcctacctctccaggctcattgccgcggagcgtgagagggagctggaagagtgccagcgaaaccacgagggtgatgaggacgacagccacgtccgggcccagcaggcctgcattgaggccaagcacgacaagtacatggcggacatggacgagcttttttctcaggtggatgagaagaggaagaagcgagacatccccgactacctgtgtggcaagatcagctttgagctgatgcgggagccgtgcatcacgcccagtggcatcacctacgaccgcaaggacatcgaggagcacctgcagcgtgtgggtcattttgaccccgtgacccggagccccctgacccaggaacagctcatccccaacttggctatgaaggaggttattgacgcattcatctctgagaatggctgggtggaggattacgtcgacggagcagactacaaggacgatgacgacaagggataa

SEQ ID NO: 176 - protein, 443 aa, Homo sapiens
QSVEESGGRLVTPGTPLTLTCTVSGIDLRRSVMAWVRQAPGKGLEWIGIISDRGNTYYASWAKGRFTISRTSTTVALKMTSPTTADTATYFCGRGNVWGPGTLVTVSSGGGGSGGGGSGGGGSMDTRAPTQLLGLLLLWLPGATFAQVLTQTPSSVSAAVGGTVTINCQSSQRLYDDNDCAWYQKKPGQPPKRLIHDASILDSGVSSRFSGSGSGTQFTLTISGVQCEDAATYYCLGDYDCSVADCSTFGGGTEVVVKGSGSGRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDYVDGA

SEQ ID NO: 177 - DNA, 1347 bp, Homo sapiens
  source 1..1347: mol_type = genomic DNA
atggccgaggtgcagctggtggagtctgggggaagcctggtcaagcctggggggtccctgagactctcctgtgcagcctctggattcaccttcagtaactatagcatgaactgggtccgccaggctccagggaaggggctggagtggatctcatccattagtggtagtagtagatacatatactacgcagacttcgtgaagggccgattcaccatctccagagacaacgccacgaactcactgtacctgcaaatgaacagcctgagagccgaggacacggctgtttattactgtgtgagatccagtattacgatttttggtggcggtatggacgtctggggcagaggcaccctggtcaccgtctcctcaggtggaggcggttcaggcggaggtggcagcggcggtggcggatcgcagtctgtgctgactcagcctgcctccgtgtctgggtctcctggacagtcgatcaccatctcctgcgctggaaccagcagtgacgttggtggttataactatgtctcctggtaccaacaacacccaggcaaagcccccaaactcatgatttatgaggacagtaagcggccctcaggggtttctaatcgcttctctggctccaagtctggcaacacggcctccctgacaatctctgggctccaggctgaggacgaggctgattattactgcagctcatatacaaccaggagcactcgagttttcggcggagggaccaagctggccgtcctaggtgcggccgcagaattcggcagcggttctggccggctgaacttcggggacgacatccccagcgctcttcgaatcgcgaagaagaagcgctggaacagcattgaggagcggcgcatccaccaggagagcgagctgcactcctacctctccaggctcattgccgcggagcgtgagagggagctggaagagtgccagcgaaaccacgagggtgatgaggacgacagccacgtccgggcccagcaggcctgcattgaggccaagcacgacaagtacatggcggacatggacgagcttttttctcaggtggatgagaagaggaagaagcgagacatccccgactacctgtgtggcaagatcagctttgagctgatgcgggagccgtgcatcacgcccagtggcatcacctacgaccgcaaggacatcgaggagcacctgcagcgtgtgggtcattttgaccccgtgacccggagccccctgacccaggaacagctcatccccaacttggctatgaaggaggttattgacgcattcatctctgagaatggctgggtggaggattacgtcgacggagcagactacaaggacgatgacgacaagggataa

SEQ ID NO: 178 - protein, 448 aa, Homo sapiens
MAEVQLVESGGSLVKPGGSLRLSCAASGFTFSNYSMNWVRQAPGKGLEWISSISGSSRYIYYADFVKGRFTISRDNATNSLYLQMNSLRAEDTAVYYCVRSSITIFGGGMDVWGRGTLVTVSSGGGGSGGGGSGGGGSQSVLTQPASVSGSPGQSITISCAGTSSDVGGYNYVSWYQQHPGKAPKLMIYEDSKRPSGVSNRFSGSKSGNTASLTISGLQAEDEADYYCSSYTTRSTRVFGGGTKLAVLGAAAEFGSGSGRLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDYVDGADYKDDDDKG

SEQ ID NO: 179 - DNA, 1473 bp, Homo sapiens
  source 1..1473: mol_type = genomic DNA
atggagactgggctgcgctggcttctcctggtcgctgtgctcaaaggtgtccagtgtcagtcggtggaggagtccgggggtcgcctggtgtcgcctggaggatccctgacactcacctgccgtgtttctggaagcgaccgcaggacctatgtaatgcgctgggtccgccaggctccggggaaggggctggaatggatcggaaccattagacttgatggtagtagaaactatgcgacctgggcgaaaggccgtttcaccatctccaagtcctcgtcgaccacgatggagctgagaatgaccagtctgacaagcgaggacacggccacttatttctgtgtcagagacgacttggggggcccaggcaccctggtcaccgtctcctcaggtggaggcggttcaggcggaggtggatctggcggtggcggatcgatggacacgagggcccccactcagctgctggggctcctgctgctctggctcccaggtgccagatgtgctgacatcgtgatgacccagactccagcctctgtggaggtagctgtgggaggcacagtcaccatcaagtgccaggccagtcagagcattagcaacctcttagcctggtatcagcagaaaccaggacagcctcccaagctcctgatctacaaggcttccactctggaatctggggtcccatcgcggttcaaaggcagtggatctgggacacagttcgctctcaccatcagcggcgtgcagtgtgacgatgctgccacttactactgtcaacagggtttcgatagtactgatgttgacaatcttttcggcggagggaccgaagtggtggtcgcaggcagcggttctggcctggccgagaggatcatcagcatgctgaactacttcctgcagcacctggtgggccccaagatgggcgccctgaaggtgaaggacttcagcgagttcgacttcaagccccagcagctggtgagcgacatctgcaccatctacctgaacctgggcgacgaggagaacttctgcgccaccgtgcccaaggacggcaggagctacagccccaccctgttcgcccagaccgtgagggtgctgaagaagatcaacaagcccggcaacatgatcatggccttcagcaacctggccgagaggatcaagagcctggccgacctgcagcagcaggaggaggagacctacgccgacgcctgcgacgagttcctggaccccatcatgagcaccctgatgtgcgaccccgtggtgctgcccagcagcagggtgaccgtggacaggagcaccatcgccaggcacctgctgagcgaccagaccgaccccttcaacaggagccccctgaccatggaccagatcaggcccaacaccgagctgaaggagaagatccagaggtggctggccgagaggaagcagcagaaggagcagctggaggactacaaggacgatgacgacaagggacatcatcatcatcatcactaa

SEQ ID NO: 180 - DNA, 1908 bp, Homo sapiens
  source 1..1908: mol_type = genomic DNA
atggactacaaggacgatgacgacaagggacatcatcatcatcatcacgtgatcaacctgtgcctgccccagttcaggcccaggatccactgcaacaagatcagcgccgacggctacgaggtggagaacctgatcagcgaggacctgaccaagaggagccacggcttcaggaccgagtacttcatcaagccccccgtgtacgtgaccgtgagcttccccttcaacgtggagatctgcaggatcaacatcgacctgaccgccggcggcggccagaacgtgaccggcctggagatgtacaccagcgccagcagcagcagggtgagctggaacaccccccagtgcaggaccctgggccccgccgagcccagcgtgcccgacaaggaggccttcaccctggtgggcaaggtgctgctgaagaaccagagccaggtggtgttcagccacaggggcttcaaggccaggccccccttcggcgccatggaggccaccctgcccagccccgccgtggtggcccaggagctgtggaacaagggcgccctgagcctgagccacgtggcccacctgaggatctgcatcacccacgtgaccggcggcggcatcccctgcatcaagaggctggaggtgtggggccagcccgccaagacctgcagccaggaggtgatcgacagcatcctgctggtgaccagcgagaacctgccccaggacgtggccctgcaggcccccgccctgcccatggagagcgactgcgaccccggcgaccagcccgagagccagcaggcccccagcagcctgcagaagctggccgagatcatccaggacgtgcccgaggagttcctggaccccatcaccctggagatcatgccctgccccatgctgctgcccagcggcaaggtgatcgaccagagcaccctggagaagtgcaacaggagcgaggccacctggggcagggtgcccagcgaccccttcaccggcgtggccttcaccccccacagccagcccctgccccaccccagcctgaaggccaggatcgaccacttcctgctgcagcacagcatccccggctgcggcagcggttctggcgagactgggctgcgctggcttctcctggtcgctgtgctcaaaggtgtccagtgtcagtcggtggaggagtccgggggtcgcctggtgtcgcctggaggatccctgacactcacctgccgtgtttctggaagcgaccgcaggacctatgtaatgcgctgggtccgccaggctccggggaaggggctggaatggatcggaaccattagacttgatggtagtagaaactatgcgacctgggcgaaaggccgtttcaccatctccaagtcctcgtcgaccacgatggagctgagaatgaccagtctgacaagcgaggacacggccacttatttctgtgtcagagacgacttggggggcccaggcaccctggtcaccgtctcctcaggtggaggcggttcaggcggaggtggatctggcggtggcggatcgatggacacgagggcccccactcagctgctggggctcctgctgctctggctcccaggtgccagatgtgctgacatcgtgatgacccagactccagcctctgtggaggtagctgtgggaggcacagtcaccatcaagtgccaggccagtcagagcattagcaacctcttagcctggtatcagcagaaaccaggacagcctcccaagctcctgatctacaaggcttccactctggaatctggggtcccatcgcggttcaaaggcagtggatctgggacacagttcgctctcaccatcagcggcgtgcagtgtgacgatgctgccacttactactgtcaacagggtttcgatagtactgatgttgacaatcttttcggcggagggaccgaagtggtggtcgcataa

SEQ ID NO: 181 - DNA, 1971 bp, Homo sapiens
  source 1..1971: mol_type = genomic DNA
atggactacaaggacgatgacgacaagggacatcatcatcatcatcacgagactgggctgcgctggcttctcctggtcgctgtgctcaaaggtgtccagtgtcagtcggtggaggagtccgggggtcgcctggtgtcgcctggaggatccctgacactcacctgccgtgtttctggaagcgaccgcaggacctatgtaatgcgctgggtccgccaggctccggggaaggggctggaatggatcggaaccattagacttgatggtagtagaaactatgcgacctgggcgaaaggccgtttcaccatctccaagtcctcgtcgaccacgatggagctgagaatgaccagtctgacaagcgaggacacggccacttatttctgtgtcagagacgacttggggggcccaggcaccctggtcaccgtctcctcaggtggaggcggttcaggcggaggtggatctggcggtggcggatcgatggacacgagggcccccactcagctgctggggctcctgctgctctggctcccaggtgccagatgtgctgacatcgtgatgacccagactccagcctctgtggaggtagctgtgggaggcacagtcaccatcaagtgccaggccagtcagagcattagcaacctcttagcctggtatcagcagaaaccaggacagcctcccaagctcctgatctacaaggcttccactctggaatctggggtcccatcgcggttcaaaggcagtggatctgggacacagttcgctctcaccatcagcggcgtgcagtgtgacgatgctgccacttactactgtcaacagggtttcgatagtactgatgttgacaatcttttcggcggagggaccgaagtggtggtcgcaggcagcggttctggcaacaggttcgagatgaagctgcacaggaacaacatcttcgaggagagctacaggaggatcatgagcgtgaagaggcccgacgtgctgaaggccaggctgtggatcgagttcgagagcgagaagggcctggactacggcggcgtggccagggagtggttcttcctgctgagcaaggagatgttcaacccctactacggcctgttcgagtacagcgccaccgacaactacaccctgcagatcaaccccaacagcggcctgtgcaacgaggaccacctgagctacttcaccttcatcggcagggtggccggcctggccgtgttccacggcaagctgctggacggcttcttcatcaggcccttctacaagatgatgctgggcaagcagatcaccctgaacgacatggagagcgtggacagcgagtactacaacagcctgaagtggatcctggagaacgaccccaccgagctggacctgatgttctgcatcgacgaggagaacttcggccagacctaccaggtggacctgaagcccaacggcagcgagatcatggtgaccaacgagaacaagagggagtacatcgacctggtgatccagtggaggttcgtgaacagggtgcagaagcagatgaacgccttcctggagggcttcaccgagctgctgcccatcgacctgatcaagatcttcgacgagaacgagctggagctgctgatgtgcggcctgggcgacgtggacgtgaacgactggaggcagcacagcatctacaagaacggctactgccccaaccaccccgtgatccagtggttctggaaggccgtgctgctgatggacgccgagaagaggatcaggctgctgcagttcgtgaccggcaccagcagggtgcccatgaacggcttcgccgagctgtacggcagcaacggcccccagctgttcaccatcgagcagtggggcagccccgagaagctgcccagggcccacacctgcttcaacaggctggacctgcccccctacgagaccttcgaggacctgagggagaagctgctgatggccgtggagaacgcccagggcttcgagggcgtggactaa

SEQ ID NO: 182 - DNA, 1926 bp, Homo sapiens
  source 1..1926: mol_type = genomic DNA
atggactacaaggacgatgacgacaagggacatcatcatcatcatcacgagactgggctgcgctggcttctcctggtcgctgtgctcaaaggtgtccagtgtcagtcggtggaggagtccgggggtcgcctggtgtcgcctggaggatccctgacactcacctgccgtgtttctggaagcgaccgcaggacctatgtaatgcgctgggtccgccaggctccggggaaggggctggaatggatcggaaccattagacttgatggtagtagaaactatgcgacctgggcgaaaggccgtttcaccatctccaagtcctcgtcgaccacgatggagctgagaatgaccagtctgacaagcgaggacacggccacttatttctgtgtcagagacgacttggggggcccaggcaccctggtcaccgtctcctcaggtggaggcggttcaggcggaggtggatctggcggtggcggatcgatggacacgagggcccccactcagctgctggggctcctgctgctctggctcccaggtgccagatgtgctgacatcgtgatgacccagactccagcctctgtggaggtagctgtgggaggcacagtcaccatcaagtgccaggccagtcagagcattagcaacctcttagcctggtatcagcagaaaccaggacagcctcccaagctcctgatctacaaggcttccactctggaatctggggtcccatcgcggttcaaaggcagtggatctgggacacagttcgctctcaccatcagcggcgtgcagtgtgacgatgctgccacttactactgtcaacagggtttcgatagtactgatgttgacaatcttttcggcggagggaccgaagtggtggtcgcaggcagcggttctggcgagaacggcgccgacagcatcctggacctgggcctggtggacagcagcgagaaggtgcagcaggagaacaggaagaggcacggcagcagcaggagcgtggtggacatggacctggacgacaccgacgacggcgacgacaacgcccccctgttctaccagcccggcaagaggggcttctacacccccaggcccggcaagaacaccgaggccaggctgaactgcttcaggaacatcggcaggatcctgggcctgtgcctgctgcagaacgagctgtgccccatcaccctgaacaggcacgtgatcaaggtgctgctgggcaggaaggtgaactggcacgacttcgccttcttcgaccccgtgatgtacgagagcctgaggcagctgatcctggccagccagagcagcgacgccgacgccgtgttcagcgccatggacctggccttcgccatcgacctgtgcaaggaggagggcggcggccaggtggagctgatccccaacggcgtgaacatccccgtgaccccccagaacgtgtacgagtacgtgaggaagtacgccgagcacaggatgctggtggtggccgagcagcccctgcacgccatgaggaagggcctgctggacgtgctgcccaagaacagcctggaggacctgaccgccgaggacttcaggctgctggtgaacggctgcggcgaggtgaacgtgcagatgctgatcagcttcaccagcttcaacgacgagagcggcgagaacgccgagaagctgctgcagttcaagaggtggttctggagcatcgtggagaagatgagcatgaccgagaggcaggacctggtgtacttctggaccagcagccccagcctgcccgccagcgaggagggcttccagcccatgcccagcatcaccatcaggccccccgacgaccagcacctgcccaccgccaacacctgcatcagcaggctgtacgtgcccctgtacagcagcaagcagatcctgaagcagaagctgctgctggccatcaagaccaagaacttcggcttcgtgtaa

SEQ ID NO: 183 - DNA, 1671 bp, Homo sapiens
  source 1..1671: mol_type = genomic DNA
atggactacaaggacgatgacgacaagggacatcatcatcatcatcacgaccccgccgaggccgtgctgcaggagaaggccctgaagttcatgtgcagcatgcccaggagcctgtggctgggctgcagcagcctggccgacagcatgcccagcctgaggtgcctgtacaaccccggcaccggcgccctgaccgccttccagaacagcagcgagagggaggactgcaacaacggcgagccccccaggaagatcatccccgagaagaacagcctgaggcagacctacaacagctgcgccaggctgtgcctgaaccaggagaccgtgtgcctggccagcaccgccatgaagaccgagaactgcgtggccaagaccaagctggccaacggcaccagcagcatgatcgtgcccaagcagaggaagctgagcgccagctacgagaaggagaaggagctgtgcgtgaagtacttcgagcagtggagcgagagcgaccaggtggagttcgtggagcacctgatcagccagatgtgccactaccagcacggccacatcaacagctacctgaagcccatgctgcagagggacttcatcaccgccctgcccgccaggggcctggaccacatcgccgagaacatcctgagctacctggacgccaagagcctgtgcgccgccgagctggtgtgcaaggagtggtacagggtgaccagcgacggcatgctgtggaagaagctgatcgagaggatggtgaggaccgacagcctgtggaggggcctggccgagaggaggggctggggccagtacctgttcaagaacaagccccccgacggcaacggcagcggttctggcgagactgggctgcgctggcttctcctggtcgctgtgctcaaaggtgtccagtgtcagtcggtggaggagtccgggggtcgcctggtgtcgcctggaggatccctgacactcacctgccgtgtttctggaagcgaccgcaggacctatgtaatgcgctgggtccgccaggctccggggaaggggctggaatggatcggaaccattagacttgatggtagtagaaactatgcgacctgggcgaaaggccgtttcaccatctccaagtcctcgtcgaccacgatggagctgagaatgaccagtctgacaagcgaggacacggccacttatttctgtgtcagagacgacttggggggcccaggcaccctggtcaccgtctcctcaggtggaggcggttcaggcggaggtggatctggcggtggcggatcgatggacacgagggcccccactcagctgctggggctcctgctgctctggctcccaggtgccagatgtgctgacatcgtgatgacccagactccagcctctgtggaggtagctgtgggaggcacagtcaccatcaagtgccaggccagtcagagcattagcaacctcttagcctggtatcagcagaaaccaggacagcctcccaagctcctgatctacaaggcttccactctggaatctggggtcccatcgcggttcaaaggcagtggatctgggacacagttcgctctcaccatcagcggcgtgcagtgtgacgatgctgccacttactactgtcaacagggtttcgatagtactgatgttgacaatcttttcggcggagggaccgaagtggtggtcgcataa

SEQ ID NO: 184 - DNA, 1629 bp, Homo sapiens
  source 1..1629: mol_type = genomic DNA
atggagactgggctgcgctggcttctcctggtcgctgtgctcaaaggtgtccagtgtcagtcggtggaggagtccgggggtcgcctggtgtcgcctggaggatccctgacactcacctgccgtgtttctggaagcgaccgcaggacctatgtaatgcgctgggtccgccaggctccggggaaggggctggaatggatcggaaccattagacttgatggtagtagaaactatgcgacctgggcgaaaggccgtttcaccatctccaagtcctcgtcgaccacgatggagctgagaatgaccagtctgacaagcgaggacacggccacttatttctgtgtcagagacgacttggggggcccaggcaccctggtcaccgtctcctcaggtggaggcggttcaggcggaggtggatctggcggtggcggatcgatggacacgagggcccccactcagctgctggggctcctgctgctctggctcccaggtgccagatgtgctgacatcgtgatgacccagactccagcctctgtggaggtagctgtgggaggcacagtcaccatcaagtgccaggccagtcagagcattagcaacctcttagcctggtatcagcagaaaccaggacagcctcccaagctcctgatctacaaggcttccactctggaatctggggtcccatcgcggttcaaaggcagtggatctgggacacagttcgctctcaccatcagcggcgtgcagtgtgacgatgctgccacttactactgtcaacagggtttcgatagtactgatgttgacaatcttttcggcggagggaccgaagtggtggtcgcaggcagcggttctggcaaggagaccagcgtggccctgcacctgatcgccaccaacagcaggaacatcacctgcatcacctgcaccgacgtgaggagccccgtgctggtgttccagtgcaacagcaggcacgtgatctgcctggactgcttccacctgtactgcgtgaccaggctgaacgacaggcagttcgtgcacgacccccagctgggctacagcctgccctgcgtggccggctgccccaacagcctgatcaaggagctgcaccacttcaggatcctgggcgaggagcagtacaacaggtaccagcagtacggcgccgaggagtgcgtgctgcagatgggcggcgtgctgtgccccaggcccggctgcggcgccggcctgctgcccgagcccgaccagaggaaggtgacctgcgagggcggcaacggcctgggctgcggcttcgccttctgcagggagtgcaaggaggcctaccacgagggcgagtgcagcgccgtgttcgaggccagcggcaccaccacccaggcctacagggtggacgagagggccgccgagcaggccaggtgggaggccgccagcaaggagaccatcaagaagaccaccaagccctgccccaggtgccacgtgcccgtggagaagaacggcggctgcatgcacatgaagtgcccccagccccagtgcaggctggagtggtgctggaactgcggctgcgagtggaacagggtgtgcatgggcgaccactggttcgacgtggactacaaggacgatgacgacaagggacatcatcatcatcatcactaa

SEQ ID NO: 185 - DNA, 1236 bp, Homo sapiens
  source 1..1236: mol_type = genomic DNA
atggagactgggctgcgctggcttctcctggtcgctgtgctcaaaggtgtccagtgtcagtcggtggaggagtccgggggtcgcctggtgtcgcctggaggatccctgacactcacctgccgtgtttctggaagcgaccgcaggacctatgtaatgcgctgggtccgccaggctccggggaaggggctggaatggatcggaaccattagacttgatggtagtagaaactatgcgacctgggcgaaaggccgtttcaccatctccaagtcctcgtcgaccacgatggagctgagaatgaccagtctgacaagcgaggacacggccacttatttctgtgtcagagacgacttggggggcccaggcaccctggtcaccgtctcctcaggtggaggcggttcaggcggaggtggatctggcggtggcggatcgatggacacgagggcccccactcagctgctggggctcctgctgctctggctcccaggtgccagatgtgctgacatcgtgatgacccagactccagcctctgtggaggtagctgtgggaggcacagtcaccatcaagtgccaggccagtcagagcattagcaacctcttagcctggtatcagcagaaaccaggacagcctcccaagctcctgatctacaaggcttccactctggaatctggggtcccatcgcggttcaaaggcagtggatctgggacacagttcgctctcaccatcagcggcgtgcagtgtgacgatgctgccacttactactgtcaacagggtttcgatagtactgatgttgacaatcttttcggcggagggaccgaagtggtggtcgcaggcagcggttctggccccaggaggaacgccaggaggctgccccaggaccacgccgacagctgcgtggtgagcagcgacgacgaggagctgagcagggacagggacgtgtacgtgaccacccacacccccaggaacgccagggacgagggcgccaccggcctgaggcccagcggcaccgtgagctgccccatctgcatggacggctacagcgagatcgtgcagaacggcaggctgatcgtgagcaccgagtgcggccacgtgttctgcagccagtgcctgagggacagcctgaagaacgccaacacctgccccacctgcaggaagaagatcaaccacaagaggtaccaccccatctacatcgactacaaggacgatgacgacaagggacatcatcatcatcatcactaa

SEQ ID NO: 186 - protein, 75 aa, Homo sapiens
DIPDYLCGKISFELMREPCITPSGITYDRKDIEEHLQRVGHFDPVTRSPLTQEQLIPNLAMKEVIDAFISENGWV

SEQ ID NO: 187 - protein, 74 aa, Homo sapiens
ACDEFLDPIMSTLMCDPVVLPSSRVTVDRSTIARHLLSDQTDPFNRSPLTMDQIRPNTELKEKIQRWLAERKQQ

SEQ ID NO: 188 - protein, 81 aa, Homo sapiens
DVPEEFLDPITLEIMPCPMLLPSGKVIDQSTLEKCNRSEATWGRVPSDPFTGVAFTPHSQPLPHPSLKARIDHFLLQHSIP

SEQ ID NO: 189 - protein, 335 aa, Homo sapiens
RPDVLKARLWIEFESEKGLDYGGVAREWFFLLSKEMFNPYYGLFEYSATDNYTLQINPNSGLCNEDHLSYFTFIGRVAGLAVFHGKLLDGFFIRPFYKMMLGKQITLNDMESVDSEYYNSLKWILENDPTELDLMFCIDEENFGQTYQVDLKPNGSEIMVTNENKREYIDLVIQWRFVNRVQKQMNAFLEGFTELLPIDLIKIFDENELELLMCGLGDVDVNDWRQHSIYKNGYCPNHPVIQWFWKAVLLMDAEKRIRLLQFVTGTSRVPMNGFAELYGSNGPQLFTIEQWGSPEKLPRAHTCFNRLDLPPYETFEDLREKLLMAVENAQGFEGV

SEQ ID NO: 190 - protein, 338 aa, Homo sapiens
LDLGLVDSSEKVQQENRKRHGSSRSVVDMDLDDTDDGDDNAPLFYQPGKRGFYTPRPGKNTEARLNCFRNIGRILGLCLLQNELCPITLNRHVIKVLLGRKVNWHDFAFFDPVMYESLRQLILASQSSDADAVFSAMDLAFAIDLCKEEGGGQVELIPNGVNIPVTPQNVYEYVRKYAEHRMLVVAEQPLHAMRKGLLDVLPKNSLEDLTAEDFRLLVNGCGEVNVQMLISFTSFNDESGENAEKLLQFKRWFWSIVEKMSMTERQDLVYFWTSSPSLPASEEGFQPMPSITIRPPDDQHLPTANTCISRLYVPLYSSKQILKQKLLLAIKTKNFGFV

SEQ ID NO: 191 - protein, 39 aa, Homo sapiens
DHIAENILSYLDAKSLCAAELVCKEWYRVTSDGMLWKKL

SEQ ID NO: 192 - protein, 212 aa, Homo sapiens
CITCTDVRSPVLVFQCNSRHVICLDCFHLYCVTRLNDRQFVHDPQLGYSLPCVAGCPNSLIKELHHFRILGEEQYNRYQQYGAEECVLQMGGVLCPRPGCGAGLLPEPDQRKVTCEGGNGLGCGFAFCRECKEAYHEGECSAVFEASGTTTQAYRVDERAAEQARWEAASKETIKKTTKPCPRCHVPVEKNGGCMHMKCPQPQCRLEWCWNC

SEQ ID NO: 193 - protein, 45 aa, Homo sapiens
PICMDGYSEIVQNGRLIVSTECGHVFCSQCLRDSLKNANTCPTCR

SEQ ID NO: 194 - protein, 13 aa, Homo sapiens
TKTGNAGSRLACG

SEQ ID NO: 195 - protein, 13 aa, Homo sapiens
NAGSRLAZGVIGI

SEQ ID NO: 196 - protein, 140 aa, Mus musculus
MYLGLNCVFIVFLLKGVQSEVKLEESGGGLVQPGGSMKLSCVASGSTFSNYWMNWVRQSPEKGLEWVAEIRLKSNTYAIHYAESVKGRFTISRDNSKSSVYLQMNNLRAEDTGIYYCTGNAMDFWGQGTSVTVSSAKTTA

SEQ ID NO: 197 - DNA, 420 bp, Mus musculus
  source 1..420: mol_type = genomic DNA
atgtacttgggactgaactgtgtattcatagtttttctcttaaaaggtgtccagagtgaagtgaagcttgaggagtctggaggaggcttggtgcaacctggaggatccatgaaactctcctgtgttgcctctggatccactttcagtaactactggatgaactgggtccgccagtctccagagaaggggcttgagtgggttgctgaaattagattgaaatctaatacttatgcaatacattatgcggagtctgtgaaagggaggttcaccatctcaagagataattccaaaagtagtgtctacctgcaaatgaacaacttaagagctgaagacactggcatttattactgtaccggcaatgctatggacttctggggtcaaggaacctcagtcaccgtctcctcagccaaaacaacagcc

SEQ ID NO: 198 - protein, 142 aa, Mus musculus
MYLGLNCVFIVFLLKGVQSEVKLEESGGGLVQPGGSMKLSCVASGSTFSNYWMNWVRQSPEKGLEWVAEIRLKSNTYAIHYAESVKGRFTISRDNSKSSVYLQMNNLRAEDTGIYYCTGNAMDFWGQGTSVTVSSAKTTAPS

SEQ ID NO: 199 - DNA, 426 bp, Mus musculus
  source 1..426: mol_type = genomic DNA
atgtacttgggactgaactgtgtattcatagtttttctcttaaaaggtgtccagagtgaagtgaagcttgaggagtctggaggaggcttggtgcaacctggaggatccatgaaactctcctgtgttgcctctggatccactttcagtaactactggatgaactgggtccgccagtctccagagaaggggcttgagtgggttgctgaaattagattgaaatctaatacttatgcaatacattatgcggagtctgtgaaagggaggttcaccatctcaagagataattccaaaagtagtgtctacctgcaaatgaacaacttaagagctgaagacactggcatttattactgtaccggcaatgctatggacttctggggtcaaggaacctcagtcaccgtctcctcagccaaaacaacagccccatcg

SEQ ID NO: 200 - protein, 165 aa, Mus musculus
MYLGLNCVFIVFLLKGVQSEVKLEESGGGLVQPGGSMKLSCVASGSTFSNYWMNWVRQSPEKGLEWVAEIRLKSNTYAIHYAESVKGRFTISRDNSKSSVYLQMNNLRAEDTGIYYCTGNAMDFWGQGTSVTVSSAKTTAPSVYPLAPVCGDTTGSSVTLKGEFV

SEQ ID NO: 201 - DNA, 495 bp, Mus musculus
  source 1..495: mol_type = genomic DNA
atgtacttgggactgaactgtgtattcatagtttttctcttaaaaggtgtccagagtgaagtgaagcttgaggagtctggaggaggcttggtgcaacctggaggatccatgaaactctcctgtgttgcctctggatccacttttagtaactactggatgaactgggtccgccagtctccagagaaggggcttgagtgggttgctgaaattagattgaaatctaatacttatgcaatacattatgcggagtctgtgaaagggaggttcaccatctcaagagataattccaaaagtagtgtctacctgcaaatgaacaacttaagagctgaagacactggcatttattactgtaccggcaatgctatggacttctggggtcaaggaacctcagtcaccgtctcctcagccaaaacaacagccccatcggtctatccactggcccctgtgtgtggagatacaactggctcctcggtgactctaaagggcgaattcgtt

SEQ ID NO: 202 - protein, 243 aa, Mus musculus
MYLGLNCVFIVFLLKGVQSEVKLEESGGGLVQPGGSMKLSCVASGSTFSNYWMNWVRQSPEKGLEWVAEIRLKSNTYAIHYAESVKGRFTISRDNSKSSVYLQMNNLRAEDTGIYYCTGNAMDFWGQGTSVTVSSAKTTAPSVYPLAPVCGDTTGSSVTLGCLVKGYFPEPVTLTWNSGSLSSGVHTFPAVLQSDLYTLSSSVTVTSSTWPSQSITCNVAHPASSTKVDKKIEQGRIRLNLQD

SEQ ID NO: 203 - DNA, 729 bp, Mus musculus
  source 1..729: mol_type = genomic DNA
atgtacttgggactgaactgtgtattcatagtttttctcttaaaaggtgtccagagtgaagtgaagcttgaggagtctggaggaggcttggtgcaacctggaggatccatgaaactctcctgtgttgcctctggatccactttcagtaactactggatgaactgggtccgccagtctccagagaaggggcttgagtgggttgctgaaattagattgaaatctaatacttatgcaatacattatgcggagtctgtgaaagggaggttcaccatctcaagagataattccaaaagtagtgtctacctgcaaatgaacaacttaagagctgaagacactggcatttattactgtaccggcaatgctatggacttctggggtcaaggaacctcagtcaccgtctcctcagccaaaacaacagccccatcggtctatccactggcccctgtgtgtggagatacaactggctcctcggtgactctaggatgcctggtcaagggttatttccctgagccagtgaccttgacctggaactctggatccctgtccagtggtgtgcacaccttcccagctgtcctgcagtctgacctctacaccctcagcagctcagtgactgtaacctcgagcacctggcccagccagtccatcacctgcaatgtggcccacccggcaagcagcaccaaggtggacaagaaaattgagcaagggcgaattcgtttaaacctgcaggac

SEQ ID NO: 204 - protein, 141 aa, Mus musculus
MYLGLNCVFIVFLLKGVQSEVKLEESGGGLVQPGGSMKLSCVASGSTFSNYWMNWVRQSPEKGLEWVAEIRLKSNTYAIHYAESVKGRFTISRDNSKSSVYLQMNNLRAEDTGIYYCTGNAMDFWGQGTSVTVSSAKTTAP

SEQ ID NO: 205 - DNA, 423 bp, Mus musculus
  source 1..423: mol_type = genomic DNA
atgtacttgggactgaactgtgtattcatagtttttctcttaaaaggtgtccagagtgaagtgaagcttgaggagtctggaggaggcttggtgcaacctggaggatccatgaaactctcctgtgttgcctctggatccactttcagtaactactggatgaactgggtccgccagtctccagagaaggggcttgagtgggttgctgaaattagattgaaatctaatacttatgcaatacattatgcggagtctgtgaaagggaggttcaccatctcaagagataattccaaaagtagtgtctacctgcaaatgaacaacttaagagctgaagacactggcatttattactgtaccggcaatgctatggacttctggggtcaaggaacctcagtcaccgtctcctcagccaaaacaacagcccca

SEQ ID NO: 206 - protein, 143 aa, Mus musculus
MYLGLNCVFIVFLLKGVQSEVKLEESGGGLVQPGGSMKLSCVASGSTFSNYWMNWVRQSPEKGLEWVAEIRLKSNTYAIHYAESVKGRFTISRDNSKSSVYLQMNNLRAEDTGIYYCTGNAMDFWGQGTSVTVSSAKTTAPSV

SEQ ID NO: 207 - DNA, 429 bp, Mus musculus
  source 1..429: mol_type = genomic DNA
atgtacttgggactgaactgtgtattcatagtttttctcttaaaaggtgtccagagtgaagtgaagcttgaggagtctggaggaggcttggtgcaacctggaggatccatgaaactctcctgtgttgcctctggatccactttcagtaactactggatgaactgggtccgccagtctccagagaaggggcttgagtgggttgctgaaattagattgaaatctaatacttatgcaatacattatgcggagtctgtgaaagggaggttcaccatctcaagagataattccaaaagtagtgtctacctgcaaatgaacaacttaagagctgaagacactggcatttattactgtaccggcaatgctatggacttctggggtcaaggaacctcagtcaccgtctcctcagccaaaacaacagccccatcggtc

SEQ ID NO: 208 - DNA, 363 bp, Mus musculus
  source 1..363: mol_type = genomic DNA
atgtacttgggactgaactgtgtattcatagtttttctcttaaaaggtgtccagagtgaagtgaagcttgaggagtctggaggaggcaactactggatgaactgggtccgccagtctccagagaaggggcttgagtgggttgctgaaattagattgaaatctaatacttatgcaatacattatgcggagtctgtgaaagggaggttcaccatctcaagagataattccaaaagtagtgtctacctgcaaatgaacaacttaagagctgaagacactggcatttattactgtaccggcaatgctatggacttctggggtcaaggaacctcagtcaccgtctcctcagccaaaacaacagcccca

SEQ ID NO: 209 - protein, 138 aa, Mus musculus
MKLPVRLLVLMFWIPASSSDVVMTQTPLSLPVSLGDQASISCRSSQSLVYSNGNTYLHWYLQKPGQSPKLLIYKVSNRFSGVPDRFSGSGSGTDFTLRISRVEAEDLGVYFCSQSTHVPPWTFGGGTKLEIKRADAAP

SEQ ID NO: 210 - DNA, 414 bp, Mus musculus
  source 1..414: mol_type = genomic DNA
atgaagttgcctgttaggctgttggtgctgatgttctggattcctgcttccagcagtgatgttgtgatgacccaaactccactctccctgcctgtcagtcttggagatcaagcctccatctcttgcagatctagtcagagccttgtatatagtaatggaaacacctatttacattggtacctgcagaagccaggccagtctccaaagctcctgatctacaaagtttccaaccgattttctggggtcccagacaggttcagtggcagtggatcagggacagatttcacactcaggatcagcagagtggaggctgaggatctgggagtttatttctgctctcaaagtacacatgttcctccgtggacgttcggtggaggcaccaagctggaaatcaaacgggctgatgctgcacca

SEQ ID NO: 211 - protein, 140 aa, Mus musculus
MKLPVRLLVLMFWIPASSSDVVMTQTPLSLPVSLGDQASISCRSSQSLVYSNGNTYLHWYLQKPGQSPKLLIYKVSNRFSGVPDRFSGSGSGTDFTLRISRVEAEDLGVYFCSQSTHVPPWTFGGGTKLEIKRADAAPTR

SEQ ID NO: 212 - DNA, 1334 bp, Mus musculus
  source 1..1334: mol_type = genomic DNA
nnnnnnnnnnnnnnnnnnnnnnnctnnnaattaaccctcncctaaagggactagtcctgcaggtttaaacgaattcgcccttctactggagctcgaggacactgacatggactgaaggagtagaaactttctaancnggnnatccgtcagggtgaagccatgttcagttcgagcgcccagatnntgaagcccaatggctagaagccggatgagttctagtctggcatctctcaggcgctgctcgagctgnaantgaactccgatctcaaggcgcagctnntngaacttaacctcnnngcacccaaggaaatcaaantnagtngngntcccaaacctatcatactttttgtactagntcctcagcttatatctttccagaaaatccaagtccggctagttcgtgaatnntngaaaaagttcagtggcaaccacgnnntcttcattntncataggaggatnnngccaataccnncnnnnaaaagccgtannaaaantanncagaatgacccnaaaanccgtaccctaacagcagtgaacgacgctgtccttgaggacttagtcttcgcnagtgaaattatggataagaggatccgtgtgaaactgantggcagacggctcataaaagttcatttatacaaagctnngcanaagcacgtgaaanncactgctgaaactttttctgttgtgtacaacagcnnncaacannaatgagtgttaaangggtganntcacgntcgctaatttcaattcgncctanaatgagnnnnnttacaattcaatgnngnctccttttacatcgttcattgactggnnantnnnntgntcgttacccnnactnactacgtctttgtagcactatccccnnttnnngccctnntggnngttnntactcnncnacgccntaaactnnatnncccctnnnccatcnnttcctccaataccnnnnnaatccggtnngtcnnnnagtcttaccanntctacntagcannnnnncnnnnnncnnncnnnnttngtagnncgtaccacnntnganntctactnnncnnccccntgcccnnanngcanngnnnanntccccntcnnnncnnangcnnnnnccnnnancgnnacaannnnnnnncnncnnnnnnnnnnncnntccnncnacggnncnnnnnnnntncnnnntnnnnnnnnnncggnnnnannnggnnactcnnncnnannnnntgnnccacnnnnnnnncggtntnccnngctattctccnnncnnnccnaatnnccntgganttncnnnncnnnnnnnnncnnnnnnatntncnaccannnatnnnanncnnttattnntnnnnnnn

SEQ ID NO: 213 - protein, 139 aa, Mus musculus
MKLPVRLLVLMFWIPASSSDVVMTQTPLSLPVSLGDQASISCRSSQSLVYSNGNTYLHWYLQKPGQSPKLLIYKVSNRFSGVPDRFSGSGSGTDFTLRISRVEAEDLGVYFCSQSTHVPPWTFGGGTKLEIKRADAAPT

SEQ ID NO: 214 - DNA, 1264 bp, Mus musculus
  source 1..1264: mol_type = genomic DNA
nnnnnnnnnnnnnnncgccagctcaganttaaccctcactaaagggactagtcctgcaggtttaaacgaattcgcccttctaacactcattcctgttgaagctcttgacaatgggtgaagttgatgtcttgtgagtggcctcacaggtatagctgttatgtcgttcatactcgtccttggtcaacgtgagggtgctgctcatgctgtaggtgctgtctttgctgtcctgatcagtccaactgttcaggacgccattttgtcgttcactgccatcaatcttccacttgacattgatgtctttggggtagaagttgttcaagaagcacacgactgaggcacctccagatgttaactgctcactggatggtgggaagatggatacagttggtgcagcatcagcccgttttatttccagcttggtcccccctccgaacgtgtaagctccctaatgtgctgacagtaataggttgcagcatcctcctcctccacaggatggatgttgagggtgaagtctgtcccagacccactgccactgaacctggcagggaccccagattctaggttggatacaagatagatgaggagtctgggtggctgtcctggtttctgttggttccagtgcatataactatagccagatgtactgacacttttgctggccctgtatgagatggtggccctctgccccagagatacagctaaggaagcaggagactgtgtcagcacaatgtcaccagtggaacctggaacccagagcagcagtacccataacaggagtgtgtctgtctccatctctgagagctggaagagaggattttctactcncttcagtccatgtcagtngtcctcgtgctccagtcgaagggcgaattcgcggccgctaaattcaattcgccctatagtgnagtcgtattacaattcactgggccgtcgtttttacaacgtcgtgactgggaaaaaccctgggcgttacccaacttaaatcgcccttgcagcacatcccccttttcgccagctggcgtaannancgaaaaaggccccgnncnantcgcccnttcccaacagttgnngcnnccctataacgtacggncagtttnaaggtttacaccntataaaannanaaangccgttaatcgtcnggtttgtggaatgtacagaagtgaanntaatnnacnngccnggnccnaacggaaggnnnatcccccctgggccagngncacgttctggctnttcnnanaaangttcnnccnnnggaantttnacn

SEQ ID NO: 215 - protein, 140 aa, Mus musculus
MKLPVRLLVLMFWIPASSSDVVMTQTPLSLPVSLGDQASISCRSSQSLVYSNGNTYLHWYLQKPGQSPKLLIYKVSNRFSGVPDRFSGSGSGTDFTLRISRVEAEDLGVYFCSQSTHVPPWTFGGGTKLEIKRADAAPTV

SEQ ID NO: 216 - DNA, 420 bp, Mus musculus
  source 1..420: mol_type = genomic DNA
atgaagttgcctgttaggctgttggtgctgatgttctggattcctgcttccagcagtgatgttgtgatgacccaaactccactctccctgcctgtcagtcttggagatcaagcctccatctcttgcagatctagtcagagccttgtatatagtaatggaaacacctatttacattggtacctgcagaagccaggccagtctccaaagctcctgatctacaaagtttccaaccgattttctggggtcccagacaggttcagtggcagtggatcagggacagatttcacactcaggatcagcagagtggaggctgaggatctgggagtttatttctgctctcaaagtacacatgttcctccgtggacgttcggtggaggcaccaagctggaaatcaaacgggctgatgctgcaccaactgta

SEQ ID NO: 217 - DNA, 20 bp, synthetic construct
  source 1..20: mol_type = other DNA
agtcaccatcaagtgccagg

SEQ ID NO: 218 - DNA, 20 bp, synthetic construct
  source 1..20: mol_type = other DNA
gctttggtgctcccactact

SEQ ID NO: 219 - DNA, 20 bp, synthetic construct
  source 1..20: mol_type = other DNA
attacgtcgacggagcagac

SEQ ID NO: 220 - DNA, 20 bp, synthetic construct
  source 1..20: mol_type = other DNA
gttaggtcgcctggaaggaa

SEQ ID NO: 221 - protein, 15 aa, synthetic construct
GGGGSGGGGSGGGGS

SEQ ID NO: 222 - protein, 5 aa, synthetic construct
GSGSG

SEQ ID NO: 223 - protein, 180 aa, synthetic construct
RLNFGDDIPSALRIAKKKRWNSIEERRIHQESELHSYLSRLIAAERERELEECQRNHEGDEDDSHVRAQQACIEAKHDKYMADMDELFSQVDEKRKKRDIPDYLCGKISFELMREPCITPSGITYDRKDIEEHLQRVGHFDPVTRSPLTQEQLIPNLAMKEVIDAFISENGWVEDYVDGA

SEQ ID NO: 224 - protein, 19 aa, Oryctolagus cuniculus
METGLRWLLLVAVLKGVQC

SEQ ID NO: 225 - protein, 23 aa, Oryctolagus cuniculus
MDTRAPTQLLGLLLLWLPGATFA

SEQ ID NO: 226 - protein, 22 aa, Oryctolagus cuniculus
MDTRAPTQLLGLLLLWLPGARC